SEQUENCE LISTING (38 sequences)

SEQ ID NO: 1 - protein, 15 aa, Mus musculus
RASESVTILGSHLIH

SEQ ID NO: 2 - protein, 7 aa, mus musculus
LASNVQT

SEQ ID NO: 3 - protein, 9 aa, mus musculus
LQSRTIPRT

SEQ ID NO: 4 - protein, 5 aa, Mus musculus
DYSIN

SEQ ID NO: 5 - protein, 17 aa, Mus musculus
WINTETREPAYAYDFRG

SEQ ID NO: 6 - protein, 8 aa, Mus musculus
DYSYAMDY

SEQ ID NO: 7 - protein, 111 aa, Mus musculus
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIK

SEQ ID NO: 8 - protein, 117 aa, Mus musculus
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 9 - protein, 493 aa, Artificial Sequence
  anti-BCMA02 CAR
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - DNA, 1485 bp, Artificial Sequence
  anti-BCMA02 CAR
atggcactccccgtcaccgcccttctcttgcccctcgccctgctgctgcatgctgccaggcccgacattgtgctcactcagtcacctcccagcctggccatgagcctgggaaaaagggccaccatctcctgtagagccagtgagtccgtcacaatcttggggagccatcttattcactggtatcagcagaagcccgggcagcctccaacccttcttattcagctcgcgtcaaacgtccagacgggtgtacctgccagattttctggtagcgggtcccgcactgattttacactgaccatagatccagtggaagaagacgatgtggccgtgtattattgtctgcagagcagaacgattcctcgcacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcaccgactacagcataaattgggtgaaacgggcccctggaaagggcctcaaatggatgggttggatcaataccgaaactagggagcctgcttatgcatatgacttccgcgggagattcgccttttcactcgagacatctgcctctactgcttacctccaaataaacaacctcaagtatgaagatacagccacttacttttgcgccctcgactatagttacgccatggactactggggacagggaacctccgttaccgtcagttccgcggccgcaaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgctaatga

SEQ ID NO: 11 - protein, 184 aa, Homo sapiens
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 12 - protein, 5 aa, Artificial Sequence
  Flexible peptide linker
DGGGS

SEQ ID NO: 13 - protein, 5 aa, Artificial Sequence
  Flexible peptide linker
TGEKP

SEQ ID NO: 14 - protein, 4 aa, Artificial Sequence
  Flexible peptide linker
GGRR

SEQ ID NO: 15 - protein, 5 aa, Artificial Sequence
  Flexible peptide linker
GGGGS

SEQ ID NO: 16 - protein, 14 aa, Artificial Sequence
  Flexible peptide linker
EGKSSGSGSESKVD

SEQ ID NO: 17 - protein, 18 aa, Artificial Sequence
  Flexible peptide linker
KESGSVSSEQLAQFRSLD

SEQ ID NO: 18 - protein, 8 aa, Artificial Sequence
  Flexible peptide linker
GGRRGGGS

SEQ ID NO: 19 - protein, 9 aa, Artificial Sequence
  Flexible peptide linker
LRQRDGERP

SEQ ID NO: 20 - protein, 12 aa, Artificial Sequence
  Flexible peptide linker
LRQKDGGGSERP

SEQ ID NO: 21 - protein, 16 aa, Artificial Sequence
  Flexible peptide linker
LRQKDGGGSGGGSERP

SEQ ID NO: 22 - protein, 18 aa, Artificial Sequence
  Flexible peptide linker
GSTSGSGKPGSGEGSTKG

SEQ ID NO: 23 - protein, 7 aa, Artificial Sequence
  protease cleavage site
  MISC_FEATURE (2)..(3): Xaa = Any amino acid
  MISC_FEATURE (5)..(5): Xaa = Any amino acid
  MISC_FEATURE (7)..(7): Xaa  is Gly or Ser
EXXYXQX

SEQ ID NO: 24 - protein, 7 aa, Artificial Sequence
  protease cleavage site
ENLYFQG

SEQ ID NO: 25 - protein, 7 aa, Artificial Sequence
  protease cleavage site
ENLYFQS

SEQ ID NO: 26 - protein, 19 aa, Artificial Sequence
  protease cleavage site
LLNFDLLKLAGDVESNPGP

SEQ ID NO: 27 - protein, 19 aa, Artificial Sequence
  protease cleavage site
TLNFDLLKLAGDVESNPGP

SEQ ID NO: 28 - protein, 14 aa, Artificial Sequence
  protease cleavage site
LLKLAGDVESNPGP

SEQ ID NO: 29 - protein, 17 aa, Artificial Sequence
  protease cleavage site
NFDLLKLAGDVESNPGP

SEQ ID NO: 30 - protein, 20 aa, Artificial Sequence
  protease cleavage site
QLLNFDLLKLAGDVESNPGP

SEQ ID NO: 31 - protein, 24 aa, Artificial Sequence
  protease cleavage site
APVKQTLNFDLLKLAGDVESNPGP

SEQ ID NO: 32 - protein, 40 aa, Artificial Sequence
  protease cleavage site
VTELLYRMKRAETYCPRPLLAIHPTEARHKQKIVAPVKQT

SEQ ID NO: 33 - protein, 18 aa, Artificial Sequence
  protease cleavage site
LNFDLLKLAGDVESNPGP

SEQ ID NO: 34 - protein, 40 aa, Artificial Sequence
  protease cleavage site
LLAIHPTEARHKQKIVAPVKQTLNFDLLKLAGDVESNPGP

SEQ ID NO: 35 - protein, 33 aa, Artificial Sequence
  protease cleavage site
EARHKQKIVAPVKQTLNFDLLKLAGDVESNPGP

SEQ ID NO: 36 - DNA, 7350 bp, Artificial Sequence
  anti-BCMA02 CAR vector
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatcatatgccagcctatggtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgggtctctctggttagaccagatctgagcctgggagctctctggctaactagggaacccactgcttaagcctcaataaagcttgccttgagtgctcaaagtagtgtgtgcccgtctgttgtgtgactctggtaactagagatccctcagacccttttagtcagtgtggaaaatctctagcagtggcgcccgaacagggacttgaaagcgaaagtaaagccagaggagatctctcgacgcaggactcggcttgctgaagcgcgcacggcaagaggcgaggggcggcgactggtgagtacgccaaaaattttgactagcggaggctagaaggagagagtagggtgcgagagcgtcggtattaagcgggggagaattagataaatgggaaaaaattcggttaaggccagggggaaagaaacaatataaactaaaacatatagttagggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcagcagctgacacaggaaacaacagccaggtcagccaaaattaccctatagtgcagaacctccaggggcaaatggtacatcaggccatatcacctagaactttaaattaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaacacatggaaaagattagtaaaacaccatagctctagagcgatcccgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccatctcgacggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgattcacctgacgcgtctacgccaccatggcactccccgtcaccgcccttctcttgcccctcgccctgctgctgcatgctgccaggcccgacattgtgctcactcagtcacctcccagcctggccatgagcctgggaaaaagggccaccatctcctgtagagccagtgagtccgtcacaatcttggggagccatcttattcactggtatcagcagaagcccgggcagcctccaacccttcttattcagctcgcgtcaaacgtccagacgggtgtacctgccagattttctggtagcgggtcccgcactgattttacactgaccatagatccagtggaagaagacgatgtggccgtgtattattgtctgcagagcagaacgattcctcgcacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcaccgactacagcataaattgggtgaaacgggcccctggaaagggcctcaaatggatgggttggatcaataccgaaactagggagcctgcttatgcatatgacttccgcgggagattcgccttttcactcgagacatctgcctctactgcttacctccaaataaacaacctcaagtatgaagatacagccacttacttttgcgccctcgactatagttacgccatggactactggggacagggaacctccgttaccgtcagttccgcggccgcaaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgctaatgacaggtacctttaagaccaatgacttacaaggcagctgtagatcttagccactttttaaaagaaaaggggggactggaagggctaattcactcccaaagaagacaagatctgctttttgcctgtactgggtctctctggttagaccagatctgagcctgggagctctctggctaactagggaacccactgcttaagcctcaataaagcttgccttgagtgcttcaatgtgtgtgttggttttttgtgtgtcgaaattctagcgattctagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggactagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgagcttgcatgccgacattgattattgactagtccctaagaaaccattcttatcatgacattaacctataaaaataggcgtatcacgaggccctttcgtc

SEQ ID NO: 37 - protein, 472 aa, Artificial Sequence
  exemplary mature BCMA CAR contemplated herein (i.e., without the signal sequence)
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - protein, 246 aa, Artificial Sequence
  amino acid sequence of BCMA02 scFv
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

(19) United States
(12) Patent Application Publication
(10) Pub. No.: US 2023/0398149 A1
(43) Pub. Date: Dec. 14, 2023

(54) CAR T CELL THERAPY IN PATIENTS WHO HAVE HAD PRIOR ANTI-CANCER ALKYLATOR THERAPY

(71) Applicants: Celgene Corporation, Summit, NJ (US); Juno Therapeutics, Inc., Seattle, WA (US)

(72) Inventors: Julie Ann RYTLEWSKI, Seattle, WA (US); Afshin MASHADI-HOSSEIN, Seattle, WA (US); Timothy CAMPBELL, San Francisco, CA (US); Jaymes FULLER, Rahway, NJ (US)

(73) Assignees: Celgene Corporation, Summit, NJ (US); Juno Therapeutics, Inc., Seattle, WA (US)

(21) Appl. No.: 18/035,280

(22) PCT Filed: Nov. 3, 2021
(86) PCT No.: PCT/US2021/057805

§ 371 Date: May 3, 2023

Related U.S. Application Data
(60) Provisional application No. 63/121,658, filed on Dec. 4, 2020.
(60) Provisional application No. 63/120,166, filed on Dec. 1, 2020.
(60) Provisional application No. 63/109,804, filed on Nov. 4, 2020.

(51) Int. Cl. A61K 35/17; A61K 31/675; G16H 20/17; C07K 16/28; C07K 14/725; C07K 14/705; A61P 35/00; A61K 39/00

(52) U.S. Cl. CPC A61K 35/17; A61K 31/675; G16H 20/17; C07K 16/2878; C07K 14/7051; C07K 14/70589; A61P 35/00; A61K 39/4611; A61K 2239/31; A61K 2239/48; A61K 2239/13

(57) ABSTRACT

Provided herein are uses of chimeric antigen receptors (CARs) for treating a tumor or a cancer (such as B cell related cancer, e.g., multiple myeloma). In addition, an optimal washout period for commencing a therapy for the treatment of a condition in a subject after a prior exposure can be determined by receiving, for each of a plurality of subjects, prior treatment history data. Left-censored data can then be derived from the prior treatment history data for each of the subjects that includes a washout period and event or censor. A time scale of the left-censored treatment data is then inverted to result in right-censored treatment data. The right-censored treatment data is then applied to a time-to-event (TTE) model that associates one or more variables of interest with a time since exposure to the prior exposure. A maximally selected log-rank statistic across a plurality of cutoffs within a pre-defined percentile range is computed for continuous variables within the one or more variables of interest. One or more variables and associated cutoffs for the continuous variables having a maximally selected log-rank statistic below a first pre-defined threshold are then identified. A test statistic of each (n−1) strata relative to a reference stratum is then computed for ordinal or categorical variables within the one or more variables of interest. One or more ordinary or categorical variables and associated strata having a test statistic below a second pre-defined threshold, relative to the reference stratum are identified. An optimal washout period is then determined for the therapy based on the cutoff having a lowest value below the pre-defined threshold and relative to a median of subject values below the pre-defined threshold and a median of subject values above the pre-defined threshold.

### CROSS-REFERENCE TO RELATED APPLICATIONS

This application claims the benefit of U.S. Application No. 63/109,804, filed Nov. 4, 2020, U.S. Application No. 63/120,166, filed Dec. 1, 2020, and U.S. Application No. 63/121,658, filed Dec. 4, 2020, the disclosure of each of which is incorporated by reference herein in its entirety.

### REFERENCE TO SEQUENCE LISTING SUBMITTED ELECTRONICALLY

This application incorporates by reference a Sequence Listing submitted with this application as an ASCII text file, entitled “14247-619-228_SEQ_LISTING_CRF.txt” created on Nov. 2, 2021 and having a size of 33,964 bytes.

### 1. FIELD

The disclosure presented herein relates to methods for treating a tumor or a cancer (such as B cell related cancer, e.g., multiple myeloma). More particularly, the disclosure relates to improved methods for treating a tumor or a cancer (such as B cell related cancer, e.g., multiple myeloma) using chimeric antigen receptors (CARs) comprising antibodies or antigen binding fragments thereof (e.g., anti-BCMA antibodies or antigen binding fragments thereof), and immune effector cells (e.g., T cells) genetically modified to express these CARs. The disclosure also relates to methods for manufacturing CARs comprising antibodies or antigen binding fragments thereof (e.g., anti-BCMA antibodies or antigen binding fragments thereof) for treating a tumor or a cancer (such as B cell related cancer, e.g., multiple myeloma). The current subject matter is also directed to techniques for determining an optimal exposure washout period of prior treatment regimens using a time-to-event model.

### 2. BACKGROUND

### 2.1. Background

Many options are currently available for approaching treatment of cancers, including, for example, traditional chemotherapeutic approaches as well as immunotherapies such as chimeric antigen receptor CAR) T cell therapies. In certain instances, use of one therapy may render administration of a second treatment less than optimal. Thus, there is a need for optimizing administration of cancer therapies, e.g., CAR-T therapies, when such therapies are administered to a patient, e.g., when administered sequentially.

In addition, in many situations, a patient may change caregivers and/or change exposure regimes in connection with the treatment of diseases such as cancer. Patients often have extensive and variable prior treatment histories and those prior exposures can affect their present state, which can subsequently affect how they respond to a contemporary exposure.

### 3. BRIEF SUMMARY

The present disclosure generally provides improved methods of treating a tumor or a cancer, such as B-cell-related cancer, e.g., multiple myeloma.

In one aspect, provided herein is a method of treating a tumor or a cancer in a subject in need thereof, comprising: (a) administering to the subject an alkylating agent; (b) isolating peripheral blood mononuclear cells (PBMCs) from the subject at least about six (6) months after step (a); (c) determining that at least about 20% of the PBMCs are T cells; (d) on the basis of the determination in step (c), subsequently manufacturing chimeric antigen receptor (CAR) T cells from the PBMCs; and (e) administering the CAR T cells to the subject. In a specific embodiment, step (b) is performed at least about seven (7) months, at least about eight (8) months, at least about nine (9) months, at least about ten (10) months, at least about eleven (11) months, at least about twelve (12) months, at least about thirteen (13) months, or at least about fourteen (14) months after step (a).

In a specific embodiment, the tumor or cancer is lymphoma, lung cancer, breast cancer, prostate cancer, adrenocortical carcinoma, thyroid carcinoma, nasopharyngeal carcinoma, melanoma, skin carcinoma, colorectal carcinoma, a desmoid tumor, a desmoplastic small round cell tumor, an endocrine tumor, a Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipomachronic lymphocytic leukemia (small lymphocytic lymphoma), B-cell prolymphocytic leukemia, lymphoplasmacytic lymphoma, Waldenström macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, a non-Hodgkin lymphoma, or multiple myeloma. In a specific embodiment, the cancer is multiple myeloma, chronic lymphocytic leukemia, or a non-Hodgkins lymphoma. In a specific embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a specific embodiment, the cancer is multiple myeloma. In a specific embodiment, the multiple myeloma is high-risk multiple myeloma or relapsed and refractory multiple myeloma. In a specific embodiment, the multiple myeloma is high risk multiple myeloma, and the high risk multiple myeloma is R-ISS stage III disease and/or a disease characterized by early relapse. In a particular embodiment, the multiple myeloma is not Revised International Staging System (R-ISS) stage III disease.

In a specific embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan, oxaliplatin, platinum, procarbazine, streptozocin, temozolomide, thiotepa, and trabectedin. In a specific embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a specific embodiment, the alkylating agent is cyclophosphamide.

In a particular embodiment, the subject is a human.

In a particular embodiment, the subject undergoes a leukapharesis procedure to collect the PBMCs for the manufacture of the CAR T cells prior to their administration to the subject.

In a particular embodiment, the CAR T cells are administered by an intravenous infusion.

In another aspect, provided herein is a method of treating a tumor or a cancer in a subject in need thereof, comprising: (a) isolating peripheral blood mononuclear cells (PBMCs) from the subject, and determining that at least about 20% of the PBMCs are T cells; (b) on the basis of the determination in step (a), subsequently manufacturing chimeric antigen receptor (CAR) T cells from the PBMCs; and (c) administering to the subject the CAR T cells, wherein, prior to step (a), the subject had previously received an alkylating agent for treatment of the cancer. In a specific embodiment, the subject had previously received the alkylating agent at least about six (6) months prior to step (a). In a specific embodiment, the subject had previously received the alkylating agent at least about seven (7) months, at least about eight (8) months, at least about nine (9) months, at least about ten (10) months, at least about eleven (11) months, at least about twelve (12) months, at least about thirteen (13) months, or at least about fourteen (14) months prior to step (a).

In a specific embodiment, the tumor or cancer is lymphoma, lung cancer, breast cancer, prostate cancer, adrenocortical carcinoma, thyroid carcinoma, nasopharyngeal carcinoma, melanoma, skin carcinoma, colorectal carcinoma, a desmoid tumor, a desmoplastic small round cell tumor, an endocrine tumor, a Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipomachronic lymphocytic leukemia (small lymphocytic lymphoma), B-cell prolymphocytic leukemia, lymphoplasmacytic lymphoma, Waldenström macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, a non-Hodgkin lymphoma, or multiple myeloma. In a specific embodiment, the cancer is multiple myeloma, chronic lymphocytic leukemia, or a non-Hodgkins lymphoma. In a specific embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a specific embodiment, the cancer is multiple myeloma. In a specific embodiment, the multiple myeloma is high-risk multiple myeloma or relapsed and refractory multiple myeloma. In a specific embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a specific embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan, oxaliplatin, platinum, procarbazine, streptozocin, temozolomide, thiotepa, and trabectedin. In a specific embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a specific embodiment, the alkylating agent is cyclophosphamide.

In a particular embodiment, the subject is a human.

In a particular embodiment, the subject undergoes a leukapharesis procedure to collect the PBMCs for the manufacture of the CAR T cells prior to their administration to the subject.

In a particular embodiment, the CAR T cells are administered by an intravenous infusion.

In another aspect, a method of treating a tumor or a cancer in a subject in need thereof, comprising: (a) isolating peripheral blood mononuclear cells (PBMCs) from the subject; (b) manufacturing chimeric antigen receptor (CAR) T cells from the PBMCs; and (c) administering to the subject the CAR T cells, wherein the subject had previously received an alkylating agent for treatment of the cancer; wherein step (a) occurs at least about six months after the subject received the alkylating agent. In a particular embodiment, step (a) further comprises determining that at least about 20% of the PBMCs are T cells; and wherein step (b) further comprises subsequently manufacturing chimeric antigen receptor (CAR) T cells from the PBMCs on the basis of the determination in step (a). In a particular embodiment, step (a) occurs at least about seven (7) months, at least about eight (8) months, at least about nine (9) months, at least about ten (10) months, at least about eleven (11) months, at least about twelve (12) months, at least about thirteen (13) months, or at least about fourteen (14) months after the subject received the alkylating agent.

In a specific embodiment, the tumor or cancer is lymphoma, lung cancer, breast cancer, prostate cancer, adrenocortical carcinoma, thyroid carcinoma, nasopharyngeal carcinoma, melanoma, skin carcinoma, colorectal carcinoma, a desmoid tumor, a desmoplastic small round cell tumor, an endocrine tumor, a Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipomachronic lymphocytic leukemia (small lymphocytic lymphoma), B-cell prolymphocytic leukemia, lymphoplasmacytic lymphoma, Waldenström macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, a non-Hodgkin lymphoma, or multiple myeloma. In a specific embodiment, the cancer is multiple myeloma, chronic lymphocytic leukemia, or a non-Hodgkins lymphoma. In a specific embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a specific embodiment, the cancer is multiple myeloma. In a specific embodiment, the multiple myeloma is high-risk multiple myeloma or relapsed and refractory multiple myeloma. In a specific embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a specific embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan, oxaliplatin, platinum, procarbazine, streptozocin, temozolomide, thiotepa, and trabectedin. In a specific embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a specific embodiment, the alkylating agent is cyclophosphamide.

In a particular embodiment, the subject is a human.

In a particular embodiment, the subject undergoes a leukapharesis procedure to collect the PBMCs for the manufacture of the CAR T cells prior to their administration to the subject.

In a particular embodiment, the CAR T cells are administered by an intravenous infusion.

In another aspect, provided herein is a method of treating a tumor or a cancer in a subject in need thereof, wherein the subject has been administered an alkylating agent, comprising: (a) determining that the subject has not been administered the alkylating agent less than about six (6) months prior to the determining step; (b) isolating peripheral blood mononuclear cells (PBMCs) from the subject; (c) manufacturing chimeric antigen receptor (CAR) T cells from the PBMCs; and (d) administering to the subject the CAR T cells. In a particular embodiment, in step (a) the subject has not been administered the alkylating agent less than about seven (7) months, less than about eight (8) months, less than about nine (9) months, less than about ten (10) months, less than about eleven (11) months, less than about twelve (12) months, less than about thirteen (13) months, or less than about fourteen (14) months prior to the determining step.

In a specific embodiment, the tumor or cancer is lymphoma, lung cancer, breast cancer, prostate cancer, adrenocortical carcinoma, thyroid carcinoma, nasopharyngeal carcinoma, melanoma, skin carcinoma, colorectal carcinoma, a desmoid tumor, a desmoplastic small round cell tumor, an endocrine tumor, a Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipomachronic lymphocytic leukemia (small lymphocytic lymphoma), B-cell prolymphocytic leukemia, lymphoplasmacytic lymphoma, Waldenström macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, a non-Hodgkin lymphoma, or multiple myeloma. In a specific embodiment, the cancer is multiple myeloma, chronic lymphocytic leukemia, or a non-Hodgkins lymphoma. In a specific embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a specific embodiment, the cancer is multiple myeloma. In a specific embodiment, the multiple myeloma is high-risk multiple myeloma or relapsed and refractory multiple myeloma. In a specific embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a specific embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan, oxaliplatin, platinum, procarbazine, streptozocin, temozolomide, thiotepa, and trabectedin. In a specific embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a specific embodiment, the alkylating agent is cyclophosphamide.

In a particular embodiment, the subject is a human.

In a particular embodiment, the subject undergoes a leukapharesis procedure to collect the PBMCs for the manufacture of the CAR T cells prior to their administration to the subject.

In a particular embodiment, the CAR T cells are administered by an intravenous infusion.

In another aspect, provided herein is a method of treating a tumor or a cancer in a subject in need thereof, wherein the subject has been administered an alkylating agent, comprising: (a) isolating peripheral blood mononuclear cells (PBMCs) from the subject; (b) manufacturing chimeric antigen receptor (CAR) T cells from the PBMCs; and (c) administering to the subject the CAR T cells, wherein, at the time of the isolating, the subject has been determined to have been administered the alkylating agent at least about six (6) months prior. In a particular embodiment, the subject has been determined to have been administered the alkylating agent at least about seven (7) months, at least about eight (8) months, at least about nine (9) months, at least about ten (10) months, at least about eleven (11) months, at least about twelve (12) months, at least about thirteen (13) months, or at least about fourteen (14) months prior.

In a specific embodiment, the tumor or cancer is lymphoma, lung cancer, breast cancer, prostate cancer, adrenocortical carcinoma, thyroid carcinoma, nasopharyngeal carcinoma, melanoma, skin carcinoma, colorectal carcinoma, a desmoid tumor, a desmoplastic small round cell tumor, an endocrine tumor, a Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipomachronic lymphocytic leukemia (small lymphocytic lymphoma), B-cell prolymphocytic leukemia, lymphoplasmacytic lymphoma, Waldenström macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, a non-Hodgkin lymphoma, or multiple myeloma. In a specific embodiment, the cancer is multiple myeloma, chronic lymphocytic leukemia, or a non-Hodgkins lymphoma. In a specific embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a specific embodiment, the cancer is multiple myeloma. In a specific embodiment, the multiple myeloma is high-risk multiple myeloma or relapsed and refractory multiple myeloma. In a specific embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a specific embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan, oxaliplatin, platinum, procarbazine, streptozocin, temozolomide, thiotepa and trabectedin. In a specific embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a specific embodiment, the alkylating agent is cyclophosphamide.

In a particular embodiment, the subject is a human.

In a particular embodiment, the subject undergoes a leukapharesis procedure to collect the PBMCs for the manufacture of the CAR T cells prior to their administration to the subject.

In a particular embodiment, the CAR T cells are administered by an intravenous infusion.

In another aspect, provided herein is a method of treating a tumor or a cancer in a subject in need thereof, wherein the subject has been administered an alkylating agent, comprising administering to the subject T cells expressing a chimeric antigen receptor (CAR T cells) manufactured from peripheral blood mononuclear cells PBMCs isolated from the patient, wherein, at the time said PBMCs are isolated, the subject has last received the alkylating agent at least about six (6) months prior to the time the PBMCs are isolated. In a particular embodiment, the subject has last received the alkylating agent at least about seven (7) months, at least about eight (8) months, at least about nine (9) months, at least about ten (10) months, at least about eleven (11) months, at least about twelve (12) months, at least about thirteen (13) months, or at least about fourteen (14) months prior to the time the PBMCs are isolated.

In a specific embodiment, the tumor or cancer is lymphoma, lung cancer, breast cancer, prostate cancer, adrenocortical carcinoma, thyroid carcinoma, nasopharyngeal carcinoma, melanoma, skin carcinoma, colorectal carcinoma, a desmoid tumor, a desmoplastic small round cell tumor, an endocrine tumor, a Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipomachronic lymphocytic leukemia (small lymphocytic lymphoma), B-cell prolymphocytic leukemia, lymphoplasmacytic lymphoma, Waldenström macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, a non-Hodgkin lymphoma, or multiple myeloma. In a specific embodiment, the cancer is multiple myeloma, chronic lymphocytic leukemia, or a non-Hodgkins lymphoma. In a specific embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a specific embodiment, the cancer is multiple myeloma. In a specific embodiment, the multiple myeloma is high-risk multiple myeloma or relapsed and refractory multiple myeloma. In a specific embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a specific embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan, oxaliplatin, platinum, procarbazine, streptozocin, temozolomide, thiotepa and trabectedin. In a specific embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a specific embodiment, the alkylating agent is cyclophosphamide.

In a particular embodiment, the subject is a human.

In a particular embodiment, the subject undergoes a leukapharesis procedure to collect the PBMCs for the manufacture of the CAR T cells prior to their administration to the subject.

In a particular embodiment, the CAR T cells are administered by an intravenous infusion.

In another aspect, provided herein is a method of treating a tumor or a cancer in a subject in need thereof, wherein the subject has been administered an alkylating agent, comprising administering to the subject T cells expressing a chimeric antigen receptor (CAR T cells) manufactured from peripheral blood mononuclear cells PBMCs isolated from the patient, wherein, at the time said PBMCs are isolated, the PBMCs comprises at least about 20% T cells.

In a specific embodiment, the tumor or cancer is lymphoma, lung cancer, breast cancer, prostate cancer, adrenocortical carcinoma, thyroid carcinoma, nasopharyngeal carcinoma, melanoma, skin carcinoma, colorectal carcinoma, a desmoid tumor, a desmoplastic small round cell tumor, an endocrine tumor, a Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipomachronic lymphocytic leukemia (small lymphocytic lymphoma), B-cell prolymphocytic leukemia, lymphoplasmacytic lymphoma, Waldenström macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, a non-Hodgkin lymphoma, or multiple myeloma. In a specific embodiment, the cancer is multiple myeloma, chronic lymphocytic leukemia, or a non-Hodgkins lymphoma. In a specific embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a specific embodiment, the cancer is multiple myeloma. In a specific embodiment, the multiple myeloma is high-risk multiple myeloma or relapsed and refractory multiple myeloma. In a specific embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a specific embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan, oxaliplatin, platinum, procarbazine, streptozocin, temozolomide, thiotepa and trabectedin. In a specific embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a specific embodiment, the alkylating agent is cyclophosphamide.

In a particular embodiment, the subject is a human.

In a particular embodiment, the subject undergoes a leukapharesis procedure to collect the PBMCs for the manufacture of the CAR T cells prior to their administration to the subject.

In a particular embodiment, the CAR T cells are administered by an intravenous infusion.

In another aspect, provided herein is a method of treating a cancer caused by B Cell Maturation Agent (BCMA) expressing cells in a subject in need thereof, comprising: (a) administering to the subject an alkylating agent; (b) isolating peripheral blood mononuclear cells (PBMCs) from the subject at least about six months after step (a); (c) determining that at least about 20% of the PBMCs are T cells; (d) on the basis of the determination in step (c), subsequently manufacturing chimeric antigen receptor (CAR) T cells directed to BCMA (BCMA CAR T cells) from the PBMCs; and (e) administering to the subject the CAR T cells to the subject. In a particular embodiment, step (b) is performed at least about seven (7) months, at least about eight (8) months, at least about nine (9) months, at least about ten (10) months, at least about eleven (11) months, at least about twelve (12) months, at least about thirteen (13) months, or at least about fourteen (14) months after step (a) of administering to the subject an alkylating agent.

In a particular embodiment, the cancer is multiple myeloma, chronic lymphocytic leukemia, or a non-Hodgkins lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is multiple myeloma. In a particular embodiment, the multiple myeloma is high-risk multiple myeloma or relapsed and refractory multiple myeloma. In a particular embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a particular embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan, oxaliplatin, platinum, procarbazine, streptozocin, temozolomide, thiotepa, and trabectedin. In a particular embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a particular embodiment, the alkylating agent is cyclophosphamide.

In a particular embodiment, the subject is a human.

In a particular embodiment, the BCMA CAR T cells comprise a CAR directed to BCMA, wherein the CAR directed to BCMA comprises an antibody or antibody fragment that targets BCMA. In a particular embodiment, the BCMA CAR T cells comprise a CAR directed to BCMA, wherein the CAR directed to BCMA comprises a single chain Fv antibody or antibody fragment (scFv). In a particular embodiment, the BCMA CAR T cells comprise a CAR directed to BCMA, wherein the CAR directed to BCMA comprises a BCMA02 scFv. In a particular embodiment, the BCMA CAR T cells are idecabtagene vicleucel cells.

In a particular embodiment, the subject undergoes a leukapharesis procedure to collect the PBMCs for the manufacture of the BCMA CAR T cells prior to their administration to the subject.

In a particular embodiment, the BCMA CAR T cells are administered by an intravenous infusion.

In another aspect, provided herein is a method of treating a cancer caused by B Cell Maturation Agent (BCMA) expressing cells in a subject in need thereof, comprising: (a) isolating peripheral blood mononuclear cells (PBMCs) from the subject, and determining that at least about 20% of the PBMCs are T cells; (b) on the basis of the determination in step (a), subsequently manufacturing chimeric antigen receptor (CAR) T cells directed to BCMA (BCMA CAR T cells) from the PBMCs; and (c) administering to the subject the CAR T cells, wherein, prior to step (a), the subject had previously received an alkylating agent for treatment of the cancer. In a particular embodiment, the subject had previously received the alkylating agent at least about six months prior to step (a). In a particular embodiment, the subject had previously received the alkylating agent at least about seven (7) months, at least about eight (8) months, at least about nine (9) months, at least about ten (10) months, at least about eleven (11) months, at least about twelve (12) months, at least about thirteen (13) months, or at least about fourteen (14) months prior to step (a).

In a particular embodiment, the cancer is multiple myeloma, chronic lymphocytic leukemia, or a non-Hodgkins lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is multiple myeloma. In a particular embodiment, the multiple myeloma is high-risk multiple myeloma or relapsed and refractory multiple myeloma. In a particular embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a particular embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan, oxaliplatin, platinum, procarbazine, streptozocin, temozolomide, thiotepa, and trabectedin. In a particular embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a particular embodiment, the alkylating agent is cyclophosphamide.

In a particular embodiment, the subject is a human.

In a particular embodiment, the BCMA CAR T cells comprise a CAR directed to BCMA, wherein the CAR directed to BCMA comprises an antibody or antibody fragment that targets BCMA. In a particular embodiment, the BCMA CAR T cells comprise a CAR directed to BCMA, wherein the CAR directed to BCMA comprises a single chain Fv antibody or antibody fragment (scFv). In a particular embodiment, the BCMA CAR T cells comprise a CAR directed to BCMA, wherein the CAR directed to BCMA comprises a BCMA02 scFv. In a particular embodiment, the BCMA CAR T cells are idecabtagene vicleucel cells.

In a particular embodiment, the subject undergoes a leukapharesis procedure to collect the PBMCs for the manufacture of the BCMA CAR T cells prior to their administration to the subject.

In a particular embodiment, the BCMA CAR T cells are administered by an intravenous infusion.

In another aspect, provided herein is a method of treating a cancer caused by B Cell Maturation Agent (BCMA) expressing cells in a subject in need thereof, comprising: (a) isolating peripheral blood mononuclear cells (PBMCs) from the subject; (b) manufacturing chimeric antigen receptor (CAR) T cells directed to BCMA (BCMA CAR T cells) from the PBMCs; (c) administering to the subject the BCMA CAR T cells, wherein the patient had previously received an alkylating agent for treatment of the cancer, and wherein step (a) occurs at least about six months after the subject received the alkylating agent. In a particular embodiment, step (a) further comprises determining that at least about 20% of the PBMCs are T cells; and wherein step (b) further comprises subsequently manufacturing chimeric antigen receptor (CAR) T cells from the PBMCs on the basis of the determination in step (a). In a particular embodiment, step (a) occurs at least about seven (7) months, at least about eight (8) months, at least about nine (9) months, at least about ten (10) months, at least about eleven (11) months, at least about twelve (12) months, at least about thirteen (13) months, or at least about fourteen (14) months after the subject received the alkylating agent.

In a particular embodiment, the cancer is multiple myeloma, chronic lymphocytic leukemia, or a non-Hodgkins lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is multiple myeloma. In a particular embodiment, the multiple myeloma is high-risk multiple myeloma or relapsed and refractory multiple myeloma. In a particular embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a particular embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan, oxaliplatin, platinum, procarbazine, streptozocin, temozolomide, thiotepa, and trabectedin. In a particular embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a particular embodiment, the alkylating agent is cyclophosphamide.

In a particular embodiment, the subject is a human.

In a particular embodiment, the BCMA CAR T cells comprise a CAR directed to BCMA, wherein the CAR directed to BCMA comprises an antibody or antibody fragment that targets BCMA. In a particular embodiment, the BCMA CAR T cells comprise a CAR directed to BCMA, wherein the CAR directed to BCMA comprises a single chain Fv antibody or antibody fragment (scFv). In a particular embodiment, the BCMA CAR T cells comprise a CAR directed to BCMA, wherein the CAR directed to BCMA comprises a BCMA02 scFv. In a particular embodiment, the BCMA CAR T cells are idecabtagene vicleucel cells.

In a particular embodiment, the subject undergoes a leukapharesis procedure to collect the PBMCs for the manufacture of the BCMA CAR T cells prior to their administration to the subject.

In a particular embodiment, the BCMA CAR T cells are administered by an intravenous infusion.

In another aspect, provided herein is a method of treating a cancer caused by B Cell Maturation Agent (BCMA) expressing cells in a subject in need thereof, wherein the subject has been administered an alkylating agent for treatment of a cancer, comprising: (a) determining that the subject has not been administered the alkylating agent less than about six (6) months prior to the determining step; (b) isolating peripheral blood mononuclear cells (PBMCs) from the subject, wherein the isolating is performed at least six (6) months after the alkylating agent has been administered to the subject; (c) manufacturing chimeric antigen receptor (CAR) T cells directed to BCMA (BCMA CAR T cells) from the PBMCs; and (d) administering to the subject the BCMA CAR T cells. In a particular embodiment, in step (a) the subject has not been administered the alkylating agent less than about seven (7) months, less than about eight (8) months, less than about nine (9) months, less than about ten (10) months, less than about eleven (11) months, less than about twelve (12) months, less than about thirteen (13) months, or less than about fourteen (14) months prior to the determining step.

In a particular embodiment, the cancer is multiple myeloma, chronic lymphocytic leukemia, or a non-Hodgkins lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is multiple myeloma. In a particular embodiment, the multiple myeloma is high-risk multiple myeloma or relapsed and refractory multiple myeloma. In a particular embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a particular embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan, oxaliplatin, platinum, procarbazine, streptozocin, temozolomide, thiotepa, and trabectedin. In a particular embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a particular embodiment, the alkylating agent is cyclophosphamide.

In a particular embodiment, the subject is a human.

In a particular embodiment, the BCMA CAR T cells comprise a CAR directed to BCMA, wherein the CAR directed to BCMA comprises an antibody or antibody fragment that targets BCMA. In a particular embodiment, the BCMA CAR T cells comprise a CAR directed to BCMA, wherein the CAR directed to BCMA comprises a single chain Fv antibody or antibody fragment (scFv). In a particular embodiment, the BCMA CAR T cells comprise a CAR directed to BCMA, wherein the CAR directed to BCMA comprises a BCMA02 scFv. In a particular embodiment, the BCMA CAR T cells are idecabtagene vicleucel cells.

In a particular embodiment, the subject undergoes a leukapharesis procedure to collect the PBMCs for the manufacture of the BCMA CAR T cells prior to their administration to the subject.

In a particular embodiment, the BCMA CAR T cells are administered by an intravenous infusion.

In another aspect, provided herein is a method of treating a cancer caused by B Cell Maturation Agent (BCMA) expressing cells in a subject in need thereof, wherein the subject has been administered an alkylating agent, comprising: (a) isolating peripheral blood mononuclear cells (PBMCs) from the subject; (b) manufacturing chimeric antigen receptor (CAR) T cells directed to BCMA (BCMA CAR T cells) from the PBMCs; and (c) administering to the subject the BCMA CAR T cells, wherein, at the time of the isolating, the subject has been determined to have been administered the alkylating agent at least about six (6) months prior. In a particular embodiment, the subject has been determined to have been administered the alkylating agent at least about seven (7) months, at least about eight (8) months, at least about nine (9) months, at least about ten (10) months, at least about eleven (11) months, at least about twelve (12) months, at least about thirteen (13) months, or at least about fourteen (14) months prior.

In a particular embodiment, the cancer is multiple myeloma, chronic lymphocytic leukemia, or a non-Hodgkins lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is multiple myeloma. In a particular embodiment, the multiple myeloma is high-risk multiple myeloma or relapsed and refractory multiple myeloma. In a particular embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a particular embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan, oxaliplatin, platinum, procarbazine, streptozocin, temozolomide, thiotepa, and trabectedin. In a particular embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a particular embodiment, the alkylating agent is cyclophosphamide.

In a particular embodiment, the subject is a human.

In a particular embodiment, the BCMA CAR T cells comprise a CAR directed to BCMA, wherein the CAR directed to BCMA comprises an antibody or antibody fragment that targets BCMA. In a particular embodiment, the BCMA CAR T cells comprise a CAR directed to BCMA, wherein the CAR directed to BCMA comprises a single chain Fv antibody or antibody fragment (scFv). In a particular embodiment, the BCMA CAR T cells comprise a CAR directed to BCMA, wherein the CAR directed to BCMA comprises a BCMA02 scFv. In a particular embodiment, the BCMA CAR T cells are idecabtagene vicleucel cells.

In a particular embodiment, the subject undergoes a leukapharesis procedure to collect the PBMCs for the manufacture of the BCMA CAR T cells prior to their administration to the subject.

In a particular embodiment, the BCMA CAR T cells are administered by an intravenous infusion.

In another aspect, provided herein is a method of treating a cancer caused by B Cell Maturation Agent (BCMA) expressing cells in a subject in need thereof, wherein the subject has been administered an alkylating agent, comprising administering to the subject chimeric antigen receptor (CAR) T cells directed to BCMA (BCMA CAR T cells) manufactured from peripheral blood mononuclear cells PBMCs isolated from the patient, wherein, at the time said PBMCs are isolated, the subject has last received the alkylating agent at least about six (6) months prior to the time the PBMCs are isolated. In a particular embodiment, the subject has last received the alkylating agent at least about seven (7) months, at least about eight (8) months, at least about nine (9) months, at least about ten (10) months, at least about eleven (11) months, at least about twelve (12) months, at least about thirteen (13) months, or at least about fourteen (14) months prior to the time the PBMCs are isolated.

In a particular embodiment, the cancer is multiple myeloma, chronic lymphocytic leukemia, or a non-Hodgkins lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is multiple myeloma. In a particular embodiment, the multiple myeloma is high-risk multiple myeloma or relapsed and refractory multiple myeloma. In a particular embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a particular embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan, oxaliplatin, platinum, procarbazine, streptozocin, temozolomide, thiotepa, and trabectedin. In a particular embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a particular embodiment, the alkylating agent is cyclophosphamide.

In a particular embodiment, the subject is a human.

In a particular embodiment, the BCMA CAR T cells comprise a CAR directed to BCMA, and, e.g., wherein the CAR directed to BCMA comprises an antibody or antibody fragment that targets BCMA. In a particular embodiment, the BCMA CAR T cells comprise a CAR directed to BCMA, wherein the CAR directed to BCMA comprises a single chain Fv antibody or antibody fragment (scFv). In a particular embodiment, the BCMA CAR T cells comprise a CAR directed to BCMA, wherein the CAR directed to BCMA comprises a BCMA02 scFv. In a particular embodiment, the BCMA CAR T cells are idecabtagene vicleucel cells.

In a particular embodiment, the subject undergoes a leukapharesis procedure to collect the PBMCs for the manufacture of the BCMA CAR T cells prior to their administration to the subject.

In a particular embodiment, the BCMA CAR T cells are administered by an intravenous infusion.

In another aspect, provided herein is a method of treating a cancer caused by B Cell Maturation Agent (BCMA) expressing cells in a subject in need thereof, wherein the subject has been administered an alkylating agent, comprising administering to the subject chimeric antigen receptor (CAR) T cells directed to BCMA (BCMA CAR T cells) manufactured from peripheral blood mononuclear cells PBMCs isolated from the patient, wherein, at the time said PBMCs are isolated, the PBMCs comprise at least about 20% T cells.

In a particular embodiment, the cancer is multiple myeloma, chronic lymphocytic leukemia, or a non-Hodgkins lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is multiple myeloma. In a particular embodiment, the multiple myeloma is high-risk multiple myeloma or relapsed and refractory multiple myeloma. In a particular embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a particular embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan, oxaliplatin, platinum, procarbazine, streptozocin, temozolomide, thiotepa, and trabectedin. In a particular embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a particular embodiment, the alkylating agent is cyclophosphamide.

In a particular embodiment, the subject is a human.

In a particular embodiment, the BCMA CAR T cells comprise a CAR directed to BCMA, wherein the CAR directed to BCMA comprises an antibody or antibody fragment that targets BCMA. In a particular embodiment, the BCMA CAR T cells comprise a CAR directed to BCMA, wherein the CAR directed to BCMA comprises a single chain Fv antibody or antibody fragment (scFv). In a particular embodiment, the BCMA CAR T cells comprise a CAR directed to BCMA, wherein the CAR directed to BCMA comprises a BCMA02 scFv. In a particular embodiment, the BCMA CAR T cells are idecabtagene vicleucel cells.

In a particular embodiment, the subject undergoes a leukapharesis procedure to collect the PBMCs for the manufacture of the BCMA CAR T cells prior to their administration to the subject.

In a particular embodiment, the BCMA CAR T cells are administered by an intravenous infusion.

In another aspect, provided herein is a method of manufacturing chimeric antigen receptor (CAR) T cells from a subject, comprising: (a) administering to the subject an alkylating agent for treatment of a tumor or a cancer; (b) isolating peripheral blood mononuclear cells (PBMCs) from the subject at least about six months after step (a); and (c) manufacturing chimeric antigen receptor (CAR) T cells from the PBMCs. In a particular embodiment, step (b) is performed at least about seven (7) months, at least about eight (8) months, at least about nine (9) months, at least about ten (10) months, at least about eleven (11) months, at least about twelve (12) months, at least about thirteen (13) months, or at least about fourteen (14) months after step (a).

In a particular embodiment, the tumor or cancer is lymphoma, lung cancer, breast cancer, prostate cancer, adrenocortical carcinoma, thyroid carcinoma, nasopharyngeal carcinoma, melanoma, skin carcinoma, colorectal carcinoma, a desmoid tumor, a desmoplastic small round cell tumor, an endocrine tumor, a Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipomachronic lymphocytic leukemia (small lymphocytic lymphoma), B-cell prolymphocytic leukemia, lymphoplasmacytic lymphoma, Waldenström macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, a non-Hodgkin lymphoma, or multiple myeloma. In a particular embodiment, the cancer is multiple myeloma, chronic lymphocytic leukemia, or a non-Hodgkins lymphoma. In a particular embodiment, the cancer is multiple myeloma, chronic lymphocytic leukemia, or a non-Hodgkins lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is multiple myeloma. In a particular embodiment, the multiple myeloma is high-risk multiple myeloma or relapsed and refractory multiple myeloma. In a particular embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a particular embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan, oxaliplatin, platinum, procarbazine, streptozocin, temozolomide, thiotepa, and trabectedin. In a particular embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a particular embodiment, the alkylating agent is cyclophosphamide.

In a particular embodiment, the subject is a human.

In a particular embodiment, the subject undergoes a leukapharesis procedure to collect the PBMCs for the manufacture of the CAR T cells prior to their administration to the subject.

In a particular embodiment, the CAR T cells are administered by an intravenous infusion.

In another aspect, provided herein is a method of manufacturing chimeric antigen receptor (CAR) T cells from a subject, comprising: (a) isolating peripheral blood mononuclear cells (PBMCs) from the subject, and determining that at least about 20% of the PBMCs are T cells; and (b) on the basis of the determination in step (b), subsequently manufacturing chimeric antigen receptor (CAR) T cells from the PBMCs; wherein, prior to step (a), the subject had previously received an alkylating agent for treatment of a tumor or a cancer. In a particular embodiment, the subject had previously received the alkylating agent at least about six months prior to step (a). In a particular embodiment, the subject had previously received the alkylating agent at least about seven (7) months, at least about eight (8) months, at least about nine (9) months, at least about ten (10) months, at least about eleven (11) months, at least about twelve (12) months, at least about thirteen (13) months, or at least about fourteen (14) months prior to step (a).

In a particular embodiment, the tumor or cancer is lymphoma, lung cancer, breast cancer, prostate cancer, adrenocortical carcinoma, thyroid carcinoma, nasopharyngeal carcinoma, melanoma, skin carcinoma, colorectal carcinoma, a desmoid tumor, a desmoplastic small round cell tumor, an endocrine tumor, a Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipomachronic lymphocytic leukemia (small lymphocytic lymphoma), B-cell prolymphocytic leukemia, lymphoplasmacytic lymphoma, Waldenström macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, a non-Hodgkin lymphoma, or multiple myeloma. In a particular embodiment, the cancer is multiple myeloma, chronic lymphocytic leukemia, or a non-Hodgkins lymphoma. In a particular embodiment, the cancer is multiple myeloma, chronic lymphocytic leukemia, or a non-Hodgkins lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is multiple myeloma. In a particular embodiment, the multiple myeloma is high-risk multiple myeloma or relapsed and refractory multiple myeloma. In a particular embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a particular embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan, oxaliplatin, platinum, procarbazine, streptozocin, temozolomide, thiotepa, and trabectedin. In a particular embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a particular embodiment, the alkylating agent is cyclophosphamide.

In a particular embodiment, the subject is a human.

In a particular embodiment, the subject undergoes a leukapharesis procedure to collect the PBMCs for the manufacture of the CAR T cells prior to their administration to the subject.

In a particular embodiment, the CAR T cells are administered by an intravenous infusion.

In another aspect, provided herein is a method of manufacturing chimeric antigen receptor (CAR) T cells from a subject, comprising: (a) isolating peripheral blood mononuclear cells (PBMCs) from the subject; and (b) manufacturing chimeric antigen receptor (CAR) T cells from the PBMCs; wherein the subject had previously received an alkylating agent for treatment of a tumor or a cancer; wherein step (a) occurs at least about six months after the subject received the alkylating agent. In a particular embodiment, step (a) further comprises determining that at least about 20% of the PBMCs are T cells; and wherein step (b) further comprises subsequently manufacturing chimeric antigen receptor (CAR) T cells from the PBMCs on the basis of the determination in step (a). In a particular embodiment, step (a) occurs at least about seven (7) months, at least about eight (8) months, at least about nine (9) months, at least about ten (10) months, at least about eleven (11) months, at least about twelve (12) months, at least about thirteen (13) months, or at least about fourteen (14) months after the subject received the alkylating agent.

In a particular embodiment, the tumor or cancer is lymphoma, lung cancer, breast cancer, prostate cancer, adrenocortical carcinoma, thyroid carcinoma, nasopharyngeal carcinoma, melanoma, skin carcinoma, colorectal carcinoma, a desmoid tumor, a desmoplastic small round cell tumor, an endocrine tumor, a Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipomachronic lymphocytic leukemia (small lymphocytic lymphoma), B-cell prolymphocytic leukemia, lymphoplasmacytic lymphoma, Waldenström macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, a non-Hodgkin lymphoma, or multiple myeloma. In a particular embodiment, the cancer is multiple myeloma, chronic lymphocytic leukemia, or a non-Hodgkins lymphoma. In a particular embodiment, the cancer is multiple myeloma, chronic lymphocytic leukemia, or a non-Hodgkins lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is multiple myeloma. In a particular embodiment, the multiple myeloma is high-risk multiple myeloma or relapsed and refractory multiple myeloma. In a particular embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a particular embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan, oxaliplatin, platinum, procarbazine, streptozocin, temozolomide, thiotepa, and trabectedin. In a particular embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a particular embodiment, the alkylating agent is cyclophosphamide.

In a particular embodiment, the subject is a human.

In a particular embodiment, the subject undergoes a leukapharesis procedure to collect the PBMCs for the manufacture of the CAR T cells prior to their administration to the subject.

In a particular embodiment, the CAR T cells are administered by an intravenous infusion.

In another aspect, provided herein is a method of manufacturing chimeric antigen receptor (CAR) T cells from a subject, wherein the subject has been administered an alkylating agent for treatment of a tumor or a cancer, comprising: a. determining that the subject has not been administered the alkylating agent less than about six (6) months prior to the determining step; b. isolating peripheral blood mononuclear cells (PBMCs) from the subject; and c. manufacturing chimeric antigen receptor (CAR) T cells from the PBMCs. In a particular embodiment, in step (a) the subject has not been administered the alkylating agent less than about seven (7) months, less than about eight (8) months, less than about nine (9) months, less than about ten (10) months, less than about eleven (11) months, less than about twelve (12) months, less than about thirteen (13) months, or less than about fourteen (14) months prior to the determining step.

In a particular embodiment, the tumor or cancer is lymphoma, lung cancer, breast cancer, prostate cancer, adrenocortical carcinoma, thyroid carcinoma, nasopharyngeal carcinoma, melanoma, skin carcinoma, colorectal carcinoma, a desmoid tumor, a desmoplastic small round cell tumor, an endocrine tumor, a Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipomachronic lymphocytic leukemia (small lymphocytic lymphoma), B-cell prolymphocytic leukemia, lymphoplasmacytic lymphoma, Waldenström macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, a non-Hodgkin lymphoma, or multiple myeloma. In a particular embodiment, the cancer is multiple myeloma, chronic lymphocytic leukemia, or a non-Hodgkins lymphoma. In a particular embodiment, the cancer is multiple myeloma, chronic lymphocytic leukemia, or a non-Hodgkins lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is multiple myeloma. In a particular embodiment, the multiple myeloma is high-risk multiple myeloma or relapsed and refractory multiple myeloma. In a particular embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a particular embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan, oxaliplatin, platinum, procarbazine, streptozocin, temozolomide, thiotepa, and trabectedin. In a particular embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a particular embodiment, the alkylating agent is cyclophosphamide.

In a particular embodiment, the subject is a human.

In a particular embodiment, the subject undergoes a leukapharesis procedure to collect the PBMCs for the manufacture of the CAR T cells prior to their administration to the subject.

In a particular embodiment, the CAR T cells are administered by an intravenous infusion.

In another aspect, provided herein is a method of manufacturing chimeric antigen receptor (CAR) T cells from a subject, wherein the subject has been administered an alkylating agent for treatment of a tumor or a cancer, comprising: (a) isolating peripheral blood mononuclear cells (PBMCs) from the subject; and (b) manufacturing chimeric antigen receptor (CAR) T cells from the PBMCs; wherein, at the time of the isolating, the subject has been determined to have been administered the alkylating agent at least about six (6) months prior. In a particular embodiment, the subject has been determined to have been administered the alkylating agent at least about seven (7) months, at least about eight (8) months, at least about nine (9) months, at least about ten (10) months, at least about eleven (11) months, at least about twelve (12) months, at least about thirteen (13) months, or at least about fourteen (14) months prior.

In a particular embodiment, the tumor or cancer is lymphoma, lung cancer, breast cancer, prostate cancer, adrenocortical carcinoma, thyroid carcinoma, nasopharyngeal carcinoma, melanoma, skin carcinoma, colorectal carcinoma, a desmoid tumor, a desmoplastic small round cell tumor, an endocrine tumor, a Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipomachronic lymphocytic leukemia (small lymphocytic lymphoma), B-cell prolymphocytic leukemia, lymphoplasmacytic lymphoma, Waldenström macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, a non-Hodgkin lymphoma, or multiple myeloma. In a particular embodiment, the cancer is multiple myeloma, chronic lymphocytic leukemia, or a non-Hodgkins lymphoma. In a particular embodiment, the cancer is multiple myeloma, chronic lymphocytic leukemia, or a non-Hodgkins lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is multiple myeloma. In a particular embodiment, the multiple myeloma is high-risk multiple myeloma or relapsed and refractory multiple myeloma. In a particular embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a particular embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan, oxaliplatin, platinum, procarbazine, streptozocin, temozolomide, thiotepa, and trabectedin. In a particular embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a particular embodiment, the alkylating agent is cyclophosphamide.

In a particular embodiment, the subject is a human.

In a particular embodiment, the subject undergoes a leukapharesis procedure to collect the PBMCs for the manufacture of the CAR T cells prior to their administration to the subject.

In a particular embodiment, the CAR T cells are administered by an intravenous infusion.

In another aspect, provided herein is a method of manufacturing chimeric antigen receptor (CAR) T cells from a subject, comprising: (a) isolating peripheral blood mononuclear cells (PBMCs) from the subject, and determining that at least about 20% of the PBMCs are T cells; and (b) on the basis of the determination in step (a), subsequently manufacturing the CAR T cells from the PBMCs; wherein the subject had previously received an alkylating agent for treatment of a tumor or a cancer. In a particular embodiment, the subject had previously received the alkylating agent at least about six months prior to step (a). In a particular embodiment, the subject had previously received the alkylating agent at least about seven (7) months, at least about eight (8) months, at least about nine (9) months, at least about ten (10) months, at least about eleven (11) months, at least about twelve (12) months, at least about thirteen (13) months, or at least about fourteen (14) months prior to step (a).

In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are T cells.

In a particular embodiment of the methods presented herein, the T cells comprise CD3+ cells. In particular embodiments, the T cells are CD3+ cells. In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T CD3+ T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are CD3+ T cells, e.g., CD45+/CD3+ T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are CD3+ T cells.

In a particular embodiment, the T cells are CD8+ effector memory RA (T_{EMRA}) (CCR7−/CD45RA+) T cells. In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T_{EMRA} T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are T_{EMRA} T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are T_{EMRA} T cells. In particular embodiments of such methods, the T cells are CD3+ cells.

In the methods presented herein, the determining may be performed using standard techniques well known to those of skill in the relevant art. For example, in the methods presented herein, the determining step may be performed by utilizing techniques such as those utilized in Example 2 (e.g., immunophenotyping of the PBMCs by polychromatic flow cytometry for markers associated with T cell differentiation, memory, senescence, and exhaustion).

In a particular embodiment, the tumor or cancer is lymphoma, lung cancer, breast cancer, prostate cancer, adrenocortical carcinoma, thyroid carcinoma, nasopharyngeal carcinoma, melanoma, skin carcinoma, colorectal carcinoma, a desmoid tumor, a desmoplastic small round cell tumor, an endocrine tumor, a Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipomachronic lymphocytic leukemia (small lymphocytic lymphoma), B-cell prolymphocytic leukemia, lymphoplasmacytic lymphoma, Waldenström macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, a non-Hodgkin lymphoma, or multiple myeloma. In a particular embodiment, the cancer is multiple myeloma, chronic lymphocytic leukemia, or a non-Hodgkins lymphoma. In a particular embodiment, the cancer is multiple myeloma, chronic lymphocytic leukemia, or a non-Hodgkins lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is multiple myeloma. In a particular embodiment, the multiple myeloma is high-risk multiple myeloma or relapsed and refractory multiple myeloma. In a particular embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a particular embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan, oxaliplatin, platinum, procarbazine, streptozocin, temozolomide, thiotepa, and trabectedin. In a particular embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a particular embodiment, the alkylating agent is cyclophosphamide.

In a particular embodiment, the subject is a human.

In a particular embodiment, the subject undergoes a leukapharesis procedure to collect the PBMCs for the manufacture of the CAR T cells prior to their administration to the subject.

In a particular embodiment, the CAR T cells are administered by an intravenous infusion.

In another aspect, provided herein is a method of manufacturing chimeric antigen receptor (CAR) T cells directed to BCMA (BCMA CAR T cells) from a subject, comprising: (a) administering to the subject an alkylating agent for treatment of a cancer; (b) isolating peripheral blood mononuclear cells (PBMCs) from the subject at least about six months after step (a); and (c) manufacturing BCMA CAR T cells from the PBMCs. In a particular embodiment, step (b) is performed at least about seven (7) months, at least about eight (8) months, at least about nine (9) months, at least about ten (10) months, at least about eleven (11) months, at least about twelve (12) months, at least about thirteen (13) months, or at least about fourteen (14) months after step (a).

In a particular embodiment, the cancer is multiple myeloma, chronic lymphocytic leukemia, or a non-Hodgkins lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is multiple myeloma. In a particular embodiment, the multiple myeloma is high-risk multiple myeloma or relapsed and refractory multiple myeloma. In a particular embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a particular embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan, oxaliplatin, platinum, procarbazine, streptozocin, temozolomide, thiotepa, and trabectedin. In a particular embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a particular embodiment, the alkylating agent is cyclophosphamide.

In a particular embodiment, the subject is a human.

In a particular embodiment, the BCMA CAR T cells comprise a CAR directed to BCMA, wherein the CAR directed to BCMA comprises an antibody or antibody fragment that targets BCMA. In a particular embodiment, the BCMA CAR T cells comprise a CAR directed to BCMA, wherein the CAR directed to BCMA comprises a single chain Fv antibody or antibody fragment (scFv). In a particular embodiment, the BCMA CAR T cells comprise a CAR directed to BCMA, wherein the CAR directed to BCMA comprises a BCMA02 scFv. In a particular embodiment, the BCMA CAR T cells are idecabtagene vicleucel cells.

In a particular embodiment, the subject undergoes a leukapharesis procedure to collect the PBMCs for the manufacture of the BCMA CAR T cells prior to their administration to the subject.

In a particular embodiment, the BCMA CAR T cells are administered by an intravenous infusion.

In another aspect, provided herein is a method of manufacturing chimeric antigen receptor (CAR) T cells directed to BCMA (BCMA CAR T cells) from a subject, comprising: (a) isolating peripheral blood mononuclear cells (PBMCs) from the subject, and determining that at least about 20% of the PBMCs are T cells; and (b) on the basis of the determination in step (a), subsequently manufacturing BCMA CAR T cells from the PBMCs; wherein, prior to step (a), the subject had previously received an alkylating agent for treatment of a cancer. In a specific embodiment, the subject had previously received the alkylating agent at least about six months prior to step (a). In a specific embodiment, the subject had previously received the alkylating agent at least about seven (7) months, at least about eight (8) months, at least about nine (9) months, at least about ten (10) months, at least about eleven (11) months, at least about twelve (12) months, at least about thirteen (13) months, or at least about fourteen (14) months prior to step (a).

In a particular embodiment, the cancer is multiple myeloma, chronic lymphocytic leukemia, or a non-Hodgkins lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is multiple myeloma. In a particular embodiment, the multiple myeloma is high-risk multiple myeloma or relapsed and refractory multiple myeloma. In a particular embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a particular embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan, oxaliplatin, platinum, procarbazine, streptozocin, temozolomide, thiotepa, and trabectedin. In a particular embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a particular embodiment, the alkylating agent is cyclophosphamide.

In a particular embodiment, the subject is a human.

In a particular embodiment, the BCMA CAR T cells comprise a CAR directed to BCMA, wherein the CAR directed to BCMA comprises an antibody or antibody fragment that targets BCMA. In a particular embodiment, the BCMA CAR T cells comprise a CAR directed to BCMA, wherein the CAR directed to BCMA comprises a single chain Fv antibody or antibody fragment (scFv). In a particular embodiment, the BCMA CAR T cells comprise a CAR directed to BCMA, wherein the CAR directed to BCMA comprises a BCMA02 scFv. In a particular embodiment, the BCMA CAR T cells are idecabtagene vicleucel cells.

In a particular embodiment, the subject undergoes a leukapharesis procedure to collect the PBMCs for the manufacture of the BCMA CAR T cells prior to their administration to the subject.

In a particular embodiment, the BCMA CAR T cells are administered by an intravenous infusion.

In another aspect, provided herein is a method of manufacturing chimeric antigen receptor (CAR) T cells directed to BCMA (BCMA CAR T cells) from a subject, comprising: (a) isolating peripheral blood mononuclear cells (PBMCs) from the subject; and (b) manufacturing BCMA CAR T cells from the PBMCs; wherein the subject had previously received an alkylating agent for treatment of a cancer; wherein step (a) occurs at least about six months after the subject received the alkylating agent. In a particular embodiment, step (a) further comprises determining that at least about 20% of the PBMCs are T cells; and wherein step (b) further comprises subsequently manufacturing chimeric antigen receptor (CAR) T cells from the PBMCs on the basis of the determination in step (a). In a particular embodiment, step (a) occurs at least about seven (7) months, at least about eight (8) months, at least about nine (9) months, at least about ten (10) months, at least about eleven (11) months, at least about twelve (12) months, at least about thirteen (13) months, or at least about fourteen (14) months after the subject received the alkylating agent.

In a particular embodiment, the cancer is multiple myeloma, chronic lymphocytic leukemia, or a non-Hodgkins lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is multiple myeloma. In a particular embodiment, the multiple myeloma is high-risk multiple myeloma or relapsed and refractory multiple myeloma. In a particular embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a particular embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan, oxaliplatin, platinum, procarbazine, streptozocin, temozolomide, thiotepa, and trabectedin. In a particular embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a particular embodiment, the alkylating agent is cyclophosphamide.

In a particular embodiment, the subject is a human.

In another aspect, provided herein is a method of manufacturing chimeric antigen receptor (CAR) T cells directed to BCMA (BCMA CAR T cells) from a subject, wherein the subject has been administered an alkylating agent for treatment of a cancer, comprising: a. determining that the subject has not been administered the alkylating agent less than about six (6) months prior to the determining step; b. isolating peripheral blood mononuclear cells (PBMCs) from the subject; and c. manufacturing BCMA CAR T cells from the PBMCs. In a particular embodiment, step (a) occurs at least about seven (7) months, at least about eight (8) months, at least about nine (9) months, at least about ten (10) months, at least about eleven (11) months, at least about twelve (12) months, at least about thirteen (13) months, or at least about fourteen (14) months after the subject received the alkylating agent.

In a particular embodiment, the cancer is multiple myeloma, chronic lymphocytic leukemia, or a non-Hodgkins lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is multiple myeloma. In a particular embodiment, the multiple myeloma is high-risk multiple myeloma or relapsed and refractory multiple myeloma. In a particular embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a particular embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan, oxaliplatin, platinum, procarbazine, streptozocin, temozolomide, thiotepa, and trabectedin. In a particular embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a particular embodiment, the alkylating agent is cyclophosphamide.

In a particular embodiment, the subject is a human.

In a particular embodiment, the BCMA CAR T cells comprise a CAR directed to BCMA, wherein the CAR directed to BCMA comprises an antibody or antibody fragment that targets BCMA. In a particular embodiment, the BCMA CAR T cells comprise a CAR directed to BCMA, wherein the CAR directed to BCMA comprises a single chain Fv antibody or antibody fragment (scFv). In a particular embodiment, the BCMA CAR T cells comprise a CAR directed to BCMA, wherein the CAR directed to BCMA comprises a BCMA02 scFv. In a particular embodiment, the BCMA CAR T cells are idecabtagene vicleucel cells.

In a particular embodiment, the subject undergoes a leukapharesis procedure to collect the PBMCs for the manufacture of the BCMA CAR T cells prior to their administration to the subject.

In a particular embodiment, the BCMA CAR T cells are administered by an intravenous infusion.

In another aspect, provided herein is a method of manufacturing chimeric antigen receptor (CAR) T cells directed to BCMA (c) from a subject, wherein the subject has been administered an alkylating agent for treatment of a cancer, comprising: (a) isolating peripheral blood mononuclear cells (PBMCs) from the subject; and (b) manufacturing BCMA T cells from the PBMCs; wherein, at the time of the isolating, the subject has been determined to have been administered the alkylating agent at least about six (6) months prior. In a particular embodiment, the subject has been determined to have been administered the alkylating agent at least about seven (7) months, at least about eight (8) months, at least about nine (9) months, at least about ten (10) months, at least about eleven (11) months, at least about twelve (12) months, at least about thirteen (13) months, or at least about fourteen (14) months prior.

In a particular embodiment, the cancer is multiple myeloma, chronic lymphocytic leukemia, or a non-Hodgkins lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is multiple myeloma. In a particular embodiment, the multiple myeloma is high-risk multiple myeloma or relapsed and refractory multiple myeloma. In a particular embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a particular embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan, oxaliplatin, platinum, procarbazine, streptozocin, temozolomide, thiotepa, and trabectedin. In a particular embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a particular embodiment, the alkylating agent is cyclophosphamide.

In a particular embodiment, the subject is a human.

In another aspect, provided herein is a method of manufacturing chimeric antigen receptor (CAR) T cells directed to BCMA (BCMA CAR T cells) from a subject, comprising: (a) isolating peripheral blood mononuclear cells (PBMCs) from the subject; and determining that at least about 20% of the PBMCs are T cells; and (b) on the basis of the determination in step (a), subsequently manufacturing the BCMA CAR T cells from the PBMCs; wherein the subject had previously received an alkylating agent for treatment of a cancer caused by BCMA-expressing cells. In a particular embodiment, the subject had previously received the alkylating agent at least about six months prior to step (a). In a particular embodiment, the subject had previously received the alkylating agent at least about seven (7) months, at least about eight (8) months, at least about nine (9) months, at least about ten (10) months, at least about eleven (11) months, at least about twelve (12) months, at least about thirteen (13) months, or at least about fourteen (14) months prior to step (a).

In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are T cells.

In a particular embodiment of the methods presented herein, the T cells comprise CD3+ cells. In particular embodiments, the T cells are CD3+ cells. In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T CD3+ T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are CD3+ T cells, e.g., CD45+/CD3+ T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are CD3+ T cells.

In a particular embodiment, the T cells are CD8+ effector memory RA (T_{EMRA}) (CCR7−/CD45RA+) T cells. In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T_{EMRA} T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are T_{EMRA} T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are T_{EMRA} T cells. In particular embodiments of such methods, the T cells are CD3+ cells.

In the methods presented herein, the determining may be performed using standard techniques well known to those of skill in the relevant art. For example, in the methods presented herein, the determining step may be performed by utilizing techniques such as those utilized in Example 2 (e.g., immunophenotyping of the PBMCs by polychromatic flow cytometry for markers associated with T cell differentiation, memory, senescence, and exhaustion).

In a particular embodiment, the cancer is multiple myeloma, chronic lymphocytic leukemia, or a non-Hodgkins lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is multiple myeloma. In a particular embodiment, the multiple myeloma is high-risk multiple myeloma or relapsed and refractory multiple myeloma. In a particular embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a particular embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan, oxaliplatin, platinum, procarbazine, streptozocin, temozolomide, thiotepa, and trabectedin. In a particular embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a particular embodiment, the alkylating agent is cyclophosphamide.

In a particular embodiment, the subject is a human.

In a particular embodiment, the BCMA CAR T cells comprise a CAR directed to BCMA, wherein the CAR directed to BCMA comprises an antibody or antibody fragment that targets BCMA. In a particular embodiment, the BCMA CAR T cells comprise a CAR directed to BCMA, wherein the CAR directed to BCMA comprises a single chain Fv antibody or antibody fragment (scFv). In a particular embodiment, the BCMA CAR T cells comprise a CAR directed to BCMA, wherein the CAR directed to BCMA comprises a BCMA02 scFv. In a particular embodiment, the BCMA CAR T cells are idecabtagene vicleucel cells.

In a particular embodiment, the subject undergoes a leukapharesis procedure to collect the PBMCs for the manufacture of the BCMA CAR T cells prior to their administration to the subject.

In a particular embodiment, the BCMA CAR T cells are administered by an intravenous infusion.

In another aspect, provided herein is a method of determining whether a subject having cancer can be treated with a CAR T therapy, comprising determining the percentage of CD3+ T cells in the subject relative to the number of PMBCs, wherein, (i) if the subject has less than about 20% CD3+ T cells relative to the number of PBMCs, not administering the CAR T therapy to the subject, and (ii) if the subject has more than about 20% CD3+ T cells relative to the number of PBMCs, then administering the CAR T therapy to the subject. In a particular embodiment, the CD3+ T cells are CD45+/CD3+ T cells.

In another aspect, provided herein is a method of treating a cancer in a subject, comprising having determined that the percentage of CD3+ T cells in the subject relative to the number of PMBCs is higher than about 20%, and administering a CAR T therapy to the subject. In a particular embodiment, the CD3+ T cells are CD45+/CD3+ T cells.

Generally, the TTE model described herein informs the time between an event and a prior event, such as the time (i.e., washout time) between a therapy and a prior exposure, e.g., a prior therapy, provided that timing data pertaining to the event and the prior event are available.

In an interrelated aspect, an optimal washout period for commencing a therapy for the treatment of a condition in a subject after a prior exposure can be determined by receiving, for each of a plurality of subjects, prior treatment history data. Left-censored data can then be derived from the prior treatment history data for each of the subjects that includes a washout period and event or censor. A time scale of the left-censored treatment data is then inverted to result in right-censored treatment data. The right-censored treatment data is then applied to a time-to-event (TTE) model that associates one or more variables of interest with a time since exposure to the prior exposure. A maximally selected log-rank statistic across a plurality of cutoffs within a pre-defined percentile range is computed for continuous variables within the one or more variables of interest. One or more variables and associated cutoffs for the continuous variables having a maximally selected log-rank statistic below a first pre-defined threshold are then identified. A test statistic (e.g., a Cox proportional-hazards statistic, etc.) of each (n−1) strata relative to a reference stratum is then computed for ordinal or categorical variables within the one or more variables of interest. One or more ordinary or categorical variables and associated strata having a test statistic (e.g., a Cox proportional-hazards statistic, etc.) below a second pre-defined threshold, relative to the reference stratum are identified. An optimal washout period is then determined for the therapy based on the cutoff having a lowest value below the pre-defined threshold and relative to a median of subject values below the pre-defined threshold and a median of subject values above the pre-defined threshold.

The determined washout period can be provided. Provided, in this regard, can include one or more of causing the determined optimal washout period to be displayed in an electronic visual display, storing the determined optimal washout period in physical persistence, loading the determined optimal washout period into memory, or transmitting the determined optimal washout period over a network to a remote computing device.

The TTE model can be a proportional-hazards model such as a Cox proportional-hazards model.

One or more of the receiving, deriving, inverting, applying, first or second computing, first or second identifying, and determining can be executed by at least one data processor forming part of at least one computing device.

The prior exposure can be a prior therapy.

The prior exposure and the therapy can be different types of therapies.

The prior exposure can be for treating a condition that is different from the condition treated with the therapy.

The prior exposure can be for treating a condition that is the same as the condition treated with the therapy.

The prior exposure and the therapy can be for treating the same condition and are the same type of therapy.

The therapy can be a second or later (e.g., 3^{rd}, 4^{th}, 5^{th} 6^{th} 7^{th} or later) line of therapy and the prior exposure is an earlier line of therapy.

The information provided by the TTE model can inform (i) the time that a subject should receive the therapy after having had the prior exposure or (ii) exclusion criteria in a clinical trial.

The therapy and/or the prior exposure can be radiotherapy, chemotherapy, immunotherapy, surgery, a transplant, gene therapy or cell therapy.

The condition can be cancer, an immune disease (e.g., an autoimmune disease), a cardiovascular disease, fibrosis, an infectious disease or a neurological condition.

The condition can be a condition that can be treated by stimulating the immune system, e.g., cancer and infectious diseases, and the therapy is a therapy that stimulates or enhances the immune system, e.g., immunotherapy and cell therapy.

The condition can be cancer and the therapy can be cell therapy, e.g., CAR T.

The prior exposure can be a prior therapy, and the prior therapy can be a prior cancer treatment.

The cancer can be multiple myeloma, the treatment can be a BCMA CAR T (e.g., a CAR comprising SEQ ID NO: 37 or a nucleic acid encoding a CAR of SEQ ID NO: 9 or 37), and the prior treatment can be a prior cancer treatment for multiple myeloma.

The prior exposure can be an alkylator therapy.

In some variations, the prior exposure is not a prior therapy.

The prior exposure can be an event that can negatively impact the therapy.

The prior exposure can be a prior condition.

The prior exposure can be an inflammatory condition or an infectious disease (e.g., a viral infection, such as COVID-19 infection).

In an interrelated aspect, a condition in a subject can be treated by administering to the subject a therapy for treating the condition, wherein the therapy is administered after a prior exposure, and wherein the time to administer the therapy after the prior exposure (i.e., the washout period) was calculated using a method as provided herein.

In a further interrelated aspect, a condition in a subject can be treated by administering to the subject a therapy for treating the condition, wherein the therapy is administered after a prior exposure, at a time after the prior exposure that was determined using a method as provided herein.

In still a further interrelated aspect, a system can include at least one data processor and memory storing instructions which, when executed by the at least one data processor, implement a method as provided herein.

In yet a further interrelated aspect, a non-transitory computer program product can stored instructions which, when executed by at least one computing device, implement a method as provided herein.

Generally, the TTE model described herein informs the time between an event and a prior event, such as the time (i.e., washout time) between a therapy and a prior exposure, e.g., a prior therapy, provided that timing data pertaining to the event and the prior event are available.

Aspects of the current subject matter can be embodied in non-transitory computer program products (i.e., physically embodied computer program products) that store instructions, which when executed by one or more data processors of one or more computing systems, cause at least one data processor to perform operations herein. Similarly, the current subject matter can be embodied in computer systems that include one or more data processors and memory coupled to the one or more data processors. The memory may temporarily or permanently store instructions that cause at least one processor to perform one or more of the operations described herein. In addition, methods can be implemented by one or more data processors either within a single computing system or distributed among two or more computing systems. Such computing systems can be connected and can exchange data and/or commands or other instructions or the like via one or more connections, including but not limited to a connection over a network (e.g., the Internet, a wireless wide area network, a local area network, a wide area network, a wired network, or the like), via a direct connection between one or more of the multiple computing systems, etc.

### 4. BRIEF DESCRIPTION OF THE DRAWINGS

FIG. 1 shows a schematic of a B cell maturation antigen (BCMA) CAR construct (anti-BCMA02 CAR).

FIGS. 2A and 2B show a correlative association between alkylator washout and CD3+ cells in peripheral blood mononuclear cell (PBMC) material. FIG. 2A shows a novel left-censored time-to-event model, in which time is inverted (i.e., day zero (0) is the day of apheresis; the curves are read from right to left; moving from the day of apheresis (i.e., day zero (0)) to the right, time is presented moving backwards into the past). The table providing “Number of Subjects” tabulates the number of patients with exposure to alkylating agents at each time interval. “Fraction Not Exposed” (on Y axis) refers to the proportion of patients not yet exposed to alkylating agents at the time indicated on the x-axis. “Above” refers to the subset of patients with ≥20% T cell content in their PBMCs isolated from apheresis material (and used as input for CAR T manufacturing); similarly, “Below” refers to the subset of patients with <20% T cell content in their PBMCs. The 20% cutoff was identified by a statistical procedure that maximizes the difference between the time-since-last-exposure curves and the p-value is adjusted to account for testing multiple cutoffs during the optimization. FIG. 2B shows Spearman's coefficient using an encoded washout. Time-since-last-exposure is transformed as 1/log(−1*days-since-last-exposure), providing a continuum that spans from 0 to −1 (−1 indicates perfectly recent exposure, 0 indicates never exposed).

FIG. 3 depicts the general protocol by which ide-cel CAR T cells were manufactured and infused into relapsed and refractory multiple myeloma (RRMM) patients, who were subsequently assessed for response to ide-cel therapy (Cy, cyclophosphamide; Flu, fludarabine; RRMM, relapsed and refractory multiple myeloma; PD, progressive disease; IMiD, immunomodulatory drug; PI, proteasome inhibitor; IMWG, International Myeloma Working Group; MM, multiple myeloma; ^{a}Defined as documented disease progression during or within 60 days from last dose of prior antimyeloma regimen; ^{b}Patients were required to be hospitalized for 14 days post-infusion, and ide-cel re-treatment was allowed at disease progression for best response of at least stable disease).

FIGS. 4A and 4B show the results of an analysis of prior alkylating agent exposure in patients undergoing CAR T cell therapy. FIG. 4A shows the percentage of patients relative to number of prior anti-myeloma regimens (top graph) and the percentage of patients relative to the average number of prior regimens per year (number of prior therapies/time since diagnosis) (bottom graph). FIG. 4B shows the percentage of patients relative to time since last alkylating agent exposure, wherein the time is indicated in months.

FIG. 5 depicts the general protocol for screening of patients for 43 variables prior to apheresis, in which PBMC's were isolated from patients for manufacturing the CAR T cell drug product (LDC, lymphodepleting chemotherapy; PBMC, peripheral blood mononuclear cell).

FIG. 6 shows a novel time-to-event model for estimating the effects of prior therapy exposure (HR, hazard ratio).

FIGS. 7A and 7B show results from an analysis of patient features associated with alkylating agent exposure. FIG. 7A shows a time-since-last-exposure model (i.e., alkylating agent exposure) for sBCMA (ng/mL), ferritin (ng/mL), BMI (kg/m²), and age (years) (Soluble factors from blood: sBCMA (soluble B-cell maturation antigen); blood chemistries: ferritin; patient: BMI (body-mass index), age; HR, hazard ratio). FIG. 7B shows the correlation with prior regimens; in particular, the ferritin at baseline (ng/mL) relative to the average number of prior regiments per year (top graph) and BMI at baseline (kg/m²) relative to the average number of prior regiments per year (bottom graph).

FIGS. 8A and 8B show results from an analysis of immune factors associated with alkylating agent exposure. FIG. 8A shows a time-since-last-exposure model (i.e., alkylating agent exposure) for granzyme B, IL-7, and CD3+ cells (soluble factors from blood: granzyme B, IL-7; PBMCs from apheresis: CD3+; HR, hazard ratio; IL, interleukin; PBMC, peripheral blood mononuclear cell). FIG. 8B shows enrichment for low T cell content in recently exposed patient subsets (^{a}The 20% threshold was the optimal cutpoint determined by the maximum statistical algorithm).

FIGS. 9A and 9B show that T cell depletion was detectable ≥6 months after last exposure to an alkylating agent. FIG. 9A shows CD45+/CD3+ T cell content by exposure bin (PBMC, peripheral blood mononuclear cell). FIG. 9B shows the correlation of T cell content in PBMCs with prior regimens (i.e., CD45+/CD3+ PBMCs, % relative to average number of prior regimens per year).

FIGS. 10A and 10B show T cell memory phenotypes associated with alkylating agent exposure. FIG. 10A shows a time-since-last-exposure model (i.e., alkylating agent exposure) for CD8+ intermediate, CD8+ T_{EMRA}, and CD8+ TEM cells. FIG. 10B shows the correlation with prior regimens; in particular, the T_{EMRA} PBMCs, % of CD8+ relative to the average number of prior regimens per year (top graph) and TEM PBMCs, % of CD8+ relative to the average number of prior regimens per year (bottom graph) (PBMCs from apheresis: CD8+ TEM, T_{EMRA}, and intermediate (CD28+/CD27−), CD8+ intermediate: CD3+/CD8+/CD28+/CD27−; HR, hazard ratio; PBMC, peripheral blood mononuclear cell; TEM, effector memory T cell; T_{EMRA}, effector memory RA T cell).

FIG. 11 is a process flow diagram illustrating treatment regimen determination using a time-to-event model.

FIG. 12 is a diagram illustrating a computing device for implementing aspects of the current subject matter.

### 5. BRIEF DESCRIPTION OF THE SEQUENCE IDENTIFIERS

SEQ ID NOs: 1-3 set forth amino acid sequences of exemplary light chain CDR sequences for BCMA CARs contemplated herein.

SEQ ID NOs: 4-6 set forth amino acid sequences of exemplary heavy chain CDR sequences for BCMA CARs contemplated herein.

SEQ ID NO: 7 sets forth an amino acid sequence of an exemplary light chain sequence for BCMA CARs contemplated herein.

SEQ ID NO: 8 sets forth an amino acid sequence of an exemplary heavy chain sequence for BCMA CARs contemplated herein.

SEQ ID NO: 9 sets forth an amino acid sequence of exemplary BCMA CAR contemplated herein, with a signal peptide (amino acids 1-22). The amino acid sequence of the mature form of BCMA02 is set forth in SEQ ID NO: 37.

SEQ ID NO: 10 sets forth a polynucleotide sequence that encodes an exemplary BCMA CAR contemplated herein.

SEQ ID NO: 11 sets forth the amino acid sequence of human BCMA.

SEQ ID NO: 12-22 set forth the amino acid sequences of various linkers.

SEQ ID NOs: 23-35 set forth the amino acid sequences of protease cleavage sites and self-cleaving polypeptide cleavage sites.

SEQ ID NO: 36 sets forth the polynucleotide sequence of a vector encoding an exemplary BCMA CAR. See Table 1.

SEQ ID NO: 37 sets forth an amino acid sequence of exemplary mature BCMA CAR contemplated herein (i.e., without the signal sequence).

SEQ ID NO: 38 sets forth an amino acid sequence of BCMA02 scFv.

TABLE 1
Listing of Sequences:

| SEQ ID NO. | Sequence |
|---|---|
| 1 | RASESVTILGSHLIH |
| 2 | LASNVQT |
| 3 | LQSRTIPRT |
| 4 | DYSIN |
| 5 | WINTETREPAYAYDFRG |
| 6 | DYSYAMDY |
| 7 | DIVLTQSPPSLAMSLGKRATISCRASESVTILGSHLIHWYQQKPGQ |
| | PPTLLIQLASNVQTGVPARFSGSGSRTDFTLTIDPVEEDDVAVYYC |
| | LQSRTIPRTFGGGTKLEIK |
| 8 | QIQLVQSGPELKKPGETVKISCKASGYTFTDYSINWVKRAPGKGL |
| | KWMGWINTETREPAYAYDFRGRFAFSLETSASTAYLQINNLKYE |
| | DTATYFCALDYSYAMDYWGQGTSVTVSS |
| 9 | MALPVTALLLPLALLLHAARPDIVLTQSPPSLAMSLGKRATISCR |
| | ASESVTILGSHLIHWYQQKPGQPPTLLIQLASNVQTGVPARFSGSG |
| | SRTDFTLTIDPVEEDDVAVYYCLQSRTIPRTFGGGTKLEIKGSTSG |
| | SGKPGSGEGSTKGQIQLVQSGPELKKPGETVKISCKASGYTFTDY |
| | SINWVKRAPGKGLKWMGWINTETREPAYAYDFRGRFAFSLETSA |
| | STAYLQINNLKYEDTATYFCALDYSYAMDYWGQGTSVTVSSAA |
| | ATTTPAPRPPTPAPTIASQPLSLRPEACRPAAGGAVHTRGLDFACD |
| | IYIWAPLAGTCGVLLLSLVITLYCKRGRKKLLYIFKQPFMRPVQTT |
| | QEEDGCCRFPEEEEGGCELRVKFSRSADAPAYQQGQNQLYNEL |
| | NLGRREEYDVLDKRRGRDPEMGGKPRRKNPQEGLYNELQKDK |
| | MAEAYSEIGMKGERRRGKGHDGLYQGLSTATKDTYDALHMQA |
| | LPPR |
| 10 | atggcactccccgtcaccgcccttctcttgcccctcgccctgctgctgcatgctgccaggcccgacattg |
| | tgctcactcagtcacctcccagcctggccatgagcctgggaaaaagggccaccatctcctgtagagcc |
| | agtgagtccgtcacaatcttggggagccatcttattcactggtatcagcagaagcccgggcagcctcca |
| | acccttcttattcagctcgcgtcaaacgtccagacgggtgtacctgccagattttctggtagcgggtcccg |
| | cactgattttacactgaccatagatccagtggaagaagacgatgtggccgtgtattattgtctgcagagca |
| | gaacgattcctcgcacatttggtgggggtactaagctggagattaagggaagcacgtccggctcaggg |
| | aagccgggctccggcgagggaagcacgaaggggcaaattcagctggtccagagcggacctgagct |
| | gaaaaaacccggcgagactgttaagatcagttgtaaagcatctggctataccttcaccgactacagcata |
| | aattgggtgaaacgggcccctggaaagggcctcaaatggatgggttggatcaataccgaaactaggg |
| | agcctgcttatgcatatgacttccgcgggagattcgccttttcactcgagacatctgcctctactgcttacct |
| | ccaaataaacaacctcaagtatgaagatacagccacttacttttgcgccctcgactatagttacgccatgg |
| | actactggggacagggaacctccgttaccgtcagttccgcggccgcaaccacaacacctgctccaag |
| | gccccccacacccgctccaactatagccagccaaccattgagcctcagacctgaagcttgcaggcccg |
| | cagcaggaggcgccgtccatacgcgaggcctggacttcgcgtgtgatatttatatttgggcccctttggc |
| | cggaacatgtggggtgttgcttctctcccttgtgatcactctgtattgtaagcgcgggagaaagaagctcc |
| | tgtacatcttcaagcagccttttatgcgacctgtgcaaaccactcaggaagaagatgggtgttcatgccg |
| | cttccccgaggaggaagaaggagggtgtgaactgagggtgaaattttctagaagcgccgatgctcccg |
| | catatcagcagggtcagaatcagctctacaatgaattgaatctcggcaggcgagaagagtacgatgttct |
| | ggacaagagacggggcagggatcccgagatggggggaaagccccggagaaaaaatcctcaggag |
| | gggttgtacaatgagctgcagaaggacaagatggctgaagcctatagcgagatcggaatgaaaggcg |
| | aaagacgcagaggcaaggggcatgacggtctgtaccagggtctctctacagccaccaaggacacttat |
| | gatgcgttgcatatgcaagccttgccaccccgctaatga |
| 11 | MLQMAGQCSQNEYFDSLLHACIPCQLRCSSNTPPLTCQRYCNAS |
| | VTNSVKGTNAILWTCLGLSLIISLAVFVLMFLLRKINSEPLKDEFK |
| | NTGSGLLGMANIDLEKSRTGDEIILPRGLEYTVEECTCEDCIKSKP |
| | KVDSDHCFPLPAMEEGATILVTTKTNDYCKSLPAALSATEIEKSIS |
| | AR |
| 12 | DGGGS |
| 13 | TGEKP |
| 14 | GGRR |
| 15 | GGGGS |
| 16 | EGKSSGSGSESKVD |
| 17 | KESGSVSSEQLAQFRSLD |
| 18 | GGRRGGGS |
| 19 | LRQRDGERP |
| 20 | LRQKDGGGSERP |
| 21 | LRQKDGGGSGGGSERP |
| 22 | GSTSGSGKPGSGEGSTKG |
| 23 | EX₁X₂YX₃QX₄ |
| | X₁ is Any amino acid |
| | X₂ is Any amino acid |
| | X₃ is Any amino acid |
| | X₄ is Gly or Ser |
| 24 | ENLYFQG |
| 25 | ENLYFQS |
| 26 | LLNFDLLKLAGDVESNPGP |
| 27 | TLNFDLLKLAGDVESNPGP |
| 28 | LLKLAGDVESNPGP |
| 29 | NFDLLKLAGDVESNPGP |
| 30 | QLLNFDLLKLAGDVESNPGP |
| 31 | APVKQTLNFDLLKLAGDVESNPGP |
| 32 | VTELLYRMKRAETYCPRPLLAIHPTEARHKQKIVAPVKQT |
| 33 | LNFDLLKLAGDVESNPGP |
| 34 | LLAIHPTEARHKQKIVAPVKQTLNFDLLKLAGDVESNPGP |
| 35 | EARHKQKIVAPVKQTLNFDLLKLAGDVESNPGP |
| 36 | tcgcgcgtttcggtgatgacggtgaaaacctctgacacatgcagctcccggagacggtcacagcttgtc |
| | tgtaagcggatgccgggagcagacaagcccgtcagggcgcgtcagcgggtgttggcgggtgtcggg |
| | gctggcttaactatgcggcatcagagcagattgtactgagagtgcaccatcatatgccagcctatggtga |
| | cattgattattgactagttattaatagtaatcaattacggggtcattagttcatagcccatatatggagttccg |
| | cgttacataacttacggtaaatggcccgcctggctgaccgcccaacgacccccgcccattgacgtcaat |
| | aatgacgtatgttcccatagtaacgccaatagggactttccattgacgtcaatgggggagtatttacggt |
| | aaactgcccacttggcagtacatcaagtgtatcatatgccaagtacgccccctattgacgtcaatgacgg |
| | taaatggcccgcctggcattatgcccagtacatgaccttatgggactttcctacttggcagtacatctacgt |
| | attagtcatcgctattaccatggtgatgcggttttggcagtacatcaatgggcgtggatagcggtttgactc |
| | acggggatttccaagtctccaccccattgacgtcaatgggagtttgttttggcaccaaaatcaacgggac |
| | tttccaaaatgtcgtaacaactccgccccattgacgcaaatgggcggtaggcgtgtacggtgggaggtc |
| | tatataagcagagctcgtttagtgaaccgggtctctctggttagaccagatctgagcctgggagctctctg |
| | gctaactagggaacccactgcttaagcctcaataaagcttgccttgagtgctcaaagtagtgtgtgcccg |
| | tctgttgtgtgactctggtaactagagatccctcagacccttttagtcagtgtggaaaatctctagcagtgg |
| | cgcccgaacagggacttgaaagcgaaagtaaagccagaggagatctctcgacgcaggactcggcttg |
| | ctgaagcgcgcacggcaagaggcgaggggcggcgactggtgagtacgccaaaaattttgactagcg |
| | gaggctagaaggagagagtagggtgcgagagcgtcggtattaagcgggggagaattagataaatgg |
| | gaaaaaattcggttaaggccagggggaaagaaacaatataaactaaaacatatagttagggcaagcag |
| | ggagctagaacgattcgcagttaatcctggccttttagagacatcagaaggctgtagacaaatactggg |
| | acagctacaaccatcccttcagacaggatcagaagaacttagatcattatataatacaatagcagtcctct |
| | attgtgtgcatcaaaggatagatgtaaaagacaccaaggaagccttagataagatagaggaagagcaa |
| | aacaaaagtaagaaaaaggcacagcaagcagcagctgacacaggaaacaacagccaggtcagcca |
| | aaattaccctatagtgcagaacctccaggggcaaatggtacatcaggccatatcacctagaactttaaatt |
| | aagacagcagtacaaatggcagtattcatccacaattttaaaagaaaaggggggattggggggtacagt |
| | gcaggggaaagaatagtagacataatagcaacagacatacaaactaaagaattacaaaaacaaattac |
| | aaaaattcaaaattttcgggtttattacagggacagcagagatccagtttggaaaggaccagcaaagctc |
| | ctctggaaaggtgaaggggcagtagtaatacaagataatagtgacataaaagtagtgccaagaagaaa |
| | agcaaagatcatcagggattatggaaaacagatggcaggtgatgattgtgtggcaagtagacaggatg |
| | aggattaacacatggaaaagattagtaaaacaccatagctctagagcgatcccgatcttcagacctgga |
| | ggaggagatatgagggacaattggagaagtgaattatataaatataaagtagtaaaaattgaaccattag |
| | gagtagcacccaccaaggcaaagagaagagtggtgcagagagaaaaaagagcagtgggaatagga |
| | gctttgttccttgggttcttgggagcagcaggaagcactatgggcgcagcgtcaatgacgctgacggta |
| | caggccagacaattattgtctggtatagtgcagcagcagaacaatttgctgagggctattgaggcgcaa |
| | cagcatctgttgcaactcacagtctggggcatcaagcagctccaggcaagaatcctggctgtggaaag |
| | atacctaaaggatcaacagctcctggggatttggggttgctctggaaaactcatttgcaccactgctgtgc |
| | cttggaatgctagttggagtaataaatctctggaacagatttggaatcacacgacctggatggagtggga |
| | cagagaaattaacaattacacaagcttggtaggtttaagaatagtttttgctgtactttctatagtgaataga |
| | gttaggcagggatattcaccattatcgtttcagacccacctcccaaccccgaggggacccgacaggcc |
| | cgaaggaatagaagaagaaggtggagagagagacagagacagatccattcgattagtgaacggatc |
| | catctcgacggaatgaaagaccccacctgtaggtttggcaagctaggatcaaggttaggaacagagag |
| | acagcagaatatgggccaaacaggatatctgtggtaagcagttcctgccccggctcagggccaagaac |
| | agttggaacagcagaatatgggccaaacaggatatctgtggtaagcagttcctgccccggctcagggc |
| | caagaacagatggtccccagatgcggtcccgccctcagcagtttctagagaaccatcagatgtttccag |
| | ggtgccccaaggacctgaaatgaccctgtgccttatttgaactaaccaatcagttcgcttctcgcttctgtt |
| | cgcgcgcttctgctccccgagctcaataaaagagcccacaacccctcacteggcgcgattcacctgac |
| | gcgtctacgccaccatggcactccccgtcaccgcccttctcttgcccctcgccctgctgctgcatgctgc |
| | caggcccgacattgtgctcactcagtcacctcccagcctggccatgagcctgggaaaaagggccacc |
| | atctcctgtagagccagtgagtccgtcacaatcttggggagccatcttattcactggtatcagcagaagc |
| | ccgggcagcctccaacccttcttattcagctcgcgtcaaacgtccagacgggtgtacctgccagattttct |
| | ggtagcgggtcccgcactgattttacactgaccatagatccagtggaagaagacgatgtggccgtgtatt |
| | attgtctgcagagcagaacgattcctcgcacatttggtgggggtactaagctggagattaagggaagca |
| | cgtccggctcagggaagccgggctccggcgagggaagcacgaaggggcaaattcagctggtccag |
| | agcggacctgagctgaaaaaacccggcgagactgttaagatcagttgtaaagcatctggctataccttc |
| | accgactacagcataaattgggtgaaacgggcccctggaaagggcctcaaatggatgggttggatcaa |
| | taccgaaactagggagcctgcttatgcatatgacttccgcgggagattcgccttttcactcgagacatctg |
| | cctctactgcttacctccaaataaacaacctcaagtatgaagatacagccacttacttttgegccctcgact |
| | atagttacgccatggactactggggacagggaacctccgttaccgtcagttccgcggccgcaaccaca |
| | acacctgctccaaggccccccacacccgctccaactatagccagccaaccattgagcctcagacctga |
| | agcttgcaggcccgcagcaggaggcgccgtccatacgcgaggcctggacttcgcgtgtgatatttatat |
| | ttgggcccctttggccggaacatgtggggtgttgcttctctcccttgtgatcactctgtattgtaagcgcgg |
| | gagaaagaagctcctgtacatcttcaagcagccttttatgcgacctgtgcaaaccactcaggaagaagat |
| | gggtgttcatgccgcttccccgaggaggaagaaggagggtgtgaactgagggtgaaattttctagaag |
| | cgccgatgctcccgcatatcagcagggtcagaatcagctctacaatgaattgaatctcggcaggcgag |
| | aagagtacgatgttctggacaagagacggggcagggatcccgagatggggggaaagccccggaga |
| | aaaaatcctcaggaggggttgtacaatgagctgcagaaggacaagatggctgaagcctatagcgagat |
| | cggaatgaaaggcgaaagacgcagaggcaaggggcatgacggtctgtaccagggtctctctacagc |
| | caccaaggacacttatgatgcgttgcatatgcaagccttgccaccccgctaatgacaggtacctttaaga |
| | ccaatgacttacaaggcagctgtagatcttagccactttttaaaagaaaaggggggactggaagggcta |
| | attcactcccaaagaagacaagatctgctttttgcctgtactgggtctctctggttagaccagatctgagcc |
| | tgggagctctctggctaactagggaacccactgcttaagcctcaataaagcttgccttgagtgcttcaatg |
| | tgtgtgttggttttttgtgtgtcgaaattctagcgattctagcttggcgtaatcatggtcatagctgtttcctgtg |
| | tgaaattgttatccgctcacaattccacacaacatacgagccggaagcataaagtgtaaagcctggggt |
| | gcctaatgagtgagctaactcacattaattgcgttgcgctcactgcccgctttccagtcgggaaacctgtc |
| | gtgccagctgcattaatgaatcggccaacgcgcggggagaggcggtttgcgtattgggcgctcttccg |
| | cttcctcgctcactgactcgctgcgctcggtcgttcggctgcggcgagcggtatcagctcactcaaagg |
| | cggtaatacggttatccacagaatcaggggataacgcaggaaagaacatgtgagcaaaaggccagca |
| | aaaggccaggaaccgtaaaaaggccgcgttgctggcgtttttccataggctccgcccccctgacgagc |
| | atcacaaaaatcgacgctcaagtcagaggtggcgaaacccgacaggactataaagataccaggcgttt |
| | ccccctggaagctccctcgtgcgctctcctgttccgaccctgccgcttaccggatacctgtccgcctttct |
| | cccttcgggaagcgtggcgctttctcatagctcacgctgtaggtatctcagttcggtgtaggtcgttcgct |
| | ccaagctgggctgtgtgcacgaaccccccgttcagcccgaccgctgcgccttatccggtaactatcgtc |
| | ttgagtccaacccggtaagacacgacttatcgccactggcagcagccactggtaacaggattagcaga |
| | gcgaggtatgtaggcggtgctacagagttcttgaagtggtggcctaactacggctacactagaagaaca |
| | gtatttggtatctgcgctctgctgaagccagttaccttcggaaaaagagttggtagctcttgatccggcaa |
| | acaaaccaccgctggtagcggtggtttttttgtttgcaagcagcagattacgcgcagaaaaaaaggatct |
| | caagaagatcctttgatcttttctacggggtctgacgctcagtggaacgaaaactcacgttaagggatttt |
| | ggtcatgagattatcaaaaaggatcttcacctagatccttttaaattaaaaatgaagttttaaatcaatctaaa |
| | gtatatatgagtaaacttggtctgacagttaccaatgcttaatcagtgaggcacctatctcagcgatctgtct |
| | atttcgttcatccatagttgcctgactccccgtcgtgtagataactacgatacgggagggcttaccatctgg |
| | ccccagtgctgcaatgataccgcgagacccacgctcaccggctccagatttatcagcaataaaccagc |
| | cagccggaagggccgagcgcagaagtggtcctgcaactttatccgcctccatccagtctattaattgttg |
| | ccgggaagctagagtaagtagttcgccagttaatagtttgcgcaacgttgttgccattgctacaggcatc |
| | gtggtgtcacgctcgtcgtttggtatggcttcattcagctccggttcccaacgatcaaggcgagttacatg |
| | atcccccatgttgtgcaaaaaagcggttagctccttcggtcctccgatcgttgtcagaagtaagttggccg |
| | cagtgttatcactcatggttatggcagcactgcataattctcttactgtcatgccatccgtaagatgcttttct |
| | gtgactggtgagtactcaaccaagtcattctgagaatagtgtatgcggcgaccgagttgctcttgcccgg |
| | cgtcaatacgggataataccgcgccacatagcagaactttaaaagtgctcatcattggaaaacgttcttc |
| | ggggcgaaaactctcaaggatcttaccgctgttgagatccagttcgatgtaacccactogtgcacccaa |
| | ctgatcttcagcatcttttactttcaccagcgtttctgggtgagcaaaaacaggaaggcaaaatgccgcaa |
| | aaaagggaataagggcgacacggaaatgttgaatactcatactcttcctttttcaatattattgaagcattta |
| | tcagggttattgtctcatgagcggatacatatttgaatgtatttagaaaaataaacaaataggggttccgcg |
| | cacatttccccgaaaagtgccacctgggactagctttttgcaaaagcctaggcctccaaaaaagcctcct |
| | cactacttctggaatagctcagaggccgaggcggcctcggcctctgcataaataaaaaaaattagtcag |
| | ccatggggggagaatgggcggaactgggcggagttaggggcgggatgggcggagttaggggcg |
| | ggactatggttgctgactaattgagatgagcttgcatgccgacattgattattgactagtccctaagaaacc |
| | attcttatcatgacattaacctataaaaataggcgtatcacgaggccctttcgtc |
| 37 | DIVLTQSPPSLAMSLGKRATISCRASESVTILGSHLIHWYQQKPGQ |
| | PPTLLIQLASNVQTGVPARFSGSGSRTDFTLTIDPVEEDDVAVYYC |
| | LQSRTIPRTFGGGTKLEIKGSTSGSGKPGSGEGSTKGQIQL VQSGP |
| | ELKKPGETVKISCKASGYTFTDYSINWVKRAPGKGLKWMGWINT |
| | ETREPAYAYDFRGRFAFSLETSASTAYLQINNLKYEDTATYFCAL |
| | DYSYAMDYWGQGTSVTVSSAAATTTPAPRPPTPAPTIASQPLSLR |
| | PEACRPAAGGAVHTRGLDFACDIYIWAPLAGTCGVLLLSLVITLY |
| | CKRGRKKLLYIFKQPFMRPVQTTQEEDGCSCRFPEEEEGGCELRV |
| | KFSRSADAPAYQQGQNQLYNELNLGRREEYDVLDKRRGRDPEM |
| | GGKPRRKNPQEGLYNELQKDKMAEAYSEIGMKGERRRGKGHDG |
| | LYQGLSTATKDTYDALHMQALPPR |
| 38 | DIVLTQSPPSLAMSLGKRATISCRASESVTILGSHLIHWYQQKPGQ |
| | PPTLLIQLASNVQTGVPARFSGSGSRTDFTLTIDPVEEDDVAVYYC |
| | LQSRTIPRTFGGGTKLEIKGSTSGSGKPGSGEGSTKGQIQLVQSGP |
| | ELKKPGETVKISCKASGYTFTDYSINWVKRAPGKGLKWMGWINT |
| | ETREPAYAYDFRGRFAFSLETSASTAYLQINNLKYEDTATYFCAL |
| | DYSYAMDYWGQGTSVTVSS |

### 6. DETAILED DESCRIPTION

### 6.1. Methods for Treating a Tumor or a Cancer Using Chimeric Antigen Receptor (Car) T Cells and Methods of Manufacturing Car T Cells

The disclosure presented herein generally relates to improved methods for treating a tumor or a cancer (e.g., B cell related disease or cancer, including multiple myeloma). The disclosure presented herein also relates to methods of manufacturing CAR T cells (e.g., CAR T cells directed to BCMA (BCMA CAR T cells)). As used herein, the term “B cell related conditions” relates to conditions involving inappropriate B cell activity and B cell malignancies.

Particular embodiments, presented herein relate to improved adoptive cell therapy of diseases (e.g., a tumor or a cancer or a B cell related disease or cancer, including multiple myeloma) using genetically modified immune effector cells. Genetic approaches offer a potential means to enhance immune recognition and elimination of cancer cells. One promising strategy is to genetically engineer immune effector cells to express chimeric antigen receptors (CAR) that redirect cytotoxicity toward cancer cells.

The improved methods of administering CAR T cell therapies for use in subjects (e.g., patients) who have been administered alkylating agents (e.g., for chemotherapy) prior to being administered a CAR T cell therapy disclosed herein include methods wherein a step of isolating peripheral blood mononuclear cells (PBMCs) from the subject is performed after a period of time (i.e., a “washout” period) after an alkylating agent has been administered to the subject. The improved methods of administering CAR T cell therapies for use in subjects who have been administered alkylating agents (e.g., for chemotherapy) prior to being administered a CAR T cell therapy disclosed herein may be used with genetically modified immune effector cells (e.g., CAR T cells) that can readily be expanded, exhibit long-term persistence in vivo, and, for example, genetically modified immune effector cells (e.g., CAR T cells) that reduce impairment of humoral immunity by targeting B cells expressing B cell maturation antigen (BCMA, also known as CD269 or tumor necrosis factor receptor superfamily, member 17; TNFRSF17). Improved methods of manufacturing CAR T cells (e.g., BCMA CAR T cells) from PBMCs isolated from patients who have been administered alkylating agents (e.g., for chemotherapy) are also disclosed herein.

BCMA is a member of the tumor necrosis factor receptor superfamily (see, e.g., Thompson et al., J. Exp. Medicine, 192(1): 129-135, 2000, and Mackay et al., Annu. Rev. Immunol, 21: 231-264, 2003. BCMA binds B-cell activating factor (BAFF) and a proliferation inducing ligand (APRIL) (see, e.g., Mackay et al., 2003 and Kalled et al., Immunological Reviews, 204: 43-54, 2005). Among nonmalignant cells, BCMA has been reported to be expressed mostly in plasma cells and subsets of mature B-cells (see, e.g., Laabi et al., EMBO J., 77(1): 3897-3904, 1992; Laabi et al., Nucleic Acids Res., 22(7): 1147-1154, 1994; Kalled et al., 2005; O'Connor et al., J. Exp. Medicine, 199(1): 91-97, 2004; and Ng et al., J. Immunol., 73(2): 807-817, 2004. Mice deficient in BCMA are healthy and have normal numbers of B cells, but the survival of long-lived plasma cells is impaired (see, e.g., O'Connor et al., 2004; Xu et al., Mol. Cell. Biol., 21(12): 4067-4074, 2001; and Schiemann et al., Science, 293(5537): 2 111-21 14, 2001). BCMA RNA has been detected universally in multiple myeloma cells and in other lymphomas, and BCMA protein has been detected on the surface of plasma cells from multiple myeloma patients by several investigators (see, e.g., Novak et al., Blood, 103(2): 689-694, 2004; Neri et al., Clinical Cancer Research, 73(19): 5903-5909, 2007; Bellucci et al., Blood, 105(10): 3945-3950, 2005; and Moreaux et al., Blood, 703(8): 3148-3157, 2004.

In one aspect, for example, provided herein is a method of treating a tumor or a cancer in a subject in need thereof, comprising: (a) administering to the subject an alkylating agent; (b) isolating peripheral blood mononuclear cells (PBMCs) from the subject at least about six (6) months after step (a); (c) determining that at least about 20% of the PBMCs are T cells; (d) on the basis of the determination in step (c), subsequently manufacturing chimeric antigen receptor (CAR) T cells from the PBMCs; and (e) administering the CAR T cells to the subject. In a specific embodiment, step (b) is performed at least about seven (7) months, at least about eight (8) months, at least about nine (9) months, at least about ten (10) months, at least about eleven (11) months, at least about twelve (12) months, at least about thirteen (13) months, or at least about fourteen (14) months after step (a).

In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are T cells.

In a particular embodiment of the methods presented herein, the T cells comprise CD3+ cells. In particular embodiments, the T cells are CD3+ cells, e.g., CD45+/CD3+ T cells. In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T CD3+ T cells, e.g., CD45+/CD3+ T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are CD3+ T cells, e.g., CD45+/CD3+ T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are CD3+ T cells, e.g., CD45+/CD3+ T cells.

In a particular embodiment, the T cells are CD8+ effector memory RA (T_{EMRA}) (CCR7−/CD45RA+) T cells. In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T_{EMRA} T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are T_{EMRA} T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are T_{EMRA} T cells. In particular embodiments of such methods, the T cells are CD3+ cells.

In the methods presented herein, the determining may be performed using standard techniques well known to those of skill in the relevant art. For example, in the methods presented herein, the determining step may be performed by utilizing techniques such as those utilized in Example 2 (e.g., immunophenotyping of the PBMCs by polychromatic flow cytometry for markers associated with T cell differentiation, memory, senescence, and exhaustion).

In a specific embodiment, step (b) is performed at least about six (6) months to about fourteen (14) months after step (a), at least about six (6) months to about thirteen (13) months after step (a), at least about six (6) months to about twelve (12) months after step (a), at least about six (6) months to about eleven (11) months after step (a), at least about six (6) months to about ten (10) months after step (a), at least about six (6) months to about nine (9) months after step (a), at least about six (6) months to about eight (8) months after step (a), or at least about six (6) months to about seven (7) months after step (a). In a specific embodiment, step (b) is performed at least about seven (7) months to about fourteen (14) months after step (a), at least about eight (8) months to about fourteen (14) months after step (a), at least about nine (9) months to about fourteen (14) months after step (a), at least about ten (10) months to about fourteen (14) months after step (a), at least about eleven (11) months to about fourteen (14) months after step (a), at least about twelve (12) months to about fourteen (14) months after step (a), or at least about thirteen (13) months to about fourteen (14) months after step (a).

In another specific embodiment, step (b) is performed at least about four (4) months or five (5) months to about fourteen (14) months after step (a), at least about four (4) months or five (5) months to about thirteen (13) months after step (a), at least about four (4) months or five (5) months to about twelve (12) months after step (a), at least about four (4) months or five (5) months to about eleven (11) months after step (a), at least about four (4) months or five (5) months to about ten (10) months after step (a), at least about four (4) months or five (5) months to about nine (9) months after step (a), at least about four (4) months or five (5) months to about eight (8) months after step (a), at least about four (4) months or five (5) months to about seven (7) months after step (a), or at least about four (4) or five (5) months to about six (6) months after step (a).

In a specific embodiment, the tumor or cancer is lymphoma, lung cancer, breast cancer, prostate cancer, adrenocortical carcinoma, thyroid carcinoma, nasopharyngeal carcinoma, melanoma, skin carcinoma, colorectal carcinoma, a desmoid tumor, a desmoplastic small round cell tumor, an endocrine tumor, a Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipomachronic lymphocytic leukemia (small lymphocytic lymphoma), B-cell prolymphocytic leukemia, lymphoplasmacytic lymphoma, Waldenström macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, a non-Hodgkin lymphoma, or multiple myeloma.

In a specific embodiment, the cancer is multiple myeloma, chronic lymphocytic leukemia, or a non-Hodgkins lymphoma. In a specific embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a specific embodiment, the cancer is multiple myeloma. In a specific embodiment, the multiple myeloma is high-risk multiple myeloma or relapsed and refractory multiple myeloma. In a specific embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a specific embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan (e.g., melphalan hydrochloride), oxaliplatin, platinum, procarbazine, streptozocin, temozolomide, thiotepa, and trabectedin. In a specific embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a specific embodiment, the alkylating agent is cyclophosphamide.

In a particular embodiment, the subject is a human.

In a particular embodiment, the subject undergoes a leukapharesis procedure to collect the PBMCs for the manufacture of the CAR T cells prior to their administration to the subject.

In a particular embodiment, the CAR T cells are administered by an intravenous infusion.

In another aspect, provided herein is a method of treating a tumor or a cancer in a subject in need thereof, comprising: (a) isolating peripheral blood mononuclear cells (PBMCs) from the subject, and determining that at least about 20% of the PBMCs are T cells; (b) on the basis of the determination in step (a), subsequently manufacturing chimeric antigen receptor (CAR) T cells from the PBMCs; and (c) administering to the subject the CAR T cells, wherein, prior to step (a), the subject had previously received an alkylating agent for treatment of the cancer. In a specific embodiment, the subject had previously received the alkylating agent at least about six (6) months prior to step (a). In a specific embodiment, the subject had previously received the alkylating agent at least about seven (7) months, at least about eight (8) months, at least about nine (9) months, at least about ten (10) months, at least about eleven (11) months, at least about twelve (12) months, at least about thirteen (13) months, or at least about fourteen (14) months prior to step (a). In a specific embodiment, the subject had previously received the alkylating agent at least about six (6) months to about fourteen (14) months prior to step (a), at least about six (6) months to about thirteen (13) months prior to step (a), at least about six (6) months to about twelve (12) months prior to step (a), at least about six (6) months to about eleven (11) months prior to step (a), at least about six (6) months to about ten (10) months prior to step (a), at least about six (6) months to about nine (9) months prior to step (a), at least about six (6) months to about eight (8) months after step (a), or at least about six (6) months to about seven (7) months prior to step (a). In a specific embodiment, the subject had previously received the alkylating agent at least about seven (7) months to about fourteen (14) months prior to step (a), at least about eight (8) months to about fourteen (14) months prior to step (a), at least about nine (9) months to about fourteen (14) months prior to step (a), at least about ten (10) months to about fourteen (14) months prior to step (a), at least about eleven (11) months to about fourteen (14) months prior to step (a), at least about twelve (12) months to about fourteen (14) months prior to step (a), or at least about thirteen (13) months to about fourteen (14) months prior to step (a).

In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are T cells.

In a particular embodiment of the methods presented herein, the T cells comprise CD3+ cells. In particular embodiments, the T cells are CD3+ cells. In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T CD3+ T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are CD3+ T cells, e.g., CD45+/CD3+ T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are CD3+ T cells.

In a particular embodiment, the T cells are CD8+ effector memory RA (T_{EMRA}) (CCR7−/CD45RA+) T cells. In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T_{EMRA} T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are T_{EMRA} T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are T_{EMRA} T cells. In particular embodiments of such methods, the T cells are CD3+ cells.

In the methods presented herein, the determining may be performed using standard techniques well known to those of skill in the relevant art. For example, in the methods presented herein, the determining step may be performed by utilizing techniques such as those utilized in Example 2 (e.g., immunophenotyping of the PBMCs by polychromatic flow cytometry for markers associated with T cell differentiation, memory, senescence, and exhaustion).

In another specific embodiment, the subject had previously received the alkylating agent at least about four (4) months or five (5) months to about fourteen (14) months prior to step (a), at least about four (4) months or five (5) months to about thirteen (13) months prior to step (a), at least about four (4) months or five (5) months to about twelve (12) months prior to step (a), at least about four (4) months or five (5) months to about eleven (11) months prior to step (a), at least about four (4) months or five (5) months to about ten (10) months prior to step (a), at least about four (4) months or five (5) months to about nine (9) months prior to step (a), at least about four (4) months or five (5) months to about eight (8) months prior to step (a), at least about four (4) months or five (5) months to about seven (7) months prior to step (a), or at least about four (4) or five (5) months to about six (6) months prior to step (a).

In a specific embodiment, the tumor or cancer is lymphoma, lung cancer, breast cancer, prostate cancer, adrenocortical carcinoma, thyroid carcinoma, nasopharyngeal carcinoma, melanoma, skin carcinoma, colorectal carcinoma, a desmoid tumor, a desmoplastic small round cell tumor, an endocrine tumor, a Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipomachronic lymphocytic leukemia (small lymphocytic lymphoma), B-cell prolymphocytic leukemia, lymphoplasmacytic lymphoma, Waldenström macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, a non-Hodgkin lymphoma, or multiple myeloma. In a specific embodiment, the cancer is multiple myeloma, chronic lymphocytic leukemia, or a non-Hodgkins lymphoma. In a specific embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a specific embodiment, the cancer is multiple myeloma. In a specific embodiment, the multiple myeloma is high-risk multiple myeloma or relapsed and refractory multiple myeloma. In a specific embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a specific embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan (e.g., melphalan hydrochloride), oxaliplatin, platinum, procarbazine, streptozocin, temozolomide, thiotepa, and trabectedin. In a specific embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a specific embodiment, the alkylating agent is cyclophosphamide.

In a particular embodiment, the subject is a human.

In a particular embodiment, the subject undergoes a leukapharesis procedure to collect the PBMCs for the manufacture of the CAR T cells prior to their administration to the subject.

In a particular embodiment, the CAR T cells are administered by an intravenous infusion.

In another aspect, a method of treating a tumor or a cancer in a subject in need thereof, comprising: (a) isolating peripheral blood mononuclear cells (PBMCs) from the subject; (b) manufacturing chimeric antigen receptor (CAR) T cells from the PBMCs; and (c) administering to the subject the CAR T cells, wherein the subject had previously received an alkylating agent for treatment of the cancer; wherein step (a) occurs at least about six months after the subject received the alkylating agent. In a particular embodiment, step (a) further comprises determining that at least about 20% of the PBMCs are T cells; and wherein step (b) further comprises subsequently manufacturing chimeric antigen receptor (CAR) T cells from the PBMCs on the basis of the determination in step (a). In a particular embodiment, step (a) occurs at least about seven (7) months, at least about eight (8) months, at least about nine (9) months, at least about ten (10) months, at least about eleven (11) months, at least about twelve (12) months, at least about thirteen (13) months, or at least about fourteen (14) months after the subject received the alkylating agent. In a specific embodiment, step (a) occurs at least about six (6) months to about fourteen (14) months after the subject received the alkylating agent, at least about six (6) months to about thirteen (13) months after the subject received the alkylating agent, at least about six (6) months to about twelve (12) months after the subject received the alkylating agent, at least about six (6) months to about eleven (11) months after the subject received the alkylating agent, at least about six (6) months to about ten (10) months after the subject received the alkylating agent, at least about six (6) months to about nine (9) months after the subject received the alkylating agent, at least about six (6) months to about eight (8) months after the subject received the alkylating agent, or at least about six (6) months to about seven (7) months after the subject received the alkylating agent. In a specific embodiment, step (a) occurs at least about seven (7) months to about fourteen (14) months after the subject received the alkylating agent, at least about eight (8) months to about fourteen (14) months after the subject received the alkylating agent, at least about nine (9) months to about fourteen (14) months after the subject received the alkylating agent, at least about ten (10) months to about fourteen (14) months after the subject received the alkylating agent, at least about eleven (11) months to about fourteen (14) months after the subject received the alkylating agent, at least about twelve (12) months to about fourteen (14) months after the subject received the alkylating agent, or at least about thirteen (13) months to about fourteen (14) months after the subject received the alkylating agent.

In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are T cells.

In a particular embodiment of the methods presented herein, the T cells comprise CD3+ cells. In particular embodiments, the T cells are CD3+ cells. In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T CD3+ T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are CD3+ T cells, e.g., CD45+/CD3+ T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are CD3+ T cells.

In a particular embodiment, the T cells are CD8+ effector memory RA (T_{EMRA}) (CCR7−/CD45RA+) T cells. In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T_{EMRA} T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are T_{EMRA} T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are T_{EMRA} T cells. In particular embodiments of such methods, the T cells are CD3+ cells.

In the methods presented herein, the determining may be performed using standard techniques well known to those of skill in the relevant art. For example, in the methods presented herein, the determining step may be performed by utilizing techniques such as those utilized in Example 2 (e.g., immunophenotyping of the PBMCs by polychromatic flow cytometry for markers associated with T cell differentiation, memory, senescence, and exhaustion).

In another specific embodiment, step (a) occurs at least about four (4) months or five (5) months to about fourteen (14) months after the subject received the alkylating agent, at least about four (4) months or five (5) months to about thirteen (13) months after the subject received the alkylating agent, at least about four (4) months or five (5) months to about twelve (12) months after the subject received the alkylating agent, at least about four (4) months or five (5) months to about eleven (11) months after the subject received the alkylating agent, at least about four (4) months or five (5) months to about ten (10) months after the subject received the alkylating agent, at least about four (4) months or five (5) months to about nine (9) months after the subject received the alkylating agent, at least about four (4) months or five (5) months to about eight (8) months after the subject received the alkylating agent, at least about four (4) months or five (5) months to about seven (7) months after the subject received the alkylating agent, or at least about four (4) or five (5) months to about six (6) months after the subject received the alkylating agent.

In a specific embodiment, the tumor or cancer is lymphoma, lung cancer, breast cancer, prostate cancer, adrenocortical carcinoma, thyroid carcinoma, nasopharyngeal carcinoma, melanoma, skin carcinoma, colorectal carcinoma, a desmoid tumor, a desmoplastic small round cell tumor, an endocrine tumor, a Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipomachronic lymphocytic leukemia (small lymphocytic lymphoma), B-cell prolymphocytic leukemia, lymphoplasmacytic lymphoma, Waldenström macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, a non-Hodgkin lymphoma, or multiple myeloma. In a specific embodiment, the cancer is multiple myeloma, chronic lymphocytic leukemia, or a non-Hodgkins lymphoma. In a specific embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a specific embodiment, the cancer is multiple myeloma. In a specific embodiment, the multiple myeloma is high-risk multiple myeloma or relapsed and refractory multiple myeloma. In a specific embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a specific embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan (e.g., melphalan hydrochloride), oxaliplatin, platinum, procarbazine, streptozocin, temozolomide, thiotepa, and trabectedin. In a specific embodiment, the alkylating agent is one or more of bendamustine, busulfan, carboplatin, carmustine, chlorambucil, cisplatin, cyclophosphamide, evofosfamide, ifosfamide, oxaliplatin, platinum, procarbazine, and thiotepa. In a specific embodiment, the alkylating agent is cyclophosphamide.

In a particular embodiment, the subject is a human.

In a particular embodiment, the subject undergoes a leukapharesis procedure to collect the PBMCs for the manufacture of the CAR T cells prior to their administration to the subject.

In a particular embodiment, the CAR T cells are administered by an intravenous infusion.

In another aspect, provided herein is a method of treating a tumor or a cancer in a subject in need thereof, wherein the subject has been administered an alkylating agent, comprising: (a) determining that the subject has not been administered the alkylating agent less than about six (6) months prior to the determining step; (b) isolating peripheral blood mononuclear cells (PBMCs) from the subject; (c) manufacturing chimeric antigen receptor (CAR) T cells from the PBMCs; and (d) administering to the subject the CAR T cells. In a particular embodiment, in step (a) the subject has not been administered the alkylating agent less than about seven (7) months, less than about eight (8) months, less than about nine (9) months, less than about ten (10) months, less than about eleven (11) months, less than about twelve (12) months, less than about thirteen (13) months, or less than about fourteen (14) months prior to the determining step.

In a specific embodiment, the tumor or cancer is lymphoma, lung cancer, breast cancer, prostate cancer, adrenocortical carcinoma, thyroid carcinoma, nasopharyngeal carcinoma, melanoma, skin carcinoma, colorectal carcinoma, a desmoid tumor, a desmoplastic small round cell tumor, an endocrine tumor, a Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipomachronic lymphocytic leukemia (small lymphocytic lymphoma), B-cell prolymphocytic leukemia, lymphoplasmacytic lymphoma, Waldenström macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, a non-Hodgkin lymphoma, or multiple myeloma. In a specific embodiment, the cancer is multiple myeloma, chronic lymphocytic leukemia, or a non-Hodgkins lymphoma. In a specific embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a specific embodiment, the cancer is multiple myeloma. In a specific embodiment, the multiple myeloma is high-risk multiple myeloma or relapsed and refractory multiple myeloma. In a specific embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a specific embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan (e.g., melphalan hydrochloride), oxaliplatin, platinum, procarbazine, streptozocin, temozolomide, thiotepa, and trabectedin. In a specific embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a specific embodiment, the alkylating agent is cyclophosphamide.

In a particular embodiment, the subject is a human.

In a particular embodiment, the subject undergoes a leukapharesis procedure to collect the PBMCs for the manufacture of the CAR T cells prior to their administration to the subject.

In a particular embodiment, the CAR T cells are administered by an intravenous infusion.

In another aspect, provided herein is a method of treating a tumor or a cancer in a subject in need thereof, wherein the subject has been administered an alkylating agent, comprising: (a) isolating peripheral blood mononuclear cells (PBMCs) from the subject; (b) manufacturing chimeric antigen receptor (CAR) T cells from the PBMCs; and (c) administering to the subject the CAR T cells, wherein, at the time of the isolating, the subject has been determined to have been administered the alkylating agent at least about six (6) months prior. In a particular embodiment, the subject has been determined to have been administered the alkylating agent at least about seven (7) months, at least about eight (8) months, at least about nine (9) months, at least about ten (10) months, at least about eleven (11) months, at least about twelve (12) months, at least about thirteen (13) months, or at least about fourteen (14) months prior. In a specific embodiment, the subject has been determined to have been administered the alkylating agent at least about six (6) months to about fourteen (14) months prior, at least about six (6) months to about thirteen (13) months prior, at least about six (6) months to about twelve (12) months prior, at least about six (6) months to about eleven (11) months prior, at least about six (6) months to about ten (10) months prior, at least about six (6) months to about nine (9) months prior, at least about six (6) months to about eight (8) months prior, or at least about six (6) months to about seven (7) months prior. In a specific embodiment, the subject has been determined to have been administered the alkylating agent at least about seven (7) months to about fourteen (14) months prior, at least about eight (8) months to about fourteen (14) months prior, at least about nine (9) months to about fourteen (14) months prior, at least about ten (10) months to about fourteen (14) months prior, at least about eleven (11) months to about fourteen (14) months prior, at least about twelve (12) months to about fourteen (14) months prior, or at least about thirteen (13) months to about fourteen (14) months prior.

In another specific embodiment, the subject has been determined to have been administered the alkylating agent at least about four (4) months or five (5) months to about fourteen (14) months prior, at least about four (4) months or five (5) months to about thirteen (13) months prior, at least about four (4) months or five (5) months to about twelve (12) months prior, at least about four (4) months or five (5) months to about eleven (11) months prior, at least about four (4) months or five (5) months to about ten (10) months prior, at least about four (4) months or five (5) months to about nine (9) months prior, at least about four (4) months or five (5) months to about eight (8) months prior, at least about four (4) months or five (5) months to about seven (7) months prior, or at least about four (4) or five (5) months to about six (6) months prior.

In a specific embodiment, the tumor or cancer is lymphoma, lung cancer, breast cancer, prostate cancer, adrenocortical carcinoma, thyroid carcinoma, nasopharyngeal carcinoma, melanoma, skin carcinoma, colorectal carcinoma, a desmoid tumor, a desmoplastic small round cell tumor, an endocrine tumor, a Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipomachronic lymphocytic leukemia (small lymphocytic lymphoma), B-cell prolymphocytic leukemia, lymphoplasmacytic lymphoma, Waldenström macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, a non-Hodgkin lymphoma, or multiple myeloma. In a specific embodiment, the cancer is multiple myeloma, chronic lymphocytic leukemia, or a non-Hodgkins lymphoma. In a specific embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a specific embodiment, the cancer is multiple myeloma. In a specific embodiment, the multiple myeloma is high-risk multiple myeloma or relapsed and refractory multiple myeloma. In a specific embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a specific embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan (e.g., melphalan hydrochloride), oxaliplatin, platinum, procarbazine, streptozocin, temozolomide, thiotepa, and trabectedin. In a specific embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a specific embodiment, the alkylating agent is cyclophosphamide.

In a particular embodiment, the subject is a human.

In a particular embodiment, the subject undergoes a leukapharesis procedure to collect the PBMCs for the manufacture of the CAR T cells prior to their administration to the subject.

In a particular embodiment, the CAR T cells are administered by an intravenous infusion.

In another aspect, provided herein is a method of treating a tumor or a cancer in a subject in need thereof, wherein the subject has been administered an alkylating agent, comprising administering to the subject T cells expressing a chimeric antigen receptor (CAR T cells) manufactured from peripheral blood mononuclear cells PBMCs isolated from the patient, wherein, at the time said PBMCs are isolated, the subject has last received the alkylating agent at least about six (6) months prior to the time the PBMCs are isolated. In a particular embodiment, the subject has last received the alkylating agent at least about seven (7) months, at least about eight (8) months, at least about nine (9) months, at least about ten (10) months, at least about eleven (11) months, at least about twelve (12) months, at least about thirteen (13) months, or at least about fourteen (14) months prior to the time the PBMCs are isolated.

In another specific embodiment, the subject has has last received the alkylating agent at least about four (4) months or five (5) months to about fourteen (14) months prior, at least about four (4) months or five (5) months to about thirteen (13) months prior to the time the PBMCs are isolated, at least about four (4) months or five (5) months to about twelve (12) months prior to the time the PBMCs are isolated, at least about four (4) months or five (5) months to about eleven (11) months prior to the time the PBMCs are isolated, at least about four (4) months or five (5) months to about ten (10) months prior to the time the PBMCs are isolated, at least about four (4) months or five (5) months to about nine (9) months prior to the time the PBMCs are isolated, at least about four (4) months or five (5) months to about eight (8) months prior to the time the PBMCs are isolated, at least about four (4) months or five (5) months to about seven (7) months prior to the time the PBMCs are isolated, or at least about four (4) or five (5) months to about six (6) months prio to the time the PBMCs are isolated.

In a specific embodiment, the tumor or cancer is lymphoma, lung cancer, breast cancer, prostate cancer, adrenocortical carcinoma, thyroid carcinoma, nasopharyngeal carcinoma, melanoma, skin carcinoma, colorectal carcinoma, a desmoid tumor, a desmoplastic small round cell tumor, an endocrine tumor, a Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipomachronic lymphocytic leukemia (small lymphocytic lymphoma), B-cell prolymphocytic leukemia, lymphoplasmacytic lymphoma, Waldenström macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, a non-Hodgkin lymphoma, or multiple myeloma. In a specific embodiment, the cancer is multiple myeloma, chronic lymphocytic leukemia, or a non-Hodgkins lymphoma. In a specific embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a specific embodiment, the cancer is multiple myeloma. In a specific embodiment, the multiple myeloma is high-risk multiple myeloma or relapsed and refractory multiple myeloma. In a specific embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a specific embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan (e.g., melphalan hydrochloride), oxaliplatin, platinum, procarbazine, streptozocin, temozolomide, thiotepa, and trabectedin. In a specific embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a specific embodiment, the alkylating agent is cyclophosphamide.

In a particular embodiment, the subject is a human.

In a particular embodiment, the subject undergoes a leukapharesis procedure to collect the PBMCs for the manufacture of the CAR T cells prior to their administration to the subject.

In a particular embodiment, the CAR T cells are administered by an intravenous infusion.

In another aspect, provided herein is a method of treating a tumor or a cancer in a subject in need thereof, wherein the subject has been administered an alkylating agent, comprising administering to the subject T cells expressing a chimeric antigen receptor (CAR T cells) manufactured from peripheral blood mononuclear cells PBMCs isolated from the patient, wherein, at the time said PBMCs are isolated, the PBMCs comprises at least about 20% T cells.

In a specific embodiment, the tumor or cancer is lymphoma, lung cancer, breast cancer, prostate cancer, adrenocortical carcinoma, thyroid carcinoma, nasopharyngeal carcinoma, melanoma, skin carcinoma, colorectal carcinoma, a desmoid tumor, a desmoplastic small round cell tumor, an endocrine tumor, a Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipomachronic lymphocytic leukemia (small lymphocytic lymphoma), B-cell prolymphocytic leukemia, lymphoplasmacytic lymphoma, Waldenström macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, a non-Hodgkin lymphoma, or multiple myeloma. In a specific embodiment, the cancer is multiple myeloma, chronic lymphocytic leukemia, or a non-Hodgkins lymphoma. In a specific embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a specific embodiment, the cancer is multiple myeloma. In a specific embodiment, the multiple myeloma is high-risk multiple myeloma or relapsed and refractory multiple myeloma. In a specific embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a specific embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan, oxaliplatin, platinum, procarbazine, streptozocin, temozolomide, thiotepa and trabectedin. In a specific embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a specific embodiment, the alkylating agent is cyclophosphamide.

In a particular embodiment, the subject is a human.

In a particular embodiment, the subject undergoes a leukapharesis procedure to collect the PBMCs for the manufacture of the CAR T cells prior to their administration to the subject.

In a particular embodiment, the CAR T cells are administered by an intravenous infusion.

In another aspect, provided herein is a method of treating a cancer caused by B Cell Maturation Agent (BCMA) expressing cells in a subject in need thereof, comprising: (a) administering to the subject an alkylating agent; (b) isolating peripheral blood mononuclear cells (PBMCs) from the subject at least about six months after step (a); (c) determining that at least about 20% of the PBMCs are T cells; (d) on the basis of the determination in step (c), subsequently manufacturing chimeric antigen receptor (CAR) T cells directed to BCMA (BCMA CAR T cells) from the PBMCs; and (e) administering to the subject the CAR T cells to the subject. In a particular embodiment, step (b) is performed at least about seven (7) months, at least about eight (8) months, at least about nine (9) months, at least about ten (10) months, at least about eleven (11) months, at least about twelve (12) months, at least about thirteen (13) months, or at least about fourteen (14) months after step (a) of administering to the subject an alkylating agent. In a specific embodiment, step (b) is performed at least about six (6) months to about fourteen (14) months after step (a), at least about six (6) months to about thirteen (13) months after step (a), at least about six (6) months to about twelve (12) months after step (a), at least about six (6) months to about eleven (11) months after step (a), at least about six (6) months to about ten (10) months after step (a), at least about six (6) months to about nine (9) months after step (a), at least about six (6) months to about eight (8) months after step (a), or at least about six (6) months to about seven (7) months after step (a). In a specific embodiment, step (b) is performed at least about seven (7) months to about fourteen (14) months after step (a), at least about eight (8) months to about fourteen (14) months after step (a), at least about nine (9) months to about fourteen (14) months after step (a), at least about ten (10) months to about fourteen (14) months after step (a), at least about eleven (11) months to about fourteen (14) months after step (a), at least about twelve (12) months to about fourteen (14) months after step (a), or at least about thirteen (13) months to about fourteen (14) months after step (a).

In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are T cells.

In a particular embodiment of the methods presented herein, the T cells comprise CD3+ cells. In particular embodiments, the T cells are CD3+ cells. In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T CD3+ T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are CD3+ T cells, e.g., CD45+/CD3+ T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are CD3+ T cells.

In a particular embodiment, the T cells are CD8+ effector memory RA (T_{EMRA}) (CCR7−/CD45RA+) T cells. In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T_{EMRA} T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are T_{EMRA} T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are T_{EMRA} T cells. In particular embodiments of such methods, the T cells are CD3+ cells.

In the methods presented herein, the determining may be performed using standard techniques well known to those of skill in the relevant art. For example, in the methods presented herein, the determining step may be performed by utilizing techniques such as those utilized in Example 2 (e.g., immunophenotyping of the PBMCs by polychromatic flow cytometry for markers associated with T cell differentiation, memory, senescence, and exhaustion).

In another specific embodiment, step (b) is performed at least about four (4) months or five (5) months to about fourteen (14) months after step (a), at least about four (4) months or five (5) months to about thirteen (13) months after step (a), at least about four (4) months or five (5) months to about twelve (12) months after step (a), at least about four (4) months or five (5) months to about eleven (11) months after step (a), at least about four (4) months or five (5) months to about ten (10) months after step (a), at least about four (4) months or five (5) months to about nine (9) months after step (a), at least about four (4) months or five (5) months to about eight (8) months after step (a), at least about four (4) months or five (5) months to about seven (7) months after step (a), or at least about four (4) or five (5) months to about six (6) months after step (a).

In a particular embodiment, the cancer is multiple myeloma, chronic lymphocytic leukemia, or a non-Hodgkins lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is multiple myeloma. In a particular embodiment, the multiple myeloma is high-risk multiple myeloma or relapsed and refractory multiple myeloma. In a particular embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a particular embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan (e.g., melphalan hydrochloride), oxaliplatin, platinum, procarbazine, streptozocin, temozolomide, thiotepa, and trabectedin. In a particular embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a particular embodiment, the alkylating agent is cyclophosphamide.

In a particular embodiment, the subject is a human.

In a particular embodiment, the BCMA CAR T cells comprise a CAR directed to BCMA, wherein the CAR directed to BCMA comprises an antibody or antibody fragment that targets BCMA. In a particular embodiment, the BCMA CAR T cells comprise a CAR directed to BCMA, wherein the CAR directed to BCMA comprises a single chain Fv antibody or antibody fragment (scFv). In a particular embodiment, the BCMA CAR T cells comprise a CAR directed to BCMA, wherein the CAR directed to BCMA comprises a BCMA02 scFv, e.g., SEQ ID NO: 38.

In a particular embodiment, the subject undergoes a leukapharesis procedure to collect the PBMCs for the manufacture of the BCMA CAR T cells prior to their administration to the subject.

In a particular embodiment, the BCMA CAR T cells are administered by an intravenous infusion.

In another aspect, provided herein is a method of treating a cancer caused by B Cell Maturation Agent (BCMA) expressing cells in a subject in need thereof, comprising: (a) isolating peripheral blood mononuclear cells (PBMCs) from the subject, and determining that at least about 20% of the PBMCs are T cells; (b) on the basis of the determination in step (a), subsequently manufacturing chimeric antigen receptor (CAR) T cells directed to BCMA (BCMA CAR T cells) from the PBMCs; and (c) administering to the subject the CAR T cells, wherein, prior to step (a), the subject had previously received an alkylating agent for treatment of the cancer. In a particular embodiment, the subject had previously received the alkylating agent at least about six months prior to step (a). In a particular embodiment, the subject had previously received the alkylating agent at least about seven (7) months, at least about eight (8) months, at least about nine (9) months, at least about ten (10) months, at least about eleven (11) months, at least about twelve (12) months, at least about thirteen (13) months, or at least about fourteen (14) months prior to step (a). In a specific embodiment, the subject had previously received the alkylating agent at least about six (6) months to about fourteen (14) months prior to step (a), at least about six (6) months to about thirteen (13) months prior to step (a), at least about six (6) months to about twelve (12) months prior to step (a), at least about six (6) months to about eleven (11) months prior to step (a), at least about six (6) months to about ten (10) months prior to step (a), at least about six (6) months to about nine (9) months prior to step (a), at least about six (6) months to about eight (8) months after step (a), or at least about six (6) months to about seven (7) months prior to step (a). In a specific embodiment, step (b) is performed at least about seven (7) months to about fourteen (14) months prior to step (a), at least about eight (8) months to about fourteen (14) months prior to step (a), at least about nine (9) months to about fourteen (14) months prior to step (a), at least about ten (10) months to about fourteen (14) months prior to step (a), at least about eleven (11) months to about fourteen (14) months prior to step (a), at least about twelve (12) months to about fourteen (14) months prior to step (a), or at least about thirteen (13) months to about fourteen (14) months prior to step (a).

In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are T cells.

In a particular embodiment of the methods presented herein, the T cells comprise CD3+ cells. In particular embodiments, the T cells are CD3+ cells. In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T CD3+ T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are CD3+ T cells, e.g., CD45+/CD3+ T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are CD3+ T cells.

In a particular embodiment, the T cells are CD8+ effector memory RA (TEMRA) (CCR7−/CD45RA+) T cells. In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T_{EMRA} T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are T_{EMRA} T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are T_{EMRA} T cells. In particular embodiments of such methods, the T cells are CD3+ cells.

In the methods presented herein, the determining may be performed using standard techniques well known to those of skill in the relevant art. For example, in the methods presented herein, the determining step may be performed by utilizing techniques such as those utilized in Example 2 (e.g., immunophenotyping of the PBMCs by polychromatic flow cytometry for markers associated with T cell differentiation, memory, senescence, and exhaustion).

In another specific embodiment, the subject had previously received the alkylating agent at least about four (4) months or five (5) months to about fourteen (14) months prior to step (a), at least about four (4) months or five (5) months to about thirteen (13) months prior to step (a), at least about four (4) months or five (5) months to about twelve (12) months prior to step (a), at least about four (4) months or five (5) months to about eleven (11) months prior to step (a), at least about four (4) months or five (5) months to about ten (10) months prior to step (a), at least about four (4) months or five (5) months to about nine (9) months prior to step (a), at least about four (4) months or five (5) months to about eight (8) months prior to step (a), at least about four (4) months or five (5) months to about seven (7) months prior to step (a), or at least about four (4) or five (5) months to about six (6) months prior to step (a).

In a particular embodiment, the cancer is multiple myeloma, chronic lymphocytic leukemia, or a non-Hodgkins lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma.

In a particular embodiment, the cancer is multiple myeloma. In a particular embodiment, the multiple myeloma is high-risk multiple myeloma or relapsed and refractory multiple myeloma. In a particular embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a particular embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan (e.g., melphalan hydrochloride), oxaliplatin, platinum, procarbazine, streptozocin, temozolomide, thiotepa, and trabectedin. In a particular embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a particular embodiment, the alkylating agent is cyclophosphamide.

In a particular embodiment, the subject is a human.

In a particular embodiment, the BCMA CAR T cells comprise a CAR directed to BCMA, wherein the CAR directed to BCMA comprises an antibody or antibody fragment that targets BCMA. In a particular embodiment, the BCMA CAR T cells comprise a CAR directed to BCMA, wherein the CAR directed to BCMA comprises a single chain Fv antibody or antibody fragment (scFv). In a particular embodiment, the BCMA CAR T cells comprise a CAR directed to BCMA, wherein the CAR directed to BCMA comprises a BCMA02 scFv, e.g., SEQ ID NO: 38.

In a particular embodiment, the subject undergoes a leukapharesis procedure to collect the PBMCs for the manufacture of the BCMA CAR T cells prior to their administration to the subject.

In a particular embodiment, the BCMA CAR T cells are administered by an intravenous infusion.

In another aspect, provided herein is a method of treating a cancer caused by B Cell Maturation Agent (BCMA) expressing cells in a subject in need thereof, comprising: (a) isolating peripheral blood mononuclear cells (PBMCs) from the subject; (b) manufacturing chimeric antigen receptor (CAR) T cells directed to BCMA (BCMA CAR T cells) from the PBMCs; (c) administering to the subject the BCMA CAR T cells, wherein the patient had previously received an alkylating agent for treatment of the cancer, and wherein step (a) occurs at least about six months after the subject received the alkylating agent. In a particular embodiment, step (a) further comprises determining that at least about 20% of the PBMCs are T cells; and wherein step (b) further comprises subsequently manufacturing chimeric antigen receptor (CAR) T cells from the PBMCs on the basis of the determination in step (a). In a particular embodiment, step (a) occurs at least about seven (7) months, at least about eight (8) months, at least about nine (9) months, at least about ten (10) months, at least about eleven (11) months, at least about twelve (12) months, at least about thirteen (13) months, or at least about fourteen (14) months after the subject received the alkylating agent. In a specific embodiment, step (a) occurs at least about six (6) months to about fourteen (14) months after the subject received the alkylating agent, at least about six (6) months to about thirteen (13) months after the subject received the alkylating agent, at least about six (6) months to about twelve (12) months after the subject received the alkylating agent, at least about six (6) months to about eleven (11) months after the subject received the alkylating agent, at least about six (6) months to about ten (10) months after the subject received the alkylating agent, at least about six (6) months to about nine (9) months after the subject received the alkylating agent, at least about six (6) months to about eight (8) months after the subject received the alkylating agent, or at least about six (6) months to about seven (7) months after the subject received the alkylating agent. In a specific embodiment, step (a) occurs at least about seven (7) months to about fourteen (14) months after the subject received the alkylating agent, at least about eight (8) months to about fourteen (14) months after the subject received the alkylating agent, at least about nine (9) months to about fourteen (14) months after the subject received the alkylating agent, at least about ten (10) months to about fourteen (14) months after the subject received the alkylating agent, at least about eleven (11) months to about fourteen (14) months after the subject received the alkylating agent, at least about twelve (12) months to about fourteen (14) months after the subject received the alkylating agent, or at least about thirteen (13) months to about fourteen (14) months after the subject received the alkylating agent.

In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are T cells.

In a particular embodiment of the methods presented herein, the T cells comprise CD3+ cells. In particular embodiments, the T cells are CD3+ cells. In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T CD3+ T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are CD3+ T cells, e.g., CD45+/CD3+ T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are CD3+ T cells.

In a particular embodiment, the T cells are CD8+ effector memory RA (T_{EMRA}) (CCR7−/CD45RA+) T cells. In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T_{EMRA} T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are T_{EMRA} T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are T_{EMRA} T cells. In particular embodiments of such methods, the T cells are CD3+ cells.

In the methods presented herein, the determining may be performed using standard techniques well known to those of skill in the relevant art. For example, in the methods presented herein, the determining step may be performed by utilizing techniques such as those utilized in Example 2 (e.g., immunophenotyping of the PBMCs by polychromatic flow cytometry for markers associated with T cell differentiation, memory, senescence, and exhaustion).

In another specific embodiment, step (a) occurs at least about four (4) months or five (5) months to about fourteen (14) months after the subject received the alkylating agent, at least about four (4) months or five (5) months to about thirteen (13) months after the subject received the alkylating agent, at least about four (4) months or five (5) months to about twelve (12) months after the subject received the alkylating agent, at least about four (4) months or five (5) months to about eleven (11) months after the subject received the alkylating agent, at least about four (4) months or five (5) months to about ten (10) months after the subject received the alkylating agent, at least about four (4) months or five (5) months to about nine (9) months after the subject received the alkylating agent, at least about four (4) months or five (5) months to about eight (8) months after the subject received the alkylating agent, at least about four (4) months or five (5) months to about seven (7) months after the subject received the alkylating agent, or at least about four (4) or five (5) months to about six (6) months after the subject received the alkylating agent.

In a particular embodiment, the cancer is multiple myeloma, chronic lymphocytic leukemia, or a non-Hodgkins lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is multiple myeloma. In a particular embodiment, the multiple myeloma is high-risk multiple myeloma or relapsed and refractory multiple myeloma. In a particular embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a particular embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan (e.g., melphalan hydrochloride), oxaliplatin, platinum, procarbazine, streptozocin, temozolomide, thiotepa, and trabectedin. In a particular embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a particular embodiment, the alkylating agent is cyclophosphamide.

In a particular embodiment, the subject is a human.

In a particular embodiment, the BCMA CAR T cells comprise a CAR directed to BCMA, wherein the CAR directed to BCMA comprises an antibody or antibody fragment that targets BCMA. In a particular embodiment, the BCMA CAR T cells comprise a CAR directed to BCMA, wherein the CAR directed to BCMA comprises a single chain Fv antibody or antibody fragment (scFv). In a particular embodiment, the BCMA CAR T cells comprise a CAR directed to BCMA, wherein the CAR directed to BCMA comprises a BCMA02 scFv, e.g., SEQ ID NO: 38.

In a particular embodiment, the subject undergoes a leukapharesis procedure to collect the PBMCs for the manufacture of the BCMA CAR T cells prior to their administration to the subject.

In a particular embodiment, the BCMA CAR T cells are administered by an intravenous infusion.

In another aspect, provided herein is a method of treating a cancer caused by B Cell Maturation Agent (BCMA) expressing cells in a subject in need thereof, wherein the subject has been administered an alkylating agent for treatment of a cancer, comprising: (a) determining that the subject has not been administered the alkylating agent less than about six (6) months prior to the determining step; (b) isolating peripheral blood mononuclear cells (PBMCs) from the subject, wherein the isolating is performed at least six (6) months after the alkylating agent has been administered to the subject; (c) manufacturing chimeric antigen receptor (CAR) T cells directed to BCMA (BCMA CAR T cells) from the PBMCs; and (d) administering to the subject the BCMA CAR T cells. In a particular embodiment, in step (a) the subject has not been administered the alkylating agent less than about seven (7) months, less than about eight (8) months, less than about nine (9) months, less than about ten (10) months, less than about eleven (11) months, less than about twelve (12) months, less than about thirteen (13) months, or less than about fourteen (14) months prior to the determining step.

In a particular embodiment, the cancer is multiple myeloma, chronic lymphocytic leukemia, or a non-Hodgkins lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is multiple myeloma. In a particular embodiment, the multiple myeloma is high-risk multiple myeloma or relapsed and refractory multiple myeloma. In a particular embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a particular embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan (e.g., melphalan hydrochloride), oxaliplatin, platinum, procarbazine, streptozocin, temozolomide, thiotepa, and trabectedin. In a particular embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a particular embodiment, the alkylating agent is cyclophosphamide.

In a particular embodiment, the subject is a human.

In a particular embodiment, the BCMA CAR T cells comprise a CAR directed to BCMA, wherein the CAR directed to BCMA comprises an antibody or antibody fragment that targets BCMA. In a particular embodiment, the BCMA CAR T cells comprise a CAR directed to BCMA, wherein the CAR directed to BCMA comprises a single chain Fv antibody or antibody fragment (scFv). In a particular embodiment, the BCMA CAR T cells comprise a CAR directed to BCMA, wherein the CAR directed to BCMA comprises a BCMA02 scFv, e.g., SEQ ID NO: 38.

In a particular embodiment, the subject undergoes a leukapharesis procedure to collect the PBMCs for the manufacture of the BCMA CAR T cells prior to their administration to the subject.

In a particular embodiment, the BCMA CAR T cells are administered by an intravenous infusion.

In another aspect, provided herein is a method of treating a cancer caused by B Cell Maturation Agent (BCMA) expressing cells in a subject in need thereof, wherein the subject has been administered an alkylating agent, comprising: (a) isolating peripheral blood mononuclear cells (PBMCs) from the subject; (b) manufacturing chimeric antigen receptor (CAR) T cells directed to BCMA (BCMA CAR T cells) from the PBMCs; and (c) administering to the subject the BCMA CAR T cells, wherein, at the time of the isolating, the subject has been determined to have been administered the alkylating agent at least about six (6) months prior. In a particular embodiment, the subject has been determined to have been administered the alkylating agent at least about seven (7) months, at least about eight (8) months, at least about nine (9) months, at least about ten (10) months, at least about eleven (11) months, at least about twelve (12) months, at least about thirteen (13) months, or at least about fourteen (14) months prior. In a specific embodiment, the subject has been determined to have been administered the alkylating agent at least about six (6) months to about fourteen (14) months prior, at least about six (6) months to about thirteen (13) months prior, at least about six (6) months to about twelve (12) months prior, at least about six (6) months to about eleven (11) months prior, at least about six (6) months to about ten (10) months prior, at least about six (6) months to about nine (9) months prior, at least about six (6) months to about eight (8) months prior, or at least about six (6) months to about seven (7) months prior. In a specific embodiment, the subject has been determined to have been administered the alkylating agent at least about seven (7) months to about fourteen (14) months prior, at least about eight (8) months to about fourteen (14) months prior, at least about nine (9) months to about fourteen (14) months prior, at least about ten (10) months to about fourteen (14) months prior, at least about eleven (11) months to about fourteen (14) months prior, at least about twelve (12) months to about fourteen (14) months prior, or at least about thirteen (13) months to about fourteen (14) months prior.

In another specific embodiment, the subject has been determined to have been administered the alkylating agent at least about four (4) months or five (5) months to about fourteen (14) months prior, at least about four (4) months or five (5) months to about thirteen (13) months prior, at least about four (4) months or five (5) months to about twelve (12) months prior, at least about four (4) months or five (5) months to about eleven (11) months prior, at least about four (4) months or five (5) months to about ten (10) months prior, at least about four (4) months or five (5) months to about nine (9) months prior, at least about four (4) months or five (5) months to about eight (8) months prior, at least about four (4) months or five (5) months to about seven (7) months prior, or at least about four (4) or five (5) months to about six (6) months prior.

In a particular embodiment, the cancer is multiple myeloma, chronic lymphocytic leukemia, or a non-Hodgkins lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is multiple myeloma. In a particular embodiment, the multiple myeloma is high-risk multiple myeloma or relapsed and refractory multiple myeloma. In a particular embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a particular embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan (e.g., melphalan hydrochloride), oxaliplatin, platinum, procarbazine, streptozocin, temozolomide, thiotepa, and trabectedin. In a particular embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a particular embodiment, the alkylating agent is cyclophosphamide.

In a particular embodiment, the subject is a human.

In a particular embodiment, the BCMA CAR T cells comprise a CAR directed to BCMA, wherein the CAR directed to BCMA comprises an antibody or antibody fragment that targets BCMA. In a particular embodiment, the BCMA CAR T cells comprise a CAR directed to BCMA, wherein the CAR directed to BCMA comprises a single chain Fv antibody or antibody fragment (scFv). In a particular embodiment, the BCMA CAR T cells comprise a CAR directed to BCMA, wherein the CAR directed to BCMA comprises a BCMA02 scFv, e.g., SEQ ID NO: 38.

In a particular embodiment, the subject undergoes a leukapharesis procedure to collect the PBMCs for the manufacture of the BCMA CAR T cells prior to their administration to the subject.

In a particular embodiment, the BCMA CAR T cells are administered by an intravenous infusion.

In another aspect, provided herein is a method of treating a cancer caused by B Cell Maturation Agent (BCMA) expressing cells in a subject in need thereof, wherein the subject has been administered an alkylating agent, comprising administering to the subject chimeric antigen receptor (CAR) T cells directed to BCMA (BCMA CAR T cells) manufactured from peripheral blood mononuclear cells PBMCs isolated from the patient, wherein, at the time said PBMCs are isolated, the subject has last received the alkylating agent at least about six (6) months prior to the time the PBMCs are isolated. In a particular embodiment, the subject has last received the alkylating agent at least about seven (7) months, at least about eight (8) months, at least about nine (9) months, at least about ten (10) months, at least about eleven (11) months, at least about twelve (12) months, at least about thirteen (13) months, or at least about fourteen (14) months prior to the time the PBMCs are isolated.

In another specific embodiment, the subject has has last received the alkylating agent at least about four (4) months or five (5) months to about fourteen (14) months prior, at least about four (4) months or five (5) months to about thirteen (13) months prior to the time the PBMCs are isolated, at least about four (4) months or five (5) months to about twelve (12) months prior to the time the PBMCs are isolated, at least about four (4) months or five (5) months to about eleven (11) months prior to the time the PBMCs are isolated, at least about four (4) months or five (5) months to about ten (10) months prior to the time the PBMCs are isolated, at least about four (4) months or five (5) months to about nine (9) months prior to the time the PBMCs are isolated, at least about four (4) months or five (5) months to about eight (8) months prior to the time the PBMCs are isolated, at least about four (4) months or five (5) months to about seven (7) months prior to the time the PBMCs are isolated, or at least about four (4) or five (5) months to about six (6) months prio to the time the PBMCs are isolated.

In a particular embodiment, the cancer is multiple myeloma, chronic lymphocytic leukemia, or a non-Hodgkins lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is multiple myeloma. In a particular embodiment, the multiple myeloma is high-risk multiple myeloma or relapsed and refractory multiple myeloma. In a particular embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a particular embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan (e.g., melphalan hydrochloride), oxaliplatin, platinum, procarbazine, streptozocin, temozolomide, thiotepa, and trabectedin. In a particular embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a particular embodiment, the alkylating agent is cyclophosphamide.

In a particular embodiment, the subject is a human.

In a particular embodiment, the BCMA CAR T cells comprise a CAR directed to BCMA, wherein the CAR directed to BCMA comprises an antibody or antibody fragment that targets BCMA. In a particular embodiment, the BCMA CAR T cells comprise a CAR directed to BCMA, wherein the CAR directed to BCMA comprises a single chain Fv antibody or antibody fragment (scFv). In a particular embodiment, the BCMA CAR T cells comprise a CAR directed to BCMA, wherein the CAR directed to BCMA comprises a BCMA02 scFv, e.g., SEQ ID NO: 38.

In a particular embodiment, the subject undergoes a leukapharesis procedure to collect the PBMCs for the manufacture of the BCMA CAR T cells prior to their administration to the subject.

In a particular embodiment, the BCMA CAR T cells are administered by an intravenous infusion.

In another aspect, provided herein is a method of treating a cancer caused by B Cell Maturation Agent (BCMA) expressing cells in a subject in need thereof, wherein the subject has been administered an alkylating agent, comprising administering to the subject chimeric antigen receptor (CAR) T cells directed to BCMA (BCMA CAR T cells) manufactured from peripheral blood mononuclear cells PBMCs isolated from the patient, wherein, at the time said PBMCs are isolated, the PBMCs comprise at least about 20% T cells.

In a particular embodiment, the cancer is multiple myeloma, chronic lymphocytic leukemia, or a non-Hodgkins lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is multiple myeloma. In a particular embodiment, the multiple myeloma is high-risk multiple myeloma or relapsed and refractory multiple myeloma. In a particular embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a particular embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan, oxaliplatin, platinum, procarbazine, streptozocin, temozolomide, thiotepa, and trabectedin. In a particular embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a particular embodiment, the alkylating agent is cyclophosphamide.

In a particular embodiment, the subject is a human.

In a particular embodiment, the BCMA CAR T cells comprise a CAR directed to BCMA, wherein the CAR directed to BCMA comprises an antibody or antibody fragment that targets BCMA. In a particular embodiment, the BCMA CAR T cells comprise a CAR directed to BCMA, wherein the CAR directed to BCMA comprises a single chain Fv antibody or antibody fragment (scFv). In a particular embodiment, the BCMA CAR T cells comprise a CAR directed to BCMA, wherein the CAR directed to BCMA comprises a BCMA02 scFv, e.g., SEQ ID NO: 38. In a particular embodiment, the BCMA CAR T cells are idecabtagene vicleucel cells.

In a particular embodiment, the subject undergoes a leukapharesis procedure to collect the PBMCs for the manufacture of the BCMA CAR T cells prior to their administration to the subject.

In a particular embodiment, the BCMA CAR T cells are administered by an intravenous infusion.

In another aspect, provided herein is a method of manufacturing chimeric antigen receptor (CAR) T cells from a subject, comprising: (a) administering to the subject an alkylating agent for treatment of a tumor or a cancer; (b) isolating peripheral blood mononuclear cells (PBMCs) from the subject at least about six months after step (a); and (c) manufacturing chimeric antigen receptor (CAR) T cells from the PBMCs. In a particular embodiment, step (b) is performed at least about seven (7) months, at least about eight (8) months, at least about nine (9) months, at least about ten (10) months, at least about eleven (11) months, at least about twelve (12) months, at least about thirteen (13) months, or at least about fourteen (14) months after step (a). In a specific embodiment, step (b) is performed at least about six (6) months to about fourteen (14) months after step (a), at least about six (6) months to about thirteen (13) months after step (a), at least about six (6) months to about twelve (12) months after step (a), at least about six (6) months to about eleven (11) months after step (a), at least about six (6) months to about ten (10) months after step (a), at least about six (6) months to about nine (9) months after step (a), at least about six (6) months to about eight (8) months after step (a), or at least about six (6) months to about seven (7) months after step (a). In a specific embodiment, step (b) is performed at least about seven (7) months to about fourteen (14) months after step (a), at least about eight (8) months to about fourteen (14) months after step (a), at least about nine (9) months to about fourteen (14) months after step (a), at least about ten (10) months to about fourteen (14) months after step (a), at least about eleven (11) months to about fourteen (14) months after step (a), at least about twelve (12) months to about fourteen (14) months after step (a), or at least about thirteen (13) months to about fourteen (14) months after step (a).

In another specific embodiment, step (b) is performed at least about four (4) months or five (5) months to about fourteen (14) months after step (a), at least about four (4) months or five (5) months to about thirteen (13) months after step (a), at least about four (4) months or five (5) months to about twelve (12) months after step (a), at least about four (4) months or five (5) months to about eleven (11) months after step (a), at least about four (4) months or five (5) months to about ten (10) months after step (a), at least about four (4) months or five (5) months to about nine (9) months after step (a), at least about four (4) months or five (5) months to about eight (8) months after step (a), at least about four (4) months or five (5) months to about seven (7) months after step (a), or at least about four (4) or five (5) months to about six (6) months after step (a).

In a particular embodiment, the tumor or cancer is lymphoma, lung cancer, breast cancer, prostate cancer, adrenocortical carcinoma, thyroid carcinoma, nasopharyngeal carcinoma, melanoma, skin carcinoma, colorectal carcinoma, a desmoid tumor, a desmoplastic small round cell tumor, an endocrine tumor, a Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipomachronic lymphocytic leukemia (small lymphocytic lymphoma), B-cell prolymphocytic leukemia, lymphoplasmacytic lymphoma, Waldenström macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, a non-Hodgkin lymphoma, or multiple myeloma. In a particular embodiment, the cancer is multiple myeloma, chronic lymphocytic leukemia, or a non-Hodgkins lymphoma. In a particular embodiment, the cancer is multiple myeloma, chronic lymphocytic leukemia, or a non-Hodgkins lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is multiple myeloma. In a particular embodiment, the multiple myeloma is high-risk multiple myeloma or relapsed and refractory multiple myeloma. In a particular embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a particular embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan (e.g., melphalan hydrochloride), oxaliplatin, platinum, procarbazine, streptozocin, temozolomide, thiotepa, and trabectedin. In a particular embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a particular embodiment, the alkylating agent is cyclophosphamide.

In a particular embodiment, the subject is a human.

In another aspect, provided herein is a method of manufacturing chimeric antigen receptor (CAR) T cells from a subject, comprising: (a) isolating peripheral blood mononuclear cells (PBMCs) from the subject, and determining that at least about 20% of the PBMCs are T cells; and (b) on the basis of the determination in step (a), subsequently manufacturing chimeric antigen receptor (CAR) T cells from the PBMCs; wherein, prior to step (a), the subject had previously received an alkylating agent for treatment of a tumor or a cancer. In a particular embodiment, the subject had previously received the alkylating agent at least about six months prior to step (a). In a particular embodiment, the subject had previously received the alkylating agent at least about seven (7) months, at least about eight (8) months, at least about nine (9) months, at least about ten (10) months, at least about eleven (11) months, at least about twelve (12) months, at least about thirteen (13) months, or at least about fourteen (14) months prior to step (a). In a specific embodiment, the subject had previously received the alkylating agent at least about six (6) months to about fourteen (14) months prior to step (a), at least about six (6) months to about thirteen (13) months prior to step (a), at least about six (6) months to about twelve (12) months prior to step (a), at least about six (6) months to about eleven (11) months prior to step (a), at least about six (6) months to about ten (10) months prior to step (a), at least about six (6) months to about nine (9) months prior to step (a), at least about six (6) months to about eight (8) months after step (a), or at least about six (6) months to about seven (7) months prior to step (a). In a specific embodiment, the subject had previously received the alkylating agent at least about seven (7) months to about fourteen (14) months prior to step (a), at least about eight (8) months to about fourteen (14) months prior to step (a), at least about nine (9) months to about fourteen (14) months prior to step (a), at least about ten (10) months to about fourteen (14) months prior to step (a), at least about eleven (11) months to about fourteen (14) months prior to step (a), at least about twelve (12) months to about fourteen (14) months prior to step (a), or at least about thirteen (13) months to about fourteen (14) months prior to step (a).

In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are T cells.

In a particular embodiment of the methods presented herein, the T cells comprise CD3+ cells. In particular embodiments, the T cells are CD3+ cells. In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T CD3+ T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are CD3+ T cells, e.g., CD45+/CD3+ T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are CD3+ T cells.

In a particular embodiment, the T cells are CD8+ effector memory RA (T_{EMRA}) (CCR7−/CD45RA+) T cells. In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T_{EMRA} T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are T_{EMRA} T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are T_{EMRA} T cells. In particular embodiments of such methods, the T cells are CD3+ cells.

In the methods presented herein, the determining may be performed using standard techniques well known to those of skill in the relevant art. For example, in the methods presented herein, the determining step may be performed by utilizing techniques such as those utilized in Example 2 (e.g., immunophenotyping of the PBMCs by polychromatic flow cytometry for markers associated with T cell differentiation, memory, senescence, and exhaustion).

In another specific embodiment, the subject had previously received the alkylating agent at least about four (4) months or five (5) months to about fourteen (14) months prior to step (a), at least about four (4) months or five (5) months to about thirteen (13) months prior to step (a), at least about four (4) months or five (5) months to about twelve (12) months prior to step (a), at least about four (4) months or five (5) months to about eleven (11) months prior to step (a), at least about four (4) months or five (5) months to about ten (10) months prior to step (a), at least about four (4) months or five (5) months to about nine (9) months prior to step (a), at least about four (4) months or five (5) months to about eight (8) months prior to step (a), at least about four (4) months or five (5) months to about seven (7) months prior to step (a), or at least about four (4) or five (5) months to about six (6) months prior to step (a).

In a particular embodiment, the tumor or cancer is lymphoma, lung cancer, breast cancer, prostate cancer, adrenocortical carcinoma, thyroid carcinoma, nasopharyngeal carcinoma, melanoma, skin carcinoma, colorectal carcinoma, a desmoid tumor, a desmoplastic small round cell tumor, an endocrine tumor, a Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipomachronic lymphocytic leukemia (small lymphocytic lymphoma), B-cell prolymphocytic leukemia, lymphoplasmacytic lymphoma, Waldenström macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, a non-Hodgkin lymphoma, or multiple myeloma. In a particular embodiment, the cancer is multiple myeloma, chronic lymphocytic leukemia, or a non-Hodgkins lymphoma. In a particular embodiment, the cancer is multiple myeloma, chronic lymphocytic leukemia, or a non-Hodgkins lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is multiple myeloma. In a particular embodiment, the multiple myeloma is high-risk multiple myeloma or relapsed and refractory multiple myeloma. In a particular embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a particular embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan (e.g., melphalan hydrochloride), oxaliplatin, platinum, procarbazine, streptozocin, temozolomide, thiotepa, and trabectedin. In a particular embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a particular embodiment, the alkylating agent is cyclophosphamide.

In a particular embodiment, the subject is a human.

In another aspect, provided herein is a method of manufacturing chimeric antigen receptor (CAR) T cells from a subject, comprising: (a) isolating peripheral blood mononuclear cells (PBMCs) from the subject; and (b) manufacturing chimeric antigen receptor (CAR) T cells from the PBMCs; wherein the subject had previously received an alkylating agent for treatment of a tumor or a cancer; wherein step (a) occurs at least about six months after the subject received the alkylating agent. In a particular embodiment, step (a) further comprises determining that at least about 20% of the PBMCs are T cells; and wherein step (b) further comprises subsequently manufacturing chimeric antigen receptor (CAR) T cells from the PBMCs on the basis of the determination in step (a). In a particular embodiment, step (a) occurs at least about seven (7) months, at least about eight (8) months, at least about nine (9) months, at least about ten (10) months, at least about eleven (11) months, at least about twelve (12) months, at least about thirteen (13) months, or at least about fourteen (14) months after the subject received the alkylating agent. In a specific embodiment, step (a) occurs at least about six (6) months to about fourteen (14) months after the subject received the alkylating agent, at least about six (6) months to about thirteen (13) months after the subject received the alkylating agent, at least about six (6) months to about twelve (12) months after the subject received the alkylating agent, at least about six (6) months to about eleven (11) months after the subject received the alkylating agent, at least about six (6) months to about ten (10) months after the subject received the alkylating agent, at least about six (6) months to about nine (9) months after the subject received the alkylating agent, at least about six (6) months to about eight (8) months after the subject received the alkylating agent, or at least about six (6) months to about seven (7) months after the subject received the alkylating agent. In a specific embodiment, step (a) occurs at least about seven (7) months to about fourteen (14) months after the subject received the alkylating agent, at least about eight (8) months to about fourteen (14) months after the subject received the alkylating agent, at least about nine (9) months to about fourteen (14) months after the subject received the alkylating agent, at least about ten (10) months to about fourteen (14) months after the subject received the alkylating agent, at least about eleven (11) months to about fourteen (14) months after the subject received the alkylating agent, at least about twelve (12) months to about fourteen (14) months after the subject received the alkylating agent, or at least about thirteen (13) months to about fourteen (14) months after the subject received the alkylating agent.

In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are T cells.

In a particular embodiment of the methods presented herein, the T cells comprise CD3+ cells. In particular embodiments, the T cells are CD3+ cells. In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T CD3+ T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are CD3+ T cells, e.g., CD45+/CD3+ T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are CD3+ T cells.

In a particular embodiment, the T cells are CD8+ effector memory RA (T_{EMRA}) (CCR7−/CD45RA+) T cells. In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T_{EMRA} T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are T_{EMRA} T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are T_{EMRA} T cells. In particular embodiments of such methods, the T cells are CD3+ cells.

In the methods presented herein, the determining may be performed using standard techniques well known to those of skill in the relevant art. For example, in the methods presented herein, the determining step may be performed by utilizing techniques such as those utilized in Example 2 (e.g., immunophenotyping of the PBMCs by polychromatic flow cytometry for markers associated with T cell differentiation, memory, senescence, and exhaustion).

In another specific embodiment, step (a) occurs at least about four (4) months or five (5) months to about fourteen (14) months after the subject received the alkylating agent, at least about four (4) months or five (5) months to about thirteen (13) months after the subject received the alkylating agent, at least about four (4) months or five (5) months to about twelve (12) months after the subject received the alkylating agent, at least about four (4) months or five (5) months to about eleven (11) months after the subject received the alkylating agent, at least about four (4) months or five (5) months to about ten (10) months after the subject received the alkylating agent, at least about four (4) months or five (5) months to about nine (9) months after the subject received the alkylating agent, at least about four (4) months or five (5) months to about eight (8) months after the subject received the alkylating agent, at least about four (4) months or five (5) months to about seven (7) months after the subject received the alkylating agent, or at least about four (4) or five (5) months to about six (6) months after the subject received the alkylating agent.

In a particular embodiment, the tumor or cancer is lymphoma, lung cancer, breast cancer, prostate cancer, adrenocortical carcinoma, thyroid carcinoma, nasopharyngeal carcinoma, melanoma, skin carcinoma, colorectal carcinoma, a desmoid tumor, a desmoplastic small round cell tumor, an endocrine tumor, a Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipomachronic lymphocytic leukemia (small lymphocytic lymphoma), B-cell prolymphocytic leukemia, lymphoplasmacytic lymphoma, Waldenström macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, a non-Hodgkin lymphoma, or multiple myeloma. In a particular embodiment, the cancer is multiple myeloma, chronic lymphocytic leukemia, or a non-Hodgkins lymphoma. In a particular embodiment, the cancer is multiple myeloma, chronic lymphocytic leukemia, or a non-Hodgkins lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is multiple myeloma. In a particular embodiment, the multiple myeloma is high-risk multiple myeloma or relapsed and refractory multiple myeloma. In a particular embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a particular embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan (e.g., melphalan hydrochloride), oxaliplatin, platinum, procarbazine, streptozocin, temozolomide, thiotepa, and trabectedin. In a particular embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a particular embodiment, the alkylating agent is cyclophosphamide.

In a particular embodiment, the subject is a human.

In another aspect, provided herein is a method of manufacturing chimeric antigen receptor (CAR) T cells from a subject, wherein the subject has been administered an alkylating agent for treatment of a tumor or a cancer, comprising: a. determining that the subject has not been administered the alkylating agent less than about six (6) months prior to the determining step; b. isolating peripheral blood mononuclear cells (PBMCs) from the subject; and c. manufacturing chimeric antigen receptor (CAR) T cells from the PBMCs. In a particular embodiment, in step (a) the subject has not been administered the alkylating agent less than about seven (7) months, less than about eight (8) months, less than about nine (9) months, less than about ten (10) months, less than about eleven (11) months, less than about twelve (12) months, less than about thirteen (13) months, or less than about fourteen (14) months prior to the determining step.

In a particular embodiment, the tumor or cancer is lymphoma, lung cancer, breast cancer, prostate cancer, adrenocortical carcinoma, thyroid carcinoma, nasopharyngeal carcinoma, melanoma, skin carcinoma, colorectal carcinoma, a desmoid tumor, a desmoplastic small round cell tumor, an endocrine tumor, a Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipomachronic lymphocytic leukemia (small lymphocytic lymphoma), B-cell prolymphocytic leukemia, lymphoplasmacytic lymphoma, Waldenström macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, a non-Hodgkin lymphoma, or multiple myeloma. In a particular embodiment, the cancer is multiple myeloma, chronic lymphocytic leukemia, or a non-Hodgkins lymphoma. In a particular embodiment, the cancer is multiple myeloma, chronic lymphocytic leukemia, or a non-Hodgkins lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is multiple myeloma. In a particular embodiment, the multiple myeloma is high-risk multiple myeloma or relapsed and refractory multiple myeloma. In a particular embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a particular embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan (e.g., melphalan hydrochloride), oxaliplatin, platinum, procarbazine, streptozocin, temozolomide, thiotepa, and trabectedin. In a particular embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a particular embodiment, the alkylating agent is cyclophosphamide.

In a particular embodiment, the subject is a human.

In another aspect, provided herein is a method of manufacturing chimeric antigen receptor (CAR) T cells from a subject, wherein the subject has been administered an alkylating agent for treatment of a tumor or a cancer, comprising: (a) isolating peripheral blood mononuclear cells (PBMCs) from the subject; and (b) manufacturing chimeric antigen receptor (CAR) T cells from the PBMCs; wherein, at the time of the isolating, the subject has been determined to have been administered the alkylating agent at least about six (6) months prior. In a particular embodiment, the subject has been determined to have been administered the alkylating agent at least about seven (7) months, at least about eight (8) months, at least about nine (9) months, at least about ten (10) months, at least about eleven (11) months, at least about twelve (12) months, at least about thirteen (13) months, or at least about fourteen (14) months prior. In a specific embodiment, the subject has been determined to have been administered the alkylating agent at least about six (6) months to about fourteen (14) months prior, at least about six (6) months to about thirteen (13) months prior, at least about six (6) months to about twelve (12) months prior, at least about six (6) months to about eleven (11) months prior, at least about six (6) months to about ten (10) months prior, at least about six (6) months to about nine (9) months prior, at least about six (6) months to about eight (8) months prior, or at least about six (6) months to about seven (7) months prior. In a specific embodiment, the subject has been determined to have been administered the alkylating agent at least about seven (7) months to about fourteen (14) months prior, at least about eight (8) months to about fourteen (14) months prior, at least about nine (9) months to about fourteen (14) months prior, at least about ten (10) months to about fourteen (14) months prior, at least about eleven (11) months to about fourteen (14) months prior, at least about twelve (12) months to about fourteen (14) months prior, or at least about thirteen (13) months to about fourteen (14) months prior.

In another specific embodiment, the subject has been determined to have been administered the alkylating agent at least about four (4) months or five (5) months to about fourteen (14) months prior, at least about four (4) months or five (5) months to about thirteen (13) months prior, at least about four (4) months or five (5) months to about twelve (12) months prior, at least about four (4) months or five (5) months to about eleven (11) months prior, at least about four (4) months or five (5) months to about ten (10) months prior, at least about four (4) months or five (5) months to about nine (9) months prior, at least about four (4) months or five (5) months to about eight (8) months prior, at least about four (4) months or five (5) months to about seven (7) months prior, or at least about four (4) or five (5) months to about six (6) months prior.

In a particular embodiment, the tumor or cancer is lymphoma, lung cancer, breast cancer, prostate cancer, adrenocortical carcinoma, thyroid carcinoma, nasopharyngeal carcinoma, melanoma, skin carcinoma, colorectal carcinoma, a desmoid tumor, a desmoplastic small round cell tumor, an endocrine tumor, a Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipomachronic lymphocytic leukemia (small lymphocytic lymphoma), B-cell prolymphocytic leukemia, lymphoplasmacytic lymphoma, Waldenström macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, a non-Hodgkin lymphoma, or multiple myeloma. In a particular embodiment, the cancer is multiple myeloma, chronic lymphocytic leukemia, or a non-Hodgkins lymphoma. In a particular embodiment, the cancer is multiple myeloma, chronic lymphocytic leukemia, or a non-Hodgkins lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is multiple myeloma. In a particular embodiment, the multiple myeloma is high-risk multiple myeloma or relapsed and refractory multiple myeloma. In a particular embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a particular embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan (e.g., melphalan hydrochloride), oxaliplatin, platinum, procarbazine, streptozocin, temozolomide, thiotepa, and trabectedin. In a particular embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a particular embodiment, the alkylating agent is cyclophosphamide.

In a particular embodiment, the subject is a human.

In another aspect, provided herein is a method of manufacturing chimeric antigen receptor (CAR) T cells from a subject, comprising: (a) isolating peripheral blood mononuclear cells (PBMCs) from the subject, and determining that at least about 20% of the PBMCs are T cells; and (b) on the basis of the determination in step (a), subsequently manufacturing the CAR T cells from the PBMCs; wherein the subject had previously received an alkylating agent for treatment of a tumor or a cancer. In a particular embodiment, the subject had previously received the alkylating agent at least about six months prior to step (a). In a particular embodiment, the subject had previously received the alkylating agent at least about seven (7) months, at least about eight (8) months, at least about nine (9) months, at least about ten (10) months, at least about eleven (11) months, at least about twelve (12) months, at least about thirteen (13) months, or at least about fourteen (14) months prior to step (a). In a specific embodiment, the subject had previously received the alkylating agent at least about six (6) months to about fourteen (14) months prior to step (a), at least about six (6) months to about thirteen (13) months prior to step (a), at least about six (6) months to about twelve (12) months prior to step (a), at least about six (6) months to about eleven (11) months prior to step (a), at least about six (6) months to about ten (10) months prior to step (a), at least about six (6) months to about nine (9) months prior to step (a), at least about six (6) months to about eight (8) months after step (a), or at least about six (6) months to about seven (7) months prior to step (a). In a specific embodiment, step (b) is performed at least about seven (7) months to about fourteen (14) months prior to step (a), at least about eight (8) months to about fourteen (14) months prior to step (a), at least about nine (9) months to about fourteen (14) months prior to step (a), at least about ten (10) months to about fourteen (14) months prior to step (a), at least about eleven (11) months to about fourteen (14) months prior to step (a), at least about twelve (12) months to about fourteen (14) months prior to step (a), or at least about thirteen (13) months to about fourteen (14) months prior to step (a).

In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are T cells.

In a particular embodiment of the methods presented herein, the T cells comprise CD3+ cells. In particular embodiments, the T cells are CD3+ cells. In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T CD3+ T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are CD3+ T cells, e.g., CD45+/CD3+ T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are CD3+ T cells.

In a particular embodiment, the T cells are CD8+ effector memory RA (T_{EMRA}) (CCR7−/CD45RA+) T cells. In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T_{EMRA} T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are T_{EMRA} T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are T_{EMRA} T cells. In particular embodiments of such methods, the T cells are CD3+ cells.

In the methods presented herein, the determining may be performed using standard techniques well known to those of skill in the relevant art. For example, in the methods presented herein, the determining step may be performed by utilizing techniques such as those utilized in Example 2 (e.g., immunophenotyping of the PBMCs by polychromatic flow cytometry for markers associated with T cell differentiation, memory, senescence, and exhaustion).

In another specific embodiment, the subject had previously received the alkylating agent at least about four (4) months or five (5) months to about fourteen (14) months prior to step (a), at least about four (4) months or five (5) months to about thirteen (13) months prior to step (a), at least about four (4) months or five (5) months to about twelve (12) months prior to step (a), at least about four (4) months or five (5) months to about eleven (11) months prior to step (a), at least about four (4) months or five (5) months to about ten (10) months prior to step (a), at least about four (4) months or five (5) months to about nine (9) months prior to step (a), at least about four (4) months or five (5) months to about eight (8) months prior to step (a), at least about four (4) months or five (5) months to about seven (7) months prior to step (a), or at least about four (4) or five (5) months to about six (6) months prior to step (a).

In a particular embodiment, the tumor or cancer is lymphoma, lung cancer, breast cancer, prostate cancer, adrenocortical carcinoma, thyroid carcinoma, nasopharyngeal carcinoma, melanoma, skin carcinoma, colorectal carcinoma, a desmoid tumor, a desmoplastic small round cell tumor, an endocrine tumor, a Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipomachronic lymphocytic leukemia (small lymphocytic lymphoma), B-cell prolymphocytic leukemia, lymphoplasmacytic lymphoma, Waldenström macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, a non-Hodgkin lymphoma, or multiple myeloma. In a particular embodiment, the cancer is multiple myeloma, chronic lymphocytic leukemia, or a non-Hodgkins lymphoma. In a particular embodiment, the cancer is multiple myeloma, chronic lymphocytic leukemia, or a non-Hodgkins lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is multiple myeloma. In a particular embodiment, the multiple myeloma is high-risk multiple myeloma or relapsed and refractory multiple myeloma. In a particular embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a particular embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan (e.g., melphalan hydrochloride), oxaliplatin, platinum, procarbazine, streptozocin, temozolomide, thiotepa, and trabectedin. In a particular embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a particular embodiment, the alkylating agent is cyclophosphamide.

In a particular embodiment, the subject is a human.

In another aspect, provided herein is a method of manufacturing chimeric antigen receptor (CAR) T cells directed to BCMA (BCMA CAR T cells) from a subject, comprising: (a) administering to the subject an alkylating agent for treatment of a cancer; (b) isolating peripheral blood mononuclear cells (PBMCs) from the subject at least about six months after step (a); and (c) manufacturing BCMA CAR T cells from the PBMCs. In a particular embodiment, step (b) is performed at least about seven (7) months, at least about eight (8) months, at least about nine (9) months, at least about ten (10) months, at least about eleven (11) months, at least about twelve (12) months, at least about thirteen (13) months, or at least about fourteen (14) months after step (a). In a specific embodiment, step (b) is performed at least about six (6) months to about fourteen (14) months after step (a), at least about six (6) months to about thirteen (13) months after step (a), at least about six (6) months to about twelve (12) months after step (a), at least about six (6) months to about eleven (11) months after step (a), at least about six (6) months to about ten (10) months after step (a), at least about six (6) months to about nine (9) months after step (a), at least about six (6) months to about eight (8) months after step (a), or at least about six (6) months to about seven (7) months after step (a). In a specific embodiment, step (b) is performed at least about seven (7) months to about fourteen (14) months after step (a), at least about eight (8) months to about fourteen (14) months after step (a), at least about nine (9) months to about fourteen (14) months after step (a), at least about ten (10) months to about fourteen (14) months after step (a), at least about eleven (11) months to about fourteen (14) months after step (a), at least about twelve (12) months to about fourteen (14) months after step (a), or at least about thirteen (13) months to about fourteen (14) months after step (a).

In another specific embodiment, step (b) is performed at least about four (4) months or five (5) months to about fourteen (14) months after step (a), at least about four (4) months or five (5) months to about thirteen (13) months after step (a), at least about four (4) months or five (5) months to about twelve (12) months after step (a), at least about four (4) months or five (5) months to about eleven (11) months after step (a), at least about four (4) months or five (5) months to about ten (10) months after step (a), at least about four (4) months or five (5) months to about nine (9) months after step (a), at least about four (4) months or five (5) months to about eight (8) months after step (a), at least about four (4) months or five (5) months to about seven (7) months after step (a), or at least about four (4) or five (5) months to about six (6) months after step (a).

In a particular embodiment, the cancer is multiple myeloma, chronic lymphocytic leukemia, or a non-Hodgkins lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is multiple myeloma. In a particular embodiment, the multiple myeloma is high-risk multiple myeloma or relapsed and refractory multiple myeloma. In a particular embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a particular embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan (e.g., melphalan hydrochloride), oxaliplatin, platinum, procarbazine, streptozocin, temozolomide, thiotepa, and trabectedin. In a particular embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a particular embodiment, the alkylating agent is cyclophosphamide.

In a particular embodiment, the subject is a human.

In a particular embodiment, the BCMA CAR T cells comprise a CAR directed to BCMA, wherein the CAR directed to BCMA comprises an antibody or antibody fragment that targets BCMA. In a particular embodiment, the BCMA CAR T cells comprise a CAR directed to BCMA, wherein the CAR directed to BCMA comprises a single chain Fv antibody or antibody fragment (scFv). In a particular embodiment, the BCMA CAR T cells comprise a CAR directed to BCMA, wherein the CAR directed to BCMA comprises a BCMA02 scFv, e.g., SEQ ID NO: 38.

In another aspect, provided herein is a method of manufacturing chimeric antigen receptor (CAR) T cells directed to BCMA (BCMA CAR T cells) from a subject, comprising: (a) isolating peripheral blood mononuclear cells (PBMCs) from the subject, and determining that at least about 20% of the PBMCs are T cells; and (b) on the basis of the determination in step (a), subsequently manufacturing BCMA CAR T cells from the PBMCs; wherein, prior to step (a), the subject had previously received an alkylating agent for treatment of a cancer. In a specific embodiment, the subject had previously received the alkylating agent at least about six months prior to step (a). In a specific embodiment, the subject had previously received the alkylating agent at least about seven (7) months, at least about eight (8) months, at least about nine (9) months, at least about ten (10) months, at least about eleven (11) months, at least about twelve (12) months, at least about thirteen (13) months, or at least about fourteen (14) months prior to step (a). In a specific embodiment, the subject had previously received the alkylating agent at least about six (6) months to about fourteen (14) months prior to step (a), at least about six (6) months to about thirteen (13) months prior to step (a), at least about six (6) months to about twelve (12) months prior to step (a), at least about six (6) months to about eleven (11) months prior to step (a), at least about six (6) months to about ten (10) months prior to step (a), at least about six (6) months to about nine (9) months prior to step (a), at least about six (6) months to about eight (8) months after step (a), or at least about six (6) months to about seven (7) months prior to step (a). In a specific embodiment, step (b) is performed at least about seven (7) months to about fourteen (14) months prior to step (a), at least about eight (8) months to about fourteen (14) months prior to step (a), at least about nine (9) months to about fourteen (14) months prior to step (a), at least about ten (10) months to about fourteen (14) months prior to step (a), at least about eleven (11) months to about fourteen (14) months prior to step (a), at least about twelve (12) months to about fourteen (14) months prior to step (a), or at least about thirteen (13) months to about fourteen (14) months prior to step (a).

In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are T cells.

In a particular embodiment of the methods presented herein, the T cells comprise CD3+ cells. In particular embodiments, the T cells are CD3+ cells. In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T CD3+ T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are CD3+ T cells, e.g., CD45+/CD3+ T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are CD3+ T cells.

In a particular embodiment, the T cells are CD8+ effector memory RA (T_{EMRA}) (CCR7−/CD45RA+) T cells. In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T_{EMRA} T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are T_{EMRA} T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are T_{EMRA} T cells. In particular embodiments of such methods, the T cells are CD3+ cells.

In the methods presented herein, the determining may be performed using standard techniques well known to those of skill in the relevant art. For example, in the methods presented herein, the determining step may be performed by utilizing techniques such as those utilized in Example 2 (e.g., immunophenotyping of the PBMCs by polychromatic flow cytometry for markers associated with T cell differentiation, memory, senescence, and exhaustion).

In another specific embodiment, the subject had previously received the alkylating agent at least about four (4) months or five (5) months to about fourteen (14) months prior to step (a), at least about four (4) months or five (5) months to about thirteen (13) months prior to step (a), at least about four (4) months or five (5) months to about twelve (12) months prior to step (a), at least about four (4) months or five (5) months to about eleven (11) months prior to step (a), at least about four (4) months or five (5) months to about ten (10) months prior to step (a), at least about four (4) months or five (5) months to about nine (9) months prior to step (a), at least about four (4) months or five (5) months to about eight (8) months prior to step (a), at least about four (4) months or five (5) months to about seven (7) months prior to step (a), or at least about four (4) or five (5) months to about six (6) months prior to step (a).

In a particular embodiment, the cancer is multiple myeloma, chronic lymphocytic leukemia, or a non-Hodgkins lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is multiple myeloma. In a particular embodiment, the multiple myeloma is high-risk multiple myeloma or relapsed and refractory multiple myeloma. In a particular embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a particular embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan (e.g., melphalan hydrochloride), oxaliplatin, platinum, procarbazine, streptozocin, temozolomide, thiotepa, and trabectedin. In a particular embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a particular embodiment, the alkylating agent is cyclophosphamide.

In a particular embodiment, the subject is a human.

In a particular embodiment, the BCMA CAR T cells comprise a CAR directed to BCMA, wherein the CAR directed to BCMA comprises an antibody or antibody fragment that targets BCMA. In a particular embodiment, the BCMA CAR T cells comprise a CAR directed to BCMA, wherein the CAR directed to BCMA comprises a single chain Fv antibody or antibody fragment (scFv). In a particular embodiment, the BCMA CAR T cells comprise a CAR directed to BCMA, wherein the CAR directed to BCMA comprises a BCMA02 scFv, e.g., SEQ ID NO: 38.

In another aspect, provided herein is a method of manufacturing chimeric antigen receptor (CAR) T cells directed to BCMA (BCMA CAR T cells) from a subject, comprising: (a) isolating peripheral blood mononuclear cells (PBMCs) from the subject; and (b) manufacturing BCMA CAR T cells from the PBMCs; wherein the subject had previously received an alkylating agent for treatment of a cancer; wherein step (a) occurs at least about six months after the subject received the alkylating agent. In a particular embodiment, step (a) further comprises determining that at least about 20% of the PBMCs are T cells; and wherein step (b) further comprises subsequently manufacturing chimeric antigen receptor (CAR) T cells from the PBMCs on the basis of the determination in step (a). In a particular embodiment, step (a) occurs at least about seven (7) months, at least about eight (8) months, at least about nine (9) months, at least about ten (10) months, at least about eleven (11) months, at least about twelve (12) months, at least about thirteen (13) months, or at least about fourteen (14) months after the subject received the alkylating agent. In a specific embodiment, step (a) occurs at least about six (6) months to about fourteen (14) months after the subject received the alkylating agent, at least about six (6) months to about thirteen (13) months after the subject received the alkylating agent, at least about six (6) months to about twelve (12) months after the subject received the alkylating agent, at least about six (6) months to about eleven (11) months after the subject received the alkylating agent, at least about six (6) months to about ten (10) months after the subject received the alkylating agent, at least about six (6) months to about nine (9) months after the subject received the alkylating agent, at least about six (6) months to about eight (8) months after the subject received the alkylating agent, or at least about six (6) months to about seven (7) months after the subject received the alkylating agent. In a specific embodiment, step (a) occurs at least about seven (7) months to about fourteen (14) months after the subject received the alkylating agent, at least about eight (8) months to about fourteen (14) months after the subject received the alkylating agent, at least about nine (9) months to about fourteen (14) months after the subject received the alkylating agent, at least about ten (10) months to about fourteen (14) months after the subject received the alkylating agent, at least about eleven (11) months to about fourteen (14) months after the subject received the alkylating agent, at least about twelve (12) months to about fourteen (14) months after the subject received the alkylating agent, or at least about thirteen (13) months to about fourteen (14) months after the subject received the alkylating agent.

In another specific embodiment, step (a) occurs at least about four (4) months or five (5) months to about fourteen (14) months after the subject received the alkylating agent, at least about four (4) months or five (5) months to about thirteen (13) months after the subject received the alkylating agent, at least about four (4) months or five (5) months to about twelve (12) months after the subject received the alkylating agent, at least about four (4) months or five (5) months to about eleven (11) months after the subject received the alkylating agent, at least about four (4) months or five (5) months to about ten (10) months after the subject received the alkylating agent, at least about four (4) months or five (5) months to about nine (9) months after the subject received the alkylating agent, at least about four (4) months or five (5) months to about eight (8) months after the subject received the alkylating agent, at least about four (4) months or five (5) months to about seven (7) months after the subject received the alkylating agent, or at least about four (4) or five (5) months to about six (6) months after the subject received the alkylating agent.

In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are T cells.

In a particular embodiment of the methods presented herein, the T cells comprise CD3+ cells. In particular embodiments, the T cells are CD3+ cells. In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T CD3+ T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are CD3+ T cells, e.g., CD45+/CD3+ T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are CD3+ T cells.

In a particular embodiment, the T cells are CD8+ effector memory RA (T_{EMRA}) (CCR7−/CD45RA+) T cells. In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T_{EMRA} T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are T_{EMRA} T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are T_{EMRA} T cells. In particular embodiments of such methods, the T cells are CD3+ cells.

In the methods presented herein, the determining may be performed using standard techniques well known to those of skill in the relevant art. For example, in the methods presented herein, the determining step may be performed by utilizing techniques such as those utilized in Example 2 (e.g., immunophenotyping of the PBMCs by polychromatic flow cytometry for markers associated with T cell differentiation, memory, senescence, and exhaustion).

In a particular embodiment, the cancer is multiple myeloma, chronic lymphocytic leukemia, or a non-Hodgkins lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is multiple myeloma. In a particular embodiment, the multiple myeloma is high-risk multiple myeloma or relapsed and refractory multiple myeloma. In a particular embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a particular embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan (e.g., melphalan hydrochloride), oxaliplatin, platinum, procarbazine, streptozocin, temozolomide, thiotepa, and trabectedin. In a particular embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a particular embodiment, the alkylating agent is cyclophosphamide.

In a particular embodiment, the subject is a human.

In a particular embodiment, the BCMA CAR T cells comprise a CAR directed to BCMA, wherein the CAR directed to BCMA comprises an antibody or antibody fragment that targets BCMA. In a particular embodiment, the BCMA CAR T cells comprise a CAR directed to BCMA, wherein the CAR directed to BCMA comprises a single chain Fv antibody or antibody fragment (scFv). In a particular embodiment, the BCMA CAR T cells comprise a CAR directed to BCMA, wherein the CAR directed to BCMA comprises a BCMA02 scFv, e.g., SEQ ID NO: 38.

In another aspect, provided herein is a method of manufacturing chimeric antigen receptor (CAR) T cells directed to BCMA (BCMA CAR T cells) from a subject, wherein the subject has been administered an alkylating agent for treatment of a cancer, comprising: a. determining that the subject has not been administered the alkylating agent less than about six (6) months prior to the determining step; b. isolating peripheral blood mononuclear cells (PBMCs) from the subject; and c. manufacturing BCMA CAR T cells from the PBMCs. In a particular embodiment, step (a) occurs at least about seven (7) months, at least about eight (8) months, at least about nine (9) months, at least about ten (10) months, at least about eleven (11) months, at least about twelve (12) months, at least about thirteen (13) months, or at least about fourteen (14) months after the subject received the alkylating agent.

In a particular embodiment, the cancer is multiple myeloma, chronic lymphocytic leukemia, or a non-Hodgkins lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is multiple myeloma. In a particular embodiment, the multiple myeloma is high-risk multiple myeloma or relapsed and refractory multiple myeloma. In a particular embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a particular embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan (e.g., melphalan hydrochloride), oxaliplatin, platinum, procarbazine, streptozocin, temozolomide, thiotepa, and trabectedin. In a particular embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a particular embodiment, the alkylating agent is cyclophosphamide.

In a particular embodiment, the subject is a human.

In a particular embodiment, the BCMA CAR T cells comprise a CAR directed to BCMA, wherein the CAR directed to BCMA comprises an antibody or antibody fragment that targets BCMA. In a particular embodiment, the BCMA CAR T cells comprise a CAR directed to BCMA, wherein the CAR directed to BCMA comprises a single chain Fv antibody or antibody fragment (scFv). In a particular embodiment, the BCMA CAR T cells comprise a CAR directed to BCMA, wherein the CAR directed to BCMA comprises a BCMA02 scFv, e.g., SEQ ID NO: 38.

In another aspect, provided herein is a method of manufacturing chimeric antigen receptor (CAR) T cells directed to BCMA (c) from a subject, wherein the subject has been administered an alkylating agent for treatment of a cancer, comprising: (a) isolating peripheral blood mononuclear cells (PBMCs) from the subject; and (b) manufacturing BCMA T cells from the PBMCs; wherein, at the time of the isolating, the subject has been determined to have been administered the alkylating agent at least about six (6) months prior. In a particular embodiment, the subject has been determined to have been administered the alkylating agent at least about seven (7) months, at least about eight (8) months, at least about nine (9) months, at least about ten (10) months, at least about eleven (11) months, at least about twelve (12) months, at least about thirteen (13) months, or at least about fourteen (14) months prior. In a specific embodiment, the subject has been determined to have been administered the alkylating agent at least about six (6) months to about fourteen (14) months prior, at least about six (6) months to about thirteen (13) months prior, at least about six (6) months to about twelve (12) months prior, at least about six (6) months to about eleven (11) months prior, at least about six (6) months to about ten (10) months prior, at least about six (6) months to about nine (9) months prior, at least about six (6) months to about eight (8) months prior, or at least about six (6) months to about seven (7) months prior. In a specific embodiment, the subject has been determined to have been administered the alkylating agent at least about seven (7) months to about fourteen (14) months prior, at least about eight (8) months to about fourteen (14) months prior, at least about nine (9) months to about fourteen (14) months prior, at least about ten (10) months to about fourteen (14) months prior, at least about eleven (11) months to about fourteen (14) months prior, at least about twelve (12) months to about fourteen (14) months prior, or at least about thirteen (13) months to about fourteen (14) months prior.

In another specific embodiment, the subject has been determined to have been administered the alkylating agent at least about four (4) months or five (5) months to about fourteen (14) months prior, at least about four (4) months or five (5) months to about thirteen (13) months prior, at least about four (4) months or five (5) months to about twelve (12) months prior, at least about four (4) months or five (5) months to about eleven (11) months prior, at least about four (4) months or five (5) months to about ten (10) months prior, at least about four (4) months or five (5) months to about nine (9) months prior, at least about four (4) months or five (5) months to about eight (8) months prior, at least about four (4) months or five (5) months to about seven (7) months prior, or at least about four (4) or five (5) months to about six (6) months prior.

In a particular embodiment, the cancer is multiple myeloma, chronic lymphocytic leukemia, or a non-Hodgkins lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is multiple myeloma. In a particular embodiment, the multiple myeloma is high-risk multiple myeloma or relapsed and refractory multiple myeloma. In a particular embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a particular embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan (e.g., melphalan hydrochloride), oxaliplatin, platinum, procarbazine, streptozocin, temozolomide, thiotepa, and trabectedin. In a particular embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a particular embodiment, the alkylating agent is cyclophosphamide.

In a particular embodiment, the subject is a human.

In a particular embodiment, the BCMA CAR T cells comprise a CAR directed to BCMA, wherein the CAR directed to BCMA comprises an antibody or antibody fragment that targets BCMA. In a particular embodiment, the BCMA CAR T cells comprise a CAR directed to BCMA, wherein the CAR directed to BCMA comprises a single chain Fv antibody or antibody fragment (scFv). In a particular embodiment, the BCMA CAR T cells comprise a CAR directed to BCMA, wherein the CAR directed to BCMA comprises a BCMA02 scFv, e.g., SEQ ID NO: 38.

In another aspect, provided herein is a method of manufacturing chimeric antigen receptor (CAR) T cells directed to BCMA (BCMA CAR T cells) from a subject, comprising: (a). isolating peripheral blood mononuclear cells (PBMCs) from the subject; and determining that at least about 20% of the PBMCs are T cells; and b. on the basis of the determination in step (a), subsequently manufacturing the BCMA CAR T cells from the PBMCs; wherein the subject had previously received an alkylating agent for treatment of a cancer caused by BCMA-expressing cells. In a particular embodiment, the subject had previously received the alkylating agent at least about six months prior to step (a). In a particular embodiment, the subject had previously received the alkylating agent at least about seven (7) months, at least about eight (8) months, at least about nine (9) months, at least about ten (10) months, at least about eleven (11) months, at least about twelve (12) months, at least about thirteen (13) months, or at least about fourteen (14) months prior to step (a). In a specific embodiment, the subject had previously received the alkylating agent at least about six (6) months to about fourteen (14) months prior to step (a), at least about six (6) months to about thirteen (13) months prior to step (a), at least about six (6) months to about twelve (12) months prior to step (a), at least about six (6) months to about eleven (11) months prior to step (a), at least about six (6) months to about ten (10) months prior to step (a), at least about six (6) months to about nine (9) months prior to step (a), at least about six (6) months to about eight (8) months after step (a), or at least about six (6) months to about seven (7) months prior to step (a). In a specific embodiment, step (b) is performed at least about seven (7) months to about fourteen (14) months prior to step (a), at least about eight (8) months to about fourteen (14) months prior to step (a), at least about nine (9) months to about fourteen (14) months prior to step (a), at least about ten (10) months to about fourteen (14) months prior to step (a), at least about eleven (11) months to about fourteen (14) months prior to step (a), at least about twelve (12) months to about fourteen (14) months prior to step (a), or at least about thirteen (13) months to about fourteen (14) months prior to step (a).

In another specific embodiment, the subject had previously received the alkylating agent at least about four (4) months or five (5) months to about fourteen (14) months prior to step (a), at least about four (4) months or five (5) months to about thirteen (13) months prior to step (a), at least about four (4) months or five (5) months to about twelve (12) months prior to step (a), at least about four (4) months or five (5) months to about eleven (11) months prior to step (a), at least about four (4) months or five (5) months to about ten (10) months prior to step (a), at least about four (4) months or five (5) months to about nine (9) months prior to step (a), at least about four (4) months or five (5) months to about eight (8) months prior to step (a), at least about four (4) months or five (5) months to about seven (7) months prior to step (a), or at least about four (4) or five (5) months to about six (6) months prior to step (a).

In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are T cells.

In a particular embodiment of the methods presented herein, the T cells comprise CD3+ cells. In particular embodiments, the T cells are CD3+ cells. In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T CD3+ T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are CD3+ T cells, e.g., CD45+/CD3+ T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are CD3+ T cells.

In a particular embodiment, the T cells are CD8+ effector memory RA (T_{EMRA}) (CCR7−/CD45RA+) T cells. In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T_{EMRA} T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are T_{EMRA} T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are T_{EMRA} T cells. In particular embodiments of such methods, the T cells are CD3+ cells.

In the methods presented herein, the determining may be performed using standard techniques well known to those of skill in the relevant art. For example, in the methods presented herein, the determining step may be performed by utilizing techniques such as those utilized in Example 2 (e.g., immunophenotyping of the PBMCs by polychromatic flow cytometry for markers associated with T cell differentiation, memory, senescence, and exhaustion).

In a particular embodiment, the cancer is multiple myeloma, chronic lymphocytic leukemia, or a non-Hodgkins lymphoma. In a particular embodiment, the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma. In a particular embodiment, the cancer is multiple myeloma. In a particular embodiment, the multiple myeloma is high-risk multiple myeloma or relapsed and refractory multiple myeloma. In a particular embodiment, the multiple myeloma is high risk multiple myeloma, and the high risk multiple myeloma is R-ISS stage III disease and/or a disease characterized by early relapse. In a particular embodiment, the multiple myeloma is not R-ISS stage III disease.

In a particular embodiment, the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan (e.g., melphalan hydrochloride), oxaliplatin, platinum, procarbazine, streptozocin, temozolomide, thiotepa, and trabectedin. In a particular embodiment, the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa. In a particular embodiment, the alkylating agent is cyclophosphamide.

In a particular embodiment, the subject is a human.

In another aspect, provided herein is a method of determining whether a subject having cancer can be treated with a CAR T therapy, comprising determining the percentage of CD3+ T cells in the subject relative to the number of PMBCs, wherein, (i) if the subject has less than about 20% CD3+ T cells relative to the number of PBMCs, not administering the CAR T therapy to the subject, and (ii) if the subject has more than about 20% CD3+ T cells relative to the number of PBMCs, then administering the CAR T therapy to the subject. In a particular embodiment, the CD3+ T cells are CD45+/CD3+ T cells.

In a particular embodiment, the method comprises determining the percentage of CD3+ T cells in the subject relative to the number of PMBCs, wherein the subject has less than about 10%, about 11%, about 12%, about 13%, about 14%, about 15%, about 16%, about 17%, about 18%, or about 19%, or about 20% CD3+ T cells relative to the number of PBMCs. In a particular embodiment, the method comprises determining the percentage of CD3+ T cells in the subject relative to the number of PMBCs, wherein the subject has about 15% to about 19%, about 16% to about 19%, about 17% to about 19%, about 18% to about 19% CD3+ T cells relative to the number of PBMCs, e.g., CD45+/CD3+ T cells. In a particular embodiment, the method comprises determining the percentage of CD3+ T cells in the subject relative to the number of PMBCs, wherein the subject has about 15% to about 16%, 15% to about 17%, 15% to about 18%, or about 15% to about 19% CD3+ T cells relative to the number of PBMCs, e.g., CD45+/CD3+ T cells.

In a particular embodiment, the method comprises determining the percentage of CD3+ T cells in the subject relative to the number of PMBCs, wherein the subject has more than about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50% CD3+ T cells relative to the number of PBMCs, e.g., CD45+/CD3+ T cells.

In a particular embodiment, the CAR T cell therapy is BCMA02, JCARH125, JNJ-68284528 (LCAR-B38M) (Janssen/Legend), P-BCMA-101 (Poseida), PBCAR269A (Poseida), P-BCMA-Allol (Poseida), Allo-715 (Pfizer/Allogene), CT053 (Carsgen), Descartes-08 (Cartesian), PHE885 (Novartis), CTX120 (CRISPR Therapeutics); a CD19 CAR T therapy, e.g., Yescarta, Kymriah, Tecartus, lisocabtagene maraleucel (liso-cel), or a CAR T therapy targeting any other cell surface marker.

In another aspect, provided herein is a method of treating a cancer in a subject, comprising having determined that the percentage of CD3+ T cells in the subject relative to the number of PMBCs is higher than about 20%, and administering a CAR T therapy to the subject. In a particular embodiment, the CD3+ T cells are CD45+/CD3+ T cells.

In a particular embodiment, the percentage of CD3+ T cells in the subject relative to the number of PMBCs is about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50%. In a particular embodiment, the percentage of CD3+ T cells in the subject relative to the number of PMBCs is about 20% to about 30%, about 25% to about 35%, about 30% to about 40%, about 45% to about 55%, about 50% to about 60%, about 55% to about 65%, about 60% to about 70%, or about 65% to about 75%.

In a particular embodiment, the CAR T cell therapy is BCMA02, JCARH125, JNJ-68284528 (LCAR-B38M) (Janssen/Legend), P-BCMA-101 (Poseida), PBCAR269A (Poseida), P-BCMA-Allol (Poseida), Allo-715 (Pfizer/Allogene), CT053 (Carsgen), Descartes-08 (Cartesian), PHE885 (Novartis), CTX120 (CRISPR Therapeutics); a CD19 CAR T therapy, e.g., Yescarta, Kymriah, Tecartus, lisocabtagene maraleucel (liso-cel), or a CAR T therapy targeting any other cell surface marker.

In a particular embodiment, the BCMA CAR T cells comprise a CAR directed to BCMA, wherein the CAR directed to BCMA comprises an antibody or antibody fragment that targets BCMA. In a particular embodiment, the BCMA CAR T cells comprise a CAR directed to BCMA, wherein the CAR directed to BCMA comprises a single chain Fv antibody or antibody fragment (scFv). In a particular embodiment, the BCMA CAR T cells comprise a CAR directed to BCMA, wherein the CAR directed to BCMA comprises a BCMA02 scFv, e.g., SEQ ID NO: 38.

In a specific embodiment of any of the above embodiments, the cancer is brain cancer, glioblastoma, bone cancer, pancreatic cancer, skin cancer, cancer of the head or neck, melanoma, lung cancer, uterine cancer, ovarian cancer, colorectal cancer, anal cancer, liver cancer, hepatocellular carcinoma, stomach cancer, testicular cancer, endometrial cancer, cervical cancer, Hodgkin's Disease, non-Hodgkin's lymphoma, esophageal cancer, intestinal cancer, thyroid cancer, adrenal cancer, bladder cancer, kidney cancer, breast cancer, multiple myeloma, sarcoma, anal cancer or squamous cell cancer.

In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are T cells.

In a particular embodiment of the methods presented herein, the T cells comprise CD3+ cells. In particular embodiments, the T cells are CD3+ cells. In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T CD3+ T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are CD3+ T cells, e.g., CD45+/CD3+ T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are CD3+ T cells.

In a particular embodiment, the T cells are CD8+ effector memory RA (T_{EMRA}) (CCR7−/CD45RA+) T cells. In a particular embodiment of the methods presented herein, the method comprises determining that at least about 15%, 16%, 17%, 18%, 19%, 20%, 21%, 22%, 23%, 24%, 25%, 26%, 27%, 28%, 29%, or 30% of the PBMCs are T_{EMRA} T cells. In a particular embodiment, the method comprises determining that at least about 15% to about 20%, 16% to about 20%, 17% to about 20%, 18% to about 20%, 19% to about 20%, 20% to about 20%, 21% to about 20%, 22% to about 20%, 23% to about 20%, 24% to about 20%, 25% to about 20%, 26% to about 20%, 27% to about 20%, 28% to about 20%, 29% to about 20%, or 30% to about 20% of the PBMCs are T_{EMRA} T cells. In a particular embodiment, the method comprises determining that at least about 15% to about 16%, 15% to about 17%, 15% to about 18%, 15% to about 19%, 15% to about 20%, 15% to about 21%, 15% to about 22%, 15% to about 23%, 15% to about 24%, 15% to about 25%, 15% to about 26%, 15% to about 27%, 15% to about 28%, 15% to about 29%, or 15% to about 30% of the PBMCs are T_{EMRA} T cells. In particular embodiments of such methods, the T cells are CD3+ cells.

In the methods presented herein, the determining may be performed using standard techniques well known to those of skill in the relevant art. For example, in the methods presented herein, the determining step may be performed by utilizing techniques such as those utilized in Example 2 (e.g., immunophenotyping of the PBMCs by polychromatic flow cytometry for markers associated with T cell differentiation, memory, senescence, and exhaustion).

In a specific embodiment, the number of T cells isolated from the PBMCs for use in the manufacturing of chimeric antigen receptor (CAR) T cells (e.g., BCMA CAR T Cells) is about at least 1×10⁶ to 1×10⁷, 1×10⁷ to 1×10⁸, 1×10⁸ to 1×10⁹, or 1×10⁹ to 1×10¹⁰. Ina specific embodiment, the number of T cells isolated from the PBMCs for use in the manufacturing of chimeric antigen receptor (CAR) T cells (e.g., BCMA CAR T Cells) is about at least 1×10⁶ to 1×10¹⁰, 1×10⁷ to 1×10¹⁰, 1×10⁸ to 1×10¹⁰, or 1×10⁹ to 1×10¹⁰. In a specific embodiment, the number of T cells isolated from the PBMCs for use in the manufacturing of chimeric antigen receptor (CAR) T cells (e.g., BCMA CAR T Cells) is about at least 1×10⁶ to 1×10⁷, 1×10⁶ to 1×10⁸, 1×10⁶ to 1×10⁹, or 1×10⁶ to 1×10¹⁰. In a specific embodiment, the number of T cells isolated from the PBMCs for use in the manufacturing of chimeric antigen receptor (CAR) T cells (e.g., BCMA CAR T Cells) is about at least 1×10⁷ to 1×10⁸, 1×10⁷ to 1×10⁹, 1×10⁷ to 1×10¹⁰, or 1×10⁸ to 1×10¹⁰.

In a specific embodiment of any of the above aspects or embodiments, the alkylating agent is a nitrogen mustard, a nitrosurea, an alkyl sulfonate, a triazine, or an ethylenimine. In a specific embodiment of any of the above aspects or embodiments, the alkylating agent is selected from the group consisting of a nitrogen mustard, a nitrosurea, an alkyl sulfonate, a triazine, or an ethylenimine. In a particular embodiment, the alkylating agent is a nitrogen mustard. In a particular embodiment, the nitrogen mustard is mechlorethamine, ifosfamide, melphalan (e.g., melphalan hydrochloride), chlorambucil, or cyclophosphamide. In a particular embodiment, the nitrogen mustard is selected from the group consisting of mechlorethamine, ifosfamide, melphalan (e.g., melphalan hydrochloride), chlorambucil, or cyclophosphamide. In a particular embodiment, the nitrogen mustard is mechlorethamine. In a particular embodiment, the nitrogen mustard is ifosfamide. In a particular embodiment, the nitrogen mustard is melphalan (e.g., melphalan hydrochloride). In a particular embodiment, the nitrogen mustard is chlorambucil. In a particular embodiment, the nitrogen mustard is cyclophosphamide. In a particular embodiment, the alkylating agent is a nitrosurea. In a particular embodiment, the nitrosurea is streptozocin, carmustine, or lomustine. In a particular embodiment, the nitrosurea is selected from the group consisting of streptozocin, carmustine, or lomustine. In a particular embodiment, the nitrosurea is streptozocin. In a particular embodiment, the nitrosurea is carmustine. In a particular embodiment, the nitrosurea is lomustine. In a particular embodiment, the alkylating agent is an alkyl sulfonate. In a particular embodiment, the alkyl sulfonate is busulfan. In a particular embodiment, the alkylating agent is a triazine. In a particular embodiment, the triazine is dacarbazine or temozolomide. In a particular embodiment, the triazine is dacarbazine. In a particular embodiment, the triazine is temozolomide. In a particular embodiment, the alkylating agent is an ethylenimine. In a particular embodiment, the ethylenimine is thiotepa or altretamine. In a particular embodiment, the ethylenimine is thiotepa. In a particular embodiment, the ethylenimine is altretamine.

In a specific embodiment of any of the above aspects or embodiments, the alkylating agent is altretamine. In a specific embodiment of any of the above aspects or embodiments, the alkylating agent is bendamustine. In a specific embodiment of any of the above aspects or embodiments, the alkylating agent is busulfan. In a specific embodiment of any of the above aspects or embodiments, the alkylating agent is carboplatin. In a specific embodiment of any of the above aspects or embodiments, the alkylating agent is carmustine. In a specific embodiment of any of the above aspects or embodiments, the alkylating agent is chlorambucil. In a specific embodiment of any of the above aspects or embodiments, the alkylating agent is cisplatin. In a specific embodiment of any of the above aspects or embodiments, the alkylating agent is cyclophosphamide. In a specific embodiment of any of the above aspects or embodiments, the alkylating agent is dacarbazine. In a specific embodiment of any of the above aspects or embodiments, the alkylating agent is evofosfamide. In a specific embodiment of any of the above aspects or embodiments, the alkylating agent is ifosfamide. In a specific embodiment of any of the above aspects or embodiments, the alkylating agent is lomustine. In a specific embodiment of any of the above aspects or embodiments, the alkylating agent is mechlorethamine. In a specific embodiment of any of the above aspects or embodiments, the alkylating agent is melphalan (e.g., melphalan hydrochloride). In a specific embodiment of any of the above aspects or embodiments, the alkylating agent is oxaliplatin. In a specific embodiment of any of the above aspects or embodiments, the alkylating agent is platinum. In a specific embodiment of any of the above aspects or embodiments, the alkylating agent is procarbazine. In a specific embodiment of any of the above aspects or embodiments, the alkylating agent is streptozocin. In a specific embodiment of any of the above aspects or embodiments, the alkylating agent is temozolomide. In a specific embodiment of any of the above aspects or embodiments, the alkylating agent is thiotepa.

In general, the disclosure of an alkylating agent herein does not include the use of the alkylating agent as part of a CAR T cell therapy. For example, the alkylating agent does not include the use of an alkylating agent (e.g., cyclophosphamide and/or fludarabine) within a period of about one (1) week prior to administering CAR T cells (e.g., BCMA CAR T cells, such as ide-cel) to the subject (e.g., on Days −6, −5, −4, −3, −2, or −1 day prior to administration of CAR T cells (e.g., BCMA CAR T cells, such as ide-cel) on Day 0).

In a specific embodiment of any of the above aspects or embodiments, the alkylating agent is altretamine, wherein the altretamine is administered to the subject at a dose of about 260 mg/m²/day for 5 days. In a specific embodiment of any of the above aspects or embodiments, the alkylating agent is bendamustine, wherein the bendamustine is administered to the subject at a dose of about 100 mg/m² infused intravenously over 30 minutes on Days 1 and 2 of a 28-day cycle, up to 6 cycles. In a specific embodiment of any of the above aspects or embodiments, the alkylating agent is bendamustine, wherein the bendamustine is administered to the subject at a dose of about 120 mg/m² infused intravenously over 60 minutes on Days 1 and 2 of a 21-day cycle, up to 8 cycles. In a specific embodiment of any of the above aspects or embodiments, the alkylating agent is busulfan, wherein the busulfan is administered to the subject at a dose of about 0.8 mg per kg of ideal body weight or actual body weight, whichever is lower, administered intravenously via a central venous catheter as a two-hour infusion every six hours for four consecutive days for a total of 16 doses. In a specific embodiment of any of the above aspects or embodiments, the alkylating agent is myleran, wherein the myleran is administered to the subject at a dose of about 4 to 8 mg, total dose, daily. In a specific embodiment of any of the above aspects or embodiments, the alkylating agent is myleran, wherein the myleran is administered to the subject at a dose of about 60 mcg/kg of body weight or 1.8 mg/m² of body surface, daily. In a specific embodiment of any of the above aspects or embodiments, the alkylating agent is carboplatin, wherein the carboplatin is administered to the subject at a dose of about 360 mg/m² IV on day 1 every 4 weeks. In a specific embodiment of any of the above aspects or embodiments, the alkylating agent is carboplatin, wherein the carboplatin is administered to the subject at a dose of about 300 mg/m² IV on day 1 every 4 weeks for 6 cycles with cyclophosphamide at a dose of about 600 mg/m² IV on day 1 every four weeks for six cycles. In a specific embodiment of any of the above aspects or embodiments, the alkylating agent is carmustine, wherein the carmustine is administered to the subject at a dose of about 150 to 200 mg/m² BiCNU intravenously every 6 weeks as a single dose. In a specific embodiment of any of the above aspects or embodiments, the alkylating agent is carmustine, wherein the carmustine is administered to the subject as a daily injection at a dose of about 75 to 100 mg/m² on 2 successive days. In a specific embodiment of any of the above aspects or embodiments, the alkylating agent is chlorambucil, wherein the chlorambucil is administered to the subject at a dose of about 0.1 to 0.2 mg/kg body weight daily for 3 to 6 weeks as required. In a specific embodiment of any of the above aspects or embodiments, the alkylating agent is chlorambucil, wherein the chlorambucil is administered to the subject at a dose of about 4 to 10 mg per day. In a specific embodiment of any of the above aspects or embodiments, the alkylating agent is chlorambucil, wherein the chlorambucil is administered to the subject at a dose of about 0.2 mg/kg daily. In a specific embodiment of any of the above aspects or embodiments, the alkylating agent is chlorambucil, wherein the chlorambucil is administered to the subject at a dose of about 0.1 mg/kg daily. In a specific embodiment of any of the above aspects or embodiments, the alkylating agent is cisplatin, wherein the cisplatin is administered to the subject at a dose of about 20 mg/m² IV daily for 5 days per cycle. In a specific embodiment of any of the above aspects or embodiments, the alkylating agent is cisplatin, wherein the cisplatin is administered to the subject at a dose of about 75 to 100 mg/m² IV per cycle once every 4 weeks (DAY 1). In a specific embodiment of any of the above aspects or embodiments, the alkylating agent is cisplatin, wherein the cisplatin is administered to the subject at a dose of about 600 mg/m² IV once every 4 weeks (DAY 1) in combination with PLATINOL, wherein the PLATINOL is administered to the subject at a dose of about 100 mg/m² IV per cycle once every 4 weeks. In a specific embodiment of any of the above aspects or embodiments, the alkylating agent is cisplatin, wherein the cisplatin is administered to the subject at a dose of about 50 to 70 mg/m² IV per cycle once every 3 to 4 weeks. In a specific embodiment of any of the above aspects or embodiments, the alkylating agent is cisplatin, wherein the cisplatin is administered to the subject at an initial dose of about 50 mg/m² per cycle repeated every 4 weeks. In a specific embodiment of any of the above aspects or embodiments, the alkylating agent is cyclophosphamide, wherein the cyclophosphamide is administered to the subject at a dose of about 40 mg per kg to 50 mg per kg in divided doses over 2 to 5 days. In a specific embodiment of any of the above aspects or embodiments, the alkylating agent is cyclophosphamide, wherein the cyclophosphamide is administered to the subject at a dose of about 10 mg per kg to 15 mg per kg given every 7 to 10 days. In a specific embodiment of any of the above aspects or embodiments, the alkylating agent is cyclophosphamide, wherein the cyclophosphamide is administered to the subject at a dose of about 3 mg per kg to 5 mg per kg twice weekly. In a specific embodiment of any of the above aspects or embodiments, the alkylating agent is cyclophosphamide, wherein the cyclophosphamide is administered to the subject at a dose of about 2 mg per kg daily for 8 to 12 weeks. In a specific embodiment of any of the above aspects or embodiments, the alkylating agent is ifosfamide, wherein the ifosfamide is administered to the subject at a dose of about 1.2 g/m² per day for 5 consecutive days. In a specific embodiment of any of the above aspects or embodiments, the alkylating agent is lomustine, wherein the lomustine is administered to the subject at a dose of about 130 mg/m² orally every 6 weeks. In a specific embodiment of any of the above aspects or embodiments, the alkylating agent ismechlorethamine, wherein the mechlorethamine is administered to the subject at a dose of about 0.4 mg/kg of body weight as a single dose. In a specific embodiment of any of the above aspects or embodiments, the alkylating agent is mechlorethamine, wherein the mechlorethamine is administered to the subject at a dose of about 0.1 to 0.2 mg/kg per day. In a specific embodiment of any of the above aspects or embodiments, the alkylating agent is melphalan (e.g., melphalan hydrochloride), wherein the melphalan (e.g., melphalan hydrochloride) is administered to the subject at a dose of about 16 mg/m² by IV. In a specific embodiment of any of the above aspects or embodiments, the alkylating agent is melphalan (e.g., melphalan hydrochloride), wherein the melphalan (e.g., melphalan hydrochloride) is administered to the subject at a dose of about 16 mg/m² by IV as a single infusion over 15 to 20 minutes. In a specific embodiment of any of the above aspects or embodiments, the alkylating agent is melphalan (e.g., melphalan hydrochloride), wherein the melphalan (e.g., melphalan hydrochloride) is administered to the subject at a dose of about 16 mg/m² by IV as a single infusion over 15 to 20 minutes at 2-week intervals for 4 doses, then, after adequate recovery from toxicity, at 4-week intervals. In a specific embodiment of any of the above aspects or embodiments, the alkylating agent is oxaliplatin, wherein the oxaliplatin is administered to the subject in combination with 5-fluorouracil/leucovorin every 2 weeks. In a specific embodiment of any of the above aspects or embodiments, the alkylating agent is oxaliplatin, wherein the oxaliplatin is administered to the subject in combination with 5-fluorouracil/leucovorin every 2 weeks, wherein on Day 1, the oxaliplatin is administered to the subject at a dose of about 85 mg/m² intravenous infusion in 250-500 mL 5% Dextrose Injection, USP and the leucovorin is administered to the subject at a dose of about 200 mg/m² intravenous infusion in 5% Dextrose Injection, USP, wherein the oxaliplatin and the leucovorin are administered to the subject over 120 minutes at the same time in separate bags using a Y-line, followed by administration of the 5-fluorouracil to the subject at a dose of about 400 mg/m² intravenous bolus administered over 2-4 minutes, followed by administration of the 5-fluorouracil to the subject at a dose of about 600 mg/m² intravenous infusion in 500 mL 5% Dextrose Injection, USP (recommended) as a 22-hour continuous infusion, and wherein on Day 2, the leucovorin is administered to the subject at a dose of about 200 mg/m² intravenous infusion over 120 minutes, followed by administration of the 5-fluorouracil to the subject at a dose of about 400 mg/m² IV bolus given over 2-4 minutes, followed by administration of the 5-fluorouracil to the subject at a dose of about 600 mg/m² intravenous infusion in 500 mL 5% Dextrose Injection, USP (recommended) as a 22-hour continuous infusion. In a specific embodiment of any of the above aspects or embodiments, the alkylating agent is procarbazine, wherein the procarbazine is administered to the subject at a dose of about 100 mg/m² daily for 14 days. In a specific embodiment of any of the above aspects or embodiments, the alkylating agent is procarbazine, wherein the procarbazine is administered to the subject at a dose of about 2 to 4 mg/kg/day in single or divided doses for the first week, followed by administration of the procarbazine to the subject at a dose of about 4 to 6 mg/kg/day until maximum response is obtained or until a white blood count falls below 4000/cmm or platelets fall below 100,000/cmm, wherein when the maximum response is obtained, the procarbazine is administered to the subject at a dose of about 1 to 2 mg/kg/day. In a specific embodiment of any of the above aspects or embodiments, the alkylating agent is streptozocin, wherein the streptozocin is administered daily to the subject intravenously at a dose of about 500 mg/m of body surface area for five consecutive days every six weeks. In a specific embodiment of any of the above aspects or embodiments, the alkylating agent is streptozocin, wherein the streptozocin is administered weekly to the subject intravenously at a dose of about 1000 mg/m of body surface area at weekly intervals for the first two courses (weeks). In a specific embodiment of any of the above aspects or embodiments, the alkylating agent is temozolomide, wherein the temozolomide is administered to the subject at a dose of about 75 mg/m² for 42 days concomitant with focal radiotherapy followed by initial maintenance dose of 150 mg/m² once daily for Days 1-5 of a 28-day cycle for 6 cycles. In a specific embodiment of any of the above aspects or embodiments, the alkylating agent is temozolomide, wherein the temozolomide is administered to the subject at an initial dose of about 150 mg/m² once daily for 5 consecutive days per 28-day treatment cycle. In a specific embodiment of any of the above aspects or embodiments, the alkylating agent is temozolomide, wherein the temozolomide is administered to the subject intravenously over 90 minutes. In a specific embodiment of any of the above aspects or embodiments, the alkylating agent is thiotepa, wherein the thiotepa is administered to the subject at a dose of about 5 mg/kg given intravenously approximately 12 hours apart on Day −6 before allogeneic HSCT in conjunction with high-dose busulfan and cyclophosphamide. In a specific embodiment of any of the above aspects or embodiments, the alkylating agent is thiotepa, wherein the thiotepa is administered to the subject at a dose of about 0.3 to 0.4 mg/kg intravenously. In a specific embodiment of any of the above aspects or embodiments, the alkylating agent is thiotepa, wherein the thiotepa is administered to the subject at a dose of about 0.6 to 0.8 mg/kg intracavitary. In a specific embodiment of any of the above aspects or embodiments, the alkylating agent is thiotepa, wherein the thiotepa is administered to the subject at a dose of about 60 mg in 30 to 60 mL of Sodium Chloride Injection into the bladder of the subject by catheter. In a specific embodiment of any of the above aspects or embodiments, the alkylating agent is trabectedin, wherein the trabectedin is administered to the subject at a dose of about 1.5 mg/m² body surface area as a 24-hour intravenous infusion, every 3 weeks through a central venous line. In a specific embodiment of any of the above aspects or embodiments, the alkylating agent is trabectedin, wherein the trabectedin is administered to the subject at a dose of about 0.9 mg/m² body surface area as a 24-hour intravenous infusion, every 3 weeks through a central venous line, wherein the subject has moderate hepatic impairment.

In a particular embodiment of any of the above aspects or embodiments, the subject is a human (e.g., a human patient). In a particular embodiment of any of the above aspects or embodiments, the subject is a mammal. In particular embodiments, the mammal is a pet, a laboratory research animal, or a farm animal. In some embodiments, the pet, research animal or farm animal is a dog, a cat, a horse, a monkey, a rabbit, a rat, a mouse, a guinea pig, a hamster, a pig, or a cow.

In a particular embodiment of any of the above aspects or embodiments, the BCMA CAR T cells comprise a CAR directed to BCMA. In specific embodiments, the CAR directed to BCMA comprises an antibody or antibody fragment that targets BCMA. In a particular embodiment of any of the above aspects or embodiments, the BCMA CAR T cells comprise a CAR directed to BCMA, wherein the CAR directed to BCMA comprises a single chain Fv antibody or antibody fragment (scFv). In a particular embodiment of any of the above aspects or embodiments, the BCMA CAR T cells comprise a CAR directed to BCMA, wherein the CAR directed to BCMA comprises SEQ ID NO: 37. In a particular embodiment of any of the above aspects or embodiments, the BCMA CAR T cells comprise a CAR directed to BCMA, wherein the CAR directed to BCMA comprises a BCMA02 scFv, e.g., SEQ ID NO: 38. In certain embodiments, the CAR directed to BCMA is encoded by SEQ ID NO: 10. In certain embodiments, a BCMA CAR T cell comprises a nucleic acid, e.g., a vector, encoding a BCMA CAR T, e.g., a BCMA CAR T comprising amino acids 22-493 or 1-493 of SEQ ID NO: 9, SEQ ID NO: 37, or SEQ ID NO: 38, or comprises a nucleic acid, e.g., a vector, comprising SEQ ID NO: 10. In a particular embodiment of any of the above aspects or embodiments, the BCMA CAR T cells are idecabtagene vicleucel cells.

The amount of soluble (i.e., non-membrane-bound) BCMA (sBCMA) after administration of a CAR T cell therapy, e.g., an anti-BCMA CAR T cell therapy, can be used to determine whether a subject can be expected to respond to the CAR T cell therapy appropriately, or whether the subject should be administered a different anticancer therapy. A greater drop in sBCMA levels in a tissue sample (e.g., serum, plasma, lymph, or blood) after administration of a CAR T cell therapy is correlated with a more clinically beneficial outcome (e.g., very good partial response, complete response or stringent complete response). In one aspect, for example, provided herein is a method of treating a disease caused by B Cell Maturation Agent (BCMA) expressing cells in a subject in need thereof, comprising: determining a first level of soluble BCMA (sBCMA) in a tissue sample from the subject; administering to the subject immune cells expressing a chimeric antigen receptor (CAR) directed to BCMA (BCMA CAR T cells), and then determining a second level of soluble BCMA in a tissue sample from the subject wherein, if said second level of sBCMA is greater than about 30% of said first level of sBCMA, the subject is subsequently provided a non-CAR T cell therapy to treat said disease. Also provided herein is a method of treating a disease caused by B Cell Maturation Agent (BCMA) expressing cells in a subject in need thereof, comprising: (a) determining a first level of soluble BCMA (sBCMA) in a tissue sample from the subject; (b) administering to the subject immune cells expressing a chimeric antigen receptor (CAR) directed to BCMA (BCMA CAR T cells); and (c) determining that a second level of soluble BCMA in a tissue sample from the subject is greater than about 30% of said first level, and on the basis of the determination in step c, subsequently providing a non-CAR T cell therapy to the subject. In a specific embodiment of either of the above embodiments, if said second level of sBCMA is greater than 40% of said first level, the subject is provided a non-CAR T cell therapy to treat said disease. In a specific embodiment of either of the above embodiments, if said second level of sBCMA is greater than about 20%, 25%, 30%, 35%, 40%, 45%, or 50% of said first level, the subject is provided a non-CAR T cell therapy to treat said disease. In another specific embodiment, said second level of sBCMA is determined at 25-35 days after said administering. In another specific embodiment, said second level of sBCMA is determined at 23-35, 24-35, 25-36, 25-37, 23-35, or 25-37 days after said administering. In another specific embodiment, said second level of sBCMA is determined at 23, 24, 25, 26, 27, 28, 29, 30, 31, 32, 33, 34, 35, 36, or 37 days after said administering. In another specific embodiment, said second level of sBCMA is determined at 28-31 days after said administering. In another specific embodiment, said second level of sBCMA is determined at 26-31, 27-31, 28-32, 28-33, 26-31, or 27-33 days after said administering. In another specific embodiment, said second level of sBCMA is determined at 26, 27, 28, 29, 30, 31, 32, or 33 days after said administering. In more specific embodiments, the subject is provided a non-CAR T cell therapy within three months, two months, or one month after said determining a second level of sBCMA.

In another aspect, provided herein is a method of treating a disease caused by B Cell Maturation Agent (BCMA) expressing cells, comprising administering to a patient diagnosed with said disease a non-CAR T cell therapy, wherein the patient has previously been administered immune cells expressing a chimeric antigen receptor (CAR) directed to BCMA (BCMA CAR T cells) and wherein a tissue sample from the patient subsequent to said administration contained a level of soluble BCMA (sBCMA) greater than 30% of a level of sBCMA found in a tissue sample obtained from the patient prior to said administration.

In another aspect, provided herein is a method of determining whether a patient diagnosed with a disease caused by B Cell Maturation Agent (BCMA) expressing cells should be administered a non-CAR T cell therapy after treatment with immune cells expressing a chimeric antigen receptor (CAR) directed to BCMA (BCMA CAR T cells), comprising determining a level of soluble BCMA (sBCMA) in a tissue sample from the patient, wherein the patient has previously been administered the immune cells expressing a chimeric antigen receptor (CAR) directed to BCMA (BCMA CAR T cells), and wherein if the level of sBCMA in the tissue sample is greater than 30% of a level of sBCMA found in a tissue sample obtained from the patient prior to said administration, then the patient is a candidate for the non-CAR T cell therapy. In a specific embodiment, the method further comprises administering the non-CAR T cell therapy to the candidate for the non-CAR T cell therapy.

The absolute level of sBCMA in a tissue sample (e.g., plasma, serum, lymph or blood) may also be used to determine whether a person administered a CAR T cell therapy, e.g., a BCMA CAR T cell therapy will appropriately benefit from that therapy, or should be administered a different anticancer therapy. Thus, provided herein is a method of treating a disease caused by B Cell Maturation Agent (BCMA) expressing cells in a subject in need thereof, comprising: administering to the subject immune cells expressing a chimeric antigen receptor (CAR) directed to BCMA (BCMA CAR T cells), and determining a level of soluble BCMA (sBCMA) in a tissue sample from the subject; wherein, if said level of sBCMA is greater than 4000 ng/L, the subject is subsequently provided a non-CAR T cell therapy to treat said disease. In a specific embodiment of either of the above embodiments, if said level of sBCMA is greater than about 3000 ng/L, 3500 ng/L, 4000 ng/L, 4500 ng/L, or 5000 ng/L the subject is subsequently provided a non-CAR T cell therapy to treat said disease. In a specific embodiment, said first level of sBCMA is determined at 50-70 days after said administering. In a specific embodiment, said first level of sBCMA is determined at 45-70, 46-70, 47-70, 48-70, 49-70, 50-70, 50-71, 50-72, 50-73, or 50-75 days after said administering. In a specific embodiment, said first level of sBCMA is determined at 50, 51, 52, 53, 54, 55, 56, 57, 58, 59 60, 61, 62, 63, 64, 65, 66, 67, 68, 69, or 70 days after said administering. In another specific embodiment, said first level of sBCMA is determined at 55-65 days after said administering. In another specific embodiment, said first level of sBCMA is determined at 50-65, 51-65, 52-65, 53-65, 54-65, 55-64, 55-63, 55-62, or 55-61 days after said administering. In another specific embodiment, said first level of sBCMA is determined at 55, 56, 57, 58, 59, 60, 61, 62, 63, 64, or 65 days after said administering. In another specific embodiment, said level of sBCMA is determined at 58-62 days after said administering. In another specific embodiment, said level of sBCMA is determined at 53-62, 54-62, 55-62, 56-62, 57-62, 58-68, 58-67, 58-66, 58-65, 58-64, or 58-63 days after said administering. In another specific embodiment, said level of sBCMA is determined at 58, 59, 60, 61, or 62 days after said administering. In a specific embodiment of the preceding embodiments, the subject is provided said non-CAR T cell therapy within three months, two months, or one month after said determining a first level of sBCMA.

The levels of certain cytokines, e.g., interleukin-6 (IL-6) and/or tumor necrosis factor alpha (TNFα) can also be used to determine whether a person administered a CAR T cell therapy, e.g., a BCMA CAR T cell therapy will appropriately benefit from that therapy, or should be administered a different anticancer therapy. Thus, provided herein is a method of treating a disease caused by B Cell Maturation Agent (BCMA) expressing cells in a subject in need thereof, comprising: determining a first level of interleukin-6 (IL-6), tumor necrosis factor alpha (TNFα) or both in a tissue sample from the subject; administering to the subject immune cells expressing a chimeric antigen receptor (CAR) directed to BCMA (BCMA CAR T cells), and subsequently determining a second level of IL-6, TNFα or both in a tissue sample from the subject; wherein, if said second level of IL-6, TNFα or both is not greater than said first level of IL-6, TNFα or both, respectively, then the subject is subsequently provided a non-CAR T cell therapy to treat said disease. In a specific embodiment, said first level is determined on the day of said administering to the subject said immune cells expressing a CAR directed to BCMA, and said second level is determined 1-4 days after said administering. In another specific embodiment, said second level is determined one day after said administering. In another specific embodiment, said second level is determined two days after said administering. In another specific embodiment, said second level is determined three days after said administering. In another specific embodiment, said second level is determined four days after said administering.

In another aspect, provided herein is a method of treating a disease caused by B Cell Maturation Agent (BCMA)-expressing cells in a subject in need thereof, comprising: administering to the subject immune cells expressing a chimeric antigen receptor (CAR) directed to BCMA (BCMA CAR T cells), and determining a level of ferritin in a tissue sample from the subject; wherein, if said level of ferritin is greater than 1500 picomoles per liter, the subject is subsequently provided a therapy to treat cytokine release syndrome (CRS). In certain embodiments, said determining is performed within 0-4 days prior to said administering. In a specific embodiment, said determining is performed on the same day as said administering. In another specific embodiment, said therapy to treat CRS is first provided to said subject 0-5 days after said administering.

In another aspect, provided herein is a method of treating a disease caused by B Cell Maturation Agent (BCMA) expressing cells in a subject in need thereof, comprising: (a) determining a first level of soluble BCMA (sBCMA) and/or a first level of interleukin-6 (IL-6), tumor necrosis factor alpha (TNFα), or both in a tissue sample from the subject; (b) administering to the subject immune cells expressing a chimeric antigen receptor (CAR) directed to BCMA (BCMA CAR T cells), and (c) determining a second level of sBCMA and/or a second level of interleukin-6 (IL-6), tumor necrosis factor alpha (TNFα), or both in a tissue sample from the subject wherein, if said second level of sBCMA is greater than 30% of said first level of sBCMA and/or if said second level of IL-6, TNFα or both is not greater than said first level of IL-6, TNFα or both, the subject is subsequently provided a non-CAR T cell therapy to treat said disease.

In another aspect, provided herein is a method of treating a disease caused by B Cell Maturation Agent (BCMA) expressing cells in a subject in need thereof, comprising: (a) determining a first level of soluble BCMA (sBCMA) and/or a first level of interleukin-6 (IL-6), tumor necrosis factor alpha (TNFα), or both in a tissue sample from the subject; (b) administering to the subject immune cells expressing a chimeric antigen receptor (CAR) directed to BCMA (BCMA CAR T cells), (c) determining that a second level of sBCMA in a tissue sample from the subject is greater than 30% of said first level of sBCMA and/or a second level of IL-6, TNFα or both is not greater than said first level of IL-6, TNFα or both, and (d) on the basis of the determination in step c, subsequently providing a non-CAR T cell therapy to the subject.

In another aspect, provided herein is a method of treating a disease caused by B Cell Maturation Agent (BCMA) expressing cells, comprising administering to a patient diagnosed with said disease a non-CAR T cell therapy, wherein the patient has previously been administered immune cells expressing a chimeric antigen receptor (CAR) directed to BCMA (BCMA CAR T cells) and wherein a tissue sample from the patient subsequent to said administration contained (i) a level of soluble BCMA (sBCMA) greater than 30% of a level of sBCMA found in a tissue sample obtained from the patient prior to said administration and/or (ii) a level of IL-6, TNFα or both not greater than a level of IL-6, TNFα or both found in a tissue sample obtained from the patient prior to said administration.

In another aspect, provided herein is a method of determining whether a patient diagnosed with a disease caused by B Cell Maturation Agent (BCMA) expressing cells should be administered a non-CAR T cell therapy after treatment with immune cells expressing a chimeric antigen receptor (CAR) directed to BCMA (BCMA CAR T cells), comprising determining a level of soluble BCMA (sBCMA) and/or a level of IL-6, TNFα or both in a tissue sample from the patient, wherein the patient has previously been administered the immune cells expressing a chimeric antigen receptor (CAR) directed to BCMA (BCMA CAR T cells), and wherein if (i) the level of sBCMA in the tissue sample is greater than 30% of a level of sBCMA found in a tissue sample obtained from the patient prior to said administration and/or (ii) the level of IL-6, TNFα or both is not greater than a level of IL-6, TNFα or both found in a tissue sample obtained from the patient prior to said administration, then the patient is a candidate for the non-CAR T cell therapy. In a specific embodiment, the method further comprises administering the non-CAR T cell therapy to the candidate for the non-CAR T cell therapy.

In a specific embodiment of any of the above aspects or embodiments, the alkylating agent is a nitrogen mustard, a nitrosurea, an alkyl sulfonate, a triazine, or an ethylenimine. In a specific embodiment, the alkylating agent is a nitrogen mustard. In a specific embodiment, the alkylating agent is a nitrosurea. In a specific embodiment, the alkylating agent is an alkyl sulfonate. In a specific embodiment, the alkylating agent is a triazine. In a specific embodiment, the alkylating agent is an ethylenimine. In a specific embodiment, the nitrogen mustard is mechlorethamine (Mustargen, Nitrogen Mustard, Mustine, Chlormethine). In a specific embodiment, the nitrogen mustard is ifosfamide (Ifex). In a specific embodiment, the nitrogen mustard is melphalan (e.g., melphalan hydrochloride, Alkeran). In a specific embodiment, the nitrogen mustard is chlorambucil (Leukeran). In a specific embodiment, the nitrogen mustard is cyclophosphamide (Cytoxan, Neosar). In a specific embodiment, the nitrosurea is streptozocin (Zanosar). In a specific embodiment, the nitrosurea is carmustine (BCNU, Gliadel, Carmubris). In a specific embodiment, the nitrosurea is lomustine (Gleostine, CeeNU, CCNU). In a specific embodiment, the alkyl sulfonate is busulfan (Myleran, Busulfex). In a specific embodiment, the triazine is Dacarbazine (Imidazole Carboxamide, DTIC-Dome). In a specific embodiment, the triazine is temozolomide (Temodar). In a specific embodiment, the ethylenimine is thiotepa (Thioplex).

In a specific embodiment, the ethylenimine is altretamine (Hexalen). In specific embodiments of any of the above aspects or embodiments, said CAR T cell therapy (e.g., immune cells expressing a chimeric antigen receptor (CAR) directed to BCMA (BCMA CAR T cells), e.g., idecabtagene vicleucel (ide-cel) cells) comprises a population of cells that comprises about 10%, 5%, 3%, 2%, or 1% activated CAR T-cells, for example, activated CD8 CAR T-cells (CD3+/CD8+/CAR+/CD25+).

In specific embodiments of any of the above aspects or embodiments, said CAR T cell therapy (e.g., immune cells expressing a chimeric antigen receptor (CAR) directed to BCMA (BCMA CAR T cells), e.g., idecabtagene vicleucel (ide-cel) cells) comprises a population of cells that comprises 10%, 5%, 3%, 2%, or 1% senescence population of CAR T-cells, for example, CD4 CAR T-cells (CD3+/CD4+/CAR+/CD57+). In a specific embodiments of any of the above aspects or embodiments, said tissue sample is blood, plasma or serum. In another specific embodiments of any of the above aspects or embodiments, said disease caused by BCMA-expressing cells is multiple myeloma, chronic lymphocytic leukemia, or a non-Hodgkins lymphoma (e.g., Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, and mantle cell lymphoma). In specific embodiments, the disease is multiple myeloma, e.g., high-risk multiple myeloma or relapsed and refractory multiple myeloma. In other specific embodiments, the high risk multiple myeloma is R-ISS stage III disease and/or a disease characterized by early relapse (e.g., progressive disease within 12 months since the date of last treatment regimen, such as last treatment regimen with a proteasome inhibitor, an immunomodulatory agent and/or dexamethasone). In a particular embodiment, the multiple myeloma is not R-ISS stage III disease. In specific embodiments, said disease caused by BCMA-expressing cells is a non-Hodgkins lymphoma, and wherein the non-Hodgkins lymphoma is selected from the group consisting of: Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, and mantle cell lymphoma.

In one embodiment, before the administration of the T cells expressing a chimeric antigen receptor (CAR) directed to B Cell Maturation Antigen (BCMA), the subject having a tumor has been assessed for expression of BCMA by the tumor.

In specific embodiments of any of the above aspects or embodiments, the immune cells are T cells, e.g., CD4+ T cells, CD8+ T cells or cytocoxic T lymphocytes (CTLs), T killer cells, or natural killer (NK) cells. In another specific embodiment specific embodiment, the immune cells are administered in a dosage of from 150×10⁶ cells to 450×10⁶ cells.

In a specific embodiment of any of the above embodiments, the non-CAR T cell therapy comprises a proteasome inhibitor, lenalidomide, pomalidomide, thalidomide, bortezomib, dexamethasone, cyclophosphamide, doxorubicin, carfilzomib, ixazomib, cisplatin, doxorubicin, etoposide, an anti-CD38 antibody panobinostat, or elotuzumab. In more specific embodiments, before said administering said subject has received one or more lines of prior therapy comprising: daratumumab, pomalidomide, and dexamethasone (DPd); daratumumab, bortezomib, and dexamethasone (DVd); ixazomib, lenalidomide, and dexamethasone (IRd); daratumumab, lenalidomide and dexamethasone; bortezomib, lenalidomide and dexamethasone (RVd); bortezomib, cyclophosphamide and dexamethasone (BCd); bortezomib, doxorubicin and dexamethasone; carfilzomib, lenalidomide and dexamethasone (CRd); bortezomib and dexamethasone; bortezomib, thalidomide and dexamethasone; lenalidomide and dexamethasone; dexamethasone, thalidomide, cisplatin, doxorubicin, cyclophosphamide, etoposide and bortezomib (VTD-PACE); lenalidomide and low-dose dexamethasone; bortezomib, cyclophosphamide and dexamethasone; carfilzomib and dexamethasone; lenalidomide alone; bortezomib alone; daratumumab alone; elotuzumab, lenalidomide, and dexamethasone; elotuzumab, lenalidomide and dexamethasone; bendamustine, bortezomib and dexamethasone; bendamustine, lenalidomide, and dexamethasone; pomalidomide and dexamethasone; pomalidomide, bortezomib and dexamethasone; pomalidomide, carfilzomib and dexamethasone; bortezomib and liposomal doxorubicin; cyclophosphamide, lenalidomide, and dexamethasone; elotuzumab, bortezomib and dexamethasone; ixazomib and dexamethasone; panobinostat, bortezomib and dexamethasone; panobinostat and carfilzomib; or pomalidomide, cyclophosphamide and dexamethasone; or any one of the other therapeutic agents listed in Section 5.9, below. In a more specific embodiment, the patient has not received said non-CAR T cell therapy prior to administration of CAR T cells.

In a specific embodiment of any of the above embodiments, the non-CAR T cell therapy comprises lenalidomide. In certain embodiments, the lenalidomide is administered to a subject as a maintenance therapy after administration of compositions comprising CAR-expressing immune effector cells. In certain embodiments, the lenalidomide may be administered immediately after administration of the compositions comprising CAR-expressing immune effector cells. In certain embodiments, the lenalidomide may be administered 1 week, 2 weeks, 3 weeks, or 4 weeks after administration of the compositions comprising CAR-expressing immune effector cells. In certain embodiments, the lenalidomide may be administered 1 month, 2 months, 3 months, 4 months, 5 months, 6 months, 7 months, 8 months, 9 months, 10 months, 11 months, or 12 months after administration of the compositions comprising CAR-expressing immune effector cells. In certain embodiments, the lenalidomide may be administered at a dosage of about 2.5 mg, 5 mg, 10 mg, 15 mg, 20 mg, or 25 mg. In certain embodiments, the lenalidomide may be administered at a dosage of about 2.5 mg, 5 mg, 10 mg, 15 mg, 20 mg, or 25 mg once daily. In certain embodiments, the lenalidomide may be administered at a dosage of about 25 mg once daily orally on Days 1-21 of repeated 28-day cycles. In certain embodiments, the lenalidomide may be administered at a dosage of about 25 mg once daily orally on Days 1-21 of repeated 28-day cycles to a subject for treating Multiple Myeloma (MM). In certain embodiments, the lenalidomide may be administered at a dosage of about 10 mg once daily continuously on Days 1-28 of repeated 28-day cycles. In certain embodiments, the lenalidomide may be administered at a dosage of about 2.5 mg once daily. In certain embodiments, the lenalidomide may be administered at a dosage of about 5 mg once daily. In certain embodiments, the lenalidomide may be administered at a dosage of about 10 mg once daily. In certain embodiments, the lenalidomide may be administered at a dosage of about 15 mg every other day. In certain embodiments, the lenalidomide may be administered at a dosage of about 25 mg once daily orally on Days 1-21 of repeated 28-day cycles. In certain embodiments, the lenalidomide may be administered at a dosage of about 20 mg once daily orally on Days 1-21 of repeated 28-day cycles for up to 12 cycles. In a certain embodiment, lenalidomide maintenance therapy is recommended for all patients. In a certain embodiment, lenalidomide maintenance therapy should be initiated upon adequate bone marrow recovery or from 90-day post-ide-cel infusion, whichever is later.

In a specific embodiment of any of the above embodiments, the non-CAR T cell therapy comprises pomalidomide. In certain embodiments, the pomalidomide is administered to a subject as a maintenance therapy after administration of compositions comprising CAR-expressing immune effector cells. In certain embodiments, the pomalidomide may be administered immediately after administration of the compositions comprising CAR-expressing immune effector cells. In certain embodiments, the pomalidomide may be administered 1 week, 2 weeks, 3 weeks, or 4 weeks after administration of the compositions comprising CAR-expressing immune effector cells. In certain embodiments, the pomalidomide may be administered 1 month, 2 months, 3 months, 4 months, 5 months, 6 months, 7 months, 8 months, 9 months, 10 months, 11 months, or 12 months after administration of the compositions comprising CAR-expressing immune effector cells. In certain embodiments, the pomalidomide may be administered at a dosage of about 1 mg, 2 mg, 3 mg, or 4 mg. In certain embodiments, the pomalidomide may be administered at a dosage of about 1 mg, 2 mg, 3 mg, or 4 mg once daily. In certain embodiments, the pomalidomide may be administered at a dosage of about 4 mg per day taken orally on days 1-21 of repeated 28-day cycles until disease progression. In certain embodiments, the pomalidomide may be administered at a dosage of about 4 mg per day taken orally on days 1-21 of repeated 28-day cycles until disease progression to a subject for treating Multiple Myeloma (MM). In a certain embodiment, pomalidomide maintenance therapy is recommended for all patients. In a certain embodiment, pomalidomide maintenance therapy should be initiated upon adequate bone marrow recovery or from 90-day post-ide-cel infusion, whichever is later.

In a specific embodiment of any of the above embodiments, the non-CAR T cell therapy comprises CC-220 (iberdomide; see, e.g., Bjorkland, C. C. et al., 2019, Leukemia, doi: 10.1038/s41375-019-0620-8; U.S. Pat. No. 9,828,361). In certain embodiments, the CC-220 is administered to a subject as a maintenance therapy after administration of compositions comprising CAR-expressing immune effector cells. In certain embodiments, the CC-220 may be administered immediately after administration of the compositions comprising CAR-expressing immune effector cells. In certain embodiments, the CC-220 may be administered 1 week, 2 weeks, 3 weeks, or 4 weeks after administration of the compositions comprising CAR-expressing immune effector cells. In certain embodiments, the CC-220 may be administered 1 month, 2 months, 3 months, 4 months, 5 months, 6 months, 7 months, 8 months, 9 months, 10 months, 11 months, or 12 months after administration of the compositions comprising CAR-expressing immune effector cells. In certain embodiments, the CC-220 may be administered at a dosage of about 0.15 mg, 0.3 mg, 0.45 mg, 0.6 mg, 0.75 mg, 0.9 mg, 1.0 mg, 1.1 mg, or 1.2 mg. In certain embodiments, the CC-220 may be administered orally. In certain embodiments, the CC-220 may be administered orally at a dosage of about 0.15 mg, 0.3 mg, 0.45 mg, 0.6 mg, 0.75 mg, 0.9 mg, 1.0 mg, 1.1 mg, or 1.2 mg daily for 21 days of a 28-day cycle, e.g., daily on days 1-21 of a 28-day cycle, with the 28-day cycles repeated as needed. In certain embodiments, the CC-220 may be administered to a subject for treating Multiple Myeloma (MM). In a certain embodiment, CC-220 maintenance therapy is recommended for all patients. In a certain embodiment, the CC-220 maintenance therapy should be initiated upon adequate bone marrow recovery or from 90-day post-ide-cel infusion, whichever is later.

In a specific embodiment of any of the above embodiments, the non-CAR T cell therapy comprises CC-220 (iberdomide) and dexamethasone. In certain embodiments, the CC-220 and dexamethasone are administered to a subject as a maintenance therapy after administration of compositions comprising CAR-expressing immune effector cells. In certain embodiments, the CC-220 and dexamethasone may be administered immediately after administration of the compositions comprising CAR-expressing immune effector cells. In certain embodiments, the CC-220 may be administered immediately after administration of the compositions comprising CAR-expressing immune effector cells. In certain embodiments, the dexamethasone may be administered immediately after administration of the compositions comprising CAR-expressing immune effector cells. In certain embodiments, the CC-220 and dexamethasone may be administered 1 week, 2 weeks, 3 weeks, or 4 weeks after administration of the compositions comprising CAR-expressing immune effector cells. In certain embodiments, the CC-220 may be administered 1 week, 2 weeks, 3 weeks, or 4 weeks after administration of the compositions comprising CAR-expressing immune effector cells. In certain embodiments, the dexamethasone may be administered 1 week, 2 weeks, 3 weeks, or 4 weeks after administration of the compositions comprising CAR-expressing immune effector cells. In certain embodiments, the CC-220 and dexamethasone may be administered 1 month, 2 months, 3 months, 4 months, 5 months, 6 months, 7 months, 8 months, 9 months, 10 months, 11 months, or 12 months after administration of the compositions comprising CAR-expressing immune effector cells. In certain embodiments, the CC-220 may be administered 1 month, 2 months, 3 months, 4 months, 5 months, 6 months, 7 months, 8 months, 9 months, 10 months, 11 months, or 12 months after administration of the compositions comprising CAR-expressing immune effector cells. In certain embodiments, the dexamethasone may be administered 1 month, 2 months, 3 months, 4 months, 5 months, 6 months, 7 months, 8 months, 9 months, 10 months, 11 months, or 12 months after administration of the compositions comprising CAR-expressing immune effector cells. In certain embodiments, the CC-220 may be administered at a dosage of about 0.15 mg, 0.3 mg, 0.45 mg, 0.6 mg, 0.75 mg, 0.9 mg, 1.0 mg, 1.1 mg, or 1.2 mg. In certain embodiments, the dexamethasone may be administered at a dosage of about 20 mg, 25 mg., 30 mg, 35 mg, 40 mg, 45 mg, 50 mg, 55 mg, or 60 mg. In certain embodiments, the dexamethasone may be administered at a dosage of about 40 mg. In certain embodiments, the CC-220 may be administered orally. In certain embodiments, the CC-220 may be administered orally at a dosage of about 15 mg, 0.3 mg, 0.45 mg, 0.6 mg, 0.75 mg, 0.9 mg, 1.0 mg, 1.1 mg, or 1.2 mg daily for 21 days of a 28-day cycle, e.g., daily on days 1-21 of a 28-day cycle, with the 28-day cycles repeated as needed. In certain embodiments, the dexamethasone may be administered orally. In certain embodiments, the dexamethasone may be administered at a dose of about 20-60 mgs. In certain embodiments, the dexamethasone may be administered orally at a dosage of about 20 mg, 25 mg, 30 mg, 35 mg, 40 mg, 45 mg, 50 mg, 55 mg, or 60 mg on days 1, 8, 15, and 22 of a 28-day cycle, with the 28-day cycles repeated as needed. In certain embodiments, the CC-220 may be administered orally at a dosage of about 15 mg, 0.3 mg, 0.45 mg, 0.6 mg, 0.75 mg, 0.9 mg, 1.0 mg, 1.1 mg, or 1.2 mg daily for 21 days of a 28-day cycle, e.g., daily on days 1-21 of a 28-day cycle, with the 28-day cycles repeated as needed, and the dexamethasone may be administered orally at a dosage of about 20 mg, 25 mg., 30 mg, 35 mg, 40 mg, 45 mg, 50 mg, 55 mg, or 60 mg on days 1, 8, 15, and 22 of a 28-day cycle, with the 28-day cycles repeated as needed. In certain embodiments, the CC-220 and dexamethasone may be administered to a subject for treating Multiple Myeloma (MM). In a certain embodiment, CC-220 and dexamethasone maintenance therapy is recommended for all patients. In a certain embodiment, the CC-220 and dexamethasone maintenance therapy should be initiated upon adequate bone marrow recovery or from 90-day post-ide-cel infusion, whichever is later.

In another specific embodiment of any of the above aspects or embodiments, before said administering said subject has received three or more lines of prior therapy, or one or more lines of prior therapy. In more specific embodiments, said lines of prior therapy comprise a proteasome inhibitor, lenalidomide, pomalidomide, thalidomide, bortezomib, dexamethasone, cyclophosphamide, doxorubicin, carfilzomib, ixazomib, cisplatin, doxorubicin, etoposide, an anti-CD38 antibody panobinostat, or elotuzumab. In more specific embodiments, before said administering said subject has received one or more lines of prior therapy comprising: daratumumab, pomalidomide, and dexamethasone (DPd); daratumumab, bortezomib, and dexamethasone (DVd); ixazomib, lenalidomide, and dexamethasone (IRd); daratumumab, lenalidomide and dexamethasone; bortezomib, lenalidomide and dexamethasone (RVd); bortezomib, cyclophosphamide and dexamethasone (BCd); bortezomib, doxorubicin and dexamethasone; carfilzomib, lenalidomide and dexamethasone (CRd); bortezomib and dexamethasone; bortezomib, thalidomide and dexamethasone; lenalidomide and dexamethasone; dexamethasone, thalidomide, cisplatin, doxorubicin, cyclophosphamide, etoposide and bortezomib (VTD-PACE); lenalidomide and low-dose dexamethasone; bortezomib, cyclophosphamide and dexamethasone; carfilzomib and dexamethasone; lenalidomide alone; bortezomib alone; daratumumab alone; elotuzumab, lenalidomide, and dexamethasone; elotuzumab, lenalidomide and dexamethasone; bendamustine, bortezomib and dexamethasone; bendamustine, lenalidomide, and dexamethasone; pomalidomide and dexamethasone; pomalidomide, bortezomib and dexamethasone; pomalidomide, carfilzomib and dexamethasone; bortezomib and liposomal doxorubicin; cyclophosphamide, lenalidomide, and dexamethasone; elotuzumab, bortezomib and dexamethasone; ixazomib and dexamethasone; panobinostat, bortezomib and dexamethasone; panobinostat and carfilzomib; or pomalidomide, cyclophosphamide and dexamethasone. In various more specific embodiments, said subject has received two, three, four, five, six, seven or more of said lines of prior therapy; no more than three of said lines of prior therapy; no more than two of said lines of prior therapy; or no more than one of said lines of prior therapy.

In specific embodiments of any of the above aspects or embodiments, the immune cells are administered at a dose ranging from 150×10⁶ cells to 450×10⁶ cells, 300×10⁶ cells to 600×10⁶ cells, 350×10⁶ cells to 600×10⁶ cells, 350×10⁶ cells to 550×10⁶ cells, 400×10⁶ cells to 600×10⁶ cells, 150×10⁶ cells to 300×10⁶ cells, or 400×10⁶ cells to 500×10⁶ cells. In some embodiments, the immune cells are administered at a dose of about 150×10⁶ cells, about 200×10⁶ cells, about 250×10⁶ cells, about 300×10⁶ cells, about 350×10⁶ cells, about 400×10⁶ cells, about 450×10⁶ cells, about 500×10⁶ cells, or about 550×10⁶ cells. In one embodiment, the immune cells are administered at a dose of about 450×10⁶ cells. In some embodiments, the subject is administered one infusion of the immune cells expressing a chimeric antigen receptor (CAR). In some embodiments, the administration of the immune cells expressing a CAR is repeated (e.g., a second dose of immune cells is administered to the subject). In some embodiments, the subject is administered one infusion of the immune cells expressing a chimeric antigen receptor (CAR) directed to B Cell Maturation Antigen (BCMA). In some embodiments, the administration of the immune cells expressing a CAR directed to BCMA is repeated (e.g., a second dose of immune cells is administered to the subject).

In specific embodiments of any of the embodiments described herein, the immune cells expressing a CAR are administered in a dosage of from about 150×10⁶ cells to about 300×10⁶ cells. In specific embodiments of any of the embodiments described herein, the immune cells expressing a CAR are administered in a dosage of from about 350×10⁶ cells to about 550×10⁶ cells. In specific embodiments of any of the embodiments described herein, the immune cells expressing a CAR are administered in a dosage of from about 400×10⁶ cells to about 500×10⁶ cells. In specific embodiments of any of the embodiments described herein, the immune cells expressing a CAR are administered in a dosage of from about 150×10⁶ cells to about 250×10⁶ cells. In specific embodiments of any of the embodiments described herein, the immune cells expressing a CAR are administered in a dosage of from about 300×10⁶ cells to about 500×10⁶ cells. In specific embodiments of any of the embodiments described herein, the immune cells expressing a CAR are administered in a dosage of from about 350×10⁶ cells to about 450×10⁶ cells. In specific embodiments of any of the embodiments described herein, the immune cells expressing a CAR are administered in a dosage of from about 300×10⁶ cells to about 450×10⁶ cells. In specific embodiments of any of the embodiments described herein, the immune cells expressing a CAR are administered in a dosage of from about 250×10⁶ cells to about 450×10⁶ cells. In specific embodiments of any of the embodiments described herein, the immune cells expressing a CAR are administered in a dosage of from about 300×10⁶ cells to about 600×10⁶ cells. In specific embodiments of any of the embodiments described herein, the immune cells expressing a CAR are administered in a dosage of from about 250×10⁶ cells to about 500×10⁶ cells. In specific embodiments of any of the embodiments described herein, the immune cells expressing a CAR are administered in a dosage of from about 350×10⁶ cells to about 500×10⁶ cells. In specific embodiments of any of the embodiments described herein, the immune cells expressing a CAR are administered in a dosage of from about 400×10⁶ cells to about 600×10⁶ cells. In specific embodiments of any of the embodiments described herein, the immune cells expressing a CAR are administered in a dosage of from about 400×10⁶ cells to about 450×10⁶ cells. In specific embodiments of any of the embodiments described herein, the immune cells expressing a CAR are administered in a dosage of from about 200×10⁶ cells to about 400×10⁶ cells. In specific embodiments of any of the embodiments described herein, the immune cells expressing a CAR are administered in a dosage of from about 200×10⁶ cells to about 350×10⁶ cells. In specific embodiments of any of the embodiments described herein, the immune cells expressing a CAR are administered in a dosage of from about 200×10⁶ cells to about 300×10⁶ cells. In specific embodiments of any of the embodiments described herein, the immune cells expressing a CAR are administered in a dosage of from about 450×10⁶ cells to about 500×10⁶ cells. In specific embodiments of any of the embodiments described herein, the immune cells expressing a CAR are administered in a dosage of from about 250×10⁶ cells to about 400×10⁶ cells. In specific embodiments of any of the embodiments described herein, the immune cells expressing a CAR are administered in a dosage of from about 250×10⁶ cells to about 350×10⁶ cells. In specific embodiments of any of the embodiments described herein, the immune cells expressing a CAR are administered in a dosage of about 450×10⁶ cells. In specific embodiments of any of the embodiments described herein, the immune cells are T cells (e.g., autologous T cells). In specific embodiments of any of the embodiments described herein, the subjects being treated undergo a leukapharesis procedure to collect autologous immune cells for the manufacture of the immune cells expressing a CAR prior to their administration to the subject. In specific embodiments of any of the embodiments described herein, the immune cells (e.g., T cells) are administered by an intravenous infusion.

In specific embodiments of any of the embodiments described herein, the immune cells expressing a CAR are administered in a dosage of from about 150×10⁶ cells to about 300×10⁶ cells. In specific embodiments of any of the embodiments described herein, the immune cells expressing a CAR are administered in a dosage of from about 350×10⁶ cells to about 550×10⁶ cells. In specific embodiments of any of the embodiments described herein, the immune cells expressing a CAR are administered in a dosage of from about 400×10⁶ cells to about 500×10⁶ cells. In specific embodiments of any of the embodiments described herein, the immune cells expressing a CAR are administered in a dosage of from about 150×10⁶ cells to about 250×10⁶ cells. In specific embodiments of any of the embodiments described herein, the immune cells expressing a CAR are administered in a dosage of from about 300×10⁶ cells to about 500×10⁶ cells. In specific embodiments of any of the embodiments described herein, the immune cells expressing a CAR are administered in a dosage of from about 350×10⁶ cells to about 450×10⁶ cells. In specific embodiments of any of the embodiments described herein, the immune cells expressing a CAR are administered in a dosage of from about 300×10⁶ cells to about 450×10⁶ cells. In specific embodiments of any of the embodiments described herein, the immune cells expressing a CAR are administered in a dosage of from about 250×10⁶ cells to about 450×10⁶ cells. In specific embodiments of any of the embodiments described herein, the immune cells expressing a CAR are administered in a dosage of from about 300×10⁶ cells to about 600×10⁶ cells. In specific embodiments of any of the embodiments described herein, the immune cells expressing a CAR are administered in a dosage of from about 250×10⁶ cells to about 500×10⁶ cells. In specific embodiments of any of the embodiments described herein, the immune cells expressing a CAR are administered in a dosage of from about 350×10⁶ cells to about 500×10⁶ cells. In specific embodiments of any of the embodiments described herein, the immune cells expressing a CAR are administered in a dosage of from about 400×10⁶ cells to about 600×10⁶ cells. In specific embodiments of any of the embodiments described herein, the immune cells expressing a CAR are administered in a dosage of from about 400×10⁶ cells to about 450×10⁶ cells. In specific embodiments of any of the embodiments described herein, the immune cells expressing a CAR are administered in a dosage of from about 200×10⁶ cells to about 400×10⁶ cells. In specific embodiments of any of the embodiments described herein, the immune cells expressing a CAR are administered in a dosage of from about 200×10⁶ cells to about 350×10⁶ cells. In specific embodiments of any of the embodiments described herein, the immune cells expressing a CAR are administered in a dosage of from about 200×10⁶ cells to about 300×10⁶ cells. In specific embodiments of any of the embodiments described herein, the immune cells expressing a CAR are administered in a dosage of from about 450×10⁶ cells to about 500×10⁶ cells. In specific embodiments of any of the embodiments described herein, the immune cells expressing a CAR are administered in a dosage of from about 250×10⁶ cells to about 400×10⁶ cells. In specific embodiments of any of the embodiments described herein, the immune cells expressing a CAR are administered in a dosage of from about 250×10⁶ cells to about 350×10⁶ cells. In specific embodiments of any of the embodiments described herein, the immune cells expressing a CAR are administered in a dosage of about 450×10⁶ cells. In specific embodiments of any of the embodiments described herein, the immune cells are T cells (e.g., autologous T cells). In specific embodiments of any of the embodiments described herein, the subjects being treated undergo a leukapharesis procedure to collect autologous immune cells for the manufacture of the immune cells expressing a CAR prior to their administration to the subject. In specific embodiments of any of the embodiments described herein, the immune cells (e.g., T cells) are administered by an intravenous infusion.

In specific embodiments of any of the embodiments described herein, the immune cells expressing a CAR directed to BCMA are administered in a dosage of from about 150×10⁶ cells to about 300×10⁶ cells. In specific embodiments of any of the embodiments described herein, the immune cells expressing a CAR directed to BCMA are administered in a dosage of from about 350×10⁶ cells to about 550×10⁶ cells. In specific embodiments of any of the embodiments described herein, the immune cells expressing a CAR directed to BCMA are administered in a dosage of from about 400×10⁶ cells to about 500×10⁶ cells. In specific embodiments of any of the embodiments described herein, the immune cells expressing a CAR directed to BCMA are administered in a dosage of from about 150×10⁶ cells to about 250×10⁶ cells. In specific embodiments of any of the embodiments described herein, the immune cells expressing a CAR directed to BCMA are administered in a dosage of from about 300×10⁶ cells to about 500×10⁶ cells. In specific embodiments of any of the embodiments described herein, the immune cells expressing a CAR directed to BCMA are administered in a dosage of from about 350×10⁶ cells to about 450×10⁶ cells. In specific embodiments of any of the embodiments described herein, the immune cells expressing a CAR directed to BCMA are administered in a dosage of from about 300×10⁶ cells to about 450×10⁶ cells. In specific embodiments of any of the embodiments described herein, the immune cells expressing a CAR directed to BCMA are administered in a dosage of from about 250×10⁶ cells to about 450×10⁶ cells. In specific embodiments of any of the embodiments described herein, the immune cells expressing a CAR directed to BCMA are administered in a dosage of from about 300×10⁶ cells to about 600×10⁶ cells. In specific embodiments of any of the embodiments described herein, the immune cells expressing a CAR directed to BCMA are administered in a dosage of from about 250×10⁶ cells to about 500×10⁶ cells. In specific embodiments of any of the embodiments described herein, the immune cells expressing a CAR directed to BCMA are administered in a dosage of from about 350×10⁶ cells to about 500×10⁶ cells. In specific embodiments of any of the embodiments described herein, the immune cells expressing a CAR directed to BCMA are administered in a dosage of from about 400×10⁶ cells to about 600×10⁶ cells. In specific embodiments of any of the embodiments described herein, the immune cells expressing a CAR directed to BCMA are administered in a dosage of from about 400×10⁶ cells to about 450×10⁶ cells. In specific embodiments of any of the embodiments described herein, the immune cells expressing a CAR directed to BCMA are administered in a dosage of from about 200×10⁶ cells to about 400×10⁶ cells. In specific embodiments of any of the embodiments described herein, the immune cells expressing a CAR directed to BCMA are administered in a dosage of from about 200×10⁶ cells to about 350×10⁶ cells. In specific embodiments of any of the embodiments described herein, the immune cells expressing a CAR directed to BCMA are administered in a dosage of from about 200×10⁶ cells to about 300×10⁶ cells. In specific embodiments of any of the embodiments described herein, the immune cells expressing a CAR directed to BCMA are administered in a dosage of from about 450×10⁶ cells to about 500×10⁶ cells. In specific embodiments of any of the embodiments described herein, the immune cells expressing a CAR directed to BCMA are administered in a dosage of from about 250×10⁶ cells to about 400×10⁶ cells. In specific embodiments of any of the embodiments described herein, the immune cells expressing a CAR directed to BCMA are administered in a dosage of from about 250×10⁶ cells to about 350×10⁶ cells. In specific embodiments of any of the embodiments described herein, the immune cells expressing a CAR directed to BCMA are administered in a dosage of about 450×10⁶ cells. In specific embodiments of any of the embodiments described herein, the immune cells are T cells (e.g., autologous T cells). In specific embodiments of any of the embodiments described herein, the subjects being treated undergo a leukapharesis procedure to collect autologous immune cells for the manufacture of the immune cells expressing a CAR directed to BCMA prior to their administration to the subject. In specific embodiments of any of the embodiments described herein, the immune cells (e.g., T cells) are administered by an intravenous infusion.

In specific embodiments of any of the aspects or embodiments disclosed herein, before administration of immune cells expressing a CAR, the subject being treated is administered a lymphodepleting (LD) chemotherapy. In specific embodiments, LD chemotherapy comprises fludarabine and/or cyclophosphamide. In specific embodiments, LD chemotherapy comprises fludarabine (e.g., about 30 mg/m² for intravenous administration) and cyclophosphamide (e.g., about 300 mg/m² for intravenous administration) for a duration of 1, 2, 3, 4, 5, 6, or 7 days (e.g., 3 days). In other specific embodiments, LD chemotherapy comprises any of the chemotherapeutic agents described in Section 5.9. In specific embodiments, the subject is administered immune cells expressing a chimeric antigen receptor (CAR) 1, 2, 3, 4, 5, 6, or 7 days after the administration of the LD chemotherapy (e.g., 2 or 3 days after the administration of the LD chemotherapy). In specific embodiments, the subject has not received any therapy prior to the initiation of the LD chemotherapy for at least or more than 1 week, at least or more than 2 weeks (at least or more than 14 days), at least or more than 3 weeks, at least or more than 4 weeks, at least or more than 5 weeks, or at least or more than 6 weeks. In specific embodiments of any of the embodiments disclosed herein, before administration of immune cells expressing a chimeric antigen receptor (CAR), the subject being treated has received only a single prior treatment regimen.

In specific embodiments of any of the aspects or embodiments disclosed herein, before administration of immune cells expressing a CAR directed to BCMA, the subject being treated is administered a lymphodepleting (LD) chemotherapy. In specific embodiments, LD chemotherapy comprises fludarabine and/or cyclophosphamide. In specific embodiments, LD chemotherapy comprises fludarabine (e.g., about 30 mg/m² for intravenous administration) and cyclophosphamide (e.g., about 300 mg/m² for intravenous administration) for a duration of 1, 2, 3, 4, 5, 6, or 7 days (e.g., 3 days). In other specific embodiments, LD chemotherapy comprises any of the chemotherapeutic agents described in Section 5.9. In specific embodiments, the subject is administered immune cells expressing a chimeric antigen receptor (CAR) directed to B Cell Maturation Antigen (BCMA) 1, 2, 3, 4, 5, 6, or 7 days after the administration of the LD chemotherapy (e.g., 2 or 3 days after the administration of the LD chemotherapy). In specific embodiments, the subject has not received any therapy prior to the initiation of the LD chemotherapy for at least or more than 1 week, at least or more than 2 weeks (at least or more than 14 days), at least or more than 3 weeks, at least or more than 4 weeks, at least or more than 5 weeks, or at least or more than 6 weeks. In specific embodiments of any of the embodiments disclosed herein, before administration of immune cells expressing a chimeric antigen receptor (CAR) directed to B Cell Maturation Antigen (BCMA), the subject being treated has received only a single prior treatment regimen.

For any of the above embodiments, the subject undergoes apheresis to collect and isolate said immune cells, e.g., T cells. In a specific embodiment of any of the above embodiments, said subject exhibits at the time of said apheresis: M-protein (serum protein electrophoresis [sPEP] or urine protein electrophoresis [uPEP]): sPEP ≥0.5 g/dL or uPEP ≥200 mg/24 hours; light chain multiple myeloma without measurable disease in the serum or urine, with serum immunoglobulin free light chain ≥10 mg/dL and abnormal serum immunoglobulin kappa lambda free light chain ratio; and/or Eastern Cooperative Oncology Group (ECOG) performance status ≤1. In a more specific embodiment, said subject at the time of apheresis additionally: has received at least three of said lines of prior treatment, including prior treatment with a proteasome inhibitor, an immunomodulatory agent (lenalidomide or pomalidomide) and an anti-CD38 antibody; has undergone at least 2 consecutive cycles of treatment for each of said at least three lines of prior treatment, unless progressive disease was the best response to a line of treatment; has evidence of progressive disease on or within 60 days of the most recent line of prior treatment; and/or has achieved a response (minimal response or better) to at least one of said prior lines of treatment. In a specific embodiment of any of the above embodiments, said subject exhibits at the time of said administration: M-protein (serum protein electrophoresis [sPEP] or urine protein electrophoresis [uPEP]): sPEP ≥0.5 g/dL or uPEP ≥200 mg/24 hours; light chain multiple myeloma without measurable disease in the serum or urine, with serum immunoglobulin free light chain ≥10 mg/dL and abnormal serum immunoglobulin kappa lambda free light chain ratio; and/or Eastern Cooperative Oncology Group (ECOG) performance status ≤1. In another more specific embodiment, said subject additionally: has received only one prior anti-myeloma treatment regimen; has the following high risk factors: R-ISS stage III, and early relapse, defined as (i) if the subject has undergone induction plus a stem cell transplant, progressive disease (PD) less than 12 months since date of first transplant; or (ii) if the subject has received only induction, PD<12 months since date of last treatment regimen which must contain at minimum, a proteasome inhibitor, an immunomodulatory agent and dexamethasone.

In a specific embodiment of any of any of the above aspects or embodiments, said CAR comprises an antibody or antibody fragment that targets BCMA. In a more specific embodiment, said CAR comprises a single chain Fv antibody fragment (scFv). In a more specific embodiment, said CAR comprises a BCMA02 scFv, e.g., SEQ ID NO: 38. In a specific embodiment of any of the above aspects or embodiments, said immune cells are idecabtagene vicleucel cells. In one embodiment, the chimeric antigen receptor comprises a murine single chain Fv antibody fragment that targets BCMA, e.g., BCMA. In one embodiment, the chimeric antigen receptor comprises a murine anti-BCMA scFv that binds a BCMA polypeptide, e.g., a human BCMA polypeptide a hinge domain comprising a CD8α polypeptide, a CD8α transmembrane domain, a CD137 (4-1BB) intracellular co-stimulatory signaling domain, and a CD3ζ primary signaling domain. In one embodiment, the chimeric antigen receptor comprises a murine scFv that targets BCMA, e.g., BCMA, wherein the scFV is that of anti-BCMA02 CAR of SEQ ID NO: 9. In one embodiment, the chimeric antigen receptor is or comprises SEQ ID NO: 9 or SEQ ID NO: 37. In one embodiment, the chimeric antigen receptor is or comprises SEQ ID NO: 9. In one embodiment, the chimeric antigen receptor is or comprises SEQ ID NO: 37. In a more specific embodiment of any embodiment herein, said immune cells are idecabtagene vicleucel (ide-cel) cells. In one embodiment, the immune cells comprise a chimeric antigen receptor which comprises a murine single chain Fv antibody fragment that targets BCMA, e.g., BCMA. In one embodiment, the immune cells comprise a chimeric antigen receptor which comprises a murine anti-BCMA scFv that binds a BCMA polypeptide, e.g., BCMA, a hinge domain comprising a CD8α polypeptide, a CD8α transmembrane domain, a CD137 (4-1BB) intracellular co-stimulatory signaling domain, and a CD3ζ primary signaling domain. In one embodiment, the immune cells comprise a chimeric antigen receptor which is or comprises SEQ ID NO: 9 or SEQ ID NO: 37. In one embodiment, the immune cells comprise a chimeric antigen receptor which is or comprises SEQ ID NO: 9. In one embodiment, the immune cells comprise a chimeric antigen receptor which is or comprises SEQ ID NO: 37.

In other embodiments, the genetically modified immune effector cells contemplated herein, are administered to a patient with a B cell related condition, e.g., a B cell malignancy.

The amount of soluble (i.e., non-membrane-bound) BCMA (sBCMA) after administration of a CAR T cell therapy, e.g., an anti-BCMA CAR T cell therapy, can be used to determine whether a subject can be expected to respond to the CAR T cell therapy appropriately, or whether the subject should be administered a different anticancer therapy. A greater drop in sBCMA levels in a tissue sample (e.g., serum, plasma, lymph, or blood) after administration of a CAR T cell therapy is correlated with a more clinically beneficial outcome (e.g., very good partial response, complete response or stringent complete response). In one aspect, for example, provided herein is a method of treating a disease caused by B Cell Maturation Agent (BCMA) expressing cells in a subject in need thereof, comprising: determining a first level of soluble BCMA (sBCMA) in a tissue sample from the subject; administering to the subject a first BCMA-based treatment modality comprising immune cells expressing a chimeric antigen receptor (CAR) directed to BCMA (BCMA CAR T cells), and then determining a second level of soluble BCMA in a tissue sample from the subject; wherein, if said second level of sBCMA is greater than about 30% of said first level of sBCMA, the subject is subsequently provided a second BCMA-based treatment modality to treat said disease; and wherein the first BCMA-based treatment modality and the second BCMA-based treatment modality are different BCMA-based treatment modalities. In a particular embodiment, the immune cells are idecabtagene vicleucel cells. In certain embodiments, the second BCMA-based treatment modality is not idecabtagene vicleucel cells. In certain embodiments, the second BCMA-based treatment modality does not comprise idecabtagene vicleucel cells.

In one aspect, for example, provided herein is a method of treating a disease caused by B Cell Maturation Agent (BCMA) expressing cells in a subject in need thereof, comprising: determining a first level of soluble BCMA (sBCMA) in a tissue sample from the subject; administering to the subject a first BCMA-based treatment modality comprising idecabtagene vicleucel cells, and then determining a second level of soluble BCMA in a tissue sample from the subject; wherein, if said second level of sBCMA is greater than about 30% of said first level of sBCMA, the subject is subsequently provided a second BCMA-based treatment modality to treat said disease; and wherein the first BCMA-based treatment modality and the second BCMA-based treatment modality are different BCMA-based treatment modalities. In certain embodiments, the second BCMA-based treatment modality is not idecabtagene vicleucel cells. In certain embodiments, the second BCMA-based treatment modality does not comprise idecabtagene vicleucel cells.

In one aspect, for example, provided herein is a method of treating a disease caused by B Cell Maturation Agent (BCMA) expressing cells in a subject in need thereof, comprising: determining a first level of soluble BCMA (sBCMA) in a tissue sample from the subject; administering to the subject a first BCMA-based treatment modality comprising immune cells expressing a chimeric antigen receptor (CAR) directed to BCMA (BCMA CAR T cells), and then determining a second level of soluble BCMA in a tissue sample from the subject; wherein, if said second level of sBCMA is greater than about 20%, 25%, or 30% of said first level of sBCMA, the subject is subsequently provided a second BCMA-based treatment modality to treat said disease; and wherein the first BCMA-based treatment modality and the second BCMA-based treatment modality are different BCMA-based treatment modalities. In a particular embodiment, the immune cells are idecabtagene vicleucel cells. In certain embodiments, the second BCMA-based treatment modality is not idecabtagene vicleucel cells. In certain embodiments, the second BCMA-based treatment modality does not comprise idecabtagene vicleucel cells.

In one aspect, for example, provided herein is a method of treating a disease caused by B Cell Maturation Agent (BCMA) expressing cells in a subject in need thereof, comprising: determining a first level of soluble BCMA (sBCMA) in a tissue sample from the subject; administering to the subject a first BCMA-based treatment modality comprising idecabtagene vicleucel cells, and then determining a second level of soluble BCMA in a tissue sample from the subject; wherein, if said second level of sBCMA is greater than about 20%, 25%, or 30% of said first level of sBCMA, the subject is subsequently provided a second BCMA-based treatment modality to treat said disease; and wherein the first BCMA-based treatment modality and the second BCMA-based treatment modality are different BCMA-based treatment modalities. In certain embodiments, the second BCMA-based treatment modality is not idecabtagene vicleucel cells. In certain embodiments, the second BCMA-based treatment modality does not comprise idecabtagene vicleucel cells.

In one aspect, for example, provided herein is a method of treating a disease caused by B Cell Maturation Agent (BCMA) expressing cells in a subject in need thereof, comprising: determining a first level of soluble BCMA (sBCMA) in a tissue sample from the subject; administering to the subject a first BCMA-based treatment modality comprising immune cells expressing a chimeric antigen receptor (CAR) directed to BCMA (BCMA CAR T cells), and then determining a second level of soluble BCMA in a tissue sample from the subject; wherein, if said second level of sBCMA is greater than about 30%, 35%, 40%, 45%, or 50% of said first level of sBCMA, the subject is subsequently provided a second BCMA-based treatment modality to treat said disease; and wherein the first BCMA-based treatment modality and the second BCMA-based treatment modality are different BCMA-based treatment modalities.

In one aspect, for example, provided herein is a method of treating a disease caused by B Cell Maturation Agent (BCMA) expressing cells in a subject in need thereof, comprising: determining a first level of soluble BCMA (sBCMA) in a tissue sample from the subject; administering to the subject a first BCMA-based treatment modality comprising immune cells expressing a chimeric antigen receptor (CAR) directed to BCMA (BCMA CAR T cells), and then determining a second level of soluble BCMA in a tissue sample from the subject; wherein, if said second level of sBCMA is greater than about 30%, 35%, 40%, 45%, or 50% of said first level of sBCMA, the subject is subsequently provided a second BCMA-based treatment modality to treat said disease; and wherein the first BCMA-based treatment modality and the second BCMA-based treatment modality are different BCMA-based treatment modalities. In a particular embodiment, the immune cells are idecabtagene vicleucel cells. In certain embodiments, the second BCMA-based treatment modality is not idecabtagene vicleucel cells. In certain embodiments, the second BCMA-based treatment modality does not comprise idecabtagene vicleucel cells.

In one aspect, for example, provided herein is a method of treating a disease caused by B Cell Maturation Agent (BCMA) expressing cells in a subject in need thereof, comprising: determining a first level of soluble BCMA (sBCMA) in a tissue sample from the subject; administering to the subject a first BCMA-based treatment modality comprising idecabtagene vicleucel cells, and then determining a second level of soluble BCMA in a tissue sample from the subject; wherein, if said second level of sBCMA is greater than about 30%, 35%, 40%, 45%, or 50% of said first level of sBCMA, the subject is subsequently provided a second BCMA-based treatment modality to treat said disease; and wherein the first BCMA-based treatment modality and the second BCMA-based treatment modality are different BCMA-based treatment modalities. In certain embodiments, the second BCMA-based treatment modality is not idecabtagene vicleucel cells. In certain embodiments, the second BCMA-based treatment modality does not comprise idecabtagene vicleucel cells.

Also provided herein is a method of treating a disease caused by B Cell Maturation Agent (BCMA) expressing cells in a subject in need thereof, comprising: (a) determining a first level of soluble BCMA (sBCMA) in a tissue sample from the subject; (b) administering to the subject a first BCMA-based treatment modality comprising immune cells expressing a chimeric antigen receptor (CAR) directed to BCMA (BCMA CAR T cells); and (c) determining that a second level of soluble BCMA in a tissue sample from the subject is greater than about 30% of said first level, and on the basis of the determination in step c, subsequently providing a second BCMA-based treatment modality to the subject; wherein the first BCMA-based treatment modality and the second BCMA-based treatment modality are different BCMA-based treatment modalities. In a particular embodiment, the immune cells are idecabtagene vicleucel cells. In certain embodiments, the second BCMA-based treatment modality is not idecabtagene vicleucel cells. In certain embodiments, the second BCMA-based treatment modality does not comprise idecabtagene vicleucel cells.

Also provided herein is a method of treating a disease caused by B Cell Maturation Agent (BCMA) expressing cells in a subject in need thereof, comprising: (a) determining a first level of soluble BCMA (sBCMA) in a tissue sample from the subject; (b) administering to the subject a first BCMA-based treatment modality comprising idecabtagene vicleucel cells; and (c) determining that a second level of soluble BCMA in a tissue sample from the subject is greater than about 30% of said first level, and on the basis of the determination in step c, subsequently providing a second BCMA-based treatment modality to the subject; wherein the first BCMA-based treatment modality and the second BCMA-based treatment modality are different BCMA-based treatment modalities. In certain embodiments, the second BCMA-based treatment modality is not idecabtagene vicleucel cells. In certain embodiments, the second BCMA-based treatment modality does not comprise idecabtagene vicleucel cells.

Also provided herein is a method of treating a disease caused by B Cell Maturation Agent (BCMA) expressing cells in a subject in need thereof, comprising: (a) determining a first level of soluble BCMA (sBCMA) in a tissue sample from the subject; (b) administering to the subject a first BCMA-based treatment modality comprising immune cells expressing a chimeric antigen receptor (CAR) directed to BCMA (BCMA CAR T cells); and (c) determining that a second level of soluble BCMA in a tissue sample from the subject is greater than about 20%, 25%, or 30% of said first level, and on the basis of the determination in step c, subsequently providing a second BCMA-based treatment modality to the subject; wherein the first BCMA-based treatment modality and the second BCMA-based treatment modality are different BCMA-based treatment modalities. In a particular embodiment, the immune cells are idecabtagene vicleucel cells. In certain embodiments, the second BCMA-based treatment modality is not idecabtagene vicleucel cells. In certain embodiments, the second BCMA-based treatment modality does not comprise idecabtagene vicleucel cells.

Also provided herein is a method of treating a disease caused by B Cell Maturation Agent (BCMA) expressing cells in a subject in need thereof, comprising: (a) determining a first level of soluble BCMA (sBCMA) in a tissue sample from the subject; (b) administering to the subject a first BCMA-based treatment modality comprising idecabtagene vicleucel cells; and (c) determining that a second level of soluble BCMA in a tissue sample from the subject is greater than about 20%, 25%, or 30% of said first level, and on the basis of the determination in step c, subsequently providing a second BCMA-based treatment modality to the subject; wherein the first BCMA-based treatment modality and the second BCMA-based treatment modality are different BCMA-based treatment modalities. In certain embodiments, the second BCMA-based treatment modality is not idecabtagene vicleucel cells. In certain embodiments, the second BCMA-based treatment modality does not comprise idecabtagene vicleucel cells.

Also provided herein is a method of treating a disease caused by B Cell Maturation Agent (BCMA) expressing cells in a subject in need thereof, comprising: (a) determining a first level of soluble BCMA (sBCMA) in a tissue sample from the subject; (b) administering to the subject a first BCMA-based treatment modality comprising immune cells expressing a chimeric antigen receptor (CAR) directed to BCMA (BCMA CAR T cells); and (c) determining that a second level of soluble BCMA in a tissue sample from the subject is greater than about 30%, 35%, 40%, 45%, or 50% of said first level, and on the basis of the determination in step c, subsequently providing a second BCMA-based treatment modality to the subject; wherein the first BCMA-based treatment modality and the second BCMA-based treatment modality are different BCMA-based treatment modalities. In a particular embodiment, the immune cells are idecabtagene vicleucel cells. In certain embodiments, the second BCMA-based treatment modality is not idecabtagene vicleucel cells. In certain embodiments, the second BCMA-based treatment modality does not comprise idecabtagene vicleucel cells.

Also provided herein is a method of treating a disease caused by B Cell Maturation Agent (BCMA) expressing cells in a subject in need thereof, comprising: (a) determining a first level of soluble BCMA (sBCMA) in a tissue sample from the subject; (b) administering to the subject a first BCMA-based treatment modality comprising idecabtagene vicleucel cells; and (c) determining that a second level of soluble BCMA in a tissue sample from the subject is greater than about 30%, 35%, 40%, 45%, or 50% of said first level, and on the basis of the determination in step c, subsequently providing a second BCMA-based treatment modality to the subject; wherein the first BCMA-based treatment modality and the second BCMA-based treatment modality are different BCMA-based treatment modalities. In certain embodiments, the second BCMA-based treatment modality is not idecabtagene vicleucel cells. In certain embodiments, the second BCMA-based treatment modality does not comprise idecabtagene vicleucel cells.

In a specific embodiment of either of the above embodiments, if said second level of sBCMA is greater than 40% of said first level, the subject is provided a second BCMA-based treatment modality to treat said disease.
In a embodiment of either of the above embodiments, said second level of sBCMA is determined at 25-35 days after said administering. In another specific embodiment, said second level of sBCMA is determined at 23-35, 24-35, 25-36, 25-37, 23-35, or 25-37 days after said administering. In another specific embodiment, said second level of sBCMA is determined at 23, 24, 25, 26, 27, 28, 29, 30, 31, 32, 33, 34, 35, 36, or 37 days after said administering. In another specific embodiment, said second level of sBCMA is determined at 28-31 days after said administering. In another specific embodiment, said second level of sBCMA is determined at 26-31, 27-31, 28-32, 28-33, 26-31, or 27-33 days after said administering. In another specific embodiment, said second level of sBCMA is determined at 26, 27, 28, 29, 30, 31, 32, or 33 days after said administering. In more specific embodiments, the subject is provided a second BCMA-based treatment modality within three months, two months, or one month after said determining a second level of sBCMA.
In another aspect, provided herein is a method of treating a disease caused by B Cell Maturation Agent (BCMA) expressing cells, comprising administering to a patient diagnosed with said disease a second BCMA-based treatment modality, wherein the patient has previously been administered a first BCMA-based treatment modality comprising immune cells expressing a chimeric antigen receptor (CAR) directed to BCMA (BCMA CAR T cells), wherein the first BCMA-based treatment modality and the second BCMA-based treatment modality are different BCMA-based treatment modalities, and wherein a tissue sample from the patient subsequent to said administration contained a level of soluble BCMA (sBCMA) greater than 30% of a level of sBCMA found in a tissue sample obtained from the patient prior to said administration. In a particular embodiment, the immune cells are idecabtagene vicleucel cells. In certain embodiments, the second BCMA-based treatment modality is not idecabtagene vicleucel cells.
In certain embodiments, the second BCMA-based treatment modality does not comprise idecabtagene vicleucel cells.
In another aspect, provided herein is a method of treating a disease caused by B Cell Maturation Agent (BCMA) expressing cells, comprising administering to a patient diagnosed with said disease a second BCMA-based treatment modality, wherein the patient has previously been administered a first BCMA-based treatment modality comprising idecabtagene vicleucel cells, wherein the first BCMA-based treatment modality and the second BCMA-based treatment modality are different BCMA-based treatment modalities, and wherein a tissue sample from the patient subsequent to said administration contained a level of soluble BCMA (sBCMA) greater than 30% of a level of sBCMA found in a tissue sample obtained from the patient prior to said administration. In certain embodiments, the second BCMA-based treatment modality is not idecabtagene vicleucel cells. In certain embodiments, the second BCMA-based treatment modality does not comprise idecabtagene vicleucel cells.
In another aspect, provided herein is a method of determining whether a patient diagnosed with a disease caused by B Cell Maturation Agent (BCMA) expressing cells should be administered a second BCMA-based treatment modality after treatment with a first BCMA-based treatment modality comprising immune cells expressing a chimeric antigen receptor (CAR) directed to BCMA (BCMA CAR T cells), wherein the first BCMA-based treatment modality and the second BCMA-based treatment modality are different BCMA-based treatment modalities, comprising determining a level of soluble BCMA (sBCMA) in a tissue sample from the patient, wherein the patient has previously been administered the first BCMA-based treatment modality comprising immune cells expressing a chimeric antigen receptor (CAR) directed to BCMA (BCMA CAR T cells), and wherein if the level of sBCMA in the tissue sample is greater than 30% of a level of sBCMA found in a tissue sample obtained from the patient prior to said administration, then the patient is a candidate for the second BCMA-based treatment modality. In a specific embodiment, the method further comprises administering the second BCMA-based treatment modality to the candidate for the second BCMA-based treatment modality. In a particular embodiment, the immune cells are idecabtagene vicleucel cells. In certain embodiments, the second BCMA-based treatment modality is not idecabtagene vicleucel cells. In certain embodiments, the second BCMA-based treatment modality does not comprise idecabtagene vicleucel cells.

In another aspect, provided herein is a method of determining whether a patient diagnosed with a disease caused by B Cell Maturation Agent (BCMA) expressing cells should be administered a second BCMA-based treatment modality after treatment with a first BCMA-based treatment modality comprising idecabtagene vicleucel cells, wherein the first BCMA-based treatment modality and the second BCMA-based treatment modality are different BCMA-based treatment modalities, comprising determining a level of soluble BCMA (sBCMA) in a tissue sample from the patient, wherein the patient has previously been administered the first BCMA-based treatment modality comprising immune cells expressing a chimeric antigen receptor (CAR) directed to BCMA (BCMA CAR T cells), and wherein if the level of sBCMA in the tissue sample is greater than 30% of a level of sBCMA found in a tissue sample obtained from the patient prior to said administration, then the patient is a candidate for the second BCMA-based treatment modality. In a specific embodiment, the method further comprises administering the second BCMA-based treatment modality to the candidate for the second BCMA-based treatment modality. In certain embodiments, the second BCMA-based treatment modality is not idecabtagene vicleucel cells. In certain embodiments, the second BCMA-based treatment modality does not comprise idecabtagene vicleucel cells.

The absolute level of sBCMA in a tissue sample (e.g., plasma, serum, lymph or blood) may also be used to determine whether a person administered a CAR T cell therapy, e.g., a BCMA CAR T cell therapy will appropriately benefit from that therapy, or should be administered a different anticancer therapy. Thus, provided herein is a method of treating a disease caused by B Cell Maturation Agent (BCMA) expressing cells in a subject in need thereof, comprising: administering to the subject a first BCMA-based treatment modality comprising immune cells expressing a chimeric antigen receptor (CAR) directed to BCMA (BCMA CAR T cells), and determining a level of soluble BCMA (sBCMA) in a tissue sample from the subject; wherein, if said level of sBCMA is greater than 4000 ng/L, the subject is subsequently provided a second BCMA-based treatment modality to treat said disease, and wherein the first BCMA-based treatment modality and the second BCMA-based treatment modality are different BCMA-based treatment modalities. In a particular embodiment, the immune cells are idecabtagene vicleucel cells. In certain embodiments, the second BCMA-based treatment modality is not idecabtagene vicleucel cells. In certain embodiments, the second BCMA-based treatment modality does not comprise idecabtagene vicleucel cells.

In another aspect, provided herein is a method of treating a disease caused by B Cell Maturation Agent (BCMA) expressing cells in a subject in need thereof, comprising: administering to the subject a first BCMA-based treatment modality comprising idecabtagene vicleucel cells, and determining a level of soluble BCMA (sBCMA) in a tissue sample from the subject; wherein, if said level of sBCMA is greater than 4000 ng/L, the subject is subsequently provided a second BCMA-based treatment modality to treat said disease, and wherein the first BCMA-based treatment modality and the second BCMA-based treatment modality are different BCMA-based treatment modalities. In certain embodiments, the second BCMA-based treatment modality is not idecabtagene vicleucel cells. In certain embodiments, the second BCMA-based treatment modality does not comprise idecabtagene vicleucel cells.

In a specific embodiment of either of the above embodiments, if said level of sBCMA is greater than about 3000 ng/L, 3500 ng/L, 4000 ng/L, 4500 ng/L, or 5000 ng/L the subject is subsequently provided a BCMA-based treatment modality to treat said disease. In a specific embodiment, said first level of sBCMA is determined at 50-70 days after said administering. In a specific embodiment, said first level of sBCMA is determined at 45-70, 46-70, 47-70, 48-70, 49-70, 50-70, 50-71, 50-72, 50-73, or 50-75 days after said administering. In a specific embodiment, said first level of sBCMA is determined at 50, 51, 52, 53, 54, 55, 56, 57, 58, 59 60, 61, 62, 63, 64, 65, 66, 67, 68, 69, or 70 days after said administering. In another specific embodiment, said first level of sBCMA is determined at 55-65 days after said administering. In another specific embodiment, said first level of sBCMA is determined at 50-65, 51-65, 52-65, 53-65, 54-65, 55-64, 55-63, 55-62, or 55-61 days after said administering. In another specific embodiment, said first level of sBCMA is determined at 55, 56, 57, 58, 59, 60, 61, 62, 63, 64, or 65 days after said administering. In another specific embodiment, said level of sBCMA is determined at 58-62 days after said administering. In another specific embodiment, said level of sBCMA is determined at 53-62, 54-62, 55-62, 56-62, 57-62, 58-68, 58-67, 58-66, 58-65, 58-64, or 58-63 days after said administering. In another specific embodiment, said level of sBCMA is determined at 58, 59, 60, 61, or 62 days after said administering. In a specific embodiment of the preceding embodiments, the subject is provided said second BCMA-based treatment modality within three months, two months, or one month after said determining a first level of sBCMA.

The levels of certain cytokines, e.g., interleukin-6 (IL-6) and/or tumor necrosis factor alpha (TNFα) can also be used to determine whether a person administered a CAR T cell therapy, e.g., a BCMA CAR T cell therapy will appropriately benefit from that therapy, or should be administered a different anticancer therapy. Thus, provided herein is a method of treating a disease caused by B Cell Maturation Agent (BCMA) expressing cells in a subject in need thereof, comprising: determining a first level of interleukin-6 (IL-6), tumor necrosis factor alpha (TNFα) or both in a tissue sample from the subject; administering to the subject a first BCMA-based treatment modality comprising immune cells expressing a chimeric antigen receptor (CAR) directed to BCMA (BCMA CAR T cells), and subsequently determining a second level of IL-6, TNFα or both in a tissue sample from the subject; wherein, if said second level of IL-6, TNFα or both is not greater than said first level of IL-6, TNFα or both, respectively, then the subject is subsequently provided a second BCMA-based treatment modality to treat said disease, and wherein the first BCMA-based treatment modality and the second BCMA-based treatment modality are different BCMA-based treatment modalities. In a particular embodiment, the immune cells are idecabtagene vicleucel cells. In certain embodiments, the second BCMA-based treatment modality is not idecabtagene vicleucel cells. In certain embodiments, the second BCMA-based treatment modality does not comprise idecabtagene vicleucel cells.

In another aspect, provided herein is a method of treating a disease caused by B Cell Maturation Agent (BCMA) expressing cells in a subject in need thereof, comprising: determining a first level of interleukin-6 (IL-6), tumor necrosis factor alpha (TNFα) or both in a tissue sample from the subject; administering to the subject a first BCMA-based treatment modality comprising idecabtagene vicleucel cells, and subsequently determining a second level of IL-6, TNFα or both in a tissue sample from the subject; wherein, if said second level of IL-6, TNFα or both is not greater than said first level of IL-6, TNFα or both, respectively, then the subject is subsequently provided a second BCMA-based treatment modality to treat said disease, and wherein the first BCMA-based treatment modality and the second BCMA-based treatment modality are different BCMA-based treatment modalities. In certain embodiments, the second BCMA-based treatment modality is not idecabtagene vicleucel cells. In certain embodiments, the second BCMA-based treatment modality does not comprise idecabtagene vicleucel cells.

In certain embodiments, said first level is determined on the day of said administering to the subject the first BCMA-based treatment modality comprising immune cells expressing a CAR directed to BCMA, and said second level is determined 1-4 days after said administering. In another specific embodiment, said second level is determined one day after said administering. In another specific embodiment, said second level is determined two days after said administering. In another specific embodiment, said second level is determined three days after said administering. In another specific embodiment, said second level is determined four days after said administering.

In another aspect, provided herein is a method of treating a disease caused by B Cell Maturation Agent (BCMA)-expressing cells in a subject in need thereof, comprising: administering to the subject immune cells expressing a chimeric antigen receptor (CAR) directed to BCMA (BCMA CAR T cells), and determining a level of ferritin in a tissue sample from the subject; wherein, if said level of ferritin is greater than 1500 picomoles per liter, the subject is subsequently provided a therapy to treat cytokine release syndrome (CRS). In certain embodiments, said determining is performed within 0-4 days prior to said administering. In a specific embodiment, said determining is performed on the same day as said administering. In another specific embodiment, said therapy to treat CRS is first provided to said subject 0-5 days after said administering.

In another aspect, provided herein is a method of treating a disease caused by B Cell Maturation Agent (BCMA) expressing cells in a subject in need thereof, comprising: (a) determining a first level of soluble BCMA (sBCMA) and/or a first level of interleukin-6 (IL-6), tumor necrosis factor alpha (TNFα), or both in a tissue sample from the subject; (b) administering to the subject a first BCMA-based treatment modality comprising immune cells expressing a chimeric antigen receptor (CAR) directed to BCMA (BCMA CAR T cells), and (c) determining a second level of sBCMA and/or a second level of interleukin-6 (IL-6), tumor necrosis factor alpha (TNFα), or both in a tissue sample from the subject wherein, if said second level of sBCMA is greater than 30% of said first level of sBCMA and/or if said second level of IL-6, TNFα or both is not greater than said first level of IL-6, TNFα or both, the subject is subsequently provided a second BCMA-based treatment modality to treat said disease, and wherein the first BCMA-based treatment modality and the second BCMA-based treatment modality are different BCMA-based treatment modalities. In a particular embodiment, the immune cells are idecabtagene vicleucel cells. In certain embodiments, the second BCMA-based treatment modality is not idecabtagene vicleucel cells. In certain embodiments, the second BCMA-based treatment modality does not comprise idecabtagene vicleucel cells.

In another aspect, provided herein is a method of treating a disease caused by B Cell Maturation Agent (BCMA) expressing cells in a subject in need thereof, comprising: (a) determining a first level of soluble BCMA (sBCMA) and/or a first level of interleukin-6 (IL-6), tumor necrosis factor alpha (TNFα), or both in a tissue sample from the subject; (b) administering to the subject a first BCMA-based treatment modality comprising idecabtagene vicleucel cells, and (c) determining a second level of sBCMA and/or a second level of interleukin-6 (IL-6), tumor necrosis factor alpha (TNFα), or both in a tissue sample from the subject wherein, if said second level of sBCMA is greater than 30% of said first level of sBCMA and/or if said second level of IL-6, TNFα or both is not greater than said first level of IL-6, TNFα or both, the subject is subsequently provided a second BCMA-based treatment modality to treat said disease, and wherein the first BCMA-based treatment modality and the second BCMA-based treatment modality are different BCMA-based treatment modalities. In certain embodiments, the second BCMA-based treatment modality is not idecabtagene vicleucel cells. In certain embodiments, the second BCMA-based treatment modality does not comprise idecabtagene vicleucel cells.

In another aspect, provided herein is a method of treating a disease caused by B Cell Maturation Agent (BCMA) expressing cells in a subject in need thereof, comprising: (a) determining a first level of soluble BCMA (sBCMA) and/or a first level of interleukin-6 (IL-6), tumor necrosis factor alpha (TNFα), or both in a tissue sample from the subject; (b) administering to the subject a first BCMA-based treatment modality comprising immune cells expressing a chimeric antigen receptor (CAR) directed to BCMA (BCMA CAR T cells), (c) determining that a second level of sBCMA in a tissue sample from the subject is greater than 30% of said first level of sBCMA and/or a second level of IL-6, TNFα or both is not greater than said first level of IL-6, TNFα or both, and (d) on the basis of the determination in step c, subsequently providing a second BCMA-based treatment modality to the subject, wherein the first BCMA-based treatment modality and the second BCMA-based treatment modality are different BCMA-based treatment modalities. In a particular embodiment, the immune cells are idecabtagene vicleucel cells. In certain embodiments, the second BCMA-based treatment modality is not idecabtagene vicleucel cells. In certain embodiments, the second BCMA-based treatment modality does not comprise idecabtagene vicleucel cells.

In another aspect, provided herein is a method of treating a disease caused by B Cell Maturation Agent (BCMA) expressing cells in a subject in need thereof, comprising: (a) determining a first level of soluble BCMA (sBCMA) and/or a first level of interleukin-6 (IL-6), tumor necrosis factor alpha (TNFα), or both in a tissue sample from the subject; (b) administering to the subject a first BCMA-based treatment modality comprising idecabtagene vicleucel cells, (c) determining that a second level of sBCMA in a tissue sample from the subject is greater than 30% of said first level of sBCMA and/or a second level of IL-6, TNFα or both is not greater than said first level of IL-6, TNFα or both, and (d) on the basis of the determination in step c, subsequently providing a second BCMA-based treatment modality to the subject, wherein the first BCMA-based treatment modality and the second BCMA-based treatment modality are different BCMA-based treatment modalities. In certain embodiments, the second BCMA-based treatment modality is not idecabtagene vicleucel cells. In certain embodiments, the second BCMA-based treatment modality does not comprise idecabtagene vicleucel cells.

In another aspect, provided herein is a method of treating a disease caused by B Cell Maturation Agent (BCMA) expressing cells, comprising administering to a patient diagnosed with said disease a second BCMA-based treatment modality, wherein the patient has previously been administered a first BCMA-based treatment modality comprising immune cells expressing a chimeric antigen receptor (CAR) directed to BCMA (BCMA CAR T cells), wherein the first BCMA-based treatment modality and the second BCMA-based treatment modality are different BCMA-based treatment modalities, and wherein a tissue sample from the patient subsequent to said administration contained (i) a level of soluble BCMA (sBCMA) greater than 30% of a level of sBCMA found in a tissue sample obtained from the patient prior to said administration and/or (ii) a level of IL-6, TNFα or both not greater than a level of IL-6, TNFα or both found in a tissue sample obtained from the patient prior to said administration. In a particular embodiment, the immune cells are idecabtagene vicleucel cells. In certain embodiments, the second BCMA-based treatment modality is not idecabtagene vicleucel cells. In certain embodiments, the second BCMA-based treatment modality does not comprise idecabtagene vicleucel cells.

In another aspect, provided herein is a method of treating a disease caused by B Cell Maturation Agent (BCMA) expressing cells, comprising administering to a patient diagnosed with said disease a second BCMA-based treatment modality, wherein the patient has previously been administered a first BCMA-based treatment modality comprising idecabtagene vicleucel cells, wherein the first BCMA-based treatment modality and the second BCMA-based treatment modality are different BCMA-based treatment modalities, and wherein a tissue sample from the patient subsequent to said administration contained (i) a level of soluble BCMA (sBCMA) greater than 30% of a level of sBCMA found in a tissue sample obtained from the patient prior to said administration and/or (ii) a level of IL-6, TNFα or both not greater than a level of IL-6, TNFα or both found in a tissue sample obtained from the patient prior to said administration. In certain embodiments, the second BCMA-based treatment modality is not idecabtagene vicleucel cells. In certain embodiments, the second BCMA-based treatment modality does not comprise idecabtagene vicleucel cells.

In another aspect, provided herein is a method of determining whether a patient diagnosed with a disease caused by B Cell Maturation Agent (BCMA) expressing cells should be administered a second BCMA-based treatment modality after treatment with a first BCMA-based treatment modality comprising immune cells expressing a chimeric antigen receptor (CAR) directed to BCMA (BCMA CAR T cells), comprising determining a level of soluble BCMA (sBCMA) and/or a level of IL-6, TNFα or both in a tissue sample from the patient, wherein the patient has previously been administered the first BCMA-based treatment modality comprising immune cells expressing a chimeric antigen receptor (CAR) directed to BCMA (BCMA CAR T cells), wherein if (i) the level of sBCMA in the tissue sample is greater than 30% of a level of sBCMA found in a tissue sample obtained from the patient prior to said administration and/or (ii) the level of IL-6, TNFα or both is not greater than a level of IL-6, TNFα or both found in a tissue sample obtained from the patient prior to said administration, then the patient is a candidate for the second BCMA-based treatment modality, and wherein the first BCMA-based treatment modality and the second BCMA-based treatment modality are different BCMA-based treatment modalities. In a specific embodiment, the method further comprises administering the second BCMA-based treatment modality to the candidate for the second BCMA-based treatment modality. In a particular embodiment, the immune cells are idecabtagene vicleucel cells. In certain embodiments, the second BCMA-based treatment modality is not idecabtagene vicleucel cells. In certain embodiments, the second BCMA-based treatment modality does not comprise idecabtagene vicleucel cells.

In another aspect, provided herein is a method of determining whether a patient diagnosed with a disease caused by B Cell Maturation Agent (BCMA) expressing cells should be administered a second BCMA-based treatment modality after treatment with a first BCMA-based treatment modality comprising immune cells expressing a chimeric antigen receptor (CAR) directed to BCMA (BCMA CAR T cells), comprising determining a level of soluble BCMA (sBCMA) and/or a level of IL-6, TNFα or both in a tissue sample from the patient, wherein the patient has previously been administered the first BCMA-based treatment modality comprising idecabtagene vicleucel cells, wherein if (i) the level of sBCMA in the tissue sample is greater than 30% of a level of sBCMA found in a tissue sample obtained from the patient prior to said administration and/or (ii) the level of IL-6, TNFα or both is not greater than a level of IL-6, TNFα or both found in a tissue sample obtained from the patient prior to said administration, then the patient is a candidate for the second BCMA-based treatment modality, and wherein the first BCMA-based treatment modality and the second BCMA-based treatment modality are different BCMA-based treatment modalities. In a specific embodiment, the method further comprises administering the second BCMA-based treatment modality to the candidate for the second BCMA-based treatment modality. In certain embodiments, the second BCMA-based treatment modality is not idecabtagene vicleucel cells. In certain embodiments, the second BCMA-based treatment modality does not comprise idecabtagene vicleucel cells.

In specific embodiments of any of the above aspects or embodiments, said CAR T cell therapy (e.g., immune cells expressing a chimeric antigen receptor (CAR) directed to BCMA (BCMA CAR T cells), e.g., idecabtagene vicleucel (ide-cel) cells) comprises a population of cells that comprises about 10%, 5%, 3%, 2%, or 1% activated CAR T-cells, for example, activated CD8 CAR T-cells (CD3+/CD8+/CAR+/CD25+).

In specific embodiments of any of the above aspects or embodiments, said CAR T cell therapy (e.g., immune cells expressing a chimeric antigen receptor (CAR) directed to BCMA (BCMA CAR T cells), e.g., idecabtagene vicleucel (ide-cel) cells) comprises a population of cells that comprises 10%, 5%, 3%, 2%, or 1% senescence population of CAR T-cells, for example, CD4 CAR T-cells (CD3+/CD4+/CAR+/CD57+). In a specific embodiments of any of the above aspects or embodiments, said tissue sample is blood, plasma or serum. In another specific embodiments of any of the above aspects or embodiments, said disease caused by BCMA-expressing cells is multiple myeloma, chronic lymphocytic leukemia, or a non-Hodgkins lymphoma (e.g., Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, and mantle cell lymphoma). In specific embodiments, the disease is multiple myeloma, e.g., high-risk multiple myeloma or relapsed and refractory multiple myeloma. In other specific embodiments, the high risk multiple myeloma is R-ISS stage III disease and/or a disease characterized by early relapse (e.g., progressive disease within 12 months since the date of last treatment regimen, such as last treatment regimen with a proteasome inhibitor, an immunomodulatory agent and/or dexamethasone). In a particular embodiment, the multiple myeloma is not R-ISS stage III disease. In specific embodiments, said disease caused by BCMA-expressing cells is a non-Hodgkins lymphoma, and wherein the non-Hodgkins lymphoma is selected from the group consisting of: Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, and mantle cell lymphoma.

In one embodiment, before the administration of the T cells expressing a chimeric antigen receptor (CAR) directed to B Cell Maturation Antigen (BCMA), the subject having a tumor has been assessed for expression of BCMA by the tumor.

In specific embodiments of any of the above aspects or embodiments, the immune cells are T cells, e.g., CD4+ T cells, CD8+ T cells or cytotoxic T lymphocytes (CTLs), T killer cells, or natural killer (NK) cells. In another specific embodiment specific embodiment, the immune cells are administered in a dosage of from 150×10⁶ cells to 450×10⁶ cells.

Generally, a BCMA-based treatment modality refers to a treatment modality that targets BCMA and/or cells expressing BCMA (e.g., cells expressing BCMA on the cell surface). For example the BCMA-based treatment modality (e.g., the first BCMA-based treatment modality or the second BCMA-based treatment modality) may be a BCMA-Antibody-Drug Conjugate (ADC), a bispecific T-cell engager (BiTE) that targets B-cell maturation antigen (BCMA), a natural killer (NK) cell engager (NKCEs) that targets B-cell maturation antigen (BCMA), or immune cells expressing a chimeric antigen receptor (CAR) directed to BCMA (BCMA CAR T cells). In a specific embodiment of any of the above embodiments, the second BCMA-based treatment modality comprises a BCMA-Antibody-Drug Conjugate (ADC), a bispecific T-cell engager (BiTE) that targets B-cell maturation antigen (BCMA), a natural killer (NK) cell engager (NKCEs) that targets B-cell maturation antigen (BCMA), or immune cells expressing a chimeric antigen receptor (CAR) directed to BCMA (BCMA CAR T cells). In certain embodiments, the second BCMA-based treatment modality comprises a BCMA-Antibody-Drug Conjugate (ADC), a bispecific T-cell engager (BiTE), natural killer (NK) cell engagers (NKCEs) that target B-cell maturation antigen (BCMA), or immune cells expressing a chimeric antigen receptor (CAR) directed to BCMA (BCMA CAR T cells), wherein the immune cells expressing a chimeric antigen receptor (CAR) directed to BCMA (BCMA CAR T cells) are not the same as the first BCMA-based treatment modality comprising immune cells expressing a chimeric antigen receptor (CAR) directed to BCMA (BCMA CAR T cells). In certain embodiments, the second BCMA-based treatment modality is a BCMA-Antibody-Drug Conjugate (ADC), a bispecific T-cell engager (BiTE) that targets B-cell maturation antigen (BCMA), a natural killer (NK) cell engager (NKCEs) that targets B-cell maturation antigen (BCMA), or immune cells expressing a chimeric antigen receptor (CAR) directed to BCMA (BCMA CAR T cells). In certain embodiments, the second BCMA-based treatment modality is selected from the group consisting of a BCMA-Antibody-Drug Conjugate (ADC), a bispecific T-cell engager (BiTE) that targets B-cell maturation antigen (BCMA), a natural killer (NK) cell engager (NKCEs) that targets B-cell maturation antigen (BCMA), and immune cells expressing a chimeric antigen receptor (CAR) directed to BCMA (BCMA CAR T cells). In certain embodiments, the second BCMA-based treatment modality is a BCMA-Antibody-Drug Conjugate (ADC), a bispecific T-cell engager (BiTE), natural killer (NK) cell engagers (NKCEs) that target B-cell maturation antigen (BCMA), or immune cells expressing a chimeric antigen receptor (CAR) directed to BCMA (BCMA CAR T cells), wherein the immune cells expressing a chimeric antigen receptor (CAR) directed to BCMA (BCMA CAR T cells) are not the same as the first BCMA-based treatment modality comprising immune cells expressing a chimeric antigen receptor (CAR) directed to BCMA (BCMA CAR T cells). In certain embodiments, the second BCMA-based treatment modality is selected from the group consisting of a BCMA-Antibody-Drug Conjugate (ADC), a bispecific T-cell engager (BiTE), natural killer (NK) cell engagers (NKCEs) that target B-cell maturation antigen (BCMA), and immune cells expressing a chimeric antigen receptor (CAR) directed to BCMA (BCMA CAR T cells), wherein the immune cells expressing a chimeric antigen receptor (CAR) directed to BCMA (BCMA CAR T cells) are not the same as the first BCMA-based treatment modality comprising immune cells expressing a chimeric antigen receptor (CAR) directed to BCMA (BCMA CAR T cells). In a more specific embodiment, the patient has not received said second BCMA-based treatment modality prior to administration of said first BCMA-based treatment modality.

In a specific embodiment of any of the above embodiments, the second BCMA-based treatment modality comprises CC99712, GSK2857916 (belantamab mafodotin), CC-93269, AMG 420, JNJ-64007957, AMG 701, PF-06863135, REGN5458, REGN5459, TNB-383B, DF3001, AFM26, CTX-4419, CTX-8573, JCARH125, KITE-585, P-BCMA-101, LCAR-B38M, CT053, anti-CD19/BCMA CAR-T cells (Hrain Biotechnology), or CTX120. In a specific embodiment of any of the above embodiments, the second BCMA-based treatment modality is CC99712, GSK2857916 (belantamab mafodotin), CC-93269, AMG 420, JNJ-64007957, AMG 701, PF-06863135, REGN5458, REGN5459, TNB-383B, DF3001, AFM26, CTX-4419, CTX-8573, JCARH125, KITE-585, P-BCMA-101, LCAR-B38M, CT053, anti-CD19/BCMA CAR-T cells (Hrain Biotechnology), or CTX120. In a specific embodiment of any of the above embodiments, the second BCMA-based treatment modality consists of CC99712, GSK2857916 (belantamab mafodotin), CC-93269, AMG 420, JNJ-64007957, AMG 701, PF-06863135, REGN5458, REGN5459, TNB-383B, DF3001, AFM26, CTX-4419, CTX-8573, JCARH125, KITE-585, P-BCMA-101, LCAR-B38M, CT053, anti-CD19/BCMA CAR-T cells (Hrain Biotechnology), or CTX120. In a specific embodiment of any of the above embodiments, the second BCMA-based treatment modality is selected from the group consisting of CC99712, GSK2857916 (belantamab mafodotin), CC-93269, AMG 420, JNJ-64007957, AMG 701, PF-06863135, REGN5458, REGN5459, TNB-383B, DF3001, AFM26, CTX-4419, CTX-8573, JCARH125, KITE-585, P-BCMA-101, LCAR-B38M, CT053, anti-CD19/BCMA CAR-T cells (Hrain Biotechnology), and CTX120.

In a specific embodiment of any of the above embodiments, the second BCMA-based treatment modality comprises a BCMA-Antibody-Drug Conjugate (ADC). In a specific embodiment of any of the above embodiments, the second BCMA-based treatment modality is a BCMA-Antibody-Drug Conjugate (ADC). In a specific embodiment of any of the above embodiments, the second BCMA-based treatment modality consists of a BCMA-Antibody-Drug Conjugate (ADC). In certain embodiments, the BCMA-Antibody-Drug Conjugate (ADC) comprises CC99712 or GSK2857916 (belantamab mafodotin). In certain embodiments, the BCMA-Antibody-Drug Conjugate (ADC) is CC99712 or GSK2857916 (belantamab mafodotin). In certain embodiments, the BCMA-Antibody-Drug Conjugate (ADC) consists of CC99712 or GSK2857916 (belantamab mafodotin). In certain embodiments, the BCMA-Antibody-Drug Conjugate (ADC) may be administered immediately after administration of the first BCMA-based treatment modality. In certain embodiments, the BCMA-Antibody-Drug Conjugate (ADC) may be administered 1 week, 2 weeks, 3 weeks, or 4 weeks after administration of the first BCMA-based treatment modality. In certain embodiments, the BCMA-Antibody-Drug Conjugate (ADC) may be administered 1 month, 2 months, 3 months, 4 months, 5 months, 6 months, 7 months, 8 months, 9 months, 10 months, 11 months, or 12 months after administration of the first BCMA-based treatment modality. In a certain embodiment, the BCMA-Antibody-Drug Conjugate (ADC) should be initiated upon adequate bone marrow recovery or from 90 days after administration of the first BCMA-based treatment modality, e.g., 90 days after administration of ide-cel, whichever is later.

In a specific embodiment of any of the above embodiments, the second BCMA-based treatment modality comprises a bispecific T-cell engager (BiTE) that targets B-cell maturation antigen (BCMA). In a specific embodiment of any of the above embodiments, the second BCMA-based treatment modality is a bispecific T-cell engager (BiTE) that targets B-cell maturation antigen (BCMA). In a specific embodiment of any of the above embodiments, the second BCMA-based treatment modality consists of a bispecific T-cell engager (BiTE) that targets B-cell maturation antigen (BCMA). In certain embodiments, the bispecific T-cell engager (BiTE) that targets B-cell maturation antigen (BCMA) comprises CC-93269, AMG 420, JNJ-64007957, AMG 701, PF-06863135, REGN5458, REGN5459, or TNB-383B. In certain embodiments, the bispecific T-cell engager (BiTE) that targets B-cell maturation antigen (BCMA) is CC-93269, AMG 420, JNJ-64007957, AMG 701, PF-06863135, REGN5458, REGN5459, or TNB-383B. In certain embodiments, the bispecific T-cell engager (BiTE) that targets B-cell maturation antigen (BCMA) consists of CC-93269, AMG 420, JNJ-64007957, AMG 701, PF-06863135, REGN5458, REGN5459, or TNB-383B. In certain embodiments, the bispecific T-cell engager (BiTE) that targets B-cell maturation antigen (BCMA) is selected from the group consisting of CC-93269, AMG 420, JNJ-64007957, AMG 701, PF-06863135, REGN5458, REGN5459, and TNB-383B. In certain embodiments, the bispecific T-cell engager (BiTE) may be administered immediately after administration of the first BCMA-based treatment modality. In certain embodiments, the bispecific T-cell engager (BiTE) may be administered 1 week, 2 weeks, 3 weeks, or 4 weeks after administration of the first BCMA-based treatment modality. In certain embodiments, the bispecific T-cell engager (BiTE) may be administered 1 month, 2 months, 3 months, 4 months, 5 months, 6 months, 7 months, 8 months, 9 months, 10 months, 11 months, or 12 months after administration of the first BCMA-based treatment modality. In a certain embodiment, the bispecific T-cell engager (BiTE) should be initiated upon adequate bone marrow recovery or from 90 days after administration of the first BCMA-based treatment modality, e.g., 90 days after administration of ide-cel, whichever is later.

In a specific embodiment of any of the above embodiments, the second BCMA-based treatment modality comprises a natural killer (NK) cell engager (NKCE) that targets B-cell maturation antigen (BCMA). In a specific embodiment of any of the above embodiments, the second BCMA-based treatment modality is a natural killer (NK) cell engager (NKCE) that targets B-cell maturation antigen (BCMA). In a specific embodiment of any of the above embodiments, the second BCMA-based treatment modality consists of a natural killer (NK) cell engager (NKCE) that targets B-cell maturation antigen (BCMA). In certain embodiments, the natural killer (NK) cell engager (NKCE) that targets B-cell maturation antigen (BCMA) comprises DF3001, AFM26, CTX-4419, or CTX-8573. In certain embodiments, the natural killer (NK) cell engager (NKCE) that targets B-cell maturation antigen (BCMA) is DF3001, AFM26, CTX-4419, or CTX-8573. In certain embodiments, the natural killer (NK) cell engager (NKCE) that targets B-cell maturation antigen (BCMA) consists of DF3001, AFM26, CTX-4419, or CTX-8573. In certain embodiments, the natural killer (NK) cell engager (NKCE) that targets B-cell maturation antigen (BCMA) is selected from the group consisting of DF3001, AFM26, CTX-4419, and CTX-8573. In certain embodiments, the natural killer (NK) cell engager (NKCE) that targets B-cell maturation antigen (BCMA) may be administered immediately after administration of the first BCMA-based treatment modality. In certain embodiments, the natural killer (NK) cell engager (NKCE) that targets B-cell maturation antigen (BCMA) may be administered 1 week, 2 weeks, 3 weeks, or 4 weeks after administration of the first BCMA-based treatment modality. In certain embodiments, the natural killer (NK) cell engager (NKCE) that targets B-cell maturation antigen (BCMA) may be administered 1 month, 2 months, 3 months, 4 months, 5 months, 6 months, 7 months, 8 months, 9 months, 10 months, 11 months, or 12 months after administration of the first BCMA-based treatment modality. In a certain embodiment, the natural killer (NK) cell engager (NKCE) that targets B-cell maturation antigen (BCMA) should be initiated upon adequate bone marrow recovery or 90 days after administration of the first BCMA-based treatment modality, e.g., 90 days after administration of ide-cel, whichever is later.

In a specific embodiment of any of the above embodiments, the second BCMA-based treatment modality comprises immune cells expressing a chimeric antigen receptor (CAR) directed to BCMA (BCMA CAR T cells). In a specific embodiment of any of the above embodiments, the second BCMA-based treatment modality comprises immune cells expressing a chimeric antigen receptor (CAR) directed to BCMA (BCMA CAR T cells), wherein the immune cells expressing a chimeric antigen receptor (CAR) directed to BCMA (BCMA CAR T cells) are not the same as the first BCMA-based treatment modality comprising immune cells expressing a chimeric antigen receptor (CAR) directed to BCMA (BCMA CAR T cells). In a specific embodiment of any of the above embodiments, the second BCMA-based treatment modality is immune cells expressing a chimeric antigen receptor (CAR) directed to BCMA (BCMA CAR T cells). In a specific embodiment of any of the above embodiments, the second BCMA-based treatment modality is immune cells expressing a chimeric antigen receptor (CAR) directed to BCMA (BCMA CAR T cells), wherein the immune cells expressing a chimeric antigen receptor (CAR) directed to BCMA (BCMA CAR T cells) are not the same as the first BCMA-based treatment modality comprising immune cells expressing a chimeric antigen receptor (CAR) directed to BCMA (BCMA CAR T cells). In a specific embodiment of any of the above embodiments, the second BCMA-based treatment modality consists of immune cells expressing a chimeric antigen receptor (CAR) directed to BCMA (BCMA CAR T cells). In a specific embodiment of any of the above embodiments, the second BCMA-based treatment modality consists of immune cells expressing a chimeric antigen receptor (CAR) directed to BCMA (BCMA CAR T cells), wherein the immune cells expressing a chimeric antigen receptor (CAR) directed to BCMA (BCMA CAR T cells) are not the same as the first BCMA-based treatment modality comprising immune cells expressing a chimeric antigen receptor (CAR) directed to BCMA (BCMA CAR T cells). In certain embodiments, the immune cells expressing a chimeric antigen receptor (CAR) directed to BCMA (BCMA CAR T cells) comprise JCARH125, KITE-585, P-BCMA-101, LCAR-B38M, CT053, anti-CD19/BCMA CAR-T cells (Hrain Biotechnology), and CTX120. In certain embodiments, the immune cells expressing a chimeric antigen receptor (CAR) directed to BCMA (BCMA CAR T cells) may be administered immediately after administration of the first BCMA-based treatment modality. In certain embodiments, the immune cells expressing a chimeric antigen receptor (CAR) directed to BCMA (BCMA CAR T cells) may be administered 1 week, 2 weeks, 3 weeks, or 4 weeks after administration of the first BCMA-based treatment modality. In certain embodiments, the immune cells expressing a chimeric antigen receptor (CAR) directed to BCMA (BCMA CAR T cells) may be administered 1 month, 2 months, 3 months, 4 months, 5 months, 6 months, 7 months, 8 months, 9 months, 10 months, 11 months, or 12 months after administration of the first BCMA-based treatment modality. In a certain embodiment, the immune cells expressing a chimeric antigen receptor (CAR) directed to BCMA (BCMA CAR T cells) should be initiated upon adequate bone marrow recovery or 90 days after administration of the first BCMA-based treatment modality, e.g., 90 days after administration of ide-cel, whichever is later.

In certain embodiments, the immune cells expressing a chimeric antigen receptor (CAR) directed to BCMA (BCMA CAR T cells), wherein the immune cells expressing a chimeric antigen receptor (CAR) directed to BCMA (BCMA CAR T cells) are not the same as the first BCMA-based treatment modality comprising immune cells expressing a chimeric antigen receptor (CAR) directed to BCMA (BCMA CAR T cells), may be administered immediately after administration of the first BCMA-based treatment modality. In certain embodiments, the immune cells expressing a chimeric antigen receptor (CAR) directed to BCMA (BCMA CAR T cells), wherein the immune cells expressing a chimeric antigen receptor (CAR) directed to BCMA (BCMA CAR T cells) are not the same as the first BCMA-based treatment modality comprising immune cells expressing a chimeric antigen receptor (CAR) directed to BCMA (BCMA CAR T cells), may be administered 1 week, 2 weeks, 3 weeks, or 4 weeks after administration of the first BCMA-based treatment modality. In certain embodiments, the immune cells expressing a chimeric antigen receptor (CAR) directed to BCMA (BCMA CAR T cells), wherein the immune cells expressing a chimeric antigen receptor (CAR) directed to BCMA (BCMA CAR T cells) are not the same as the first BCMA-based treatment modality comprising immune cells expressing a chimeric antigen receptor (CAR) directed to BCMA (BCMA CAR T cells), may be administered 1 month, 2 months, 3 months, 4 months, 5 months, 6 months, 7 months, 8 months, 9 months, 10 months, 11 months, or 12 months after administration of the first BCMA-based treatment modality. In a certain embodiment, the immune cells expressing a chimeric antigen receptor (CAR) directed to BCMA (BCMA CAR T cells), wherein the immune cells expressing a chimeric antigen receptor (CAR) directed to BCMA (BCMA CAR T cells) are not the same as the first BCMA-based treatment modality comprising immune cells expressing a chimeric antigen receptor (CAR) directed to BCMA (BCMA CAR T cells), should be initiated upon adequate bone marrow recovery or 90 days after administration of the first BCMA-based treatment modality, e.g., 90 days after administration of ide-cel, whichever is later.

In specific embodiments of any of the above aspects or embodiments, the immune cells in the first BCMA-based treatment modality comprising immune cells expressing a chimeric antigen receptor (CAR) directed to BCMA (BCMA CAR T cells) are idecabtagene vicleucel cells.

In specific embodiments of any of the above aspects or embodiments, the second BCMA-based treatment modality does not comprise idecabtagene vicleucel cells. In specific embodiments of any of the above aspects or embodiments, the second BCMA-based treatment modality is not idecabtagene vicleucel cells.

In specific embodiments of any of the above aspects or embodiments, the immune cells are T cells, e.g., CD4+ T cells, CD8+ T cells or cytotoxic T lymphocytes (CTLs), T killer cells, or natural killer (NK) cells. In another specific embodiment specific embodiment, the immune cells are administered in a dosage of from 150×10⁶ cells to 450×10⁶ cells.

In a specific embodiment of any of the above embodiments, the non-CAR T cell therapy comprises a proteasome inhibitor, lenalidomide, pomalidomide, thalidomide, bortezomib, dexamethasone, cyclophosphamide, doxorubicin, carfilzomib, ixazomib, cisplatin, doxorubicin, etoposide, an anti-CD38 antibody panobinostat, or elotuzumab. In more specific embodiments, before said administering said subject has received one or more lines of prior therapy comprising: daratumumab, pomalidomide, and dexamethasone (DPd); daratumumab, bortezomib, and dexamethasone (DVd); ixazomib, lenalidomide, and dexamethasone (IRd); daratumumab, lenalidomide and dexamethasone; bortezomib, lenalidomide and dexamethasone (RVd); bortezomib, cyclophosphamide and dexamethasone (BCd); bortezomib, doxorubicin and dexamethasone; carfilzomib, lenalidomide and dexamethasone (CRd); bortezomib and dexamethasone; bortezomib, thalidomide and dexamethasone; lenalidomide and dexamethasone; dexamethasone, thalidomide, cisplatin, doxorubicin, cyclophosphamide, etoposide and bortezomib (VTD-PACE); lenalidomide and low-dose dexamethasone; bortezomib, cyclophosphamide and dexamethasone; carfilzomib and dexamethasone; lenalidomide alone; bortezomib alone; daratumumab alone; elotuzumab, lenalidomide, and dexamethasone; elotuzumab, lenalidomide and dexamethasone; bendamustine, bortezomib and dexamethasone; bendamustine, lenalidomide, and dexamethasone; pomalidomide and dexamethasone; pomalidomide, bortezomib and dexamethasone; pomalidomide, carfilzomib and dexamethasone; bortezomib and liposomal doxorubicin; cyclophosphamide, lenalidomide, and dexamethasone; elotuzumab, bortezomib and dexamethasone; ixazomib and dexamethasone; panobinostat, bortezomib and dexamethasone; panobinostat and carfilzomib; or pomalidomide, cyclophosphamide and dexamethasone; or any one of the other therapeutic agents listed in Section 5.9, below. In a more specific embodiment, the patient has not received said non-CAR T cell therapy prior to administration of CAR T cells.

In a specific embodiment of any of the above embodiments, the non-CAR T cell therapy comprises lenalidomide. In certain embodiments, the lenalidomide is administered to a subject as a maintenance therapy after administration of compositions comprising CAR-expressing immune effector cells. In certain embodiments, the lenalidomide may be administered immediately after administration of the compositions comprising CAR-expressing immune effector cells. In certain embodiments, the lenalidomide may be administered 1 week, 2 weeks, 3 weeks, or 4 weeks after administration of the compositions comprising CAR-expressing immune effector cells. In certain embodiments, the lenalidomide may be administered 1 month, 2 months, 3 months, 4 months, 5 months, 6 months, 7 months, 8 months, 9 months, 10 months, 11 months, or 12 months after administration of the compositions comprising CAR-expressing immune effector cells. In certain embodiments, the lenalidomide may be administered at a dosage of about 2.5 mg, 5 mg, 10 mg, 15 mg, 20 mg, or 25 mg. In certain embodiments, the lenalidomide may be administered at a dosage of about 2.5 mg, 5 mg, 10 mg, 15 mg, 20 mg, or 25 mg once daily. In certain embodiments, the lenalidomide may be administered at a dosage of about 25 mg once daily orally on Days 1-21 of repeated 28-day cycles. In certain embodiments, the lenalidomide may be administered at a dosage of about 25 mg once daily orally on Days 1-21 of repeated 28-day cycles to a subject for treating Multiple Myeloma (MM). In certain embodiments, the lenalidomide may be administered at a dosage of about 10 mg once daily continuously on Days 1-28 of repeated 28-day cycles. In certain embodiments, the lenalidomide may be administered at a dosage of about 2.5 mg once daily. In certain embodiments, the lenalidomide may be administered at a dosage of about 5 mg once daily. In certain embodiments, the lenalidomide may be administered at a dosage of about 10 mg once daily. In certain embodiments, the lenalidomide may be administered at a dosage of about 15 mg every other day. In certain embodiments, the lenalidomide may be administered at a dosage of about 25 mg once daily orally on Days 1-21 of repeated 28-day cycles. In certain embodiments, the lenalidomide may be administered at a dosage of about 20 mg once daily orally on Days 1-21 of repeated 28-day cycles for up to 12 cycles. In a certain embodiment, lenalidomide maintenance therapy is recommended for all patients. In a certain embodiment, lenalidomide maintenance therapy should be initiated upon adequate bone marrow recovery or from 90-day post-ide-cel infusion, whichever is later.

In a specific embodiment of any of the above embodiments, the non-CAR T cell therapy comprises pomalidomide. In certain embodiments, the pomalidomide is administered to a subject as a maintenance therapy after administration of compositions comprising CAR-expressing immune effector cells. In certain embodiments, the pomalidomide may be administered immediately after administration of the compositions comprising CAR-expressing immune effector cells. In certain embodiments, the pomalidomide may be administered 1 week, 2 weeks, 3 weeks, or 4 weeks after administration of the compositions comprising CAR-expressing immune effector cells. In certain embodiments, the pomalidomide may be administered 1 month, 2 months, 3 months, 4 months, 5 months, 6 months, 7 months, 8 months, 9 months, 10 months, 11 months, or 12 months after administration of the compositions comprising CAR-expressing immune effector cells. In certain embodiments, the pomalidomide may be administered at a dosage of about 1 mg, 2 mg, 3 mg, or 4 mg. In certain embodiments, the pomalidomide may be administered at a dosage of about 1 mg, 2 mg, 3 mg, or 4 mg once daily. In certain embodiments, the pomalidomide may be administered at a dosage of about 4 mg per day taken orally on days 1-21 of repeated 28-day cycles until disease progression. In certain embodiments, the pomalidomide may be administered at a dosage of about 4 mg per day taken orally on days 1-21 of repeated 28-day cycles until disease progression to a subject for treating Multiple Myeloma (MM). In a certain embodiment, pomalidomide maintenance therapy is recommended for all patients. In a certain embodiment, pomalidomide maintenance therapy should be initiated upon adequate bone marrow recovery or from 90-day post-ide-cel infusion, whichever is later.

In a specific embodiment of any of the above embodiments, the non-CAR T cell therapy comprises CC-220 (iberdomide; see, e.g., Bjorkland, C. C. et al., 2019, Leukemia, doi: 10.1038/s41375-019-0620-8; U.S. Pat. No. 9,828,361). In certain embodiments, the CC-220 is administered to a subject as a maintenance therapy after administration of compositions comprising CAR-expressing immune effector cells. In certain embodiments, the CC-220 may be administered immediately after administration of the compositions comprising CAR-expressing immune effector cells. In certain embodiments, the CC-220 may be administered 1 week, 2 weeks, 3 weeks, or 4 weeks after administration of the compositions comprising CAR-expressing immune effector cells. In certain embodiments, the CC-220 may be administered 1 month, 2 months, 3 months, 4 months, 5 months, 6 months, 7 months, 8 months, 9 months, 10 months, 11 months, or 12 months after administration of the compositions comprising CAR-expressing immune effector cells. In certain embodiments, the CC-220 may be administered at a dosage of about 0.15 mg, 0.3 mg, 0.45 mg, 0.6 mg, 0.75 mg, 0.9 mg, 1.0 mg, 1.1 mg, or 1.2 mg. In certain embodiments, the CC-220 may be administered orally. In certain embodiments, the CC-220 may be administered orally at a dosage of about 0.15 mg, 0.3 mg, 0.45 mg, 0.6 mg, 0.75 mg, 0.9 mg, 1.0 mg, 1.1 mg, or 1.2 mg daily for 21 days of a 28-day cycle, e.g., daily on days 1-21 of a 28-day cycle, with the 28-day cycles repeated as needed. In certain embodiments, the CC-220 may be administered to a subject for treating Multiple Myeloma (MM). In a certain embodiment, CC-220 maintenance therapy is recommended for all patients. In a certain embodiment, the CC-220 maintenance therapy should be initiated upon adequate bone marrow recovery or from 90-day post-ide-cel infusion, whichever is later.

In a specific embodiment of any of the above embodiments, the non-CAR T cell therapy comprises CC-220 (iberdomide) and dexamethasone. In certain embodiments, the CC-220 and dexamethasone are administered to a subject as a maintenance therapy after administration of compositions comprising CAR-expressing immune effector cells. In certain embodiments, the CC-220 and dexamethasone may be administered immediately after administration of the compositions comprising CAR-expressing immune effector cells. In certain embodiments, the CC-220 may be administered immediately after administration of the compositions comprising CAR-expressing immune effector cells. In certain embodiments, the dexamethasone may be administered immediately after administration of the compositions comprising CAR-expressing immune effector cells. In certain embodiments, the CC-220 and dexamethasone may be administered 1 week, 2 weeks, 3 weeks, or 4 weeks after administration of the compositions comprising CAR-expressing immune effector cells. In certain embodiments, the CC-220 may be administered 1 week, 2 weeks, 3 weeks, or 4 weeks after administration of the compositions comprising CAR-expressing immune effector cells. In certain embodiments, the dexamethasone may be administered 1 week, 2 weeks, 3 weeks, or 4 weeks after administration of the compositions comprising CAR-expressing immune effector cells. In certain embodiments, the CC-220 and dexamethasone may be administered 1 month, 2 months, 3 months, 4 months, 5 months, 6 months, 7 months, 8 months, 9 months, 10 months, 11 months, or 12 months after administration of the compositions comprising CAR-expressing immune effector cells. In certain embodiments, the CC-220 may be administered 1 month, 2 months, 3 months, 4 months, 5 months, 6 months, 7 months, 8 months, 9 months, 10 months, 11 months, or 12 months after administration of the compositions comprising CAR-expressing immune effector cells. In certain embodiments, the dexamethasone may be administered 1 month, 2 months, 3 months, 4 months, 5 months, 6 months, 7 months, 8 months, 9 months, 10 months, 11 months, or 12 months after administration of the compositions comprising CAR-expressing immune effector cells. In certain embodiments, the CC-220 may be administered at a dosage of about 0.15 mg, 0.3 mg, 0.45 mg, 0.6 mg, 0.75 mg, 0.9 mg, 1.0 mg, 1.1 mg, or 1.2 mg. In certain embodiments, the dexamethasone may be administered at a dosage of about 20 mg, 25 mg., 30 mg, 35 mg, 40 mg, 45 mg, 50 mg, 55 mg, or 60 mg. In certain embodiments, the dexamethasone may be administered at a dosage of about 40 mg. In certain embodiments, the CC-220 may be administered orally. In certain embodiments, the CC-220 may be administered orally at a dosage of about 15 mg, 0.3 mg, 0.45 mg, 0.6 mg, 0.75 mg, 0.9 mg, 1.0 mg, 1.1 mg, or 1.2 mg daily for 21 days of a 28-day cycle, e.g., daily on days 1-21 of a 28-day cycle, with the 28-day cycles repeated as needed. In certain embodiments, the dexamethasone may be administered orally. In certain embodiments, the dexamethasone may be administered at a dose of about 20-60 mgs. In certain embodiments, the dexamethasone may be administered orally at a dosage of about 20 mg, 25 mg, 30 mg, 35 mg, 40 mg, 45 mg, 50 mg, 55 mg, or 60 mg on days 1, 8, 15, and 22 of a 28-day cycle, with the 28-day cycles repeated as needed. In certain embodiments, the CC-220 may be administered orally at a dosage of about 15 mg, 0.3 mg, 0.45 mg, 0.6 mg, 0.75 mg, 0.9 mg, 1.0 mg, 1.1 mg, or 1.2 mg daily for 21 days of a 28-day cycle, e.g., daily on days 1-21 of a 28-day cycle, with the 28-day cycles repeated as needed, and the dexamethasone may be administered orally at a dosage of about 20 mg, 25 mg., 30 mg, 35 mg, 40 mg, 45 mg, 50 mg, 55 mg, or 60 mg on days 1, 8, 15, and 22 of a 28-day cycle, with the 28-day cycles repeated as needed. In certain embodiments, the CC-220 and dexamethasone may be administered to a subject for treating Multiple Myeloma (MM). In a certain embodiment, CC-220 and dexamethasone maintenance therapy is recommended for all patients. In a certain embodiment, the CC-220 and dexamethasone maintenance therapy should be initiated upon adequate bone marrow recovery or from 90-day post-ide-cel infusion, whichever is later.

In another specific embodiment of any of the above aspects or embodiments, before said administering said subject has received three or more lines of prior therapy, or one or more lines of prior therapy. In more specific embodiments, said lines of prior therapy comprise a proteasome inhibitor, lenalidomide, pomalidomide, thalidomide, bortezomib, dexamethasone, cyclophosphamide, doxorubicin, carfilzomib, ixazomib, cisplatin, doxorubicin, etoposide, an anti-CD38 antibody panobinostat, or elotuzumab. In more specific embodiments, before said administering said subject has received one or more lines of prior therapy comprising: daratumumab, pomalidomide, and dexamethasone (DPd); daratumumab, bortezomib, and dexamethasone (DVd); ixazomib, lenalidomide, and dexamethasone (IRd); daratumumab, lenalidomide and dexamethasone; bortezomib, lenalidomide and dexamethasone (RVd); bortezomib, cyclophosphamide and dexamethasone (BCd); bortezomib, doxorubicin and dexamethasone; carfilzomib, lenalidomide and dexamethasone (CRd); bortezomib and dexamethasone; bortezomib, thalidomide and dexamethasone; lenalidomide and dexamethasone; dexamethasone, thalidomide, cisplatin, doxorubicin, cyclophosphamide, etoposide and bortezomib (VTD-PACE); lenalidomide and low-dose dexamethasone; bortezomib, cyclophosphamide and dexamethasone; carfilzomib and dexamethasone; lenalidomide alone; bortezomib alone; daratumumab alone; elotuzumab, lenalidomide, and dexamethasone; elotuzumab, lenalidomide and dexamethasone; bendamustine, bortezomib and dexamethasone; bendamustine, lenalidomide, and dexamethasone; pomalidomide and dexamethasone; pomalidomide, bortezomib and dexamethasone; pomalidomide, carfilzomib and dexamethasone; bortezomib and liposomal doxorubicin; cyclophosphamide, lenalidomide, and dexamethasone; elotuzumab, bortezomib and dexamethasone; ixazomib and dexamethasone; panobinostat, bortezomib and dexamethasone; panobinostat and carfilzomib; or pomalidomide, cyclophosphamide and dexamethasone. In various more specific embodiments, said subject has received two, three, four, five, six, seven or more of said lines of prior therapy; no more than three of said lines of prior therapy; no more than two of said lines of prior therapy; or no more than one of said lines of prior therapy.

In specific embodiments of any of the above aspects or embodiments, the immune cells are administered at a dose ranging from 150×10⁶ cells to 450×10⁶ cells, 300×10⁶ cells to 600×10⁶ cells, 350×10⁶ cells to 600×10⁶ cells, 350×10⁶ cells to 550×10⁶ cells, 400×10⁶ cells to 600×10⁶ cells, 150×10⁶ cells to 300×10⁶ cells, or 400×10⁶ cells to 500×10⁶ cells. In some embodiments, the immune cells are administered at a dose of about 150×10⁶ cells, about 200×10⁶ cells, about 250×10⁶ cells, about 300×10⁶ cells, about 350×10⁶ cells, about 400×10⁶ cells, about 450×10⁶ cells, about 500×10⁶ cells, or about 550×10⁶ cells. In one embodiment, the immune cells are administered at a dose of about 450×10⁶ cells. In some embodiments, the subject is administered one infusion of the immune cells expressing a chimeric antigen receptor (CAR). In some embodiments, the administration of the immune cells expressing a CAR is repeated (e.g., a second dose of immune cells is administered to the subject). In some embodiments, the subject is administered one infusion of the immune cells expressing a chimeric antigen receptor (CAR) directed to B Cell Maturation Antigen (BCMA). In some embodiments, the administration of the immune cells expressing a CAR directed to BCMA is repeated (e.g., a second dose of immune cells is administered to the subject).

In specific embodiments of any of the embodiments described herein, the immune cells expressing a CAR are administered in a dosage of from about 150×10⁶ cells to about 300×10⁶ cells. In specific embodiments of any of the embodiments described herein, the immune cells expressing a CAR are administered in a dosage of from about 350×10⁶ cells to about 550×10⁶ cells. In specific embodiments of any of the embodiments described herein, the immune cells expressing a CAR are administered in a dosage of from about 400×10⁶ cells to about 500×10⁶ cells. In specific embodiments of any of the embodiments described herein, the immune cells expressing a CAR are administered in a dosage of from about 150×10⁶ cells to about 250×10⁶ cells. In specific embodiments of any of the embodiments described herein, the immune cells expressing a CAR are administered in a dosage of from about 300×10⁶ cells to about 500×10⁶ cells. In specific embodiments of any of the embodiments described herein, the immune cells expressing a CAR are administered in a dosage of from about 350×10⁶ cells to about 450×10⁶ cells. In specific embodiments of any of the embodiments described herein, the immune cells expressing a CAR are administered in a dosage of from about 300×10⁶ cells to about 450×10⁶ cells. In specific embodiments of any of the embodiments described herein, the immune cells expressing a CAR are administered in a dosage of from about 250×10⁶ cells to about 450×10⁶ cells. In specific embodiments of any of the embodiments described herein, the immune cells expressing a CAR are administered in a dosage of from about 300×10⁶ cells to about 600×10⁶ cells. In specific embodiments of any of the embodiments described herein, the immune cells expressing a CAR are administered in a dosage of from about 250×10⁶ cells to about 500×10⁶ cells. In specific embodiments of any of the embodiments described herein, the immune cells expressing a CAR are administered in a dosage of from about 350×10⁶ cells to about 500×10⁶ cells. In specific embodiments of any of the embodiments described herein, the immune cells expressing a CAR are administered in a dosage of from about 400×10⁶ cells to about 600×10⁶ cells. In specific embodiments of any of the embodiments described herein, the immune cells expressing a CAR are administered in a dosage of from about 400×10⁶ cells to about 450×10⁶ cells. In specific embodiments of any of the embodiments described herein, the immune cells expressing a CAR are administered in a dosage of from about 200×10⁶ cells to about 400×10⁶ cells. In specific embodiments of any of the embodiments described herein, the immune cells expressing a CAR are administered in a dosage of from about 200×10⁶ cells to about 350×10⁶ cells. In specific embodiments of any of the embodiments described herein, the immune cells expressing a CAR are administered in a dosage of from about 200×10⁶ cells to about 300×10⁶ cells. In specific embodiments of any of the embodiments described herein, the immune cells expressing a CAR are administered in a dosage of from about 450×10⁶ cells to about 500×10⁶ cells. In specific embodiments of any of the embodiments described herein, the immune cells expressing a CAR are administered in a dosage of from about 250×10⁶ cells to about 400×10⁶ cells. In specific embodiments of any of the embodiments described herein, the immune cells expressing a CAR are administered in a dosage of from about 250×10⁶ cells to about 350×10⁶ cells. In specific embodiments of any of the embodiments described herein, the immune cells expressing a CAR are administered in a dosage of about 450×10⁶ cells. In specific embodiments of any of the embodiments described herein, the immune cells are T cells (e.g., autologous T cells). In specific embodiments of any of the embodiments described herein, the subjects being treated undergo a leukapharesis procedure to collect autologous immune cells for the manufacture of the immune cells expressing a CAR prior to their administration to the subject. In specific embodiments of any of the embodiments described herein, the immune cells (e.g., T cells) are administered by an intravenous infusion.

In specific embodiments of any of the embodiments described herein, the immune cells expressing a CAR directed to BCMA are administered in a dosage of from about 150×10⁶ cells to about 300×10⁶ cells. In specific embodiments of any of the embodiments described herein, the immune cells expressing a CAR directed to BCMA are administered in a dosage of from about 350×10⁶ cells to about 550×10⁶ cells. In specific embodiments of any of the embodiments described herein, the immune cells expressing a CAR directed to BCMA are administered in a dosage of from about 400×10⁶ cells to about 500×10⁶ cells. In specific embodiments of any of the embodiments described herein, the immune cells expressing a CAR directed to BCMA are administered in a dosage of from about 150×10⁶ cells to about 250×10⁶ cells. In specific embodiments of any of the embodiments described herein, the immune cells expressing a CAR directed to BCMA are administered in a dosage of from about 300×10⁶ cells to about 500×10⁶ cells. In specific embodiments of any of the embodiments described herein, the immune cells expressing a CAR directed to BCMA are administered in a dosage of from about 350×10⁶ cells to about 450×10⁶ cells. In specific embodiments of any of the embodiments described herein, the immune cells expressing a CAR directed to BCMA are administered in a dosage of from about 300×10⁶ cells to about 450×10⁶ cells. In specific embodiments of any of the embodiments described herein, the immune cells expressing a CAR directed to BCMA are administered in a dosage of from about 250×10⁶ cells to about 450×10⁶ cells. In specific embodiments of any of the embodiments described herein, the immune cells expressing a CAR directed to BCMA are administered in a dosage of from about 300×10⁶ cells to about 600×10⁶ cells. In specific embodiments of any of the embodiments described herein, the immune cells expressing a CAR directed to BCMA are administered in a dosage of from about 250×10⁶ cells to about 500×10⁶ cells. In specific embodiments of any of the embodiments described herein, the immune cells expressing a CAR directed to BCMA are administered in a dosage of from about 350×10⁶ cells to about 500×10⁶ cells. In specific embodiments of any of the embodiments described herein, the immune cells expressing a CAR directed to BCMA are administered in a dosage of from about 400×10⁶ cells to about 600×10⁶ cells. In specific embodiments of any of the embodiments described herein, the immune cells expressing a CAR directed to BCMA are administered in a dosage of from about 400×10⁶ cells to about 450×10⁶ cells. In specific embodiments of any of the embodiments described herein, the immune cells expressing a CAR directed to BCMA are administered in a dosage of from about 200×10⁶ cells to about 400×10⁶ cells. In specific embodiments of any of the embodiments described herein, the immune cells expressing a CAR directed to BCMA are administered in a dosage of from about 200×10⁶ cells to about 350×10⁶ cells. In specific embodiments of any of the embodiments described herein, the immune cells expressing a CAR directed to BCMA are administered in a dosage of from about 200×10⁶ cells to about 300×10⁶ cells. In specific embodiments of any of the embodiments described herein, the immune cells expressing a CAR directed to BCMA are administered in a dosage of from about 450×10⁶ cells to about 500×10⁶ cells. In specific embodiments of any of the embodiments described herein, the immune cells expressing a CAR directed to BCMA are administered in a dosage of from about 250×10⁶ cells to about 400×10⁶ cells. In specific embodiments of any of the embodiments described herein, the immune cells expressing a CAR directed to BCMA are administered in a dosage of from about 250×10⁶ cells to about 350×10⁶ cells. In specific embodiments of any of the embodiments described herein, the immune cells expressing a CAR directed to BCMA are administered in a dosage of about 450×10⁶ cells. In specific embodiments of any of the embodiments described herein, the immune cells are T cells (e.g., autologous T cells). In specific embodiments of any of the embodiments described herein, the subjects being treated undergo a leukapharesis procedure to collect autologous immune cells for the manufacture of the immune cells expressing a CAR directed to BCMA prior to their administration to the subject. In specific embodiments of any of the embodiments described herein, the immune cells (e.g., T cells) are administered by an intravenous infusion.

In specific embodiments of any of the aspects or embodiments disclosed herein, before administration of immune cells expressing a CAR, the subject being treated is administered a lymphodepleting (LD) chemotherapy. In specific embodiments, LD chemotherapy comprises fludarabine and/or cyclophosphamide. In specific embodiments, LD chemotherapy comprises fludarabine (e.g., about 30 mg/m² for intravenous administration) and cyclophosphamide (e.g., about 300 mg/m² for intravenous administration) for a duration of 1, 2, 3, 4, 5, 6, or 7 days (e.g., 3 days). In other specific embodiments, LD chemotherapy comprises any of the chemotherapeutic agents described in Section 5.9. In specific embodiments, the subject is administered immune cells expressing a chimeric antigen receptor (CAR) 1, 2, 3, 4, 5, 6, or 7 days after the administration of the LD chemotherapy (e.g., 2 or 3 days after the administration of the LD chemotherapy). In specific embodiments, the subject has not received any therapy prior to the initiation of the LD chemotherapy for at least or more than 1 week, at least or more than 2 weeks (at least or more than 14 days), at least or more than 3 weeks, at least or more than 4 weeks, at least or more than 5 weeks, or at least or more than 6 weeks. In specific embodiments of any of the embodiments disclosed herein, before administration of immune cells expressing a chimeric antigen receptor (CAR), the subject being treated has received only a single prior treatment regimen.

In specific embodiments of any of the aspects or embodiments disclosed herein, before administration of immune cells expressing a CAR directed to BCMA, the subject being treated is administered a lymphodepleting (LD) chemotherapy. In specific embodiments, LD chemotherapy comprises fludarabine and/or cyclophosphamide. In specific embodiments, LD chemotherapy comprises fludarabine (e.g., about 30 mg/m² for intravenous administration) and cyclophosphamide (e.g., about 300 mg/m² for intravenous administration) for a duration of 1, 2, 3, 4, 5, 6, or 7 days (e.g., 3 days). In other specific embodiments, LD chemotherapy comprises any of the chemotherapeutic agents described in Section 5.9. In specific embodiments, the subject is administered immune cells expressing a chimeric antigen receptor (CAR) directed to B Cell Maturation Antigen (BCMA) 1, 2, 3, 4, 5, 6, or 7 days after the administration of the LD chemotherapy (e.g., 2 or 3 days after the administration of the LD chemotherapy). In specific embodiments, the subject has not received any therapy prior to the initiation of the LD chemotherapy for at least or more than 1 week, at least or more than 2 weeks (at least or more than 14 days), at least or more than 3 weeks, at least or more than 4 weeks, at least or more than 5 weeks, or at least or more than 6 weeks. In specific embodiments of any of the embodiments disclosed herein, before administration of immune cells expressing a chimeric antigen receptor (CAR) directed to B Cell Maturation Antigen (BCMA), the subject being treated has received only a single prior treatment regimen.

For any of the above embodiments, the subject undergoes apheresis to collect and isolate said immune cells, e.g., T cells. In a specific embodiment of any of the above embodiments, said subject exhibits at the time of said apheresis: M-protein (serum protein electrophoresis [sPEP] or urine protein electrophoresis [uPEP]): sPEP ≥0.5 g/dL or uPEP ≥200 mg/24 hours; light chain multiple myeloma without measurable disease in the serum or urine, with serum immunoglobulin free light chain ≥10 mg/dL and abnormal serum immunoglobulin kappa lambda free light chain ratio; and/or Eastern Cooperative Oncology Group (ECOG) performance status ≤1. In a more specific embodiment, said subject at the time of apheresis additionally: has received at least three of said lines of prior treatment, including prior treatment with a proteasome inhibitor, an immunomodulatory agent (lenalidomide or pomalidomide) and an anti-CD38 antibody; has undergone at least 2 consecutive cycles of treatment for each of said at least three lines of prior treatment, unless progressive disease was the best response to a line of treatment; has evidence of progressive disease on or within 60 days of the most recent line of prior treatment; and/or has achieved a response (minimal response or better) to at least one of said prior lines of treatment. In a specific embodiment of any of the above embodiments, said subject exhibits at the time of said administration: M-protein (serum protein electrophoresis [sPEP] or urine protein electrophoresis [uPEP]): sPEP ≥0.5 g/dL or uPEP ≥200 mg/24 hours; light chain multiple myeloma without measurable disease in the serum or urine, with serum immunoglobulin free light chain ≥10 mg/dL and abnormal serum immunoglobulin kappa lambda free light chain ratio; and/or Eastern Cooperative Oncology Group (ECOG) performance status ≤1. In another more specific embodiment, said subject additionally: has received only one prior anti-myeloma treatment regimen; has the following high risk factors: R-ISS stage III, and early relapse, defined as (i) if the subject has undergone induction plus a stem cell transplant, progressive disease (PD) less than 12 months since date of first transplant; or (ii) if the subject has received only induction, PD<12 months since date of last treatment regimen which must contain at minimum, a proteasome inhibitor, an immunomodulatory agent and dexamethasone.

In a specific embodiment of any of any of the above aspects or embodiments, said CAR comprises an antibody or antibody fragment that targets an antigen of interest. The antigen of interest can be any antigen of interest, e.g., can be an antigen on a tumor cell. The tumor cell may be, e.g., a cell in a solid tumor, or a cell of a blood cancer. The antigen can be any antigen that is expressed on a cell of any tumor or cancer type, e.g., cells of a lymphoma, a lung cancer, a breast cancer, a prostate cancer, an adrenocortical carcinoma, a thyroid carcinoma, a nasopharyngeal carcinoma, a melanoma, e.g., a malignant melanoma, a skin carcinoma, a colorectal carcinoma, a desmoid tumor, a desmoplastic small round cell tumor, an endocrine tumor, an Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipoma, or the like. In more specific embodiments, said lymphoma can be chronic lymphocytic leukemia (small lymphocytic lymphoma), B-cell prolymphocytic leukemia, lymphoplasmacytic lymphoma, Waldenström macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, a non-Hodgkin lymphoma, or multiple myeloma.

In certain embodiments, the antigen is a tumor-associated antigen (TAA) or a tumor-specific antigen (TSA). In various specific embodiments, without limitation, the tumor-associated antigen or tumor-specific antigen is Her2, prostate stem cell antigen (PSCA), alpha-fetoprotein (AFP), carcinoembryonic antigen (CEA), cancer antigen-125 (CA-125), CA19-9, calretinin, MUC-1, epithelial membrane protein (EMA), epithelial tumor antigen (ETA), tyrosinase, melanoma-associated antigen (MAGE), CD19, CD20, CD34, CD45, CD99, CD117, chromogranin, cytokeratin, desmin, glial fibrillary acidic protein (GFAP), gross cystic disease fluid protein (GCDFP-15), HMB-45 antigen, high molecular weight melanoma-associated antigen (HMW-MAA), protein melan-A (MART-1), myo-D1, muscle-specific actin (MSA), neurofilament, neuron-specific enolase (NSE), placental alkaline phosphatase, synaptophysis, thyroglobulin, thyroid transcription factor-1, the dimeric form of the pyruvate kinase isoenzyme type M2 (tumor M2-PK), an abnormal ras protein, or an abnormal p53 protein.

In certain embodiments, the TAA or TSA is a cancer/testis (CT) antigen, e.g., BAGE, CAGE, CTAGE, FATE, GAGE, HCA661, HOM-TES-85, MAGEA, MAGEB, MAGEC, NA88, NY-ESO-1, NY-SAR-35, OY-TES-1, SPANXB1, SPA17, SSX, SYCP1, or TPTE.

In certain other embodiments, the TAA or TSA is a carbohydrate or ganglioside, e.g., fuc-GM1, GM2 (oncofetal antigen-immunogenic-1; OFA-I-1); GD2 (OFA-I-2), GM3, GD3, and the like.

In certain other embodiments, the TAA or TSA is alpha-actinin-4, Bage-1, BCR-ABL, Bcr-Abl fusion protein, beta-catenin, CA 125, CA 15-3 (CA 27.29\BCAA), CA 195, CA 242, CA-50, CAM43, Casp-8, cdc27, cdk4, cdkn2a, CEA, coa-1, dek-can fusion protein, EBNA, EF2, Epstein Barr virus antigens, ETV6-AML1 fusion protein, HLA-A2, HLA-A11, hsp70-2, KIAAO205, Mart2, Mum-1, 2, and 3, neo-PAP, myosin class I, OS-9, pml-RARα fusion protein, PTPRK, K-ras, N-ras, triosephosphate isomerase, Gage 3,4,5,6,7, GnTV, Herv-K-mel, Lage-1, NA-88, NY-Eso-1/Lage-2, SP17, SSX-2, TRP2-Int2, gp100 (Pmel 17), tyrosinase, TRP-1, TRP-2, MAGE-1, MAGE-3, RAGE, GAGE-1, GAGE-2, p15(58), RAGE, SCP-1, Hom/Mel-40, PRAME, p53, H-Ras, HER-2/neu, E2A-PRL, H4-RET, IGH-IGK, MYL-RAR, human papillomavirus (HPV) antigens E6 and E7, TSP-180, MAGE-4, MAGE-5, MAGE-6, pi85erbB2, pi80erbB-3, c-met, nm-23H1, PSA, TAG-72-4, CA 19-9, CA 72-4, CAM 17.1, NuMa, K-ras, 13-Catenin, Mum-1, p16, TAGE, PSMA, CT7, telomerase, 43-9F, 5T4, 791Tgp72, 13HCG, BCA225, BTAA, CD68\KP1, CO-029, FGF-5, G250, Ga733 (EpCAM), HTgp-175, M344, MA-50, MG7-Ag, MOV18, NB70K, NY-CO-1, RCAS1, SDCCAG16, TA-90, TAAL6, TAG72, TLP, TPS, CD19, CD22, CD27, CD30, CD70, GD2 (ganglioside G2), EGFRvIII (epidermal growth factor variant III), sperm protein 17 (Sp17), mesothelin, PAP (prostatic acid phosphatase), prostein, TARP (T cell receptor gamma alternate reading frame protein), Trp-p8, STEAP1 (six-transmembrane epithelial antigen of the prostate 1), an abnormal ras protein, or an abnormal p53 protein. In another specific embodiment, said tumor-associated antigen or tumor-specific antigen is integrin αvβ (CD61), galactin, K-Ras (V-Ki-ras2 Kirsten rat sarcoma viral oncogene), or Ral-B.

In specific embodiments, the TAA or TSA is CD20, CD123, CLL-1, CD38, CS-1, CD138, ROR1, FAP, MUC1, PSCA, EGFRvIII, EPHA2, or GD2. In further specific embodiments, the TAA or TSA is CD123, CLL-1, CD38, or CS-1. In a specific embodiment, the extracellular domain of the CAR binds CS-1. In a further specific embodiment, the extracellular domain comprises a single-chain version of elotuzumab and/or an antigen-binding fragment of elotuzumab. In a specific embodiment, the extracellular domain of the CAR binds CD20. In a more specific embodiment, the extracellular domain of the CAR is an scFv or antigen-binding fragment thereof binds to CD20.

Other tumor-associated and tumor-specific antigens are known to those in the art.

Antibodies, and scFvs, that bind to TSAs and TAAs are known in the art, as are nucleotide sequences that encode them.

In certain specific embodiments, the antigen is an antigen not considered to be a TSA or a TAA, but which is nevertheless associated with tumor cells, or damage caused by a tumor. In specific embodiments, the antigen is a tumor microenvironment-associated antigen (TMAA). In certain embodiments, for example, the TMAA is, e.g., a growth factor, cytokine or interleukin, e.g., a growth factor, cytokine, or interleukin associated with angiogenesis or vasculogenesis. Such growth factors, cytokines, or interleukins can include, e.g., vascular endothelial growth factor (VEGF), basic fibroblast growth factor (bFGF), platelet-derived growth factor (PDGF), hepatocyte growth factor (HGF), insulin-like growth factor (IGF), or interleukin-8 (IL-8). Tumors can also create a hypoxic environment local to the tumor. As such, in other specific embodiments, the TMAA is a hypoxia-associated factor, e.g., HIF-1α, HIF-1β, HIF-2α, HIF-2β, HIF-3α, or HIF-3β. Tumors can also cause localized damage to normal tissue, causing the release of molecules known as damage associated molecular pattern molecules (DAMPs; also known as alarmins). In certain other specific embodiments, therefore, the TMAA is a DAMP, e.g., a heat shock protein, chromatin-associated protein high mobility group box 1 (HMGB1), S100A8 (MRP8, calgranulin A), S100A9 (MRP14, calgranulin B), serum amyloid A (SAA), or can be a deoxyribonucleic acid, adenosine triphosphate, uric acid, or heparin sulfate. In specific embodiments, the TMAA is VEGF-A, EGF, PDGF, IGF, or bFGF.

In a specific embodiment of any of any of the above aspects or embodiments, said CAR comprises an antibody or antibody fragment that targets an antigen of interest. In a more specific embodiment, said CAR comprises a single chain Fv antibody fragment (scFv). In one embodiment, the chimeric antigen receptor comprises an scFv that binds an antigen of interest, e.g., an antigen on a tumor cell, a hinge domain comprising a CD8α polypeptide, a CD8α transmembrane domain, a CD137 (4-1BB) intracellular co-stimulatory signaling domain, and a CD3ζ primary signaling domain. The tumor cell may be, e.g., a cell in a solid tumor, or a cell of a blood cancer. The antigen can be any antigen that is expressed on a cell of any tumor or cancer type. In one embodiment, the immune cells comprise a chimeric antigen receptor which comprises a single chain Fv antibody fragment that targets an antigen of interest. In one embodiment, the immune cells comprise a chimeric antigen receptor which comprises a scFv that binds an antigen of interest, a hinge domain comprising a CD8α polypeptide, a CD8α transmembrane domain, a CD137 (4-1BB) intracellular co-stimulatory signaling domain, and a CD3ζ primary signaling domain.

In a specific embodiment of any of any of the above aspects or embodiments, said CAR comprises an antibody or antibody fragment that targets BCMA. In a more specific embodiment, said CAR comprises a single chain Fv antibody fragment (scFv). In a more specific embodiment, said CAR comprises a BCMA02 scFv, e.g., SEQ ID NO: 38. In a specific embodiment of any of the above aspects or embodiments, said immune cells are idecabtagene vicleucel cells. In one embodiment, the chimeric antigen receptor comprises a murine single chain Fv antibody fragment that targets BCMA, e.g., BCMA. In one embodiment, the chimeric antigen receptor comprises a murine anti-BCMA scFv that binds a BCMA polypeptide, e.g., a human BCMA polypeptide a hinge domain comprising a CD8α polypeptide, a CD8α transmembrane domain, a CD137 (4-1BB) intracellular co-stimulatory signaling domain, and a CD3ζ primary signaling domain. In one embodiment, the chimeric antigen receptor comprises a murine scFv that targets BCMA, e.g., BCMA, wherein the scFV is that of anti-BCMA02 CAR of SEQ ID NO: 9 or SEQ ID NO: 37. In one embodiment, the chimeric antigen receptor is or comprises SEQ ID NO: 9. In one embodiment, the chimeric antigen receptor is or comprises SEQ ID NO: 37. In a more specific embodiment of any embodiment herein, said immune cells are idecabtagene vicleucel (ide-cel) cells. In one embodiment, the immune cells comprise a chimeric antigen receptor which comprises a murine single chain Fv antibody fragment that targets BCMA, e.g., BCMA. In one embodiment, the immune cells comprise a chimeric antigen receptor which comprises a murine anti-BCMA scFv that binds a BCMA polypeptide, e.g., BCMA, a hinge domain comprising a CD8α polypeptide, a CD8α transmembrane domain, a CD137 (4-1BB) intracellular co-stimulatory signaling domain, and a CD3ζ primary signaling domain. In one embodiment, the immune cells comprise a chimeric antigen receptor which is or comprises SEQ ID NO: 9. In one embodiment, the immune cells comprise a chimeric antigen receptor which is or comprises SEQ ID NO: 37.

In other embodiments, the genetically modified immune effector cells contemplated herein, are administered to a patient with a B cell related condition, e.g., an autoimmune disease associated with B cells or a B cell malignancy.

The practice of the subject matter presented herein employs, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Such techniques are explained fully in the literature. See, e.g., Sambrook, et al., Molecular Cloning: A Laboratory Manual (3rd Edition, 2001); Sambrook, et al., Molecular Cloning: A Laboratory Manual (2nd Edition, 1989); Maniatis et al., Molecular Cloning: A Laboratory Manual (1982); Ausubel et al., Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford, 1985); Anand, Techniques for the Analysis of Complex Genomes, (Academic Press, New York, 1992); Transcription and Translation (B. Hames & S. Higgins, Eds., 1984); Perbal, A Practical Guide to Molecular Cloning (1984); Harlow and Lane, Antibodies, (Cold Spring Harbor Laboratory Press, Cold Spring Harbor, N.Y., 1998) Current Protocols in Immunology Q. E. Coligan, A. M. Kruisbeek, D. H. Margulies, E. M. Shevach and W. Strober, eds., 1991); Annual Review of Immunology; as well as monographs in journals such as Advances in Immunology.

### 6.2. Definitions

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, preferred embodiments of compositions, methods and materials are described herein. For the purposes of the present disclosure, the following terms are defined below.

The articles “a,” “an,” and “the” are used herein to refer to one or to more than one (i.e., to at least one, or to one or more) of the grammatical object of the article. By way of example, “an element” means one element or one or more elements.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are present that materially affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the disclosure presented herein. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

### 6.3. Chimeric Antigen Receptors

In various embodiments, genetically engineered receptors that redirect cytotoxicity of immune effector cells toward B cells are provided. These genetically engineered receptors referred to herein as chimeric antigen receptors (CARs). CARs are molecules that combine antibody-based specificity for a desired antigen (e.g., BCMA) with a T cell receptor-activating intracellular domain to generate a chimeric protein that exhibits a specific anti-BCMA cellular immune activity. As used herein, the term, “chimeric,” describes being composed of parts of different proteins or DNAs from different origins.

CAR T cell therapies to which the embodiments described herein apply include any CAR T therapy, such as BCMA CAR T cell therapies, such as BCMA02, JCARH125, JNJ-68284528 (LCAR-B38M) (Janssen/Legend), P-BCMA-101 (Poseida), PBCAR269A (Poseida), P-BCMA-Allol (Poseida), Allo-715 (Pfizer/Allogene), CT053 (Carsgen), Descartes-08 (Cartesian), PHE885 (Novartis), CTX120 (CRISPR Therapeutics); CD19 CAR T therapies, e.g., Yescarta, Kymriah, Tecartus, lisocabtagene maraleucel (liso-cel), and CAR T therapies targeting any other cell surface marker.

The extracellular domain (also referred to as a binding domain or antigen-specific binding domain) of the polypeptide binds to an antigen of interest. In certain embodiments, the extracellular domain comprises a receptor, or a portion of a receptor, that binds to said antigen. The extracellular domain may be, e.g., a receptor, or a portion of a receptor, that binds to said antigen. In certain embodiments, the extracellular domain comprises, or is, an antibody or an antigen-binding portion thereof. In specific embodiments, the extracellular domain comprises, or is, a single-chain Fv domain. The single-chain Fv domain can comprise, for example, a V_{L} linked to V_{H} by a flexible linker, wherein said V_{L} and V_{H} are from an antibody that binds said antigen.

The antigen to which the extracellular domain of the polypeptide binds can be any antigen of interest, e.g., can be an antigen on a tumor cell. The tumor cell may be, e.g., a cell in a solid tumor, or a cell of a blood cancer. The antigen can be any antigen that is expressed on a cell of any tumor or cancer type, e.g., cells of a lymphoma, a lung cancer, a breast cancer, a prostate cancer, an adrenocortical carcinoma, a thyroid carcinoma, a nasopharyngeal carcinoma, a melanoma, e.g., a malignant melanoma, a skin carcinoma, a colorectal carcinoma, a desmoid tumor, a desmoplastic small round cell tumor, an endocrine tumor, an Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipoma, or the like. In more specific embodiments, said lymphoma can be chronic lymphocytic leukemia (small lymphocytic lymphoma), B-cell prolymphocytic leukemia, lymphoplasmacytic lymphoma, Waldenström macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, a non-Hodgkin lymphoma, or multiple myeloma.

In certain embodiments, the antigen is a tumor-associated antigen (TAA) or a tumor-specific antigen (TSA). In various specific embodiments, without limitation, the tumor-associated antigen or tumor-specific antigen is Her2, prostate stem cell antigen (PSCA), alpha-fetoprotein (AFP), carcinoembryonic antigen (CEA), cancer antigen-125 (CA-125), CA19-9, calretinin, MUC-1, epithelial membrane protein (EMA), epithelial tumor antigen (ETA), tyrosinase, melanoma-associated antigen (MAGE), CD19, CD20, CD34, CD45, CD99, CD117, chromogranin, cytokeratin, desmin, glial fibrillary acidic protein (GFAP), gross cystic disease fluid protein (GCDFP-15), HMB-45 antigen, high molecular weight melanoma-associated antigen (HMW-MAA), protein melan-A (MART-1), myo-D1, muscle-specific actin (MSA), neurofilament, neuron-specific enolase (NSE), placental alkaline phosphatase, synaptophysis, thyroglobulin, thyroid transcription factor-1, the dimeric form of the pyruvate kinase isoenzyme type M2 (tumor M2-PK), an abnormal ras protein, or an abnormal p53 protein.

In certain embodiments, the TAA or TSA is a cancer/testis (CT) antigen, e.g., BAGE, CAGE, CTAGE, FATE, GAGE, HCA661, HOM-TES-85, MAGEA, MAGEB, MAGEC, NA88, NY-ESO-1, NY-SAR-35, OY-TES-1, SPANXB1, SPA17, SSX, SYCP1, or TPTE.

In certain other embodiments, the TAA or TSA is a carbohydrate or ganglioside, e.g., fuc-GM1, GM2 (oncofetal antigen-immunogenic-1; OFA-I-1); GD2 (OFA-I-2), GM3, GD3, and the like.

In certain other embodiments, the TAA or TSA is alpha-actinin-4, Bage-1, BCR-ABL, Bcr-Abl fusion protein, beta-catenin, CA 125, CA 15-3 (CA 27.29\BCAA), CA 195, CA 242, CA-50, CAM43, Casp-8, cdc27, cdk4, cdkn2a, CEA, coa-1, dek-can fusion protein, EBNA, EF2, Epstein Barr virus antigens, ETV6-AML1 fusion protein, HLA-A2, HLA-A11, hsp70-2, KIAAO205, Mart2, Mum-1, 2, and 3, neo-PAP, myosin class I, OS-9, pml-RARα fusion protein, PTPRK, K-ras, N-ras, triosephosphate isomerase, Gage 3,4,5,6,7, GnTV, Herv-K-mel, Lage-1, NA-88, NY-Eso-1/Lage-2, SP17, SSX-2, TRP2-Int2, gp100 (Pmel 17), tyrosinase, TRP-1, TRP-2, MAGE-1, MAGE-3, RAGE, GAGE-1, GAGE-2, p15(58), RAGE, SCP-1, Hom/Mel-40, PRAME, p53, H-Ras, HER-2/neu, E2A-PRL, H4-RET, IGH-IGK, MYL-RAR, human papillomavirus (HPV) antigens E6 and E7, TSP-180, MAGE-4, MAGE-5, MAGE-6, pi85erbB2, pi80erbB-3, c-met, nm-23H1, PSA, TAG-72-4, CA 19-9, CA 72-4, CAM 17.1, NuMa, K-ras, 13-Catenin, Mum-1, p16, TAGE, PSMA, CT7, telomerase, 43-9F, 5T4, 791Tgp72, 13HCG, BCA225, BTAA, CD68\KP1, CO-029, FGF-5, G250, Ga733 (EpCAM), HTgp-175, M344, MA-50, MG7-Ag, MOV18, NB\70K, NY-CO-1, RCAS1, SDCCAG16, TA-90, TAAL6, TAG72, TLP, TPS, CD19, CD22, CD27, CD30, CD70, GD2 (ganglioside G2), EGFRvIII (epidermal growth factor variant III), sperm protein 17 (Sp17), mesothelin, PAP (prostatic acid phosphatase), prostein, TARP (T cell receptor gamma alternate reading frame protein), Trp-p8, STEAP1 (six-transmembrane epithelial antigen of the prostate 1), an abnormal ras protein, or an abnormal p53 protein. In another specific embodiment, said tumor-associated antigen or tumor-specific antigen is integrin αvβ (CD61), galactin, K-Ras (V-Ki-ras2 Kirsten rat sarcoma viral oncogene), or Ral-B.

In specific embodiments, the TAA or TSA is CD20, CD123, CLL-1, CD38, CS-1, CD138, ROR1, FAP, MUC1, PSCA, EGFRvIII, EPHA2, or GD2. In further specific embodiments, the TAA or TSA is CD123, CLL-1, CD38, or CS-1. In a specific embodiment, the extracellular domain of the CAR binds CS-1. In a further specific embodiment, the extracellular domain comprises a single-chain version of elotuzumab and/or an antigen-binding fragment of elotuzumab. In a specific embodiment, the extracellular domain of the CAR binds CD20. In a more specific embodiment, the extracellular domain of the CAR is an scFv or antigen-binding fragment thereof binds to CD20.

Other tumor-associated and tumor-specific antigens are known to those in the art.

Antibodies, and scFvs, that bind to TSAs and TAAs are known in the art, as are nucleotide sequences that encode them.

In certain specific embodiments, the antigen is an antigen not considered to be a TSA or a TAA, but which is nevertheless associated with tumor cells, or damage caused by a tumor. In specific embodiments, the antigen is a tumor microenvironment-associated antigen (TMAA). In certain embodiments, for example, the TMAA is, e.g., a growth factor, cytokine or interleukin, e.g., a growth factor, cytokine, or interleukin associated with angiogenesis or vasculogenesis. Such growth factors, cytokines, or interleukins can include, e.g., vascular endothelial growth factor (VEGF), basic fibroblast growth factor (bFGF), platelet-derived growth factor (PDGF), hepatocyte growth factor (HGF), insulin-like growth factor (IGF), or interleukin-8 (IL-8). Tumors can also create a hypoxic environment local to the tumor. As such, in other specific embodiments, the TMAA is a hypoxia-associated factor, e.g., HIF-1α, HIF-1β, HIF-2α, HIF-2β, HIF-3α, or HIF-3β. Tumors can also cause localized damage to normal tissue, causing the release of molecules known as damage associated molecular pattern molecules (DAMPs; also known as alarmins). In certain other specific embodiments, therefore, the TMAA is a DAMP, e.g., a heat shock protein, chromatin-associated protein high mobility group box 1 (HMGB1), S100A8 (MRP8, calgranulin A), S100A9 (MRP14, calgranulin B), serum amyloid A (SAA), or can be a deoxyribonucleic acid, adenosine triphosphate, uric acid, or heparin sulfate. In specific embodiments, the TMAA is VEGF-A, EGF, PDGF, IGF, or bFGF.

In certain embodiments, the extracellular domain is joined to said transmembrane domain by a linker, spacer or hinge polypeptide sequence, e.g., a sequence from CD28.

In certain embodiments, CARs contemplated herein, comprise an extracellular domain that binds to BCMA, a transmembrane domain, and an intracellular signaling domain. Engagement of the anti-BCMA antigen binding domain of the CAR with BCMA on the surface of a target cell results in clustering of the CAR and delivers an activation stimulus to the CAR-containing cell. The main characteristic of CARs are their ability to redirect immune effector cell specificity, thereby triggering proliferation, cytokine production, phagocytosis or production of molecules that can mediate cell death of the target antigen expressing cell in a major histocompatibility (MHC) independent manner, exploiting the cell specific targeting abilities of monoclonal antibodies, soluble ligands or cell specific co-receptors.

In various embodiments, a CAR comprises an extracellular binding domain that comprises a murine anti-BCMA (e.g., human BCMA)-specific binding domain; a transmembrane domain; one or more intracellular co-stimulatory signaling domains; and a primary signaling domain.

In particular embodiments, a CAR comprises an extracellular binding domain that comprises a murine anti-BCMA (e.g., human BCMA) antibody or antigen binding fragment thereof, one or more hinge domains or spacer domains; a transmembrane domain including; one or more intracellular co-stimulatory signaling domains; and a primary signaling domain.

6.3.1. Binding Domain

In particular embodiments, CARs contemplated herein comprise an extracellular binding domain that comprises a murine anti-BCMA antibody or antigen binding fragment thereof that specifically binds to a human BCMA polypeptide expressed on a B cell. As used herein, the terms, “binding domain,” “extracellular domain,” “extracellular binding domain,” “antigen-specific binding domain,” and “extracellular antigen specific binding domain,” are used interchangeably and provide a CAR with the ability to specifically bind to the target antigen of interest, e.g., BCMA. The binding domain may be derived either from a natural, synthetic, semi-synthetic, or recombinant source.

The terms “specific binding affinity” or “specifically binds” or “specifically bound” or “specific binding” or “specifically targets” as used herein, describe binding of an anti-BCMA antibody or antigen binding fragment thereof (or a CAR comprising the same) to BCMA at greater binding affinity than background binding. A binding domain (or a CAR comprising a binding domain or a fusion protein containing a binding domain) “specifically binds” to a BCMA if it binds to or associates with BCMA with an affinity or Kₐ (i.e., an equilibrium association constant of a particular binding interaction with units of 1/M) of, for example, greater than or equal to about 10⁵ M⁻¹. In certain embodiments, a binding domain (or a fusion protein thereof) binds to a target with a Kₐ greater than or equal to about 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹. “High affinity” binding domains (or single chain fusion proteins thereof) refers to those binding domains with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, at least 10¹³ M⁻¹, or greater.

Alternatively, affinity may be defined as an equilibrium dissociation constant (Kₐ) of a particular binding interaction with units of M (e.g., 10⁻⁵ M to 10⁻¹³ M, or less). Affinities of binding domain polypeptides and CAR proteins according to the present disclosure can be readily determined using conventional techniques, e.g., by competitive ELISA (enzyme-linked immunosorbent assay), or by binding association, or displacement assays using labeled ligands, or using a surface-plasmon resonance device such as the Biacore T100, which is available from Biacore, Inc., Piscataway, NJ, or optical biosensor technology such as the EPIC system or EnSpire that are available from Corning and Perkin Elmer respectively (see also, e.g., Scatchard et al. (1949) Ann. N.Y. Acad. Sci. 51:660; and U.S. Pat. Nos. 5,283,173; 5,468,614, or the equivalent).

In one embodiment, the affinity of specific binding is about 2 times greater than background binding, about 5 times greater than background binding, about 10 times greater than background binding, about 20 times greater than background binding, about 50 times greater than background binding, about 100 times greater than background binding, or about 1000 times greater than background binding or more.

In particular embodiments, the extracellular binding domain of a CAR comprises an antibody or antigen binding fragment thereof. An “antibody” refers to a binding agent that is a polypeptide comprising at least a light chain or heavy chain immunoglobulin variable region which specifically recognizes and binds an epitope of an antigen, such as a peptide, lipid, polysaccharide, or nucleic acid containing an antigenic determinant, such as those recognized by an immune cell.

An “antigen (Ag)” refers to a compound, composition, or substance that can stimulate the production of antibodies or a T cell response in an animal, including compositions (such as one that includes a cancer-specific protein) that are injected or absorbed into an animal. An antigen reacts with the products of specific humoral or cellular immunity, including those induced by heterologous antigens, such as the disclosed antigens. In particular embodiments, the target antigen is an epitope of a BCMA polypeptide.

An “epitope” or “antigenic determinant” refers to the region of an antigen to which a binding agent binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, or about 8-10 amino acids in a unique spatial conformation.

Antibodies include antigen binding fragments thereof, such as Camel Ig, Ig NAR, Fab fragments, Fab′ fragments, F(ab)′₂ fragments, F(ab)′₃ fragments, Fv, single chain Fv proteins (“scFv”), bis-scFv, (scFv)₂, minibodies, diabodies, triabodies, tetrabodies, disulfide stabilized Fv proteins (“dsFv”), and single-domain antibody (sdAb, Nanobody) and portions of full length antibodies responsible for antigen binding. The term also includes genetically engineered forms such as chimeric antibodies (for example, humanized murine antibodies), heteroconjugate antibodies (such as, bispecific antibodies) and antigen binding fragments thereof. See also, Pierce Catalog and Handbook, 1994-1995 (Pierce Chemical Co., Rockford, IL); Kuby, J., Immunology, 3_{rd} Ed., W. H. Freeman & Co., New York, 1997.

As would be understood by the skilled person and as described elsewhere herein, a complete antibody comprises two heavy chains and two light chains. Each heavy chain consists of a variable region and a first, second, and third constant region, while each light chain consists of a variable region and a constant region. Mammalian heavy chains are classified as α, δ, ε, γ, and μ. Mammalian light chains are classified as λ or κ. Immunoglobulins comprising the α, δ, ε, γ, and μ heavy chains are classified as immunoglobulin (Ig)A, IgD, IgE, IgG, and IgM. The complete antibody forms a “Y” shape. The stem of the Y consists of the second and third constant regions (and for IgE and IgM, the fourth constant region) of two heavy chains bound together and disulfide bonds (inter-chain) are formed in the hinge. Heavy chains γ, α and δ have a constant region composed of three tandem (in a line) Ig domains, and a hinge region for added flexibility; heavy chains μ and ε have a constant region composed of four immunoglobulin domains. The second and third constant regions are referred to as “CH2 domain” and “CH3 domain”, respectively. Each arm of the Y includes the variable region and first constant region of a single heavy chain bound to the variable and constant regions of a single light chain. The variable regions of the light and heavy chains are responsible for antigen binding.

Light and heavy chain variable regions contain a “framework” region interrupted by three hypervariable regions, also called “complementarity-determining regions” or “CDRs”. The CDRs can be defined or identified by conventional methods, such as by sequence according to Kabat et al (Wu, T T and Kabat, E. A., J Exp Med. 132(2):211-50, (1970); Borden, P. and Kabat E. A., PNAS, 84: 2440-2443 (1987); (see, Kabat et al., Sequences of Proteins of Immunological Interest, U.S. Department of Health and Human Services, 1991, which is hereby incorporated by reference), or by structure according to Chothia et al (Chothia, C. and Lesk, A. M., J Mol. Biol., 196(4): 901-917 (1987), Chothia, C. et al, Nature, 342: 877-883 (1989)).

The sequences of the framework regions of different light or heavy chains are relatively conserved within a species, such as humans. The framework region of an antibody, that is the combined framework regions of the constituent light and heavy chains, serves to position and align the CDRs in three-dimensional space. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, the CDRs located in the variable domain of the heavy chain of the antibody are referred to as CDRH1, CDRH2, and CDRH3, whereas the CDRs located in the variable domain of the light chain of the antibody are referred to as CDRL1, CDRL2, and CDRL3. Antibodies with different specificities (i.e., different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs). Illustrative examples of light chain CDRs that are suitable for constructing humanized BCMA CARs contemplated herein include, but are not limited to the CDR sequences set forth in SEQ ID NOs: 1-3. Illustrative examples of heavy chain CDRs that are suitable for constructing humanized BCMA CARs contemplated herein include, but are not limited to the CDR sequences set forth in SEQ ID NOs: 4-6.

References to “V_{H}” or “VH” refer to the variable region of an immunoglobulin heavy chain, including that of an antibody, Fv, scFv, dsFv, Fab, or other antibody fragment as disclosed herein. References to “V_{L}” or “VL” refer to the variable region of an immunoglobulin light chain, including that of an antibody, Fv, scFv, dsFv, Fab, or other antibody fragment as disclosed herein.

A “monoclonal antibody” is an antibody produced by a single clone of B lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

A “chimeric antibody” has framework residues from one species, such as human, and CDRs (which generally confer antigen binding) from another species, such as a mouse. In particular embodiments, a CAR contemplated herein comprises antigen-specific binding domain that is a chimeric antibody or antigen binding fragment thereof.

A “humanized” antibody is an immunoglobulin including a human framework region and one or more CDRs from a non-human (for example a mouse, rat, or synthetic) immunoglobulin. The non-human immunoglobulin providing the CDRs is termed a “donor,” and the human immunoglobulin providing the framework is termed an “acceptor.”

In particular embodiments, a murine anti-BCMA (e.g., human BCMA) antibody or antigen binding fragment thereof, includes but is not limited to a Camel Ig (a camelid antibody (VHH)), Ig NAR, Fab fragments, Fab′ fragments, F(ab)′₂ fragments, F(ab)′₃ fragments, Fv, single chain Fv antibody (“scFv”), bis-scFv, (scFv)₂, minibody, diabody, triabody, tetrabody, disulfide stabilized Fv protein (“dsFv”), and single-domain antibody (sdAb, Nanobody).

“Camel Ig” or “camelid VHH” as used herein refers to the smallest known antigen-binding unit of a heavy chain antibody (Koch-Nolte, et al, FASEB J., 21: 3490-3498 (2007)). A “heavy chain antibody” or a “camelid antibody” refers to an antibody that contains two VH domains and no light chains (Riechmann L. et al, J. Immunol. Methods 231:25-38 (1999); WO94/04678; WO94/25591; U.S. Pat. No. 6,005,079).

“IgNAR” of “immunoglobulin new antigen receptor” refers to class of antibodies from the shark immune repertoire that consist of homodimers of one variable new antigen receptor (VNAR) domain and five constant new antigen receptor (CNAR) domains. IgNARs represent some of the smallest known immunoglobulin-based protein scaffolds and are highly stable and possess efficient binding characteristics. The inherent stability can be attributed to both (i) the underlying Ig scaffold, which presents a considerable number of charged and hydrophilic surface exposed residues compared to the conventional antibody VH and VL domains found in murine antibodies; and (ii) stabilizing structural features in the complementary determining region (CDR) loops including inter-loop disulphide bridges, and patterns of intra-loop hydrogen bonds.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Fv” is the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three hypervariable regions (HVRs) of each variable domain interact to define an antigen-binding site on the surface of the VH-VL dimer. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

The Fab fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO 1993/01161; Hudson et al., Nat. Med. 9:129-134 (2003); and Hollinger et al., PNAS USA 90: 6444-6448 (1993). Triabodies and tetrabodies are also described in Hudson et al., Nat. Med. 9:129-134 (2003).

“Single domain antibody” or “sdAb” or “nanobody” refers to an antibody fragment that consists of the variable region of an antibody heavy chain (VH domain) or the variable region of an antibody light chain (VL domain) (Holt, L., et al, 2003, Trends in Biotechnology, 21(11): 484-490).

“Single-chain Fv” or “scFv” antibody fragments comprise the VH and VL domains of antibody, wherein these domains are present in a single polypeptide chain and in either orientation (e.g., VL-VH or VH-VL). Generally, the scFv polypeptide further comprises a polypeptide linker between the VH and VL domains which enables the scFv to form the desired structure for antigen binding. For a review of scFv, see, e.g., Pluckthun, in The Pharmacology of Monoclonal Antibodies, vol. 113, Rosenburg and Moore eds., (Springer-Verlag, New York, 1994), pp. 269-315.

In certain embodiments, a CAR contemplated herein comprises antigen-specific binding domain that is a murine scFv. Single chain antibodies may be cloned form the V region genes of a hybridoma specific for a desired target. The production of such hybridomas has become routine. A technique which can be used for cloning the variable region heavy chain (V_{H}) and variable region light chain (V_{L}) has been described, for example, in Orlandi et al., PNAS, 1989; 86: 3833-3837.

In particular embodiments, the antigen-specific binding domain that is a murine scFv that binds a human BCMA polypeptide. Illustrative examples of variable heavy chains that are suitable for constructing BCMA CARs contemplated herein include, but are not limited to the amino acid sequences set forth in SEQ ID NO: 8. Illustrative examples of variable light chains that are suitable for constructing BCMA CARs contemplated herein include, but are not limited to the amino acid sequences set forth in SEQ ID NO: 7.

BCMA-specific binding domains provided herein also comprise one, two, three, four, five, or six CDRs. Such CDRs may be nonhuman CDRs or altered nonhuman CDRs selected from CDRL1, CDRL2 and CDRL3 of the light chain and CDRH1, CDRH2 and CDRH3 of the heavy chain. In certain embodiments, a BCMA-specific binding domain comprises (a) a light chain variable region that comprises a light chain CDRL1, a light chain CDRL2, and a light chain CDRL3, and (b) a heavy chain variable region that comprises a heavy chain CDRH1, a heavy chain CDRH2, and a heavy chain CDRH3.

6.3.2. Linkers

In certain embodiments, the CARs contemplated herein may comprise linker residues between the various domains, e.g., added for appropriate spacing and conformation of the molecule. In particular embodiments the linker is a variable region linking sequence. A “variable region linking sequence” is an amino acid sequence that connects the V_{H} and V_{L} domains and provides a spacer function compatible with interaction of the two sub-binding domains so that the resulting polypeptide retains a specific binding affinity to the same target molecule as an antibody that comprises the same light and heavy chain variable regions. CARs contemplated herein, may comprise one, two, three, four, or five or more linkers. In particular embodiments, the length of a linker is about 1 to about 25 amino acids, about 5 to about 20 amino acids, or about 10 to about 20 amino acids, or any intervening length of amino acids. In some embodiments, the linker is 1, 2, 3, 4, 5, 6, 7, 8, 9, 10, 11, 12, 13, 14, 15, 16, 17, 18, 19, 20, 21, 22, 23, 24, 25, or more amino acids long.

Illustrative examples of linkers include glycine polymers (G)ₙ; glycine-serine polymers (G₁₋₅S₁₋₅)ₙ, where n is an integer of at least one, two, three, four, or five; glycine-alanine polymers; alanine-serine polymers; and other flexible linkers known in the art. Glycine and glycine-serine polymers are relatively unstructured, and therefore may be able to serve as a neutral tether between domains of fusion proteins such as the CARs described herein. Glycine accesses significantly more phi-psi space than even alanine, and is much less restricted than residues with longer side chains (see Scheraga, Rev. Computational Chem. 11173-142 (1992)). The ordinarily skilled artisan will recognize that design of a CAR in particular embodiments can include linkers that are all or partially flexible, such that the linker can include a flexible linker as well as one or more portions that confer less flexible structure to provide for a desired CAR structure.

Other exemplary linkers include, but are not limited to the following amino acid sequences: GGG; DGGGS (SEQ ID NO: 12); TGEKP (SEQ ID NO: 13) (see, e.g., Liu et al., PNAS 5525-5530 (1997)); GGRR (SEQ ID NO: 14) (Pomerantz et al. 1995, supra); (GGGGS)n wherein n=1, 2, 3, 4 or 5, and where GGGGS is identified as SEQ ID NO: 15 (Kim et al., PNAS 93, 1156-1160 (1996.); EGKSSGSGSESKVD (SEQ ID NO: 16) (Chaudhary et al., 1990, Proc. Natl. Acad. Sci. U.S.A. 87:1066-1070); KESGSVSSEQLAQFRSLD (SEQ ID NO: 17) (Bird et al., 1988, Science 242:423-426), GGRRGGGS (SEQ ID NO: 18); LRQRDGERP (SEQ ID NO: 19); LRQKDGGGSERP (SEQ ID NO: 20); LRQKd(GGGS)₂ ERP (SEQ ID NO: 21). Alternatively, flexible linkers can be rationally designed using a computer program capable of modeling both DNA-binding sites and the peptides themselves (Desjarlais & Berg, PNAS 90:2256-2260 (1993), PNAS 91:11099-11103 (1994) or by phage display methods. In one embodiment, the linker comprises the following amino acid sequence: GSTSGSGKPGSGEGSTKG (SEQ ID NO: 22) (Cooper et al., Blood, 101(4): 1637-1644 (2003)).

6.3.3. Spacer Domain

In particular embodiments, the binding domain of the CAR is followed by one or more “spacer domains,” which refers to the region that moves the antigen binding domain away from the effector cell surface to enable proper cell/cell contact, antigen binding and activation (Patel et al., Gene Therapy, 1999; 6: 412-419). The spacer domain may be derived either from a natural, synthetic, semi-synthetic, or recombinant source. In certain embodiments, a spacer domain is a portion of an immunoglobulin, including, but not limited to, one or more heavy chain constant regions, e.g., CH2 and CH3. The spacer domain can include the amino acid sequence of a naturally occurring immunoglobulin hinge region or an altered immunoglobulin hinge region.

In one embodiment, the spacer domain comprises the CH2 and CH3 domains of IgG1 or IgG4.

6.3.4. Hinge Domain

The binding domain of the CAR is generally followed by one or more “hinge domains,” which play a role in positioning the antigen binding domain away from the effector cell surface to enable proper cell/cell contact, antigen binding and activation. A CAR generally comprises one or more hinge domains between the binding domain and the transmembrane domain (TM). The hinge domain may be derived either from a natural, synthetic, semi-synthetic, or recombinant source. The hinge domain can include the amino acid sequence of a naturally occurring immunoglobulin hinge region or an altered immunoglobulin hinge region.

An “altered hinge region” refers to (a) a naturally occurring hinge region with up to 30% amino acid changes (e.g., up to 25%, 20%, 15%, 10%, or 5% amino acid substitutions or deletions), (b) a portion of a naturally occurring hinge region that is at least 10 amino acids (e.g., at least 12, 13, 14 or 15 amino acids) in length with up to 30% amino acid changes (e.g., up to 25%, 20%, 15%, 10%, or 5% amino acid substitutions or deletions), or (c) a portion of a naturally occurring hinge region that comprises the core hinge region (which may be 4, 5, 6, 7, 8, 9, 10, 11, 12, 13, 14, or 15, or at least 4, 5, 6, 7, 8, 9, 10, 11, 12, 13, 14, or 15 amino acids in length). In certain embodiments, one or more cysteine residues in a naturally occurring immunoglobulin hinge region may be substituted by one or more other amino acid residues (e.g., one or more serine residues). An altered immunoglobulin hinge region may alternatively or additionally have a proline residue of a wild type immunoglobulin hinge region substituted by another amino acid residue (e.g., a serine residue).

Other illustrative hinge domains suitable for use in the CARs described herein include the hinge region derived from the extracellular regions of type 1 membrane proteins such as CD8a, CD4, CD28 and CD7, which may be wild-type hinge regions from these molecules or may be altered. In another embodiment, the hinge domain comprises a CD8α hinge region.

6.3.5. Transmembrane Domain

The transmembrane (TM) domain is the portion of the CAR that fuses the extracellular binding portion and intracellular signaling domain and anchors the CAR to the plasma membrane of the immune effector cell. The TM domain may be derived either from a natural, synthetic, semi-synthetic, or recombinant source. The TM domain may be derived from (i.e., comprise at least the transmembrane region(s) of) the alpha, beta or zeta chain of the T-cell receptor, CD3ε, CD3ζ, CD4, CD5, CD8α, CD9, CD16, CD22, CD27, CD28, CD33, CD37, CD45, CD64, CD80, CD86, CD134, CD137, CD152, CD154, and PD-1. In a particular embodiment, the TM domain is synthetic and predominantly comprises hydrophobic residues such as leucine and valine.

In one embodiment, the CARs contemplated herein comprise a TM domain derived from CD8α. In another embodiment, a CAR contemplated herein comprises a TM domain derived from CD8α and a short oligo- or polypeptide linker, preferably between 1, 2, 3, 4, 5, 6, 7, 8, 9, or 10 amino acids in length that links the TM domain and the intracellular signaling domain of the CAR. A glycine-serine based linker provides a particularly suitable linker.

6.3.6. Intracellular Signaling Domain

In particular embodiments, CARs contemplated herein comprise an intracellular signaling domain. An “intracellular signaling domain” refers to the part of a CAR that participates in transducing the message of effective BCMA CAR binding to a human BCMA polypeptide into the interior of the immune effector cell to elicit effector cell function, e.g., activation, cytokine production, proliferation and cytotoxic activity, including the release of cytotoxic factors to the CAR-bound target cell, or other cellular responses elicited with antigen binding to the extracellular CAR domain.

The term “effector function” refers to a specialized function of an immune effector cell. Effector function of the T cell, for example, may be cytolytic activity or helper activity including the secretion of a cytokine. Thus, the term “intracellular signaling domain” refers to the portion of a protein which transduces the effector function signal and that directs the cell to perform a specialized function. While usually the entire intracellular signaling domain can be employed, in many cases it is not necessary to use the entire domain. To the extent that a truncated portion of an intracellular signaling domain is used, such truncated portion may be used in place of the entire domain as long as it transduces the effector function signal. The term intracellular signaling domain is meant to include any truncated portion of the intracellular signaling domain sufficient to transducing effector function signal.

It is known that signals generated through the TCR alone are insufficient for full activation of the T cell and that a secondary or co-stimulatory signal is also required. Thus, T cell activation can be said to be mediated by two distinct classes of intracellular signaling domains: primary signaling domains that initiate antigen-dependent primary activation through the TCR (e.g., a TCR/CD3 complex) and co-stimulatory signaling domains that act in an antigen-independent manner to provide a secondary or co-stimulatory signal. In certain embodiments, a CAR contemplated herein comprises an intracellular signaling domain that comprises one or more “co-stimulatory signaling domain” and a “primary signaling domain.”

Primary signaling domains regulate primary activation of the TCR complex either in a stimulatory way, or in an inhibitory way. Primary signaling domains that act in a stimulatory manner may contain signaling motifs which are known as immunoreceptor tyrosine-based activation motifs or ITAMs.

Illustrative examples of ITAM containing primary signaling domains that are of particular use in the subject matter presented herein include those derived from TCRζ, FcRγ, FcRβ, CD3γ, CD3δ, CD3ε, CD3ζ, CD22, CD79a, CD79b, and CD66d. In particular embodiments, a CAR comprises a CD3ζ primary signaling domain and one or more co-stimulatory signaling domains. The intracellular primary signaling and co-stimulatory signaling domains may be linked in any order in tandem to the carboxyl terminus of the transmembrane domain.

CARs contemplated herein comprise one or more co-stimulatory signaling domains to enhance the efficacy and expansion of T cells expressing CAR receptors. As used herein, the term, “co-stimulatory signaling domain,” or “co-stimulatory domain”, refers to an intracellular signaling domain of a co-stimulatory molecule. Co-stimulatory molecules are cell surface molecules other than antigen receptors or Fc receptors that provide a second signal required for efficient activation and function of T lymphocytes upon binding to antigen. Illustrative examples of such co-stimulatory molecules include CARD11, CD2, CD7, CD27, CD28, CD30, CD40, CD54 (ICAM), CD83, CD134 (OX40), CD137 (4-1BB), CD150 (SLAMF1), CD152 (CTLA4), CD223 (LAG3), CD270 (HVEM), CD273 (PD-L2), CD274 (PD-L1), CD278 (ICOS), DAP10, LAT, NKD2C SLP76, TRIM, and ZAP70. In one embodiment, a CAR comprises one or more co-stimulatory signaling domains selected from the group consisting of CD28, CD137, and CD134, and a CD3ζ primary signaling domain.

In another embodiment, a CAR comprises CD28 and CD137 co-stimulatory signaling domains and a CD3ζ primary signaling domain.

In yet another embodiment, a CAR comprises CD28 and CD134 co-stimulatory signaling domains and a CD3ζ primary signaling domain.

In one embodiment, a CAR comprises CD137 and CD134 co-stimulatory signaling domains and a CD3ζ primary signaling domain.

In particular embodiments, CARs contemplated herein comprise a human anti-BCMA antibody or antigen binding fragment thereof that specifically binds to a BCMA polypeptide expressed on B cells, e.g., a human BCMA expressed on human B cells.

In particular embodiments, CARs contemplated herein comprise a murine anti-BCMA antibody or antigen binding fragment thereof that specifically binds to a BCMA polypeptide expressed on B cells, e.g., a human BCMA expressed on human B cells.

In one embodiment, a CAR comprises a murine anti-BCMA scFv that binds a BCMA polypeptide, e.g., a human BCMA polypeptide; a transmembrane domain derived from a polypeptide selected from the group consisting of: alpha, beta or zeta chain of the T-cell receptor, CD3ε, CD3ζ, CD4, CD5, CD8α, CD9, CD 16, CD22, CD27, CD28, CD33, CD37, CD45, CD64, CD80, CD86, CD 134, CD137, CD152, CD 154, and PD1; and one or more intracellular co-stimulatory signaling domains from a co-stimulatory molecule selected from the group consisting of: CARD11, CD2, CD7, CD27, CD28, CD30, CD40, CD54 (ICAM), CD83, CD134 (OX40), CD137 (4-1BB), CD150 (SLAMF1), CD152 (CTLA4), CD223 (LAG3), CD270 (HVEM), CD273 (PD-L2), CD274 (PD-L1), CD278 (ICOS), DAP10, LAT, NKD2C SLP76, TRIM, and ZAP70; and a primary signaling domain from TCRζ, FcRγ, FcRβ, CD3γ, CD3δ, CD3δ, CD3ζ, CD22, CD79a, CD79b, and CD66d.

In one embodiment, a CAR comprises a murine anti-BCMA scFv that binds a BCMA polypeptide, e.g., a human BCMA polypeptide; a transmembrane domain derived from a polypeptide selected from the group consisting of: alpha, beta or zeta chain of the T-cell receptor, CD3ε, CD3ζ, CD4, CD5, CD8α, CD9, CD 16, CD22, CD27, CD28, CD33, CD37, CD45, CD64, CD80, CD86, CD 134, CD137, CD152, CD 154, and PD1; and one or more intracellular co-stimulatory signaling domains from a co-stimulatory molecule selected from the group consisting of: CARD11, CD2, CD7, CD27, CD28, CD30, CD40, CD54 (ICAM), CD83, CD134 (OX40), CD137 (4-1BB), CD150 (SLAMF1), CD152 (CTLA4), CD223 (LAG3), CD270 (HVEM), CD273 (PD-L2), CD274 (PD-L1), CD278 (ICOS), DAP10, LAT, NKD2C SLP76, TRIM, and ZAP70; and one or more primary signaling domains from a polypeptide selected from the group consisting of: TCRζ, FcRγ, FcRβ, CD3γ, CD3δ, CD3ε, CD3ζ, CD22, CD79a, CD79b, and CD66d.

In one embodiment, a CAR comprises a murine anti-BCMA scFv that binds a BCMA polypeptide; e.g., a human BCMA polypeptide, a hinge domain selected from the group consisting of: IgG1 hinge/CH2/CH3, IgG4 hinge/CH2/CH3, and a CD8α hinge; a transmembrane domain derived from a polypeptide selected from the group consisting of: alpha, beta or zeta chain of the T-cell receptor, CD3ε, CD3ζ, CD4, CD5, CD8α, CD9, CD 16, CD22, CD27, CD28, CD33, CD37, CD45, CD64, CD80, CD86, CD 134, CD137, CD152, CD 154, and PD1; and one or more intracellular co-stimulatory signaling domains from a co-stimulatory molecule selected from the group consisting of: CARD11, CD2, CD7, CD27, CD28, CD30, CD40, CD54 (ICAM), CD83, CD134 (OX40), CD137 (4-1BB), CD150 (SLAMF1), CD152 (CTLA4), CD223 (LAG3), CD270 (HVEM), CD273 (PD-L2), CD274 (PD-L1), CD278 (ICOS), DAP10, LAT, NKD2C SLP76, TRIM, and ZAP70; and a primary signaling domain from TCRζ, FcRγ, FcRβ, CD3γ, CD3δ, CD3ε, CD3ζ, CD22, CD79a, CD79b, and CD66d.

In one embodiment, a CAR comprises a murine anti-BCMA scFv that binds a BCMA polypeptide, e.g., a human BCMA polypeptide; a hinge domain selected from the group consisting of: IgG1 hinge/CH2/CH3, IgG4 hinge/CH2/CH3, and a CD8α hinge; a transmembrane domain derived from a polypeptide selected from the group consisting of: alpha, beta or zeta chain of the T-cell receptor, CD3ε, CD3ζ, CD4, CD5, CD8α, CD9, CD 16, CD22, CD27, CD28, CD33, CD37, CD45, CD64, CD80, CD86, CD 134, CD137, CD152, CD 154, and PD1; and one or more intracellular co-stimulatory signaling domains from a co-stimulatory molecule selected from the group consisting of: CARD11, CD2, CD7, CD27, CD28, CD30, CD40, CD54 (ICAM), CD83, CD134 (OX40), CD137 (4-1BB), CD150 (SLAMF1), CD152 (CTLA4), CD223 (LAG3), CD270 (HVEM), CD273 (PD-L2), CD274 (PD-L1), CD278 (ICOS), DAP10, LAT, NKD2C SLP76, TRIM, and ZAP70; and one or more primary signaling domains from a polypeptide selected from the group consisting of: TCRζ, FcRγ, FcRβ, CD3γ, CD3δ, CD3δ, CD3ζ, CD22, CD79a, CD79b, and CD66d.

In one embodiment, a CAR comprises a murine anti-BCMA scFv that binds a BCMA polypeptide, e.g., a human BCMA polypeptide; a hinge domain selected from the group consisting of: IgG1 hinge/CH2/CH3, IgG4 hinge/CH2/CH3, and a CD8α hinge; a transmembrane domain derived from a polypeptide selected from the group consisting of: alpha, beta or zeta chain of the T-cell receptor, CD3ε, CD3ζ, CD4, CD5, CD8α, CD9, CD 16, CD22, CD27, CD28, CD33, CD37, CD45, CD64, CD80, CD86, CD 134, CD137, CD152, CD 154, and PD1; a short oligo- or polypeptide linker, preferably between 1, 2, 3, 4, 5, 6, 7, 8, 9, or 10 amino acids in length that links the TM domain to the intracellular signaling domain of the CAR; and one or more intracellular co-stimulatory signaling domains from a co-stimulatory molecule selected from the group consisting of: CARD11, CD2, CD7, CD27, CD28, CD30, CD40, CD54 (ICAM), CD83, CD134 (OX40), CD137 (4-1BB), CD150 (SLAMF1), CD152 (CTLA4), CD223 (LAG3), CD270 (HVEM), CD273 (PD-L2), CD274 (PD-L1), CD278 (ICOS), DAP10, LAT, NKD2C SLP76, TRIM, and ZAP70; and a primary signaling domain from TCRζ, FcRγ, FcRβ, CD3γ, CD3δ, CD3δ, CD3ζ, CD22, CD79a, CD79b, and CD66d.

In one embodiment, a CAR comprises a murine anti-BCMA scFv that binds a BCMA polypeptide, e.g., a human BCMA polypeptide; a hinge domain selected from the group consisting of: IgG1 hinge/CH2/CH3, IgG4 hinge/CH2/CH3, and a CD8α hinge; a transmembrane domain derived from a polypeptide selected from the group consisting of: alpha, beta or zeta chain of the T-cell receptor, CD3ε, CD3ζ, CD4, CD5, CD8α, CD9, CD 16, CD22, CD27, CD28, CD33, CD37, CD45, CD64, CD80, CD86, CD 134, CD137, CD152, CD 154, and PD1; a short oligo- or polypeptide linker, preferably between 1, 2, 3, 4, 5, 6, 7, 8, 9, or 10 amino acids in length that links the TM domain to the intracellular signaling domain of the CAR; and one or more intracellular co-stimulatory signaling domains from a co-stimulatory molecule selected from the group consisting of: CARD11, CD2, CD7, CD27, CD28, CD30, CD40, CD54 (ICAM), CD83, CD134 (OX40), CD137 (4-1BB), CD150 (SLAMF1), CD152 (CTLA4), CD223 (LAG3), CD270 (HVEM), CD273 (PD-L2), CD274 (PD-L1), CD278 (ICOS), DAP10, LAT, NKD2C SLP76, TRIM, and ZAP70; and one or more primary signaling domains from a polypeptide selected from the group consisting of: TCRζ, FcRγ, FcRβ, CD3γ, CD3δ, CD3ε, CD3ζ, CD22, CD79a, CD79b, and CD66d.

In a particular embodiment, a CAR comprises a murine anti-BCMA scFv that binds a BCMA polypeptide, e.g., a human BCMA polypeptide; a hinge domain comprising an IgG1 hinge/CH2/CH3 polypeptide and a CD8α polypeptide; a CD8α transmembrane domain comprising a polypeptide linker of about 3 to about 10 amino acids; a CD137 intracellular co-stimulatory signaling domain; and a CD3ζ primary signaling domain.

In a particular embodiment, a CAR comprises a murine anti-BCMA scFv that binds a BCMA polypeptide, e.g., a human BCMA polypeptide; a hinge domain comprising a CD8α polypeptide; a CD8α transmembrane domain comprising a polypeptide linker of about 3 to about 10 amino acids; a CD134 intracellular co-stimulatory signaling domain; and a CD3ζ primary signaling domain.

In a particular embodiment, a CAR comprises a murine anti-BCMA scFv that binds a BCMA polypeptide, e.g., a human BCMA polypeptide; a hinge domain comprising a CD8α polypeptide; a CD8α transmembrane domain comprising a polypeptide linker of about 3 to about 10 amino acids; a CD28 intracellular co-stimulatory signaling domain; and a CD3ζ primary signaling domain.

In a particular embodiment, a CAR comprises a murine anti-BCMA scFv that binds a BCMA polypeptide, e.g., a human BCMA polypeptide; a hinge domain comprising a CD8α polypeptide; a CD8α transmembrane domain; a CD137 (4-1BB) intracellular co-stimulatory signaling domain; and a CD3ζ primary signaling domain.

Moreover, the design of the CARs contemplated herein enable improved expansion, long-term persistence, and tolerable cytotoxic properties in T cells expressing the CARs compared to non-modified T cells or T cells modified to express other CARs.

6.4. Polypeptides

The present disclosure contemplates, in part, CAR polypeptides and fragments thereof, cells and compositions comprising the same, and vectors that express polypeptides. In particular embodiments, a polypeptide comprising one or more CARs as set forth in SEQ ID NO:9 is provided.

“Polypeptide,” “polypeptide fragment,” “peptide” and “protein” are used interchangeably, unless specified to the contrary, and according to conventional meaning, i.e., as a sequence of amino acids. Polypeptides are not limited to a specific length, e.g., they may comprise a full length protein sequence or a fragment of a full length protein, and may include post-translational modifications of the polypeptide, for example, glycosylations, acetylations, phosphorylations and the like, as well as other modifications known in the art, both naturally occurring and non-naturally occurring. In various embodiments, the CAR polypeptides contemplated herein comprise a signal (or leader) sequence at the N-terminal end of the protein, which co-translationally or post-translationally directs transfer of the protein. Illustrative examples of suitable signal sequences useful in CARs disclosed herein include, but are not limited to, the IgG1 heavy chain signal sequence and the CD8α signal sequence. Polypeptides can be prepared using any of a variety of well-known recombinant and/or synthetic techniques. Polypeptides contemplated herein specifically encompass the CARs of the present disclosure, or sequences that have deletions from, additions to, and/or substitutions of one or more amino acid of a CAR as disclosed herein.

An “isolated peptide” or an “isolated polypeptide” and the like, as used herein, refer to in vitro isolation and/or purification of a peptide or polypeptide molecule from a cellular environment, and from association with other components of the cell, i.e., it is not significantly associated with in vivo substances. Similarly, an “isolated cell” refers to a cell that has been obtained from an in vivo tissue or organ and is substantially free of extracellular matrix.

Polypeptides include “polypeptide variants.” Polypeptide variants may differ from a naturally occurring polypeptide in one or more substitutions, deletions, additions and/or insertions. Such variants may be naturally occurring or may be synthetically generated, for example, by modifying one or more of the above polypeptide sequences. For example, in particular embodiments, it may be desirable to improve the binding affinity and/or other biological properties of the CARs by introducing one or more substitutions, deletions, additions and/or insertions into a binding domain, hinge, TM domain, co-stimulatory signaling domain or primary signaling domain of a CAR polypeptide. In certain embodiments, such polypeptides include polypeptides having at least about 65%, 70%, 75%, 85%, 90%, 95%, 98%, or 99% amino acid identity thereto.

Polypeptides include “polypeptide fragments.” Polypeptide fragments refer to a polypeptide, which can be monomeric or multimeric, that has an amino-terminal deletion, a carboxyl-terminal deletion, and/or an internal deletion or substitution of a naturally-occurring or recombinantly-produced polypeptide. In certain embodiments, a polypeptide fragment can comprise an amino acid chain at least 5 to about 500 amino acids long. It will be appreciated that in certain embodiments, fragments are at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 150, 200, 250, 300, 350, 400, or 450 amino acids long. Particularly useful polypeptide fragments include functional domains, including antigen-binding domains or fragments of antibodies. In the case of a murine anti-BCMA (e.g., human BCMA) antibody, useful fragments include, but are not limited to: a CDR region, a CDR3 region of the heavy or light chain; a variable region of a heavy or light chain; a portion of an antibody chain or variable region including two CDRs; and the like.

The polypeptide may also be fused in-frame or conjugated to a linker or other sequence for ease of synthesis, purification or identification of the polypeptide (e.g., poly-His), or to enhance binding of the polypeptide to a solid support.

As noted above, polypeptides of the present disclosure may be altered in various ways including amino acid substitutions, deletions, truncations, and insertions. Methods for such manipulations are generally known in the art. For example, amino acid sequence variants of a reference polypeptide can be prepared by mutations in the DNA. Methods for mutagenesis and nucleotide sequence alterations are well known in the art. See, for example, Kunkel (1985, Proc. Natl. Acad. Sci. USA. 82: 488-492), Kunkel et al., (1987, Methods in Enzymol, 154: 367-382), U.S. Pat. No. 4,873,192, Watson, J. D. et al., (Molecular Biology of the Gene, Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.).

In certain embodiments, a variant will contain conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Modifications may be made in the structure of the polynucleotides and polypeptides of the present disclosure and still obtain a functional molecule that encodes a variant or derivative polypeptide with desirable characteristics. When it is desired to alter the amino acid sequence of a polypeptide to create an equivalent, or even an improved, variant polypeptide, one skilled in the art, for example, can change one or more of the codons of the encoding DNA sequence, e.g., according to Table 2.

TABLE 2
Amino Acid Codons

| | One | Three | | | | | | |
|---|---|---|---|---|---|---|---|---|
| | letter | letter | | | | | | |
| Amino Acids | code | code | Codons | | | | | |
| Alanine | A | Ala | GCA | GCC | GCG | GCU | | |
| Cysteine | C | Cys | UGC | UGU | | | | |
| Aspartic acid | D | Asp | GAC | GAU | | | | |
| Glutamic acid | E | Glu | GAA | GAG | | | | |
| Phenylalanine | F | Phe | UUC | UUU | | | | |
| Glycine | G | Gly | GGA | GGC | GGG | GGU | | |
| Histidine | H | His | CAC | CAU | | | | |
| Isoleucine | I | Ile | AUA | AUC | AUU | | | |
| Lysine | K | Lys | AAA | AAG | | | | |
| Leucine | L | Leu | UUA | UUG | CUA | CUC | CUG | CUU |
| Methionine | M | Met | AUG | | | | | |
| Asparagine | N | Asn | AAC | AAU | | | | |
| Proline | P | Pro | CCA | CCC | CCG | CCU | | |
| Glutamine | Q | Gln | CAA | CAG | | | | |
| Arginine | R | Arg | AGA | AGG | CGA | CGC | CGG | CGU |
| Serine | S | Ser | AGC | AGU | UCA | UCC | UCG | UCU |
| Threonine | T | Thr | ACA | ACC | ACG | ACU | | |
| Valine | V | Val | GUA | GUC | GUG | GUU | | |
| Tryptophan | W | Trp | UGG | | | | | |
| Tyrosine | Y | Tyr | UAC | UAU | | | | |

Guidance in determining which amino acid residues can be substituted, inserted, or deleted without abolishing biological activity can be found using computer programs well known in the art, such as DNASTAR™ software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Exemplary conservative substitutions are described in U.S. Provisional Patent Application No. 61/241,647, the disclosure of which is herein incorporated by reference.

In making such changes, the hydropathic index of amino acids may be considered. The importance of the hydropathic amino acid index in conferring interactive biologic function on a protein is generally understood in the art (Kyte and Doolittle, 1982, incorporated herein by reference). Each amino acid has been assigned a hydropathic index on the basis of its hydrophobicity and charge characteristics (Kyte and Doolittle, 1982). These values are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like.

Polypeptide variants further include glycosylated forms, aggregative conjugates with other molecules, and covalent conjugates with unrelated chemical moieties (e.g., pegylated molecules). Covalent variants can be prepared by linking functionalities to groups which are found in the amino acid chain or at the N- or C-terminal residue, as is known in the art. Variants also include allelic variants, species variants, and muteins. Truncations or deletions of regions which do not affect functional activity of the proteins are also variants.

In one embodiment, where expression of two or more polypeptides is desired, the polynucleotide sequences encoding them can be separated by and IRES sequence as discussed elsewhere herein. In another embodiment, two or more polypeptides can be expressed as a fusion protein that comprises one or more self-cleaving polypeptide sequences.

Polypeptides disclosed herein include fusion polypeptides. In certain embodiments, fusion polypeptides and polynucleotides encoding fusion polypeptides are provided, e.g., CARs. Fusion polypeptides and fusion proteins refer to a polypeptide having at least two, three, four, five, six, seven, eight, nine, or ten or more polypeptide segments. Fusion polypeptides are typically linked C-terminus to N-terminus, although they can also be linked C-terminus to C-terminus, N-terminus to N-terminus, or N-terminus to C-terminus. The polypeptides of the fusion protein can be in any order or a specified order. Fusion polypeptides or fusion proteins can also include conservatively modified variants, polymorphic variants, alleles, mutants, subsequences, and interspecies homologs, so long as the desired transcriptional activity of the fusion polypeptide is preserved. Fusion polypeptides may be produced by chemical synthetic methods or by chemical linkage between the two moieties or may generally be prepared using other standard techniques. Ligated DNA sequences comprising the fusion polypeptide are operably linked to suitable transcriptional or translational control elements as discussed elsewhere herein.

In one embodiment, a fusion partner comprises a sequence that assists in expressing the protein (an expression enhancer) at higher yields than the native recombinant protein. Other fusion partners may be selected so as to increase the solubility of the protein or to enable the protein to be targeted to desired intracellular compartments or to facilitate transport of the fusion protein through the cell membrane.

Fusion polypeptides may further comprise a polypeptide cleavage signal between each of the polypeptide domains described herein. In addition, a polypeptide site can be put into any linker peptide sequence. Exemplary polypeptide cleavage signals include polypeptide cleavage recognition sites such as protease cleavage sites, nuclease cleavage sites (e.g., rare restriction enzyme recognition sites, self-cleaving ribozyme recognition sites), and self-cleaving viral oligopeptides (see deFelipe and Ryan, 2004. Traffic, 5(8); 616-26).

Suitable protease cleavages sites and self-cleaving peptides are known to the skilled person (see, e.g., in Ryan et al., 1997. J. Gener. Virol. 78, 699-722; Scymczak et al. (2004) Nature Biotech. 5, 589-594). Exemplary protease cleavage sites include, but are not limited to, the cleavage sites of potyvirus NIa proteases (e.g., tobacco etch virus protease), potyvirus HC proteases, potyvirus P1 (P35) proteases, byovirus NIa proteases, byovirus RNA-2-encoded proteases, aphthovirus L proteases, enterovirus 2A proteases, rhinovirus 2A proteases, picorna 3C proteases, comovirus 24K proteases, nepovirus 24K proteases, RTSV (rice tungro spherical virus) 3C-like protease, PYVF (parsnip yellow fleck virus) 3C-like protease, heparin, thrombin, factor Xa and enterokinase. Due to its high cleavage stringency, TEV (tobacco etch virus) protease cleavage sites are preferred in one embodiment, e.g., EXXYXQ(G/S) (SEQ ID NO: 23), for example, ENLYFQG (SEQ ID NO: 24) and ENLYFQS (SEQ ID NO: 25), wherein X represents any amino acid (cleavage by TEV occurs between Q and G or Q and S).

In a particular embodiment, self-cleaving peptides include those polypeptide sequences obtained from potyvirus and cardiovirus 2A peptides, FMDV (foot-and-mouth disease virus), equine rhinitis A virus, Thosea asigna virus and porcine teschovirus.

In certain embodiments, the self-cleaving polypeptide site comprises a 2A or 2A-like site, sequence or domain (Donnelly et al., 2001. J. Gen. Virol. 82:1027-1041).

TABLE 3
Exemplary 2A sites include the following
sequences:

| | |
|---|---|
| SEQ ID NO: 26 | LLNFDLLKLAGDVESNPGP |
| SEQ ID NO: 27 | TLNFDLLKLAGDVESNPGP |
| SEQ ID NO: 28 | LLKLAGDVESNPGP |
| SEQ ID NO: 29 | NFDLLKLAGDVESNPGP |
| SEQ ID NO: 30 | QLLNFDLLKLAGDVESNPGP |
| SEQ ID NO: 31 | APVKQTLNFDLLKLAGDVESNPGP |
| SEQ ID NO: 32 | VTELLYRMKRAETYCPRPLLAIHPTEARHKQKI |
| | VAPVKQT |
| SEQ ID NO: 33 | LNFDLLKLAGDVESNPGP |
| SEQ ID NO: 34 | LLAIHPTEARHKQKIVAPVKQTLNFDLLKLAGD |
| | VESNPGP |
| SEQ ID NO: 35 | EARHKQKIVAPVKQTLNFDLLKLAGDVESNPGP |

In certain embodiments, a polypeptide contemplated herein comprises a CAR polypeptide.

6.5. Polynucleotides

In certain embodiments, a polynucleotide encoding one or more CAR polypeptides is provided, e.g., SEQ ID NO: 10. As used herein, the terms “polynucleotide” or “nucleic acid” refers to messenger RNA (mRNA), RNA, genomic RNA (gRNA), plus strand RNA (RNA(+)), minus strand RNA (RNA(−)), genomic DNA (gDNA), complementary DNA (cDNA) or recombinant DNA. Polynucleotides include single and double stranded polynucleotides. Preferably, polynucleotides disclosed herein include polynucleotides or variants having at least about 50%, 55%, 60%, 65%, 70%, 75%, 80%, 85%, 90%, 95%, 96%, 97%, 98%, 99% or 100% sequence identity to any of the reference sequences described herein (see, e.g., Sequence Listing), typically where the variant maintains at least one biological activity of the reference sequence. In various illustrative embodiments, the present disclosure contemplates, in part, polynucleotides comprising expression vectors, viral vectors, and transfer plasmids, and compositions, and cells comprising the same.

In particular embodiments, polynucleotides are provided by this disclosure that encode at least about 5, 10, 25, 50, 100, 150, 200, 250, 300, 350, 400, 500, 1000, 1250, 1500, 1750, or 2000 or more contiguous amino acid residues of a polypeptide, as well as all intermediate lengths. It will be readily understood that “intermediate lengths,” in this context, means any length between the quoted values, such as 6, 7, 8, 9, etc., 101, 102, 103, etc.; 151, 152, 153, etc.; 201, 202, 203, etc.

As used herein, the terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms include polynucleotides in which one or more nucleotides have been added or deleted, or replaced with different nucleotides compared to a reference polynucleotide. In this regard, it is well understood in the art that certain alterations inclusive of mutations, additions, deletions and substitutions can be made to a reference polynucleotide whereby the altered polynucleotide retains the biological function or activity of the reference polynucleotide.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 50%, 55%, 60%, 65%, 70%, 75%, 80%, 85%, 90%, 95%, 96%, 97%, 98%, 99% or 100% sequence identity to any of the reference sequences described herein, typically where the polypeptide variant maintains at least one biological activity of the reference polypeptide.

Terms used to describe sequence relationships between two or more polynucleotides or polypeptides include “reference sequence,” “comparison window,” “sequence identity,” “percentage of sequence identity,” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, WI, USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., Current Protocols in Molecular Biology, John Wiley & Sons Inc, 1994-1998, Chapter 15.

As used herein, “isolated polynucleotide” refers to a polynucleotide that has been purified from the sequences which flank it in a naturally-occurring state, e.g., a DNA fragment that has been removed from the sequences that are normally adjacent to the fragment. An “isolated polynucleotide” also refers to a complementary DNA (cDNA), a recombinant DNA, or other polynucleotide that does not exist in nature and that has been made by the hand of man.

Terms that describe the orientation of polynucleotides include: 5′ (normally the end of the polynucleotide having a free phosphate group) and 3′ (normally the end of the polynucleotide having a free hydroxyl (OH) group). Polynucleotide sequences can be annotated in the 5′ to 3′ orientation or the 3′ to 5′ orientation. For DNA and mRNA, the 5′ to 3′ strand is designated the “sense,” “plus,” or “coding” strand because its sequence is identical to the sequence of the premessenger (premRNA) [except for uracil (U) in RNA, instead of thymine (T) in DNA]. For DNA and mRNA, the complementary 3′ to 5′ strand which is the strand transcribed by the RNA polymerase is designated as “template,” “antisense,” “minus,” or “non-coding” strand. As used herein, the term “reverse orientation” refers to a 5′ to 3′ sequence written in the 3′ to 5′ orientation or a 3′ to 5′ sequence written in the 5′ to 3′ orientation.

The terms “complementary” and “complementarity” refer to polynucleotides (i.e., a sequence of nucleotides) related by the base-pairing rules. For example, the complementary strand of the DNA sequence 5′ A G T C A T G 3′ is 3′ T C A G T A C 5′. The latter sequence is often written as the reverse complement with the 5′ end on the left and the 3′ end on the right, 5′ C A T G A C T 3′. A sequence that is equal to its reverse complement is said to be a palindromic sequence. Complementarity can be “partial,” in which only some of the nucleic acids' bases are matched according to the base pairing rules. Or, there can be “complete” or “total” complementarity between the nucleic acids.

Moreover, it will be appreciated by those of ordinary skill in the art that, as a result of the degeneracy of the genetic code, there are many nucleotide sequences that encode a polypeptide, or fragment of variant thereof, as described herein. Some of these polynucleotides bear minimal homology to the nucleotide sequence of any native gene. Nonetheless, polynucleotides that vary due to differences in codon usage are specifically contemplated by the present disclosure, for example polynucleotides that are optimized for human and/or primate codon selection. Further, alleles of the genes comprising the polynucleotide sequences provided herein may also be used. Alleles are endogenous genes that are altered as a result of one or more mutations, such as deletions, additions and/or substitutions of nucleotides.

The term “nucleic acid cassette” as used herein refers to genetic sequences within a vector which can express a RNA, and subsequently a protein. The nucleic acid cassette contains the gene of interest, e.g., a CAR. The nucleic acid cassette is positionally and sequentially oriented within the vector such that the nucleic acid in the cassette can be transcribed into RNA, and when necessary, translated into a protein or a polypeptide, undergo appropriate post-translational modifications required for activity in the transformed cell, and be translocated to the appropriate compartment for biological activity by targeting to appropriate intracellular compartments or secretion into extracellular compartments. Preferably, the cassette has its 3′ and 5′ ends adapted for ready insertion into a vector, e.g., it has restriction endonuclease sites at each end. In one embodiment, the nucleic acid cassette contains the sequence of a chimeric antigen receptor used to treat a tumor or a cancer. In one embodiment, the nucleic acid cassette contains the sequence of a chimeric antigen receptor used to treat a B cell malignancy. The cassette can be removed and inserted into a plasmid or viral vector as a single unit.

In particular embodiments, polynucleotides include at least one polynucleotide-of-interest. As used herein, the term “polynucleotide-of-interest” refers to a polynucleotide encoding a polypeptide (i.e., a polypeptide-of-interest), inserted into an expression vector that is desired to be expressed. A vector may comprise 1, 2, 3, 4, 5, 6, 7, 8, 9, or 10 polynucleotides-of-interest. In certain embodiments, the polynucleotide-of-interest encodes a polypeptide that provides a therapeutic effect in the treatment or prevention of a disease or disorder. Polynucleotides-of-interest, and polypeptides encoded therefrom, include both polynucleotides that encode wild-type polypeptides, as well as functional variants and fragments thereof. In particular embodiments, a functional variant has at least 80%, at least 90%, at least 95%, or at least 99% identity to a corresponding wild-type reference polynucleotide or polypeptide sequence. In certain embodiments, a functional variant or fragment has at least 50%, at least 60%, at least 70%, at least 80%, or at least 90% of a biological activity of a corresponding wild-type polypeptide.

In one embodiment, the polynucleotide-of-interest does not encode a polypeptide but serves as a template to transcribe miRNA, siRNA, or shRNA, ribozyme, or other inhibitory RNA. In various other embodiments, a polynucleotide comprises a polynucleotide-of-interest encoding a CAR and one or more additional polynucleotides-of-interest including but not limited to an inhibitory nucleic acid sequence including, but not limited to: an siRNA, an miRNA, an shRNA, and a ribozyme.

As used herein, the terms “siRNA” or “short interfering RNA” refer to a short polynucleotide sequence that mediates a process of sequence-specific post-transcriptional gene silencing, translational inhibition, transcriptional inhibition, or epigenetic RNAi in animals (Zamore et al., 2000, Cell, 101, 25-33; Fire et al., 1998, Nature, 391, 806; Hamilton et al., 1999, Science, 286, 950-951; Lin et al., 1999, Nature, 402, 128-129; Sharp, 1999, Genes & Dev., 13, 139-141; and Strauss, 1999, Science, 286, 886). In certain embodiments, an siRNA comprises a first strand and a second strand that have the same number of nucleosides; however, the first and second strands are offset such that the two terminal nucleosides on the first and second strands are not paired with a residue on the complimentary strand. In certain instances, the two nucleosides that are not paired are thymidine resides. The siRNA should include a region of sufficient homology to the target gene, and be of sufficient length in terms of nucleotides, such that the siRNA, or a fragment thereof, can mediate down regulation of the target gene. Thus, an siRNA includes a region which is at least partially complementary to the target RNA. It is not necessary that there be perfect complementarity between the siRNA and the target, but the correspondence must be sufficient to enable the siRNA, or a cleavage product thereof, to direct sequence specific silencing, such as by RNAi cleavage of the target RNA. Complementarity, or degree of homology with the target strand, is most critical in the antisense strand. While perfect complementarity, particularly in the antisense strand, is often desired, some embodiments include one or more, but preferably 10, 8, 6, 5, 4, 3, 2, or fewer mismatches with respect to the target RNA. The mismatches are most tolerated in the terminal regions, and if present are preferably in a terminal region or regions, e.g., within 6, 5, 4, or 3 nucleotides of the 5′ and/or 3′ terminus. The sense strand need only be sufficiently complementary with the antisense strand to maintain the overall double-strand character of the molecule.

In addition, an siRNA may be modified or include nucleoside analogs. Single stranded regions of an siRNA may be modified or include nucleoside analogs, e.g., the unpaired region or regions of a hairpin structure, e.g., a region which links two complementary regions, can have modifications or nucleoside analogs. Modification to stabilize one or more 3′- or 5′-terminus of an siRNA, e.g., against exonucleases, or to favor the antisense siRNA agent to enter into RISC are also useful. Modifications can include C3 (or C6, C7, C12) amino linkers, thiol linkers, carboxyl linkers, non-nucleotidic spacers (C3, C6, C9, C12, abasic, triethylene glycol, hexaethylene glycol), special biotin or fluorescein reagents that come as phosphoramidites and that have another DMT-protected hydroxyl group, allowing multiple couplings during RNA synthesis. Each strand of an siRNA can be equal to or less than 30, 25, 24, 23, 22, 21, or 20 nucleotides in length. The strand is preferably at least 19 nucleotides in length. For example, each strand can be between 21 and 25 nucleotides in length. Preferred siRNAs have a duplex region of 17, 18, 19, 29, 21, 22, 23, 24, or 25 nucleotide pairs, and one or more overhangs of 2-3 nucleotides, preferably one or two 3′ overhangs, of 2-3 nucleotides.

As used herein, the terms “miRNA” or “microRNA” refer to small non-coding RNAs of 20-22 nucleotides, typically excised from ˜70 nucleotide fold-back RNA precursor structures known as pre-miRNAs. miRNAs negatively regulate their targets in one of two ways depending on the degree of complementarity between the miRNA and the target. First, miRNAs that bind with perfect or nearly perfect complementarity to protein-coding mRNA sequences induce the RNA-mediated interference (RNAi) pathway. miRNAs that exert their regulatory effects by binding to imperfect complementary sites within the 3′ untranslated regions (UTRs) of their mRNA targets, repress target-gene expression post-transcriptionally, apparently at the level of translation, through a RISC complex that is similar to, or possibly identical with, the one that is used for the RNAi pathway. Consistent with translational control, miRNAs that use this mechanism reduce the protein levels of their target genes, but the mRNA levels of these genes are only minimally affected. miRNAs encompass both naturally occurring miRNAs as well as artificially designed miRNAs that can specifically target any mRNA sequence. For example, in one embodiment, the skilled artisan can design short hairpin RNA constructs expressed as human miRNA (e.g., miR-30 or miR-21) primary transcripts. This design adds a Drosha processing site to the hairpin construct and has been shown to greatly increase knockdown efficiency (Pusch et al., 2004). The hairpin stem consists of 22-nt of dsRNA (e.g., antisense has perfect complementarity to desired target) and a 15-19-nt loop from a human miR. Adding the miR loop and miR30 flanking sequences on either or both sides of the hairpin results in greater than 10-fold increase in Drosha and Dicer processing of the expressed hairpins when compared with conventional shRNA designs without microRNA. Increased Drosha and Dicer processing translates into greater siRNA/miRNA production and greater potency for expressed hairpins.

As used herein, the terms “shRNA” or “short hairpin RNA” refer to double-stranded structure that is formed by a single self-complementary RNA strand. shRNA constructs containing a nucleotide sequence identical to a portion, of either coding or non-coding sequence, of the target gene are preferred for inhibition. RNA sequences with insertions, deletions, and single point mutations relative to the target sequence have also been found to be effective for inhibition. Greater than 90% sequence identity, or even 100% sequence identity, between the inhibitory RNA and the portion of the target gene is preferred. In certain preferred embodiments, the length of the duplex-forming portion of an shRNA is at least 20, 21 or 22 nucleotides in length, e.g., corresponding in size to RNA products produced by Dicer-dependent cleavage. In certain embodiments, the shRNA construct is at least 25, 50, 100, 200, 300 or 400 bases in length. In certain embodiments, the shRNA construct is 400-800 bases in length. shRNA constructs are highly tolerant of variation in loop sequence and loop size.

As used herein, the term “ribozyme” refers to a catalytically active RNA molecule capable of site-specific cleavage of target mRNA. Several subtypes have been described, e.g., hammerhead and hairpin ribozymes. Ribozyme catalytic activity and stability can be improved by substituting deoxyribonucleotides for ribonucleotides at noncatalytic bases. While ribozymes that cleave mRNA at site-specific recognition sequences can be used to destroy particular mRNAs, the use of hammerhead ribozymes is preferred. Hammerhead ribozymes cleave mRNAs at locations dictated by flanking regions that form complementary base pairs with the target mRNA. The sole requirement is that the target mRNA has the following sequence of two bases: 5′-UG-3′. The construction and production of hammerhead ribozymes is well known in the art.

In certain embodiments, a method of delivery of a polynucleotide-of-interest that comprises an siRNA, an miRNA, an shRNA, or a ribozyme comprises one or more regulatory sequences, such as, for example, a strong constitutive pol III, e.g., human U6 snRNA promoter, the mouse U6 snRNA promoter, the human and mouse H1 RNA promoter and the human tRNA-val promoter, or a strong constitutive pol II promoter, as described elsewhere herein.

The polynucleotides disclosed herein, regardless of the length of the coding sequence itself, may be combined with other DNA sequences, such as promoters and/or enhancers, untranslated regions (UTRs), signal sequences, Kozak sequences, polyadenylation signals, additional restriction enzyme sites, multiple cloning sites, internal ribosomal entry sites (IRES), recombinase recognition sites (e.g., LoxP, FRT, and Att sites), termination codons, transcriptional termination signals, and polynucleotides encoding self-cleaving polypeptides, epitope tags, as disclosed elsewhere herein or as known in the art, such that their overall length may vary considerably. It is therefore contemplated that a polynucleotide fragment of almost any length may be employed, with the total length preferably being limited by the ease of preparation and use in the intended recombinant DNA protocol.

Polynucleotides can be prepared, manipulated and/or expressed using any of a variety of well-established techniques known and available in the art. In order to express a desired polypeptide, a nucleotide sequence encoding the polypeptide, can be inserted into appropriate vector. Examples of vectors are plasmid, autonomously replicating sequences, and transposable elements. Additional exemplary vectors include, without limitation, plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Examples of categories of animal viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40). Examples of expression vectors are pClneo vectors (Promega) for expression in mammalian cells; pLenti4/V5-DEST™, pLenti6/V5-DEST™, and pLenti6.2/V5-GW/lacZ (Invitrogen) for lentivirus-mediated gene transfer and expression in mammalian cells. In particular embodiments, he coding sequences of the chimeric proteins disclosed herein can be ligated into such expression vectors for the expression of the chimeric protein in mammalian cells.

In one embodiment, a vector encoding a CAR contemplated herein comprises the polynucleotide sequence set forth in SEQ ID NO: 36.

In particular embodiments, the vector is an episomal vector or a vector that is maintained extrachromosomally. As used herein, the term “episomal” refers to a vector that is able to replicate without integration into host's chromosomal DNA and without gradual loss from a dividing host cell also meaning that said vector replicates extrachromosomally or episomally. The vector is engineered to harbor the sequence coding for the origin of DNA replication or “ori” from a lymphotrophic herpes virus or a gamma herpesvirus, an adenovirus, SV40, a bovine papilloma virus, or a yeast, specifically a replication origin of a lymphotrophic herpes virus or a gamma herpesvirus corresponding to oriP of EBV. In a particular aspect, the lymphotrophic herpes virus may be Epstein Barr virus (EBV), Kaposi's sarcoma herpes virus (KSHV), Herpes virus saimiri (HS), or Marek's disease virus (MDV). Epstein Barr virus (EBV) and Kaposi's sarcoma herpes virus (KSHV) are also examples of a gamma herpesvirus. Typically, the host cell comprises the viral replication transactivator protein that activates the replication.

The “control elements” or “regulatory sequences” present in an expression vector are those non-translated regions of the vector—origin of replication, selection cassettes, promoters, enhancers, translation initiation signals (Shine Dalgarno sequence or Kozak sequence) introns, a polyadenylation sequence, 5′ and 3′ untranslated regions—which interact with host cellular proteins to carry out transcription and translation. Such elements may vary in their strength and specificity. Depending on the vector system and host utilized, any number of suitable transcription and translation elements, including ubiquitous promoters and inducible promoters may be used.

In particular embodiments, a vector for utilization herein include, but are not limited to expression vectors and viral vectors, will include exogenous, endogenous, or heterologous control sequences such as promoters and/or enhancers. An “endogenous” control sequence is one which is naturally linked with a given gene in the genome. An “exogenous” control sequence is one which is placed in juxtaposition to a gene by means of genetic manipulation (i.e., molecular biological techniques) such that transcription of that gene is directed by the linked enhancer/promoter. A “heterologous” control sequence is an exogenous sequence that is from a different species than the cell being genetically manipulated.

The term “promoter” as used herein refers to a recognition site of a polynucleotide (DNA or RNA) to which an RNA polymerase binds. An RNA polymerase initiates and transcribes polynucleotides operably linked to the promoter. In particular embodiments, promoters operative in mammalian cells comprise an AT-rich region located approximately 25 to 30 bases upstream from the site where transcription is initiated and/or another sequence found 70 to 80 bases upstream from the start of transcription, a CNCAAT region where N may be any nucleotide.

The term “enhancer” refers to a segment of DNA which contains sequences capable of providing enhanced transcription and in some instances can function independent of their orientation relative to another control sequence. An enhancer can function cooperatively or additively with promoters and/or other enhancer elements. The term “promoter/enhancer” refers to a segment of DNA which contains sequences capable of providing both promoter and enhancer functions.

The term “operably linked” refers to a juxtaposition wherein the components described are in a relationship permitting them to function in their intended manner. In one embodiment, the term refers to a functional linkage between a nucleic acid expression control sequence (such as a promoter, and/or enhancer) and a second polynucleotide sequence, e.g., a polynucleotide-of-interest, wherein the expression control sequence directs transcription of the nucleic acid corresponding to the second sequence.

As used herein, the term “constitutive expression control sequence” refers to a promoter, enhancer, or promoter/enhancer that continually or continuously allows for transcription of an operably linked sequence. A constitutive expression control sequence may be a “ubiquitous” promoter, enhancer, or promoter/enhancer that allows expression in a wide variety of cell and tissue types or a “cell specific,” “cell type specific,” “cell lineage specific,” or “tissue specific” promoter, enhancer, or promoter/enhancer that allows expression in a restricted variety of cell and tissue types, respectively.

Illustrative ubiquitous expression control sequences suitable for use in particular embodiments presented herein include, but are not limited to, a cytomegalovirus (CMV) immediate early promoter, a viral simian virus 40 (SV40) (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus (Irions et al., Nature Biotechnology 25, 1477-1482 (2007)), a Ubiquitin C promoter (UBC), a phosphoglycerate kinase-1 (PGK) promoter, a cytomegalovirus enhancer/chicken β-actin (CAG) promoter, a β-actin promoter and a myeloproliferative sarcoma virus enhancer, negative control region deleted, dl587rev primer-binding site substituted (MND) promoter (Challita et al., J Virol. 69(2):748-55 (1995)).

In one embodiment, a vector of the present disclosure comprises a MND promoter.

In one embodiment, a vector of the present disclosure comprises an EF1α promoter comprising the first intron of the human EF1α gene.

In one embodiment, a vector of the present disclosure comprises an EF1α promoter that lacks the first intron of the human EF1α gene.

In a particular embodiment, it may be desirable to express a polynucleotide comprising a CAR from a T cell specific promoter.

As used herein, “conditional expression” may refer to any type of conditional expression including, but not limited to, inducible expression; repressible expression; expression in cells or tissues having a particular physiological, biological, or disease state, etc. This definition is not intended to exclude cell type or tissue specific expression. Certain embodiments provide conditional expression of a polynucleotide-of-interest, e.g., expression is controlled by subjecting a cell, tissue, organism, etc., to a treatment or condition that causes the polynucleotide to be expressed or that causes an increase or decrease in expression of the polynucleotide encoded by the polynucleotide-of-interest.

Illustrative examples of inducible promoters/systems include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-1 promoter (inducible by interferon), the “GeneSwitch” mifepristone-regulatable system (Sirin et al., 2003, Gene, 323:67), the cumate inducible gene switch (WO 2002/088346), tetracycline-dependent regulatory systems, etc.

Conditional expression can also be achieved by using a site specific DNA recombinase. According to certain embodiments, the vector comprises at least one (typically two) site(s) for recombination mediated by a site specific recombinase. As used herein, the terms “recombinase” or “site specific recombinase” include excusive or integrative proteins, enzymes, co-factors or associated proteins that are involved in recombination reactions involving one or more recombination sites (e.g., two, three, four, five, seven, ten, twelve, fifteen, twenty, thirty, fifty, etc.), which may be wild-type proteins (see Landy, Current Opinion in Biotechnology 3:699-707 (1993)), or mutants, derivatives (e.g., fusion proteins containing the recombination protein sequences or fragments thereof), fragments, and variants thereof. Illustrative examples of recombinases suitable for use herein include, but are not limited to: Cre, Int, IHF, Xis, Flp, Fis, Hin, Gin, ΦC31, Cin, Tn3 resolvase, TndX, XerC, XerD, TnpX, Hjc, Gin, SpCCE1, and ParA.

The vectors may comprise one or more recombination sites for any of a wide variety of site specific recombinases. It is to be understood that the target site for a site specific recombinase is in addition to any site(s) required for integration of a vector, e.g., a retroviral vector or lentiviral vector. As used herein, the terms “recombination sequence,” “recombination site,” or “site specific recombination site” refer to a particular nucleic acid sequence to which a recombinase recognizes and binds.

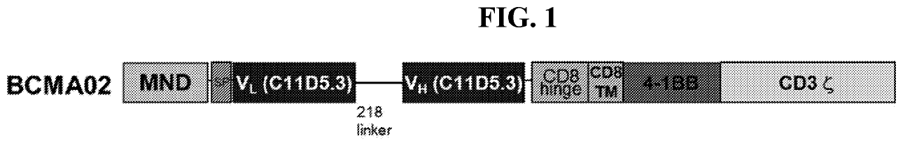

For example, one recombination site for Cre recombinase is loxP which is a 34 base pair sequence comprising two 13 base pair inverted repeats (serving as the recombinase binding sites) flanking an 8 base pair core sequence (see FIG. 1 of Sauer, B., Current Opinion in Biotechnology 5:521-527 (1994)). Other exemplary loxP sites include, but are not limited to: lox511 (Hoess et al., 1996; Bethke and Sauer, 1997), lox5171 (Lee and Saito, 1998), lox2272 (Lee and Saito, 1998), m2 (Langer et al., 2002), lox71 (Albert et al., 1995), and lox66 (Albert et al., 1995).

Suitable recognition sites for the FLP recombinase include, but are not limited to: FRT (McLeod, et al., 1996), F₁, F₂, F₃ (Schlake and Bode, 1994), F₄, F₅ (Schlake and Bode, 1994), FRT(LE) (Senecoff et al., 1988), FRT(RE) (Senecoff et al., 1988).

Other examples of recognition sequences are the attB, attP, attL, and attR sequences, which are recognized by the recombinase enzyme X Integrase, e.g., phi-c31. The φC31 SSR mediates recombination only between the heterotypic sites attB (34 bp in length) and attP (39 bp in length) (Groth et al., 2000). attB and attP, named for the attachment sites for the phage integrase on the bacterial and phage genomes, respectively, both contain imperfect inverted repeats that are likely bound by φC31 homodimers (Groth et al., 2000). The product sites, attL and attR, are effectively inert to further φC31-mediated recombination (Belteki et al., 2003), making the reaction irreversible. For catalyzing insertions, it has been found that attB-bearing DNA inserts into a genomic attP site more readily than an attP site into a genomic attB site (Thyagarajan et al., 2001; Belteki et al., 2003). Thus, typical strategies position by homologous recombination an attP-bearing “docking site” into a defined locus, which is then partnered with an attB-bearing incoming sequence for insertion.

As used herein, an “internal ribosome entry site” or “IRES” refers to an element that promotes direct internal ribosome entry to the initiation codon, such as ATG, of a cistron (a protein encoding region), thereby leading to the cap-independent translation of the gene. See, e.g., Jackson et al., 1990. Trends Biochem Sci 15(12):477-83) and Jackson and Kaminski. 1995. RNA 1(10):985-1000. In particular embodiments, the vectors contemplated herein include one or more polynucleotides-of-interest that encode one or more polypeptides. In particular embodiments, to achieve efficient translation of each of the plurality of polypeptides, the polynucleotide sequences can be separated by one or more IRES sequences or polynucleotide sequences encoding self-cleaving polypeptides.

As used herein, the term “Kozak sequence” refers to a short nucleotide sequence that greatly facilitates the initial binding of mRNA to the small subunit of the ribosome and increases translation. The consensus Kozak sequence is (GCC)RCCATGG, where R is a purine (A or G) (Kozak, 1986. Cell. 44(2):283-92, and Kozak, 1987. Nucleic Acids Res. 15(20):8125-48). In particular embodiments, the vectors contemplated herein comprise polynucleotides that have a consensus Kozak sequence and that encode a desired polypeptide, e.g., a CAR.

In some embodiments, a polynucleotide or cell harboring the polynucleotide utilizes a suicide gene, including an inducible suicide gene to reduce the risk of direct toxicity and/or uncontrolled proliferation. In specific aspects, the suicide gene is not immunogenic to the host harboring the polynucleotide or cell. A certain example of a suicide gene that may be used is caspase-9 or caspase-8 or cytosine deaminase. Caspase-9 can be activated using a specific chemical inducer of dimerization (CID).

In certain embodiments, vectors comprise gene segments that cause the immune effector cells of the present disclosure, e.g., T cells, to be susceptible to negative selection in vivo. By “negative selection” is meant that the infused cell can be eliminated as a result of a change in the in vivo condition of the individual. The negative selectable phenotype may result from the insertion of a gene that confers sensitivity to an administered agent, for example, a compound. Negative selectable genes are known in the art, and include, inter alia the following: the Herpes simplex virus type I thymidine kinase (HSV-I TK) gene (Wigler et al., Cell 11:223, 1977) which confers ganciclovir sensitivity; the cellular hypoxanthine phosphoribosyltransferase (HPRT) gene, the cellular adenine phosphoribosyltransferase (APRT) gene, and bacterial cytosine deaminase, (Mullen et al., Proc. Natl. Acad. Sci. USA. 89:33 (1992)).

In some embodiments, genetically modified immune effector cells, such as T cells, comprise a polynucleotide further comprising a positive marker that enables the selection of cells of the negative selectable phenotype in vitro. The positive selectable marker may be a gene which, upon being introduced into the host cell expresses a dominant phenotype permitting positive selection of cells carrying the gene. Genes of this type are known in the art, and include, inter alia, hygromycin-B phosphotransferase gene (hph) which confers resistance to hygromycin B, the amino glycoside phosphotransferase gene (neo or aph) from Tn5 which codes for resistance to the antibiotic G418, the dihydrofolate reductase (DHFR) gene, the adenosine deaminase gene (ADA), and the multi-drug resistance (MDR) gene.

Preferably, the positive selectable marker and the negative selectable element are linked such that loss of the negative selectable element necessarily also is accompanied by loss of the positive selectable marker. Even more preferably, the positive and negative selectable markers are fused so that loss of one obligatorily leads to loss of the other. An example of a fused polynucleotide that yields as an expression product a polypeptide that confers both the desired positive and negative selection features described above is a hygromycin phosphotransferase thymidine kinase fusion gene (HyTK). Expression of this gene yields a polypeptide that confers hygromycin B resistance for positive selection in vitro, and ganciclovir sensitivity for negative selection in vivo. See Lupton S. D., et al, Mol. and Cell. Biology 1 1:3374-3378, 1991. In addition, in certain embodiments, polynucleotides encoding the chimeric receptors are in retroviral vectors containing the fused gene, particularly those that confer hygromycin B resistance for positive selection in vitro, and ganciclovir sensitivity for negative selection in vivo, for example the HyTK retroviral vector described in Lupton, S. D. et al. (1991), supra. See also the publications of PCT US91/08442 and PCT/US94/05601, by S. D. Lupton, describing the use of bifunctional selectable fusion genes derived from fusing a dominant positive selectable markers with negative selectable markers.

Positive selectable markers can, for example, be derived from genes selected from the group consisting of hph, nco, and gpt, and negative selectable markers can, for example, be derived from genes selected from the group consisting of cytosine deaminase, HSV-I TK, VZV TK, HPRT, APRT and gpt. In specific embodiments, markers are bifunctional selectable fusion genes wherein the positive selectable marker is derived from hph or neo, and the negative selectable marker is derived from cytosine deaminase or a TK gene or selectable marker.

Viral Vectors

In particular embodiments, a cell (e.g., an immune effector cell) is transduced with a retroviral vector, e.g., a lentiviral vector, encoding a CAR. For example, an immune effector cell is transduced with a vector encoding a CAR that comprises a murine anti-BCMA antibody or antigen binding fragment thereof that binds a BCMA polypeptide, e.g., a human BCMA polypeptide, with an intracellular signaling domain of CD3ζ, CD28, 4-1BB, Ox40, or any combinations thereof. Alternatively, an immune effector cell is transduced with a vector encoding a CAR that comprises an antibody or antigen binding fragment thereof that binds an extracellular antigen, e.g., a tumor antigen, with an intracellular signaling domain of CD3ζ, CD28, 4-1BB, Ox40, or any combinations thereof. Thus, these transduced cells can elicit a CAR-mediated cytotoxic response.

Retroviruses are a common tool for gene delivery (Miller, 2000, Nature. 357: 455-460). In particular embodiments, a retrovirus is used to deliver a polynucleotide encoding a chimeric antigen receptor (CAR) to a cell. As used herein, the term “retrovirus” refers to an RNA virus that reverse transcribes its genomic RNA into a linear double-stranded DNA copy and subsequently covalently integrates its genomic DNA into a host genome. Once the virus is integrated into the host genome, it is referred to as a “provirus.” The provirus serves as a template for RNA polymerase II and directs the expression of RNA molecules which encode the structural proteins and enzymes needed to produce new viral particles.

Illustrative retroviruses suitable for use in particular embodiments, include, but are not limited to: Moloney murine leukemia virus (MMuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus.

As used herein, the term “lentivirus” refers to a group (or genus) of complex retroviruses. Illustrative lentiviruses include, but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In one embodiment, HIV based vector backbones (i.e., HIV cis-acting sequence elements) are utilized. In particular embodiments, a lentivirus is used to deliver a polynucleotide comprising a CAR to a cell.

Retroviral vectors and more particularly lentiviral vectors may be used in practicing particular embodiments disclosed herein. Accordingly, the term “retrovirus” or “retroviral vector”, as used herein is meant to include “lentivirus” and “lentiviral vectors” respectively.

The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses.

As will be evident to one of skill in the art, the term “viral vector” is widely used to refer either to a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s).

The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus. The term “hybrid vector” refers to a vector, LTR or other nucleic acid containing both retroviral, e.g., lentiviral, sequences and non-lentiviral viral sequences. In one embodiment, a hybrid vector refers to a vector or transfer plasmid comprising retroviral e.g., lentiviral, sequences for reverse transcription, replication, integration and/or packaging.

In particular embodiments, the terms “lentiviral vector” and “lentiviral expression vector” may be used to refer to lentiviral transfer plasmids and/or infectious lentiviral particles. Where reference is made herein to elements such as cloning sites, promoters, regulatory elements, heterologous nucleic acids, etc., it is to be understood that the sequences of these elements are present in RNA form in the lentiviral particles of the present disclosure and are present in DNA form in the DNA plasmids of the present disclosure.

At each end of the provirus are structures called “long terminal repeats” or “LTRs.” The term “long terminal repeat (LTR)” refers to domains of base pairs located at the ends of retroviral DNAs which, in their natural sequence context, are direct repeats and contain U3, R and U5 regions. LTRs generally provide functions fundamental to the expression of retroviral genes (e.g., promotion, initiation and polyadenylation of gene transcripts) and to viral replication. The LTR contains numerous regulatory signals including transcriptional control elements, polyadenylation signals and sequences needed for replication and integration of the viral genome. The viral LTR is divided into three regions called U3, R and U5. The U3 region contains the enhancer and promoter elements. The U5 region is the sequence between the primer binding site and the R region and contains the polyadenylation sequence. The R (repeat) region is flanked by the U3 and U5 regions. The LTR composed of U3, R and U5 regions and appears at both the 5′ and 3′ ends of the viral genome. Adjacent to the 5′ LTR are sequences necessary for reverse transcription of the genome (the tRNA primer binding site) and for efficient packaging of viral RNA into particles (the Psi site).

As used herein, the term “packaging signal” or “packaging sequence” refers to sequences located within the retroviral genome which are required for insertion of the viral RNA into the viral capsid or particle, see e.g., Clever et al., 1995. J. of Virology, Vol. 69, No. 4; pp. 2101-2109. Several retroviral vectors use the minimal packaging signal (also referred to as the psi [Ψ] sequence) needed for encapsidation of the viral genome. Thus, as used herein, the terms “packaging sequence,” “packaging signal,” “psi” and the symbol “Ψ,” are used in reference to the non-coding sequence required for encapsidation of retroviral RNA strands during viral particle formation.

In various embodiments, vectors comprise modified 5′ LTR and/or 3′ LTRs. Either or both of the LTR may comprise one or more modifications including, but not limited to, one or more deletions, insertions, or substitutions. Modifications of the 3′ LTR are often made to improve the safety of lentiviral or retroviral systems by rendering viruses replication-defective. As used herein, the term “replication-defective” refers to virus that is not capable of complete, effective replication such that infective virions are not produced (e.g., replication-defective lentiviral progeny). The term “replication-competent” refers to wild-type virus or mutant virus that is capable of replication, such that viral replication of the virus is capable of producing infective virions (e.g., replication-competent lentiviral progeny).

“Self-inactivating” (SIN) vectors refers to replication-defective vectors, e.g., retroviral or lentiviral vectors, in which the right (3′) LTR enhancer-promoter region, known as the U3 region, has been modified (e.g., by deletion or substitution) to prevent viral transcription beyond the first round of viral replication. This is because the right (3′) LTR U3 region is used as a template for the left (5′) LTR U3 region during viral replication and, thus, the viral transcript cannot be made without the U3 enhancer-promoter. In a further embodiment, the 3′ LTR is modified such that the U5 region is replaced, for example, with an ideal poly(A) sequence. It should be noted that modifications to the LTRs such as modifications to the 3′ LTR, the 5′ LTR, or both 3′ and 5′ LTRs, are also included herein.

An additional safety enhancement is provided by replacing the U3 region of the 5′ LTR with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. Typical promoters are able to drive high levels of transcription in a Tat-independent manner. This replacement reduces the possibility of recombination to generate replication-competent virus because there is no complete U3 sequence in the virus production system. In certain embodiments, the heterologous promoter has additional advantages in controlling the manner in which the viral genome is transcribed. For example, the heterologous promoter can be inducible, such that transcription of all or part of the viral genome will occur only when the induction factors are present. Induction factors include, but are not limited to, one or more chemical compounds or the physiological conditions such as temperature or pH, in which the host cells are cultured.

In some embodiments, viral vectors comprise a TAR element. The term “TAR” refers to the “trans-activation response” genetic element located in the R region of lentiviral (e.g., HIV) LTRs. This element interacts with the lentiviral trans-activator (tat) genetic element to enhance viral replication. However, this element is not required in embodiments wherein the U3 region of the 5′ LTR is replaced by a heterologous promoter.

The “R region” refers to the region within retroviral LTRs beginning at the start of the capping group (i.e., the start of transcription) and ending immediately prior to the start of the poly A tract. The R region is also defined as being flanked by the U3 and U5 regions. The R region plays a role during reverse transcription in permitting the transfer of nascent DNA from one end of the genome to the other.

As used herein, the term “FLAP element” refers to a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou, et al., 2000, Cell, 101:173. During HIV-1 reverse transcription, central initiation of the plus-strand DNA at the central polypurine tract (cPPT) and central termination at the central termination sequence (CTS) lead to the formation of a three-stranded DNA structure: the HIV-1 central DNA flap. While not wishing to be bound by any theory, the DNA flap may act as a cis-active determinant of lentiviral genome nuclear import and/or may increase the titer of the virus. In particular embodiments, the retroviral or lentiviral vector backbones comprise one or more FLAP elements upstream or downstream of the heterologous genes of interest in the vectors. For example, in particular embodiments a transfer plasmid includes a FLAP element. In one embodiment, a vector comprises a FLAP element isolated from HIV-1.

In one embodiment, retroviral or lentiviral transfer vectors comprise one or more export elements. The term “export element” refers to a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J. Virol. 65: 1053; and Cullen et al., 1991. Cell 58: 423), and the hepatitis B virus post-transcriptional regulatory element (HPRE). Generally, the RNA export element is placed within the 3′ UTR of a gene, and can be inserted as one or multiple copies.

In particular embodiments, expression of heterologous sequences in viral vectors is increased by incorporating posttranscriptional regulatory elements, efficient polyadenylation sites, and optionally, transcription termination signals into the vectors. A variety of posttranscriptional regulatory elements can increase expression of a heterologous nucleic acid at the protein, e.g., woodchuck hepatitis virus posttranscriptional regulatory element (WPRE; Zufferey et al., 1999, J. Virol., 73:2886); the posttranscriptional regulatory element present in hepatitis B virus (HPRE) (Huang et al., Mol. Cell. Biol., 5:3864); and the like (Liu et al., 1995, Genes Dev., 9:1766). In particular embodiments, a vector can comprise a posttranscriptional regulatory element such as a WPRE or HPRE

In particular embodiments, vectors lack or do not comprise a posttranscriptional regulatory element (PTE) such as a WPRE or HPRE because in some instances these elements increase the risk of cellular transformation and/or do not substantially or significantly increase the amount of mRNA transcript or increase mRNA stability. Therefore, in some embodiments, vectors lack or do not comprise a PTE. In other embodiments, vectors lack or do not comprise a WPRE or HPRE as an added safety measure.

Elements directing the efficient termination and polyadenylation of the heterologous nucleic acid transcripts increases heterologous gene expression. Transcription termination signals are generally found downstream of the polyadenylation signal. In particular embodiments, vectors comprise a polyadenylation sequence 3′ of a polynucleotide encoding a polypeptide to be expressed. The term “polyA site” or “polyA sequence” as used herein denotes a DNA sequence which directs both the termination and polyadenylation of the nascent RNA transcript by RNA polymerase II. Polyadenylation sequences can promote mRNA stability by addition of a polyA tail to the 3′ end of the coding sequence and thus, contribute to increased translational efficiency. Efficient polyadenylation of the recombinant transcript is desirable as transcripts lacking a poly A tail are unstable and are rapidly degraded. Illustrative examples of polyA signals that can be used in a vector herein, include an ideal polyA sequence (e.g., AATAAA, ATTAAA, AGTAAA), a bovine growth hormone polyA sequence (BGHpA), a rabbit β-globin polyA sequence (rpgpA), or another suitable heterologous or endogenous polyA sequence known in the art.

In certain embodiments, a retroviral or lentiviral vector further comprises one or more insulator elements. Insulators elements may contribute to protecting lentivirus-expressed sequences, e.g., therapeutic polypeptides, from integration site effects, which may be mediated by cis-acting elements present in genomic DNA and lead to deregulated expression of transferred sequences (i.e., position effect; see, e.g., Burgess-Beusse et al., 2002, Proc. Natl. Acad. Sci., USA, 99:16433; and Zhan et al., 2001, Hum. Genet., 109:471). In some embodiments, transfer vectors comprise one or more insulator element the 3′ LTR and upon integration of the provirus into the host genome, the provirus comprises the one or more insulators at both the 5′ LTR or 3′ LTR, by virtue of duplicating the 3′ LTR. Suitable insulators for use herein include, but are not limited to, the chicken β-globin insulator (see Chung et al., 1993. Cell 74:505; Chung et al., 1997. PNAS 94:575; and Bell et al., 1999. Cell 98:387, incorporated by reference herein). Examples of insulator elements include, but are not limited to, an insulator from an β-globin locus, such as chicken HS4.

According to certain specific embodiments, most or all of the viral vector backbone sequences are derived from a lentivirus, e.g., HIV-1. However, it is to be understood that many different sources of retroviral and/or lentiviral sequences can be used, or combined and numerous substitutions and alterations in certain of the lentiviral sequences may be accommodated without impairing the ability of a transfer vector to perform the functions described herein. Moreover, a variety of lentiviral vectors are known in the art, see Naldini et al., (1996a, 1996b, and 1998); Zufferey et al., (1997); Dull et al., 1998, U.S. Pat. Nos. 6,013,516; and 5,994,136, many of which may be adapted to produce a viral vector or transfer plasmid of the present disclosure.

In various embodiments, a vector described herein can comprise a promoter operably linked to a polynucleotide encoding a CAR polypeptide. The vectors may have one or more LTRs, wherein either LTR comprises one or more modifications, such as one or more nucleotide substitutions, additions, or deletions. The vectors may further comprise one of more accessory elements to increase transduction efficiency (e.g., a cPPT/FLAP), viral packaging (e.g., a Psi (Ψ) packaging signal, RRE), and/or other elements that increase therapeutic gene expression (e.g., poly (A) sequences), and may optionally comprise a WPRE or HPRE.

In a particular embodiment, the transfer vector comprises a left (5′) retroviral LTR; a central polypurine tract/DNA flap (cPPT/FLAP); a retroviral export element; a promoter active in a T cell, operably linked to a polynucleotide encoding CAR polypeptide contemplated herein; and a right (3′) retroviral LTR; and optionally a WPRE or HPRE.

In a particular embodiment, the transfer vector comprises a left (5′) retroviral LTR; a retroviral export element; a promoter active in a T cell, operably linked to a polynucleotide encoding CAR polypeptide contemplated herein; a right (3′) retroviral LTR; and a poly (A) sequence; and optionally a WPRE or HPRE. In another particular embodiment, provided herein i a lentiviral vector comprising: a left (5′) LTR; a cPPT/FLAP; an RRE; a promoter active in a T cell, operably linked to a polynucleotide encoding CAR polypeptide contemplated herein; a right (3′) LTR; and a polyadenylation sequence; and optionally a WPRE or HPRE.

In a certain embodiment, provide herein is a lentiviral vector comprising: a left (5′) HIV-1 LTR; a Psi (Ψ) packaging signal; a cPPT/FLAP; an RRE; a promoter active in a T cell, operably linked to a polynucleotide encoding CAR polypeptide contemplated herein; a right (3′) self-inactivating (SIN) HIV-1 LTR; and a rabbit β-globin polyadenylation sequence; and optionally a WPRE or HPRE.

In another embodiment, provided herein is a vector comprising: at least one LTR; a central polypurine tract/DNA flap (cPPT/FLAP); a retroviral export element; and a promoter active in a T cell, operably linked to a polynucleotide encoding CAR polypeptide contemplated herein; and optionally a WPRE or HPRE.

In particular embodiment, provided herein is a vector comprising at least one LTR; a cPPT/FLAP; an RRE; a promoter active in a T cell, operably linked to a polynucleotide encoding CAR polypeptide contemplated herein; and a polyadenylation sequence; and optionally a WPRE or HPRE.

In a certain embodiment, provided herein is at least one SIN HIV-1 LTR; a Psi (Ψ) packaging signal; a cPPT/FLAP; an RRE; a promoter active in a T cell, operably linked to a polynucleotide encoding CAR polypeptide contemplated herein; and a rabbit β-globin polyadenylation sequence; and optionally a WPRE or HPRE.

In various embodiments, the vector is an integrating viral vector.

In various other embodiments, the vector is an episomal or non-integrating viral vector.

In various embodiments, vectors contemplated herein, comprise non-integrating or integration defective retrovirus. In one embodiment, an “integration defective” retrovirus or lentivirus refers to retrovirus or lentivirus having an integrase that lacks the capacity to integrate the viral genome into the genome of the host cells. In various embodiments, the integrase protein is mutated to specifically decrease its integrase activity. Integration-incompetent lentiviral vectors are obtained by modifying the pol gene encoding the integrase protein, resulting in a mutated pol gene encoding an integrative deficient integrase. Such integration-incompetent viral vectors have been described in patent application WO 2006/010834, which is herein incorporated by reference in its entirety.

Illustrative mutations in the HIV-1 pol gene suitable to reduce integrase activity include, but are not limited to: H12N, H12C, H16C, H16V, S81 R, D41A, K42A, H51A, Q53C, D55V, D64E, D64V, E69A, K71A, E85A, E87A, D116N, D1161, D116A, N120G, N1201, N120E, E152G, E152A, D35E, K156E, K156A, E157A, K159E, K159A, K160A, R166A, D167A, E170A, H171A, K173A, K186Q, K186T, K188T, E198A, R199c, R199T, R199A, D202A, K211A, Q214L, Q216L, Q221 L, W235F, W235E, K236S, K236A, K246A, G247 W, D253A, R262A, R263A and K264H.

Illustrative mutations in the HIV-1 pol gene suitable to reduce integrase activity include, but are not limited to: D64E, D64V, E92K, D116N, D1161, D116A, N120G, N1201, N120E, E152G, E152A, D35E, K156E, K156A, E157A, K159E, K159A, W235F, and W235E.

In a particular embodiment, an integrase comprises a mutation in one or more of amino acids, D64, D116 or E152. In one embodiment, an integrase comprises a mutation in the amino acids, D64, D116 and E152. In a particular embodiment, a defective HIV-1 integrase comprises a D64V mutation.

A “host cell” includes cells electroporated, transfected, infected, or transduced in vivo, ex vivo, or in vitro with a recombinant vector or a polynucleotide disclosed herein. Host cells may include packaging cells, producer cells, and cells infected with viral vectors. In particular embodiments, host cells infected with a viral vector disclosed herein are administered to a subject in need of therapy. In certain embodiments, the term “target cell” is used interchangeably with host cell and refers to transfected, infected, or transduced cells of a desired cell type. In particular embodiments, the target cell is a T cell.

Large scale viral particle production is often necessary to achieve a reasonable viral titer. Viral particles are produced by transfecting a transfer vector into a packaging cell line that comprises viral structural and/or accessory genes, e.g., gag, pol, env, tat, rev, vif, vpr, vpu, vpx, or nef genes or other retroviral genes.

As used herein, the term “packaging vector” refers to an expression vector or viral vector that lacks a packaging signal and comprises a polynucleotide encoding one, two, three, four or more viral structural and/or accessory genes. Typically, the packaging vectors are included in a packaging cell, and are introduced into the cell via transfection, transduction or infection. Methods for transfection, transduction or infection are well known by those of skill in the art. A retroviral/lentiviral transfer vector disclosed herein can be introduced into a packaging cell line, via transfection, transduction or infection, to generate a producer cell or cell line. The packaging vectors disclosed herein can be introduced into human cells or cell lines by standard methods including, e.g., calcium phosphate transfection, lipofection or electroporation. In some embodiments, the packaging vectors are introduced into the cells together with a dominant selectable marker, such as neomycin, hygromycin, puromycin, blastocidin, zeocin, thymidine kinase, DHFR, Gln synthetase or ADA, followed by selection in the presence of the appropriate drug and isolation of clones. A selectable marker gene can be linked physically to genes encoding by the packaging vector, e.g., by IRES or self-cleaving viral peptides.

Viral envelope proteins (env) determine the range of host cells which can ultimately be infected and transformed by recombinant retroviruses generated from the cell lines. In the case of lentiviruses, such as HIV-1, HIV-2, SIV, FIV and EIV, the env proteins include gp41 and gp120. Preferably, the viral env proteins expressed by packaging cells disclosed herein are encoded on a separate vector from the viral gag and pol genes, as has been previously described.

Illustrative examples of retroviral-derived env genes which can be employed herein include, but are not limited to: MLV envelopes, 10A1 envelope, BAEV, FeLV-B, RD 114, SSAV, Ebola, Sendai, FPV (Fowl plague virus), and influenza virus envelopes. Similarly, genes encoding envelopes from RNA viruses (e.g., RNA virus families of Picornaviridae, Calciviridae, Astroviridae, Togaviridae, Flaviviridae, Coronaviridae, Paramyxoviridae, Rhabdoviridae, Filoviridae, Orthomyxoviridae, Bunyaviridae, Arenaviridae, Reoviridae, Birnaviridae, Retroviridae) as well as from the DNA viruses (families of Hepadnaviridae, Circoviridae, Parvoviridae, Papovaviridae, Adenoviridae, Herpesviridae, Poxyiridae, and Iridoviridae) may be utilized. Representative examples include FeLV, VEE, HFVW, WDSV, SFV, Rabies, ALV, BIV, BLV, EBV, CAEV, SNV, ChTLV, STLV, MPMV, SMRV, RAV, FuSV, MH2, AEV, AMV, CT10, and EIAV.

In other embodiments, envelope proteins for pseudotyping a virus in connection with the present disclosure include, but are not limited to, any from the following viruses: Influenza A such as H1N1, H1N2, H3N2 and H5N1 (bird flu), Influenza B, Influenza C virus, Hepatitis A virus, Hepatitis B virus, Hepatitis C virus, Hepatitis D virus, Hepatitis E virus, Rotavirus, any virus of the Norwalk virus group, enteric adenoviruses, parvovirus, Dengue fever virus, Monkey pox, Mononegavirales, Lyssavirus such as rabies virus, Lagos bat virus, Mokola virus, Duvenhage virus, European bat virus 1 & 2 and Australian bat virus, Ephemerovirus, Vesiculovirus, Vesicular Stomatitis Virus (VSV), Herpesviruses such as Herpes simplex virus types 1 and 2, varicella zoster, cytomegalovirus, Epstein-Bar virus (EBV), human herpesviruses (HHV), human herpesvirus type 6 and 8, Human immunodeficiency virus (HIV), papilloma virus, murine gammaherpesvirus, Arenaviruses such as Argentine hemorrhagic fever virus, Bolivian hemorrhagic fever virus, Sabia-associated hemorrhagic fever virus, Venezuelan hemorrhagic fever virus, Lassa fever virus, Machupo virus, Lymphocytic choriomeningitis virus (LCMV), Bunyaviridiae such as Crimean-Congo hemorrhagic fever virus, Hantavirus, hemorrhagic fever with renal syndrome causing virus, Rift Valley fever virus, Filoviridae (filovirus) including Ebola hemorrhagic fever and Marburg hemorrhagic fever, Flaviviridae including Kaysanur Forest disease virus, Omsk hemorrhagic fever virus, Tick-borne encephalitis causing virus and Paramyxoviridae such as Hendra virus and Nipah virus, variola major and variola minor (smallpox), alphaviruses such as Venezuelan equine encephalitis virus, eastern equine encephalitis virus, western equine encephalitis virus, SARS-associated coronavirus (SARS-CoV), West Nile virus, and any encephalitis causing virus.

In one embodiment, provided herein are packaging cells which produce recombinant retrovirus, e.g., lentivirus, pseudotyped with the VSV-G glycoprotein.

The terms “pseudotype” or “pseudotyping” as used herein, refer to a virus whose viral envelope proteins have been substituted with those of another virus possessing preferable characteristics. For example, HIV can be pseudotyped with vesicular stomatitis virus G-protein (VSV-G) envelope proteins, which allows HIV to infect a wider range of cells because HIV envelope proteins (encoded by the env gene) normally target the virus to CD4+ presenting cells. In one embodiment, lentiviral envelope proteins are pseudotyped with VSV-G. In one embodiment, provided herein are packaging cells which produce recombinant retrovirus, e.g., lentivirus, pseudotyped with the VSV-G envelope glycoprotein.

As used herein, the term “packaging cell lines” is used in reference to cell lines that do not contain a packaging signal, but do stably or transiently express viral structural proteins and replication enzymes (e.g., gag, pol and env) which are necessary for the correct packaging of viral particles. Any suitable cell line can be employed to prepare packaging cells in connection with the present disclosure. Generally, the cells are mammalian cells. In a particular embodiment, the cells used to produce the packaging cell line are human cells. Suitable cell lines which can be used include, for example, CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells. In specific embodiments, the packaging cells are 293 cells, 293T cells, or A549 cells. In another specific embodiment, the cells are A549 cells.

As used herein, the term “producer cell line” refers to a cell line which is capable of producing recombinant retroviral particles, comprising a packaging cell line and a transfer vector construct comprising a packaging signal. The production of infectious viral particles and viral stock solutions may be carried out using conventional techniques. Methods of preparing viral stock solutions are known in the art and are illustrated by, e.g., Y. Soneoka et al. (1995) Nucl. Acids Res. 23:628-633, and N. R. Landau et al. (1992) J. Virol. 66:5110-5113. Infectious virus particles may be collected from the packaging cells using conventional techniques. For example, the infectious particles can be collected by cell lysis, or collection of the supernatant of the cell culture, as is known in the art. Optionally, the collected virus particles may be purified if desired. Suitable purification techniques are well known to those skilled in the art.

The delivery of a gene(s) or other polynucleotide sequence using a retroviral or lentiviral vector by means of viral infection rather than by transfection is referred to as “transduction.” In one embodiment, retroviral vectors are transduced into a cell through infection and provirus integration. In certain embodiments, a target cell, e.g., a T cell, is “transduced” if it comprises a gene or other polynucleotide sequence delivered to the cell by infection using a viral or retroviral vector. In particular embodiments, a transduced cell comprises one or more genes or other polynucleotide sequences delivered by a retroviral or lentiviral vector in its cellular genome.

In particular embodiments, host cells transduced with a viral vector as disclosed herein that expresses one or more polypeptides are administered to a subject to treat and/or prevent a B cell malignancy. Other methods relating to the use of viral vectors in gene therapy, which may be utilized according to certain embodiments herein, can be found in, e.g., Kay, M. A. (1997) Chest 111(6 Supp.):138S-142S; Ferry, N. and Heard, J. M. (1998) Hum. Gene Ther. 9:1975-81; Shiratory, Y. et al. (1999) Liver 19:265-74; Oka, K. et al. (2000) Curr. Opin. Lipidol. 11:179-86; Thule, P. M. and Liu, J. M. (2000) Gene Ther. 7:1744-52; Yang, N. S. (1992) Crit. Rev. Biotechnol. 12:335-56; Alt, M. (1995) J. Hepatol. 23:746-58; Brody, S. L. and Crystal, R. G. (1994) Ann. N.Y. Acad. Sci. 716:90-101; Strayer, D. S. (1999) Expert Opin. Investig. Drugs 8:2159-2172; Smith-Arica, J. R. and Bartlett, J. S. (2001) Curr. Cardiol. Rep. 3:43-49; and Lee, H. C. et al. (2000) Nature 408:483-8.

6.6. Genetically Modified Cells

In particular embodiments, disclosed herein are cells genetically modified to express the CARs contemplated herein, for use in the treatment of a tumor or a cancer. In particular embodiments, disclosed herein are cells genetically modified to express the CARs contemplated herein, for use in the treatment of B cell related conditions. As used herein, the term “genetically engineered” or “genetically modified” refers to the addition of extra genetic material in the form of DNA or RNA into the total genetic material in a cell. The terms, “genetically modified cells,” “modified cells,” and, “redirected cells,” are used interchangeably. As used herein, the term “gene therapy” refers to the introduction of extra genetic material in the form of DNA or RNA into the total genetic material in a cell that restores, corrects, or modifies expression of a gene, or for the purpose of expressing a therapeutic polypeptide, e.g., a CAR.

In particular embodiments, the CARs contemplated herein are introduced and expressed in immune effector cells so as to redirect their specificity to a target antigen of interest, e.g., a BCMA polypeptide. An “immune effector cell,” is any cell of the immune system that has one or more effector functions (e.g., cytotoxic cell killing activity, secretion of cytokines, induction of ADCC and/or CDC).

Immune effector cells of the present disclosure can be autologous/autogeneic (“self”) or non-autologous (“non-self,” e.g., allogeneic, syngeneic or xenogeneic).

“Autologous,” as used herein, refers to cells from the same subject.

“Allogeneic,” as used herein, refers to cells of the same species that differ genetically to the cell in comparison.

“Syngeneic,” as used herein, refers to cells of a different subject that are genetically identical to the cell in comparison.

“Xenogeneic,” as used herein, refers to cells of a different species to the cell in comparison. In certain embodiments, the cells of the present disclosure are allogeneic.

Illustrative immune effector cells used with the CARs contemplated herein include T lymphocytes. The terms “T cell” or “T lymphocyte” are art-recognized and are intended to include thymocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4⁺ T cell) CD4⁺ T cell, a cytotoxic T cell (CTL; CD8+ T cell), CD4⁺CD8⁺ T cell, CD4⁻CD8⁻ T cell, or any other subset of T cells. Other illustrative populations of T cells suitable for use in particular embodiments include naïve T cells and memory T cells.

As would be understood by the skilled person, other cells may also be used as immune effector cells with the CARs as described herein. In particular, immune effector cells also include NK cells, NKT cells, neutrophils, and macrophages. Immune effector cells also include progenitors of effector cells wherein such progenitor cells can be induced to differentiate into an immune effector cells in vivo or in vitro. Thus, in particular embodiments, immune effector cell includes progenitors of immune effectors cells such as hematopoietic stem cells (HSCs) contained within the CD34+ population of cells derived from cord blood, bone marrow or mobilized peripheral blood which upon administration in a subject differentiate into mature immune effector cells, or which can be induced in vitro to differentiate into mature immune effector cells.

As used herein, immune effector cells genetically engineered to contain a BCMA-specific CAR may be referred to as, “BCMA-specific redirected immune effector cells.”

The term, “CD34⁺ cell” as used herein refers to a cell expressing the CD34 protein on its cell surface. “CD34” as used herein refers to a cell surface glycoprotein (e.g., sialomucin protein) that often acts as a cell-cell adhesion factor and is involved in T cell entrance into lymph nodes. The CD34⁺ cell population contains hematopoietic stem cells (HSC), which upon administration to a patient differentiate and contribute to all hematopoietic lineages, including T cells, NK cells, NKT cells, neutrophils and cells of the monocyte/macrophage lineage.

In certain embodiments, provided herein are methods for making the immune effector cells which express the CAR contemplated herein. In one embodiment, the method comprises transfecting or transducing immune effector cells isolated from an individual such that the immune effector cells express one or more CAR as described herein. In certain embodiments, the immune effector cells are isolated from an individual and genetically modified without further manipulation in vitro. Such cells can then be directly re-administered into the individual. In further embodiments, the immune effector cells are first activated and stimulated to proliferate in vitro prior to being genetically modified to express a CAR. In this regard, the immune effector cells may be cultured before and/or after being genetically modified (i.e., transduced or transfected to express a CAR contemplated herein).

In particular embodiments, prior to in vitro manipulation or genetic modification of the immune effector cells described herein, the source of cells is obtained from a subject. In particular embodiments, the CAR-modified immune effector cells comprise T cells. T cells can be obtained from a number of sources including, but not limited to, peripheral blood mononuclear cells, bone marrow, lymph nodes tissue, cord blood, thymus issue, tissue from a site of infection, ascites, pleural effusion, spleen tissue, and tumors. In certain embodiments, T cells can be obtained from a unit of blood collected from a subject using any number of techniques known to the skilled person, such as sedimentation, e.g., FICOLL™ separation. In one embodiment, cells from the circulating blood of an individual are obtained by apheresis. The apheresis product typically contains lymphocytes, including T cells, monocytes, granulocyte, B cells, other nucleated white blood cells, red blood cells, and platelets. In one embodiment, the cells collected by apheresis may be washed to remove the plasma fraction and to place the cells in an appropriate buffer or media for subsequent processing. The cells can be washed with PBS or with another suitable solution that lacks calcium, magnesium, and most, if not all other, divalent cations. As would be appreciated by those of ordinary skill in the art, a washing step may be accomplished by methods known to those in the art, such as by using a semiautomated flowthrough centrifuge. For example, the Cobe 2991 cell processor, the Baxter CytoMate, or the like. After washing, the cells may be resuspended in a variety of biocompatible buffers or other saline solution with or without buffer. In certain embodiments, the undesirable components of the apheresis sample may be removed in the cell directly resuspended culture media.

In certain embodiments, T cells are isolated from peripheral blood mononuclear cells (PBMCs) by lysing the red blood cells and depleting the monocytes, for example, by centrifugation through a PERCOLL™ gradient. A specific subpopulation of T cells, expressing one or more of the following markers: CD3, CD28, CD4, CD8, CD45RA, and CD45RO, can be further isolated by positive or negative selection techniques. In one embodiment, a specific subpopulation of T cells, expressing CD3, CD28, CD4, CD8, CD45RA, and CD45RO is further isolated by positive or negative selection techniques. For example, enrichment of a T cell population by negative selection can be accomplished with a combination of antibodies directed to surface markers unique to the negatively selected cells. One method for use herein is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8. Flow cytometry and cell sorting may also be used to isolate cell populations of interest for use accordance with the present disclosure.

PBMC may be directly genetically modified to express CARs using methods contemplated herein. In certain embodiments, after isolation of PBMC, T lymphocytes are further isolated and in certain embodiments, both cytotoxic and helper T lymphocytes can be sorted into naïve, memory, and effector T cell subpopulations either before or after genetic modification and/or expansion.

CD8⁺ cells can be obtained by using standard methods. In some embodiments, CD8⁺ cells are further sorted into naïve, central memory, and effector cells by identifying cell surface antigens that are associated with each of those types of CD8⁺ cells.

In certain embodiments, naïve CD8+ T lymphocytes are characterized by the expression of phenotypic markers of naïve T cells including CD62L, CCR7, CD28, CD3, CD 127, and CD45RA.

In particular embodiments, memory T cells are present in both CD62L⁺ and CD62L⁻ subsets of CD8⁺ peripheral blood lymphocytes. PBMC are sorted into CD62L⁻CD8⁺ and CD62L⁺CD8⁺ fractions after staining with anti-CD8 and anti-CD62L antibodies. I n some embodiments, the expression of phenotypic markers of central memory T cells include CD45RO, CD62L, CCR7, CD28, CD3, and CD127 and are negative for granzyme B. In some embodiments, central memory T cells are CD45RO⁺, CD62L⁺, CD8⁺ T cells.

In some embodiments, effector T cells are negative for CD62L, CCR7, CD28, and CD127, and positive for granzyme B and perforin.

In certain embodiments, CD4⁺ T cells are further sorted into subpopulations. For example, CD4⁺ T helper cells can be sorted into naïve, central memory, and effector cells by identifying cell populations that have cell surface antigens. CD4⁺ lymphocytes can be obtained by standard methods. In some embodiments, naïve CD4⁺ T lymphocytes are CD45RO⁻, CD45RA⁺, CD62L⁺CD4⁺ T cell. In some embodiments, central memory CD4⁺ cells are CD62L positive and CD45RO positive. In some embodiments, effector CD4⁺ cells are CD62L and CD45RO negative.

The immune effector cells, such as T cells, can be genetically modified following isolation using known methods, or the immune effector cells can be activated and expanded (or differentiated in the case of progenitors) in vitro prior to being genetically modified. In a particular embodiment, the immune effector cells, such as T cells, are genetically modified with the chimeric antigen receptors contemplated herein (e.g., transduced with a viral vector comprising a nucleic acid encoding a CAR) and then are activated and expanded in vitro. In various embodiments, T cells can be activated and expanded before or after genetic modification to express a CAR, using methods as described, for example, in U.S. Pat. Nos. 6,352,694; 6,534,055; 6,905,680; 6,692,964; 5,858,358; 6,887,466; 6,905,681; 7,144,575; 7,067,318; 7, 172,869; 7,232,566; 7, 175,843; 5,883,223; 6,905,874; 6,797,514; 6,867,041; and U.S. Patent Application Publication No. 20060121005.

Generally, the T cells are expanded by contact with a surface having attached thereto an agent that stimulates a CD3 TCR complex associated signal and a ligand that stimulates a co-stimulatory molecule on the surface of the T cells. T cell populations may be stimulated by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. Co-stimulation of accessory molecules on the surface of T cells, is also contemplated.

In particular embodiments, PBMCs or isolated T cells are contacted with a stimulatory agent and costimulatory agent, such as anti-CD3 and anti-CD28 antibodies, generally attached to a bead or other surface, in a culture medium with appropriate cytokines, such as IL-2, IL-7, and/or IL-15. To stimulate proliferation of either CD4⁺ T cells or CD8⁺ T cells, an anti-CD3 antibody and an anti-CD28 antibody. Examples of an anti-CD28 antibody include 9.3, B-T3, XR-CD28 (Diacione, Besancon, France) can be used as can other methods commonly known in the art (Berg et al., Transplant Proc. 30(8):3975-3977, 1998; Haanen et al., J. Exp. Med. 190(9): 13191328, 1999; Garland et al., J. Immunol Meth. 227(1-2):53-63, 1999). Anti-CD3 and anti-CD28 antibodies attached to the same bead serve as a “surrogate” antigen presenting cell (APC). In other embodiments, the T cells may be activated and stimulated to proliferate with feeder cells and appropriate antibodies and cytokines using methods such as those described in U.S. Pat. Nos. 6,040,177; 5,827,642; and WO2012129514.

In other embodiments, artificial APC (aAPC) made by engineering K562, U937, 721.221, T2, and C1R cells to direct the stable expression and secretion, of a variety of co-stimulatory molecules and cytokines. In a particular embodiment K32 or U32 aAPCs are used to direct the display of one or more antibody-based stimulatory molecules on the AAPC cell surface. Expression of various combinations of genes on the aAPC enables the precise determination of human T-cell activation requirements, such that aAPCs can be tailored for the optimal propagation of T-cell subsets with specific growth requirements and distinct functions. The aAPCs support ex vivo growth and long-term expansion of functional human CD8 T cells without requiring the addition of exogenous cytokines, in contrast to the use of natural APCs. Populations of T cells can be expanded by aAPCs expressing a variety of costimulatory molecules including, but not limited to, CD137L (4-1BBL), CD134L (OX40L), and/or CD80 or CD86. Finally, the aAPCs provide an efficient platform to expand genetically modified T cells and to maintain CD28 expression on CD8 T cells. aAPCs provided in WO 03/057171 and US2003/0147869 are hereby incorporated by reference in their entirety.

In one embodiment, CD34⁺ cells are transduced with a nucleic acid construct in accordance with the present disclosure. In certain embodiments, the transduced CD34⁺ cells differentiate into mature immune effector cells in vivo following administration into a subject, generally the subject from whom the cells were originally isolated. In another embodiment, CD34⁺ cells may be stimulated in vitro prior to exposure to or after being genetically modified with a CAR as described herein, with one or more of the following cytokines: Flt-3 ligand (FLT3), stem cell factor (SCF), megakaryocyte growth and differentiation factor (TPO), IL-3 and IL-6 according to the methods described previously (Asheuer et al., 2004, PNAS 101(10):3557-3562; Imren, et al., 2004).

In certain embodiments, provided herein is a population of modified immune effector cells for the treatment of a tumor or a cancer, the modified immune effector cells comprising a CAR as disclosed herein. For example, a population of modified immune effector cells are prepared from peripheral blood mononuclear cells (PBMCs) obtained from a patient diagnosed with B cell malignancy described herein (autologous donors). The PBMCs form a heterogeneous population of T lymphocytes that can be CD4⁺, CD8⁺, or CD4⁺ and CD8⁺.

The PBMCs also can include other cytotoxic lymphocytes such as NK cells or NKT cells. An expression vector carrying the coding sequence of a CAR contemplated herein can be introduced into a population of human donor T cells, NK cells or NKT cells. Successfully transduced T cells that carry the expression vector can be sorted using flow cytometry to isolate CD3 positive T cells and then further propagated to increase the number of these CAR protein expressing T cells in addition to cell activation using anti-CD3 antibodies and or anti-CD28 antibodies and IL-2 or any other methods known in the art as described elsewhere herein. Standard procedures are used for cryopreservation of T cells expressing the CAR protein T cells for storage and/or preparation for use in a human subject. In one embodiment, the in vitro transduction, culture and/or expansion of T cells are performed in the absence of non-human animal derived products such as fetal calf serum and fetal bovine serum. Since a heterogeneous population of PBMCs is genetically modified, the resultant transduced cells are a heterogeneous population of modified cells comprising a CAR (e.g., a BCMA targeting CAR) as contemplated herein.

In a further embodiment, a mixture of, e.g., one, two, three, four, five or more, different expression vectors can be used in genetically modifying a donor population of immune effector cells wherein each vector encodes a different chimeric antigen receptor protein as contemplated herein. The resulting modified immune effector cells forms a mixed population of modified cells, with a proportion of the modified cells expressing more than one different CAR proteins.

In one embodiment, provided herein is a method of storing genetically modified murine, human or humanized CAR protein expressing immune effector cells which target a BCMA protein, comprising cryopreserving the immune effector cells such that the cells remain viable upon thawing. A fraction of the immune effector cells expressing the CAR proteins can be cryopreserved by methods known in the art to provide a permanent source of such cells for the future treatment of patients afflicted with a a tumor or a cancer or the B cell related condition. When needed, the cryopreserved transformed immune effector cells can be thawed, grown and expanded for more such cells.

As used herein, “cryopreserving,” refers to the preservation of cells by cooling to sub-zero temperatures, such as (typically) 77 K or −196° C. (the boiling point of liquid nitrogen). Cryoprotective agents are often used at sub-zero temperatures to prevent the cells being preserved from damage due to freezing at low temperatures or warming to room temperature. Cryopreservative agents and optimal cooling rates can protect against cell injury. Cryoprotective agents which can be used include but are not limited to dimethyl sulfoxide (DMSO) (Lovelock and Bishop, Nature, 1959; 183: 1394-1395; Ashwood-Smith, Nature, 1961; 190: 1204-1205), glycerol, polyvinylpyrrolidone (Rinfret, Ann. N.Y. Acad. Sci., 1960; 85: 576), and polyethylene glycol (Sloviter and Ravdin, Nature, 1962; 196: 48). The preferred cooling rate is 1° to 3° C./minute. After at least two hours, the T cells have reached a temperature of −80° C. and can be placed directly into liquid nitrogen (−196° C.) for permanent storage such as in a long-term cryogenic storage vessel.

6.7. T Cell Manufacturing Process

The T cells manufactured by the methods contemplated herein provide improved adoptive immunotherapy compositions. Without wishing to be bound to any particular theory, it is believed that the T cell compositions manufactured by the methods contemplated herein are imbued with superior properties, including increased survival, expansion in the relative absence of differentiation, and persistence in vivo. In one embodiment, a method of manufacturing T cells comprises contacting the cells with one or more agents that modulate a PI3K cell signaling pathway. In one embodiment, a method of manufacturing T cells comprises contacting the cells with one or more agents that modulate a PI3K/Akt/mTOR cell signaling pathway. In various embodiments, the T cells may be obtained from any source and contacted with the agent during the activation and/or expansion phases of the manufacturing process. The resulting T cell compositions are enriched in developmentally potent T cells that have the ability to proliferate and express one or more of the following biomarkers: CD62L, CCR7, CD28, CD27, CD122, CD127, CD197, and CD38. In one embodiment, populations of cell comprising T cells, that have been treated with one or more PI3K inhibitors is enriched for a population of CD8+ T cells co-expressing one or more or, or all of, the following biomarkers: CD62L, CD127, CD197, and CD38.

In one embodiment, modified T cells comprising maintained levels of proliferation and decreased differentiation are manufactured. In a particular embodiment, T cells are manufactured by stimulating T cells to become activated and to proliferate in the presence of one or more stimulatory signals and an agent that is an inhibitor of a PI3K cell signaling pathway.

The T cells can then be modified to express CARs (e.g., BCMA targeting CARs). In one embodiment, the T cells are modified by transducing the T cells with a viral vector comprising a CAR (e.g., an anti-BCMA CAR) contemplated herein. In a certain embodiment, the T cells are modified prior to stimulation and activation in the presence of an inhibitor of a PI3K cell signaling pathway. In another embodiment, T cells are modified after stimulation and activation in the presence of an inhibitor of a PI3K cell signaling pathway. In a particular embodiment, T cells are modified within 12 hours, 24 hours, 36 hours, or 48 hours of stimulation and activation in the presence of an inhibitor of a PI3K cell signaling pathway.

After T cells are activated, the cells are cultured to proliferate. T cells may be cultured for at least 1, 2, 3, 4, 5, 6, or 7 days, at least 2 weeks, at least 1, 2, 3, 4, 5, or 6 months or more with 1, 2, 3, 4, 5, 6, 7, 8, 9, or 10 or more rounds of expansion.

In various embodiments, T cell compositions are manufactured in the presence of one or more inhibitors of the PI3K pathway. The inhibitors may target one or more activities in the pathway or a single activity. Without wishing to be bound to any particular theory, it is contemplated that treatment or contacting T cells with one or more inhibitors of the PI3K pathway during the stimulation, activation, and/or expansion phases of the manufacturing process preferentially increases young T cells, thereby producing superior therapeutic T cell compositions.

In a particular embodiment, a method for increasing the proliferation of T cells expressing an engineered T cell receptor is provided. Such methods may comprise, for example, harvesting a source of T cells from a subject, stimulating and activating the T cells in the presence of one or more inhibitors of the PI3K pathway, modification of the T cells to express a CAR (e.g., an anti-BCMA CAR, more particularly an anti-BCMA02 CAR), and expanding the T cells in culture.

In a certain embodiment, a method for producing populations of T cells enriched for expression of one or more of the following biomarkers: CD62L, CCR7, CD28, CD27, CD122, CD127, CD197, and CD38. In one embodiment, young T cells comprise one or more of, or all of the following biological markers: CD62L, CD127, CD197, and CD38. In one embodiment, the young T cells lack expression of CD57, CD244, CD160, PD-1, CTLA4, TIM3, and LAG3 are provided. As discussed elsewhere herein, the expression levels young T cell biomarkers is relative to the expression levels of such markers in more differentiated T cells or immune effector cell populations.

In one embodiment, peripheral blood mononuclear cells (PBMCs) are used as the source of T cells in the T cell manufacturing methods contemplated herein. PBMCs form a heterogeneous population of T lymphocytes that can be CD4⁺, CD8⁺, or CD4⁺ and CD8⁺ and can include other mononuclear cells such as monocytes, B cells, NK cells and NKT cells. An expression vector comprising a polynucleotide encoding an engineered TCR or CAR contemplated herein can be introduced into a population of human donor T cells, NK cells or NKT cells. Successfully transduced T cells that carry the expression vector can be sorted using flow cytometry to isolate CD3 positive T cells and then further propagated to increase the number of the modified T cells in addition to cell activation using anti-CD3 antibodies and or anti-CD28 antibodies and IL-2, IL-7, and/or IL-15 or any other methods known in the art as described elsewhere herein.

Manufacturing methods contemplated herein may further comprise cryopreservation of modified T cells for storage and/or preparation for use in a human subject. T cells are cryopreserved such that the cells remain viable upon thawing. When needed, the cryopreserved transformed immune effector cells can be thawed, grown and expanded for more such cells. As used herein, “cryopreserving,” refers to the preservation of cells by cooling to sub-zero temperatures, such as (typically) 77 K or −196° C. (the boiling point of liquid nitrogen). Cryoprotective agents are often used at sub-zero temperatures to prevent the cells being preserved from damage due to freezing at low temperatures or warming to room temperature. Cryopreservative agents and optimal cooling rates can protect against cell injury. Cryoprotective agents which can be used include but are not limited to dimethyl sulfoxide (DMSO) (Lovelock and Bishop, Nature, 1959; 183: 1394-1395; Ashwood-Smith, Nature, 1961; 190: 1204-1205), glycerol, polyvinylpyrrolidone (Rinfret, Ann. N.Y. Acad. Sci., 1960; 85: 576), and polyethylene glycol (Sloviter and Ravdin, Nature, 1962; 196: 48). The preferred cooling rate is 1° to 3° C./minute. After at least two hours, the T cells have reached a temperature of −80° C. and can be placed directly into liquid nitrogen (−196° C.) for permanent storage such as in a long-term cryogenic storage vessel.

6.8. T Cells

The present disclosure contemplates the manufacture of improved CAR T cell compositions. T cells used for CAR T cell production may be autologous/autogeneic (“self”) or non-autologous (“non-self,” e.g., allogeneic, syngeneic or xenogeneic). In certain embodiments, the T cells are obtained from a mammalian subject. In a more specific embodiment, the T cells are obtained from a primate subject. In a preferred embodiment, the T cells are obtained from a human subject.

T cells can be obtained from a number of sources including, but not limited to, peripheral blood mononuclear cells, bone marrow, lymph nodes tissue, cord blood, thymus issue, tissue from a site of infection, ascites, pleural effusion, spleen tissue, and tumors. In certain embodiments, T cells can be obtained from a unit of blood collected from a subject using any number of techniques known to the skilled person, such as sedimentation, e.g., FICOLL™ separation. In one embodiment, cells from the circulating blood of an individual are obtained by apheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing. The cells can be washed with PBS or with another suitable solution that lacks calcium, magnesium, and most, if not all other, divalent cations. As would be appreciated by those of ordinary skill in the art, a washing step may be accomplished by methods known to those in the art, such as by using a semiautomated flowthrough centrifuge. For example, the Cobe 2991 cell processor, the Baxter CytoMate, or the like. After washing, the cells may be resuspended in a variety of biocompatible buffers or other saline solution with or without buffer. In certain embodiments, the undesirable components of the apheresis sample may be removed in the cell directly resuspended culture media.

In particular embodiments, a population of cells comprising T cells, e.g., PBMCs, is used in the manufacturing methods contemplated herein. In other embodiments, an isolated or purified population of T cells is used in the manufacturing methods contemplated herein. Cells can be isolated from peripheral blood mononuclear cells (PBMCs) by lysing the red blood cells and depleting the monocytes, for example, by centrifugation through a PERCOLL™ gradient. In some embodiments, after isolation of PBMC, both cytotoxic and helper T lymphocytes can be sorted into naïve, memory, and effector T cell subpopulations either before or after activation, expansion, and/or genetic modification.

A specific subpopulation of T cells, expressing one or more of the following markers: CD3, CD4, CD8, CD28, CD45RA, CD45RO, CD62, CD127, and HLA-DR can be further isolated by positive or negative selection techniques. In one embodiment, a specific subpopulation of T cells, expressing one or more of the markers selected from the group consisting of (i) CD62L, CCR7, CD28, CD27, CD122, CD127, CD197; or (ii) CD38 or CD62L, CD127, CD197, and CD38, is further isolated by positive or negative selection techniques. In various embodiments, the manufactured T cell compositions do not express or do not substantially express one or more of the following markers: CD57, CD244, CD160, PD-1, CTLA4, TIM3, and LAG3.

In one embodiment, expression of one or more of the markers selected from the group consisting of CD62L, CD127, CD197, and CD38 is increased at least 1.5 fold, at least 2 fold, at least 3 fold, at least 4 fold, at least 5 fold, at least 6 fold, at least 7 fold, at least 8 fold, at least 9 fold, at least 10 fold, at least 25 fold, or more compared to a population of T cells activated and expanded without a PI3K inhibitor.

In one embodiment, expression of one or more of the markers selected from the group consisting of CD57, CD244, CD160, PD-1, CTLA4, TIM3, and LAG3 is decreased at least 1.5 fold, at least 2 fold, at least 3 fold, at least 4 fold, at least 5 fold, at least 6 fold, at least 7 fold, at least 8 fold, at least 9 fold, at least 10 fold, at least 25 fold, or more compared to a population of T cells activated and expanded with a PI3K inhibitor.

In one embodiment, the manufacturing methods contemplated herein increase the number CAR T cells comprising one or more markers of naïve or developmentally potent T cells. Without wishing to be bound to any particular theory, the present inventors believe that treating a population of cells comprising T cells with one or more PI3K inhibitors results in an increase an expansion of developmentally potent T cells and provides a more robust and efficacious adoptive CAR T cell immunotherapy compared to existing CAR T cell therapies.

Illustrative examples of markers of naïve or developmentally potent T cells increased in T cells manufactured using the methods contemplated herein include, but are not limited to CD62L, CD127, CD197, and CD38. In particular embodiments, naïve T cells do not express do not express or do not substantially express one or more of the following markers: CD57, CD244, CD160, PD-1, BTLA, CD45RA, CTLA4, TIM3, and LAG3.

With respect to T cells, the T cell populations resulting from the various expansion methodologies contemplated herein may have a variety of specific phenotypic properties, depending on the conditions employed. In various embodiments, expanded T cell populations comprise one or more of the following phenotypic markers: CD62L, CD127, CD197, CD38, and HLA-DR.

In one embodiment, such phenotypic markers include enhanced expression of one or more of, or all of CD62L, CD127, CD197, and CD38. In particular embodiments, CD8+ T lymphocytes characterized by the expression of phenotypic markers of naïve T cells including CD62L, CD127, CD197, and CD38 are expanded.

In particular embodiments, T cells characterized by the expression of phenotypic markers of central memory T cells including CD45RO, CD62L, CD127, CD197, and CD38 and negative for granzyme B are expanded. In some embodiments, the central memory T cells are CD45RO⁺, CD62L⁺, CD8⁺ T cells.

In certain embodiments, CD4⁺ T lymphocytes characterized by the expression of phenotypic markers of naïve CD4+ cells including CD62L and negative for expression of CD45RA and/or CD45RO are expanded. In some embodiments, CD4⁺ cells characterized by the expression of phenotypic markers of central memory CD4⁺ cells including CD62L and CD45RO positive. In some embodiments, effector CD4⁺ cells are CD62L positive and CD45RO negative.

In certain embodiments, the T cells are isolated from an individual and activated and stimulated to proliferate in vitro prior to being genetically modified to express a CAR (e.g., an anti-BCMA CAR). In this regard, the T cells may be cultured before and/or after being genetically modified (i.e., transduced or transfected to express a CAR, e.g., an anti-BCMA CAR contemplated herein).

6.8.1. Activation and Expansion

In order to achieve sufficient therapeutic doses of T cell compositions, T cells are often subject to one or more rounds of stimulation, activation and/or expansion. T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety. T cells modified to express a CAR (e.g., an anti-BCMA CAR) can be activated and expanded before and/or after the T cells are modified. In addition, T cells may be contacted with one or more agents that modulate the PI3K cell signaling pathway before, during, and/or after activation and/or expansion. In one embodiment, T cells manufactured by the methods contemplated herein undergo one, two, three, four, or five or more rounds of activation and expansion, each of which may include one or more agents that modulate the PI3K cell signaling pathway.

In one embodiment, a costimulatory ligand is presented on an antigen presenting cell (e.g., an aAPC, dendritic cell, B cell, and the like) that specifically binds a cognate costimulatory molecule on a T cell, thereby providing a signal which, in addition to the primary signal provided by, for instance, binding of a TCR/CD3 complex, mediates a desired T cell response. Suitable costimulatory ligands include, but are not limited to, CD7, B7-1 (CD80), B7-2 (CD86), PD-L 1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, ILT3, ILT4, an agonist or antibody that binds Toll ligand receptor, and a ligand that specifically binds with B7-H3.

In a particular embodiment, a costimulatory ligand comprises an antibody or antigen binding fragment thereof that specifically binds to a costimulatory molecule present on a T cell, including but not limited to, CD27, CD28, 4-IBB, OX40, CD30, CD40, PD-1, 1COS, lymphocyte function-associated antigen 1 (LFA-1), CD7, LIGHT, NKG2C, B7-H3, and a ligand that specifically binds with CD83.

Suitable costimulatory ligands further include target antigens, which may be provided in soluble form or expressed on APCs or aAPCs that bind engineered TCRs or CARs expressed on modified T cells.

In various embodiments, a method for manufacturing T cells contemplated herein comprises activating a population of cells comprising T cells and expanding the population of T cells. T cell activation can be accomplished by providing a primary stimulation signal through the T cell TCR/CD3 complex or via stimulation of the CD2 surface protein and by providing a secondary costimulation signal through an accessory molecule, e.g., CD28.

The TCR/CD3 complex may be stimulated by contacting the T cell with a suitable CD3 binding agent, e.g., a CD3 ligand or an anti-CD3 monoclonal antibody. Illustrative examples of CD3 antibodies include, but are not limited to, OKT3, G19-4, BC3, and 64.1.

In another embodiment, a CD2 binding agent may be used to provide a primary stimulation signal to the T cells. Illustrative examples of CD2 binding agents include, but are not limited to, CD2 ligands and anti-CD2 antibodies, e.g., the T11.3 antibody in combination with the T11.1 or T11.2 antibody (Meuer, S. C. et al. (1984) Cell 36:897-906) and the 9.6 antibody (which recognizes the same epitope as TI 1.1) in combination with the 9-1 antibody (Yang, S. Y. et al. (1986) J. Immunol. 137:1097-1100). Other antibodies which bind to the same epitopes as any of the above described antibodies can also be used. Additional antibodies, or combinations of antibodies, can be prepared and identified by standard techniques as disclosed elsewhere herein.

In addition to the primary stimulation signal provided through the TCR/CD3 complex, or via CD2, induction of T cell responses requires a second, costimulatory signal. In particular embodiments, a CD28 binding agent can be used to provide a costimulatory signal. Illustrative examples of CD28 binding agents include but are not limited to: natural CD 28 ligands, e.g., a natural ligand for CD28 (e.g., a member of the B7 family of proteins, such as B7-1 (CD80) and B7-2 (CD86); and anti-CD28 monoclonal antibody or fragment thereof capable of crosslinking the CD28 molecule, e.g., monoclonal antibodies 9.3, B-T3, XR-CD28, KOLT-2, 15E8, 248.23.2, and EX5.3D10.

In one embodiment, the molecule providing the primary stimulation signal, for example a molecule which provides stimulation through the TCR/CD3 complex or CD2, and the costimulatory molecule are coupled to the same surface.

In certain embodiments, binding agents that provide stimulatory and costimulatory signals are localized on the surface of a cell. This can be accomplished by transfecting or transducing a cell with a nucleic acid encoding the binding agent in a form suitable for its expression on the cell surface or alternatively by coupling a binding agent to the cell surface.

In another embodiment, the molecule providing the primary stimulation signal, for example a molecule which provides stimulation through the TCR/CD3 complex or CD2, and the costimulatory molecule are displayed on antigen presenting cells.

In one embodiment, the molecule providing the primary stimulation signal, for example a molecule which provides stimulation through the TCR/CD3 complex or CD2, and the costimulatory molecule are provided on separate surfaces.

In a certain embodiment, one of the binding agents that provide stimulatory and costimulatory signals is soluble (provided in solution) and the other agent(s) is provided on one or more surfaces.

In a particular embodiment, the binding agents that provide stimulatory and costimulatory signals are both provided in a soluble form (provided in solution).

In various embodiments, the methods for manufacturing T cells contemplated herein comprise activating T cells with anti-CD3 and anti-CD28 antibodies.

T cell compositions manufactured by the methods contemplated herein comprise T cells activated and/or expanded in the presence of one or more agents that inhibit a PI3K cell signaling pathway. T cells modified to express a CAR (e.g., an anti-BCMA CAR) can be activated and expanded before and/or after the T cells are modified. In particular embodiments, a population of T cells is activated, modified to express a CAR (e.g., an anti-BCMA CAR), and then cultured for expansion.

In one embodiment, T cells manufactured by the methods contemplated herein comprise an increased number of T cells expressing markers indicative of high proliferative potential and the ability to self-renew but that do not express or express substantially undetectable markers of T cell differentiation. These T cells may be repeatedly activated and expanded in a robust fashion and thereby provide an improved therapeutic T cell composition.

In one embodiment, a population of T cells activated and expanded in the presence of one or more agents that inhibit a PI3K cell signaling pathway is expanded at least 1.5 fold, at least 2 fold, at least 3 fold, at least 4 fold, at least 5 fold, at least 6 fold, at least 7 fold, at least 8 fold, at least 9 fold, at least 10 fold, at least 25 fold, at least 50 fold, at least 100 fold, at least 250 fold, at least 500 fold, at least 1000 fold, or more compared to a population of T cells activated and expanded without a PI3K inhibitor.

In one embodiment, a population of T cells characterized by the expression of markers young T cells are activated and expanded in the presence of one or more agents that inhibit a PI3K cell signaling pathway is expanded at least 1.5 fold, at least 2 fold, at least 3 fold, at least 4 fold, at least 5 fold, at least 6 fold, at least 7 fold, at least 8 fold, at least 9 fold, at least 10 fold, at least 25 fold, at least 50 fold, at least 100 fold, at least 250 fold, at least 500 fold, at least 1000 fold, or more compared the population of T cells activated and expanded without a PI3K inhibitor.

In one embodiment, expanding T cells activated by the methods contemplated herein further comprises culturing a population of cells comprising T cells for several hours (about 3 hours) to about 7 days to about 28 days or any hourly integer value in between. In another embodiment, the T cell composition may be cultured for 14 days. In a particular embodiment, T cells are cultured for about 21 days. In another embodiment, the T cell compositions are cultured for about 2-3 days. Several cycles of stimulation/activation/expansion may also be desired such that culture time of T cells can be 60 days or more.

In particular embodiments, conditions appropriate for T cell culture include an appropriate media (e.g., Minimal Essential Media or RPMI Media 1640 or, X-vivo 15, (Lonza)) and one or more factors necessary for proliferation and viability including, but not limited to serum (e.g., fetal bovine or human serum), interleukin-2 (IL-2), insulin, IFN-7, IL-4, IL-7, IL-21, GM-CSF, IL-10, IL-12, IL-15, TGFβ, and TNF-α or any other additives suitable for the growth of cells known to the skilled artisan.

Further illustrative examples of cell culture media include, but are not limited to RPMI 1640, Clicks, AIM-V, DMEM, MEM, a-MEM, F-12, X-Vivo 1 5, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells.

Illustrative examples of other additives for T cell expansion include, but are not limited to, surfactant, piasmanate, pH buffers such as HEPES, and reducing agents such as N-acetyl-cysteine and 2-mercaptoethanol.

Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% C02).

In particular embodiments, PBMCs or isolated T cells are contacted with a stimulatory agent and costimulatory agent, such as anti-CD3 and anti-CD28 antibodies, generally attached to a bead or other surface, in a culture medium with appropriate cytokines, such as IL-2, IL-7, and/or IL-15.

In other embodiments, artificial APC (aAPC) may be made by engineering K562, U937, 721.221, T2, and C1R cells to direct the stable expression and secretion, of a variety of costimulatory molecules and cytokines. In a particular embodiment K32 or U32 aAPCs are used to direct the display of one or more antibody-based stimulatory molecules on the AAPC cell surface. Populations of T cells can be expanded by aAPCs expressing a variety of costimulatory molecules including, but not limited to, CD137L (4-1BBL), CD134L (OX40L), and/or CD80 or CD86. Finally, the aAPCs provide an efficient platform to expand genetically modified T cells and to maintain CD28 expression on CD8 T cells. aAPCs provided in WO 03/057171 and US2003/0147869 are hereby incorporated by reference in their entirety.

6.8.2. Agents

In various embodiments, a method for manufacturing T cells is provided that expands undifferentiated or developmentally potent T cells comprising contacting T cells with an agent that modulates a PI3K pathway in the cells. In various embodiments, a method for manufacturing T cells is provided that expands undifferentiated or developmentally potent T cells comprising contacting T cells with an agent that modulates a PI3K/AKT/mTOR pathway in the cells. The cells may be contacted prior to, during, and/or after activation and expansion. The T cell compositions retain sufficient T cell potency such that they may undergo multiple rounds of expansion without a substantial increase in differentiation.

As used herein, the terms “modulate,” “modulator,” or “modulatory agent” or comparable term refer to an agent's ability to elicit a change in a cell signaling pathway. A modulator may increase or decrease an amount, activity of a pathway component or increase or decrease a desired effect or output of a cell signaling pathway. In one embodiment, the modulator is an inhibitor. In another embodiment, the modulator is an activator.

An “agent” refers to a compound, small molecule, e.g., small organic molecule, nucleic acid, polypeptide, or a fragment, isoform, variant, analog, or derivative thereof used in the modulation of a PI3K/AKT/mTOR pathway.

A “small molecule” refers to a composition that has a molecular weight of less than about 5 kD, less than about 4 kD, less than about 3 kD, less than about 2 kD, less than about 1 kD, or less than about 0.5 kD. Small molecules may comprise nucleic acids, peptides, polypeptides, peptidomimetics, peptoids, carbohydrates, lipids, components thereof or other organic or inorganic molecules. Libraries of chemical and/or biological mixtures, such as fungal, bacterial, or algal extracts, are known in the art and can be screened with any of the assays of the present disclosure. Methods for the synthesis of molecular libraries are known in the art (see, e.g., Carell et al., 1994a; Carell et al., 1994b; Cho et al., 1993; DeWitt et al., 1993; Gallop et al., 1994; Zuckermann et al., 1994).

An “analog” refers to a small organic compound, a nucleotide, a protein, or a polypeptide that possesses similar or identical activity or function(s) as the compound, nucleotide, protein or polypeptide or compound having the desired activity of the present disclosure, but need not necessarily comprise a sequence or structure that is similar or identical to the sequence or structure of a preferred embodiment.

A “derivative” refers to either a compound, a protein or polypeptide that comprises an amino acid sequence of a parent protein or polypeptide that has been altered by the introduction of amino acid residue substitutions, deletions or additions, or a nucleic acid or nucleotide that has been modified by either introduction of nucleotide substitutions or deletions, additions or mutations. The derivative nucleic acid, nucleotide, protein or polypeptide possesses a similar or identical function as the parent polypeptide.

In various embodiments, the agent that modulates a PI3K pathway activates a component of the pathway. An “activator,” or “agonist” refers to an agent that promotes, increases, or induces one or more activities of a molecule in a PI3K/AKT/mTOR pathway including, without limitation, a molecule that inhibits one or more activities of a PI3K.

In various embodiments, the agent that modulates a PI3K pathway inhibits a component of the pathway. An “inhibitor” or “antagonist” refers to an agent that inhibits, decreases, or reduces one or more activities of a molecule in a PI3K pathway including, without limitation, a PI3K. In one embodiment, the inhibitor is a dual molecule inhibitor. In particular embodiment, the inhibitor may inhibit a class of molecules have the same or substantially similar activities (a pan-inhibitor) or may specifically inhibit a molecule's activity (a selective or specific inhibitor). Inhibition may also be irreversible or reversible.

In one embodiment, the inhibitor has an IC50 of at least lnM, at least 2 nM, at least 5 nM, at least 10 nM, at least 50 nM, at least 100 nM, at least 200 nM, at least 500 nM, at least 1 μM, at least 10 μM, at least 50 μM, or at least 100 M. IC50 determinations can be accomplished using any conventional techniques known in the art. For example, an IC50 can be determined by measuring the activity of a given enzyme in the presence of a range of concentrations of the inhibitor under study. The experimentally obtained values of enzyme activity then are plotted against the inhibitor concentrations used. The concentration of the inhibitor that shows 50% enzyme activity (as compared to the activity in the absence of any inhibitor) is taken as the “IC50” value. Analogously, other inhibitory concentrations can be defined through appropriate determinations of activity.

In various embodiments, T cells are contacted or treated or cultured with one or more modulators of a PI3K pathway at a concentration of at least 1 nM, at least 2 nM, at least 5 nM, at least 10 nM, at least 50 nM, at least 100 nM, at least 200 nM, at least 500 nM, at least 1 μM, at least 10 μM, at least 50 μM, at least 100 μM, or at least 1 M.

In particular embodiments, T cells may be contacted or treated or cultured with one or more modulators of a PI3K pathway for at least 12 hours, 18 hours, at least 1, 2, 3, 4, 5, 6, or 7 days, at least 2 weeks, at least 1, 2, 3, 4, 5, or 6 months or more with 1, 2, 3, 4, 5, 6, 7, 8, 9, or 10 or more rounds of expansion.

6.8.3. PI3K/Akt/mTOR Pathway

The phosphatidyl-inositol-3 kinase/Akt/mammalian target of rapamycin pathway serves as a conduit to integrate growth factor signaling with cellular proliferation, differentiation, metabolism, and survival. PI3Ks are a family of highly conserved intracellular lipid kinases. Class IA PI3Ks are activated by growth factor receptor tyrosine kinases (RTKs), either directly or through interaction with the insulin receptor substrate family of adaptor molecules. This activity results in the production of phosphatidyl-inositol-3,4,5-trisphospate (PIP3) a regulator of the serine/threonine kinase Akt. mTOR acts through the canonical PI3K pathway via 2 distinct complexes, each characterized by different binding partners that confer distinct activities. mTORC1 (mTOR in complex with PRAS40, raptor, and mLST8/GbL) acts as a downstream effector of PI3K/Akt signaling, linking growth factor signals with protein translation, cell growth, proliferation, and survival. mTORC2 (mTOR in complex with rictor, mSIN1, protor, and mLST8) acts as an upstream activator of Akt.

Upon growth factor receptor-mediated activation of PI3K, Akt is recruited to the membrane through the interaction of its pleckstrin homology domain with PIP3, thus exposing its activation loop and enabling phosphorylation at threonine 308 (Thr308) by the constitutively active phosphoinositide-dependent protein kinase 1 (PDK1). For maximal activation, Akt is also phosphorylated by mTORC2, at serine 473 (Ser473) of its C-terminal hydrophobic motif. DNA-PK and HSP have also been shown to be important in the regulation of Akt activity. Akt activates mTORC1 through inhibitory phosphorylation of TSC2, which along with TSC1, negatively regulates mTORC1 by inhibiting the Rheb GTPase, a positive regulator of mTORC1. mTORC1 has 2 well-defined substrates, p70S6K (referred to hereafter as S6K1) and 4E-BP1, both of which critically regulate protein synthesis. Thus, mTORC1 is an important downstream effector of PI3K, linking growth factor signaling with protein translation and cellular proliferation.

6.8.4. PI3K Inhibitors

As used herein, the term “PI3K inhibitor” refers to a nucleic acid, peptide, compound, or small organic molecule that binds to and inhibits at least one activity of PI3K. The PI3K proteins can be divided into three classes, class 1 PI3Ks, class 2 PI3Ks, and class 3 PI3Ks. Class 1 PI3Ks exist as heterodimers consisting of one of four p110 catalytic subunits (p110α, p110β, p110δ, and p110γ) and one of two families of regulatory subunits. In a particular embodiment, a PI3K inhibitor of the present disclosure targets the class 1 PI3K inhibitors. In one embodiment, a PI3K inhibitor will display selectivity for one or more isoforms of the class 1 PI3K inhibitors (i.e., selectivity for p110α, p110β, p110δ, and p110γ or one or more of p110α, p110β, p110δ, and p110γ). In another aspect, a PI3K inhibitor will not display isoform selectivity and be considered a “pan-PI3K inhibitor.” In one embodiment, a PI3K inhibitor will compete for binding with ATP to the PI3K catalytic domain.

In certain embodiments, a PI3K inhibitor can, for example, target PI3K as well as additional proteins in the PI3K-AKT-mTOR pathway. In particular embodiments, a PI3K inhibitor that targets both mTOR and PI3K can be referred to as either an mTOR inhibitor or a PI3K inhibitor. A PI3K inhibitor that only targets PI3K can be referred to as a selective PI3K inhibitor. In one embodiment, a selective PI3K inhibitor can be understood to refer to an agent that exhibits a 50% inhibitory concentration with respect to PI3K that is at least 10-fold, at least 20-fold, at least 30-fold, at least 50-fold, at least 100-fold, at least 1000-fold, or more, lower than the inhibitor's IC50 with respect to mTOR and/or other proteins in the pathway.

In a particular embodiment, exemplary PI3K inhibitors inhibit PI3K with an IC50 (concentration that inhibits 50% of the activity) of about 200 nM or less, preferably about 100 nm or less, even more preferably about 60 nM or less, about 25 nM, about 10 nM, about 5 nM, about 1 nM, 100 μM, 50 μM, 25 μM, 10 μM, 1 μM, or less. In one embodiment, a PI3K inhibitor inhibits PI3K with an IC50 from about 2 nM to about 100 nm, more preferably from about 2 nM to about 50 nM, even more preferably from about 2 nM to about 15 nM.

Illustrative examples of PI3K inhibitors suitable for use in the T cell manufacturing methods contemplated herein include, but are not limited to, BKM120 (class 1 PI3K inhibitor, Novartis), XL147 (class 1 PI3K inhibitor, Exelixis), (pan-PI3K inhibitor, GlaxoSmithKline), and PX-866 (class 1 PI3K inhibitor; p110α, p110β, and p110γ isoforms, Oncothyreon).

Other illustrative examples of selective PI3K inhibitors include, but are not limited to BYL719, GSK2636771, TGX-221, AS25242, CAL-101, ZSTK474, and IPI-145.

Further illustrative examples of pan-PI3K inhibitors include, but are not limited to BEZ235, LY294002, GSK1059615, TG100713, and GDC-0941.

6.8.5. AKT Inhibitors

As used herein, the term “AKT inhibitor” refers to a nucleic acid, peptide, compound, or small organic molecule that inhibits at least one activity of AKT. AKT inhibitors can be grouped into several classes, including lipid-based inhibitors (e.g., inhibitors that target the pleckstrin homology domain of AKT which prevents AKT from localizing to plasma membranes), ATP-competitive inhibitors, and allosteric inhibitors. In one embodiment, AKT inhibitors act by binding to the AKT catalytic site. In a particular embodiment, Akt inhibitors act by inhibiting phosphorylation of downstream AKT targets such as mTOR. In another embodiment, AKT activity is inhibited by inhibiting the input signals to activate Akt by inhibiting, for example, DNA-PK activation of AKT, PDK-1 activation of AKT, and/or mTORC2 activation of Akt.

AKT inhibitors can target all three AKT isoforms, AKT1, AKT2, AKT3 or may be isoform selective and target only one or two of the AKT isoforms. In one embodiment, an AKT inhibitor can target AKT as well as additional proteins in the PI3K-AKT-mTOR pathway. An AKT inhibitor that only targets AKT can be referred to as a selective AKT inhibitor. In one embodiment, a selective AKT inhibitor can be understood to refer to an agent that exhibits a 50% inhibitory concentration with respect to AKT that is at least 10-fold, at least 20-fold, at least 30-fold, at least 50-fold, at least 100-fold, at least 1000-fold, or more lower than the inhibitor's IC50 with respect to other proteins in the pathway.

In a particular embodiment, exemplary AKT inhibitors inhibit AKT with an IC50 (concentration that inhibits 50% of the activity) of about 200 nM or less, preferably about 100 nm or less, even more preferably about 60 nM or less, about 25 nM, about 10 nM, about 5 nM, about 1 nM, 100 μM, 50 μM, 25 μM, 10 μM, 1 μM, or less. In one embodiment, an AKT inhibits AKT with an IC50 from about 2 nM to about 100 nm, more preferably from about 2 nM to about 50 nM, even more preferably from about 2 nM to about 15 nM.

Illustrative examples of AKT inhibitors for use in combination with auristatin based antibody-drug conjugates include, for example, perifosine (Keryx), MK2206 (Merck), VQD-002 (VioQuest), XL418 (Exelixis), GSK690693, GDC-0068, and PX316 (PROLX Pharmaceuticals).

An illustrative, non-limiting example of a selective Akt1 inhibitor is A-674563.

An illustrative, non-limiting example of a selective Akt2 inhibitor is CCT128930.

In particular embodiments, the Akt inhibitor DNA-PK activation of Akt, PDK-1 activation of Akt, mTORC2 activation of Akt, or HSP activation of Akt.

Illustrative examples of DNA-PK inhibitors include, but are not limited to, NU7441, PI-103, NU7026, PIK-75, and PP-121.

6.8.6. mTOR Inhibitors

The terms “mTOR inhibitor” or “agent that inhibits mTOR” refers to a nucleic acid, peptide, compound, or small organic molecule that inhibits at least one activity of an mTOR protein, such as, for example, the serine/threonine protein kinase activity on at least one of its substrates (e.g., p70S6 kinase 1, 4E-BP1, AKT/PKB and eEF2). mTOR inhibitors are able to bind directly to and inhibit mTORC1, mTORC2 or both mTORC1 and mTORC2.

Inhibition of mTORC1 and/or mTORC2 activity can be determined by a reduction in signal transduction of the PI3K/Akt/mTOR pathway. A wide variety of readouts can be utilized to establish a reduction of the output of such signaling pathway. Some non-limiting exemplary readouts include (1) a decrease in phosphorylation of Akt at residues, including but not limited to 5473 and T308; (2) a decrease in activation of Akt as evidenced, for example, by a reduction of phosphorylation of Akt substrates including but not limited to Fox01/O3a T24/32, GSK3α/β; S21/9, and TSC2 T1462; (3) a decrease in phosphorylation of signaling molecules downstream of mTOR, including but not limited to ribosomal S6 S240/244, 70S6K T389, and 4EBP1 T37/46; and (4) inhibition of proliferation of cancerous cells.

In one embodiment, the mTOR inhibitors are active site inhibitors. These are mTOR inhibitors that bind to the ATP binding site (also referred to as ATP binding pocket) of mTOR and inhibit the catalytic activity of both mTORC1 and mTORC2. One class of active site inhibitors suitable for use in the T cell manufacturing methods contemplated herein are dual specificity inhibitors that target and directly inhibit both PI3K and mTOR. Dual specificity inhibitors bind to both the ATP binding site of mTOR and PI3K. Illustrative examples of such inhibitors include, but are not limited to: imidazoquinazolines, wortmannin, LY294002, PI-103 (Cayman Chemical), SF1126 (Semafore), BGT226 (Novartis), XL765 (Exelixis) and NVP-BEZ235 (Novartis).

Another class of mTOR active site inhibitors suitable for use in the methods contemplated herein selectively inhibit mTORC1 and mTORC2 activity relative to one or more type I phosphatidylinositol 3-kinases, e.g., PI3 kinase α, β, γ, or δ. These active site inhibitors bind to the active site of mTOR but not PI3K. Illustrative examples of such inhibitors include, but are not limited to: pyrazolopyrimidines, Torin1 (Guertin and Sabatini), PP242 (2-(4-Amino-1-isopropyl-1H-pyrazolo[3,4-d]pyrimidin-3-yl)-1H-indol-5-ol), PP30, Ku-0063794, WAY-600 (Wyeth), WAY-687 (Wyeth), WAY-354 (Wyeth), and AZD8055 (Liu et al., Nature Review, 8, 627-644, 2009).

In one embodiment, a selective mTOR inhibitor refers to an agent that exhibits a 50% inhibitory concentration (IC50) with respect to mTORC1 and/or mTORC2, that is at least 10-fold, at least 20-fold, at least 50-fold, at least 100-fold, at least 1000-fold, or more, lower than the inhibitor's IC50 with respect to one, two, three, or more type I PI3-kinases or to all of the type I PI3-kinases.

Another class of mTOR inhibitors for use in the present disclosure is referred to herein as “rapalogs.” As used herein the term “rapalogs” refers to compounds that specifically bind to the mTOR FRB domain (FKBP rapamycin binding domain), are structurally related to rapamycin, and retain the mTOR inhibiting properties. The term rapalogs excludes rapamycin. Rapalogs include esters, ethers, oximes, hydrazones, and hydroxylamines of rapamycin, as well as compounds in which functional groups on the rapamycin core structure have been modified, for example, by reduction or oxidation. Pharmaceutically acceptable salts of such compounds are also considered to be rapamycin derivatives. Illustrative examples of rapalogs suitable for use in the methods contemplated herein include, without limitation, temsirolimus (CC1779), everolimus (RAD001), deforolimus (AP23573), AZD8055 (AstraZeneca), and OSI-027 (OSI).

In one embodiment, the agent is the mTOR inhibitor rapamycin (sirolimus).

In a particular embodiment, exemplary mTOR inhibitors for use herein inhibit either mTORC1, mTORC2 or both mTORC1 and mTORC2 with an IC50 (concentration that inhibits 50% of the activity) of about 200 nM or less, preferably about 100 nm or less, even more preferably about 60 nM or less, about 25 nM, about 10 nM, about 5 nM, about 1 nM, 100 μM, 50 M, 25 μM, 10 μM, 1 μM, or less. In one aspect, a mTOR inhibitor for use herein inhibits either mTORC1, mTORC2 or both mTORC1 and mTORC2 with an IC50 from about 2 nM to about 100 nm, more preferably from about 2 nM to about 50 nM, even more preferably from about 2 nM to about 15 nM.

In one embodiment, exemplary mTOR inhibitors inhibit either PI3K and mTORC1 or mTORC2 or both mTORC1 and mTORC2 and PI3K with an IC50 (concentration that inhibits 50% of the activity) of about 200 nM or less, preferably about 100 nm or less, even more preferably about 60 nM or less, about 25 nM, about 10 nM, about 5 nM, about 1 nM, 100 μM, 50 μM, 25 μM, 10 μM, 1 μM, or less. In one aspect, a mTOR inhibitor for use herein inhibits PI3K and mTORC1 or mTORC2 or both mTORC1 and mTORC2 and PI3K with an IC50 from about 2 nM to about 100 nm, more preferably from about 2 nM to about 50 nM, even more preferably from about 2 nM to about 15 nM.

Further illustrative examples of mTOR inhibitors suitable for use in particular embodiments contemplated herein include, but are not limited to AZD8055, INK128, rapamycin, PF-04691502, and everolimus.

mTOR has been shown to demonstrate a robust and specific catalytic activity toward the physiological substrate proteins, p70 S6 ribosomal protein kinase I (p70S6K1) and eIF4E binding protein 1 (4EBP1) as measured by phosphor-specific antibodies in Western blotting.

In one embodiment, the inhibitor of the PI3K/AKT/mTOR pathway is a s6 kinase inhibitor selected from the group consisting of: BI-D1870, H89, PF-4708671, FMK, and AT7867.

6.9. Compositions and Formulations

The compositions contemplated herein may comprise one or more polypeptides, polynucleotides, vectors comprising same, genetically modified immune effector cells, etc., as contemplated herein. Compositions include, but are not limited to pharmaceutical compositions. A “pharmaceutical composition” refers to a composition formulated in pharmaceutically-acceptable or physiologically-acceptable solutions for administration to a cell or an animal, either alone, or in combination with one or more other modalities of therapy. It will also be understood that, if desired, the compositions of the present disclosure may be administered in combination with other agents as well, such as, e.g., cytokines, growth factors, hormones, small molecules, chemotherapeutics, pro-drugs, drugs, antibodies, or other various pharmaceutically-active agents. There is virtually no limit to other components that may also be included in the compositions, provided that the additional agents do not adversely affect the ability of the composition to deliver the intended therapy.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

In particular embodiments, compositions presented herein comprise an amount of CAR-expressing immune effector cells contemplated herein. As used herein, the term “amount” refers to “an amount effective” or “an effective amount” of a genetically modified therapeutic cell, e.g., T cell, to achieve a beneficial or desired prophylactic or therapeutic result, including clinical results.

A “prophylactically effective amount” refers to an amount of a genetically modified therapeutic cell effective to achieve the desired prophylactic result. Typically but not necessarily, since a prophylactic dose is used in subjects prior to or at an earlier stage of disease, the prophylactically effective amount is less than the therapeutically effective amount.

A “therapeutically effective amount” of a genetically modified therapeutic cell may vary according to factors such as the disease state, age, sex, and weight of the individual, and the ability of the stem and progenitor cells to elicit a desired response in the individual. A therapeutically effective amount is also one in which any toxic or detrimental effects of the virus or transduced therapeutic cells are outweighed by the therapeutically beneficial effects. The term “therapeutically effective amount” includes an amount that is effective to “treat” a subject (e.g., a patient). When a therapeutic amount is indicated, the precise amount of a compositions of the present disclosure to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10² to 10¹⁰ cells/kg body weight, preferably 10⁵ to 10⁶ cells/kg body weight, including all integer values within those ranges. The number of cells will depend upon the ultimate use for which the composition is intended as will the type of cells included therein. For uses provided herein, the cells are generally in a volume of a liter or less, can be 500 mL or less, even 250 mL or 100 mL or less. Hence the density of the desired cells is typically greater than 10⁶ cells/ml and generally is greater than 10⁷ cells/ml, generally 10⁸ cells/ml or greater. The clinically relevant number of immune cells can be apportioned into multiple infusions that cumulatively equal or exceed 10⁵, 10⁶, 10⁷, 10⁸, 10⁹, 10¹⁰, 10¹¹, or 10¹² cells. In some aspects, particularly since all the infused cells will be redirected to a particular target antigen (e.g., x or X light chain), lower numbers of cells, in the range of 10⁶/kilogram (10⁶-10¹¹ per patient) may be administered. CAR expressing cell compositions may be administered multiple times at dosages within these ranges. The cells may be allogeneic, syngeneic, xenogeneic, or autologous to the patient undergoing therapy. If desired, the treatment may also include administration of mitogens (e.g., PHA) or lymphokines, cytokines, and/or chemokines (e.g., IFN-γ, IL-2, IL-12, TNF-alpha, IL-18, and TNF-beta, GM-CSF, IL-4, IL-13, Flt3-L, RANTES, MIP1α, etc.) as described herein to enhance induction of the immune response.

Generally, compositions comprising the cells activated and expanded as described herein may be utilized in the treatment and prevention of diseases that arise in individuals who are immunocompromised. In particular, compositions comprising the CAR-modified T cells contemplated herein are used in the treatment of a tumor or a cancer, or in the treatment of B cell malignancies. The CAR-modified T cells of the present disclosure may be administered either alone, or as a pharmaceutical composition in combination with carriers, diluents, excipients, and/or with other components such as IL-2 or other cytokines or cell populations. In particular embodiments, pharmaceutical compositions contemplated herein comprise an amount of genetically modified T cells, in combination with one or more pharmaceutically or physiologically acceptable carriers, diluents or excipients.

Pharmaceutical compositions of the present disclosure comprising a CAR-expressing immune effector cell population, such as T cells, may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. In certain aspects, compositions of the present disclosure are formulated for parenteral administration, e.g., intravascular (intravenous or intraarterial), intraperitoneal or intramuscular administration.

The liquid pharmaceutical compositions, whether they be solutions, suspensions or other like form, may include one or more of the following: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile.

In a particular embodiment, compositions contemplated herein comprise an effective amount of CAR-expressing immune effector cells, alone or in combination with one or more therapeutic agents. Thus, the CAR-expressing immune effector cell compositions may be administered alone or in combination with other known cancer treatments, such as radiation therapy, chemotherapy, transplantation, immunotherapy, hormone therapy, photodynamic therapy, etc. The compositions may also be administered in combination with antibiotics. Such therapeutic agents may be accepted in the art as a standard treatment for a particular disease state as described herein, such as a particular cancer. Exemplary therapeutic agents contemplated include cytokines, growth factors, steroids, NSAIDs, DMARDs, anti-inflammatories, chemotherapeutics, radiotherapeutics, therapeutic antibodies, or other active and ancillary agents.

In certain embodiments, compositions comprising CAR-expressing immune effector cells disclosed herein may be administered to a subject in conjunction with any number of chemotherapeutic, e.g., anti-cancer, agents. In certain embodiments, a chemotherapeutic, e.g., anti-cancer, agent, is administered to a subject after the administration of a CAR T cell therapy, e.g., BCMA CAR T cell therapy, if certain conditions, described elsewhere herein, occur that indicate the CAR T cell therapy will not be therapeutically beneficial to the subject. Illustrative examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine resume; nitrogen mustards such as chlorambucil, chlornaphazine, cholophosphamide, estramustine, ifosfamide, mechlorethamine, mechlorethamine oxide hydrochloride, melphalan (e.g., melphalan hydrochloride), novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2, 2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERE®, Rhone-Poulenc Rohrer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylomithine (DMFO); retinoic acid derivatives such as Targretin™ (bexarotene), Panretin™ (alitretinoin); ONTAK™ (denileukin diftitox); esperamicins; capecitabine; and pharmaceutically acceptable salts, acids or derivatives of any of the above. Also included in this definition are anti-hormonal agents that act to regulate or inhibit hormone action on cancers such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In certain embodiments, compositions comprising CAR-expressing immune effector cells (e.g., immune cells expressing a chimeric antigen receptor (CAR) directed to BCMA (BCMA CAR T cells), e.g., idecabtagene vicleucel (ide-cel) cells) disclosed herein may be administered to a subject in conjunction with lenalidomide as a maintenance therapy after administration of compositions comprising CAR-expressing immune effector cells. In certain embodiments, the lenalidomide may be administered immediately after administration of the compositions comprising CAR-expressing immune effector cells. In certain embodiments, the lenalidomide may be administered 1 week, 2 weeks, 3 weeks, or 4 weeks after administration of the compositions comprising CAR-expressing immune effector cells. In certain embodiments, the lenalidomide may be administered 1 month, 2 months, 3 months, 4 months, 5 months, 6 months, 7 months, 8 months, 9 months, 10 months, 11 months, or 12 months after administration of the compositions comprising CAR-expressing immune effector cells. In certain embodiments, the lenalidomide may be administered at a dosage of about 2.5 mg, 5 mg, 10 mg, 15 mg, 20 mg, or 25 mg. In certain embodiments, the lenalidomide may be administered at a dosage of about 2.5 mg, 5 mg, 10 mg, 15 mg, 20 mg, or 25 mg once daily. In certain embodiments, the lenalidomide may be administered at a dosage of about 25 mg once daily orally on Days 1-21 of repeated 28-day cycles. In certain embodiments, the lenalidomide may be administered at a dosage of about 25 mg once daily orally on Days 1-21 of repeated 28-day cycles to a subject for treating Multiple Myeloma (MM). In certain embodiments, the lenalidomide may be administered at a dosage of about 10 mg once daily continuously on Days 1-28 of repeated 28-day cycles. In certain embodiments, the lenalidomide may be administered at a dosage of about 2.5 mg once daily. In certain embodiments, the lenalidomide may be administered at a dosage of about 5 mg once daily. In certain embodiments, the lenalidomide may be administered at a dosage of about 10 mg once daily. In certain embodiments, the lenalidomide may be administered at a dosage of about 15 mg every other day. In certain embodiments, the lenalidomide may be administered at a dosage of about 25 mg once daily orally on Days 1-21 of repeated 28-day cycles. In certain embodiments, the lenalidomide may be administered at a dosage of about 20 mg once daily orally on Days 1-21 of repeated 28-day cycles for up to 12 cycles. In a certain embodiment, lenalidomide maintenance therapy is recommended for all patients. In a certain embodiment, lenalidomide maintenance therapy should be initiated upon adequate bone marrow recovery or from 90-day post-ide-cel infusion, whichever is later.

In certain embodiments, compositions comprising CAR-expressing immune effector cells (e.g., immune cells expressing a chimeric antigen receptor (CAR) directed to BCMA (BCMA CAR T cells), e.g., idecabtagene vicleucel (ide-cel) cells) disclosed herein may be administered to a subject in conjunction with pomalidomide as a maintenance therapy after administration of compositions comprising CAR-expressing immune effector cells. In certain embodiments, the pomalidomide may be administered immediately after administration of the compositions comprising CAR-expressing immune effector cells. In certain embodiments, the pomalidomide may be administered 1 week, 2 weeks, 3 weeks, or 4 weeks after administration of the compositions comprising CAR-expressing immune effector cells. In certain embodiments, the pomalidomide may be administered 1 month, 2 months, 3 months, 4 months, 5 months, 6 months, 7 months, 8 months, 9 months, 10 months, 11 months, or 12 months after administration of the compositions comprising CAR-expressing immune effector cells. In certain embodiments, the pomalidomide may be administered at a dosage of about 1 mg, 2 mg, 3 mg, or 4 mg. In certain embodiments, the pomalidomide may be administered at a dosage of about 1 mg, 2 mg, 3 mg, or 4 mg once daily. In certain embodiments, the pomalidomide may be administered at a dosage of about 4 mg per day taken orally on days 1-21 of repeated 28-day cycles until disease progression. In certain embodiments, the pomalidomide may be administered at a dosage of about 4 mg per day taken orally on days 1-21 of repeated 28-day cycles until disease progression to a subject for treating Multiple Myeloma (MM). In a certain embodiment, pomalidomide maintenance therapy is recommended for all patients. In a certain embodiment, pomalidomide maintenance therapy should be initiated upon adequate bone marrow recovery or from 90-day post-ide-cel infusion, whichever is later.

In certain embodiments, compositions comprising CAR-expressing immune effector cells (e.g., immune cells expressing a chimeric antigen receptor (CAR) directed to BCMA (BCMA CAR T cells), e.g., idecabtagene vicleucel (ide-cel) cells) disclosed herein may be administered to a subject in conjunction with CC-220 (iberdomide) as a maintenance therapy after administration of compositions comprising CAR-expressing immune effector cells. In certain embodiments, the CC-220 may be administered immediately after administration of the compositions comprising CAR-expressing immune effector cells. In certain embodiments, the CC-220 may be administered 1 week, 2 weeks, 3 weeks, or 4 weeks after administration of the compositions comprising CAR-expressing immune effector cells. In certain embodiments, the CC-220 may be administered 1 month, 2 months, 3 months, 4 months, 5 months, 6 months, 7 months, 8 months, 9 months, 10 months, 11 months, or 12 months after administration of the compositions comprising CAR-expressing immune effector cells. In certain embodiments, the CC-220 may be administered at a dosage of about 0.15 mg, 0.3 mg, 0.45 mg, 0.6 mg, 0.75 mg, 0.9 mg, 1.0 mg, 1.1 mg, or 1.2 mg. In certain embodiments, the CC-220 may be administered orally. In certain embodiments, the CC-220 may be administered orally at a dosage of about 0.15 mg, 0.3 mg, 0.45 mg, 0.6 mg, 0.75 mg, 0.9 mg, 1.0 mg, 1.1 mg, or 1.2 mg daily for 21 days of a 28-day cycle, e.g., daily on days 1-21 of a 28-day cycle, with the 28-day cycles repeated as needed. In certain embodiments, the CC-220 may be administered to a subject for treating Multiple Myeloma (MM). In a certain embodiment, CC-220 maintenance therapy is recommended for all patients. In a certain embodiment, the CC-220 maintenance therapy should be initiated upon adequate bone marrow recovery or from 90-day post-ide-cel infusion, whichever is later.

In certain embodiments, compositions comprising CAR-expressing immune effector cells (e.g., immune cells expressing a chimeric antigen receptor (CAR) directed to BCMA (BCMA CAR T cells), e.g., idecabtagene vicleucel (ide-cel) cells) disclosed herein may be administered to a subject in conjunction with CC-220 (iberdomide) and dexamethasone as a maintenance therapy after administration of compositions comprising CAR-expressing immune effector cells. In certain embodiments, the CC-220 and dexamethasone may be administered immediately after administration of the compositions comprising CAR-expressing immune effector cells. In certain embodiments, the CC-220 may be administered immediately after administration of the compositions comprising CAR-expressing immune effector cells. In certain embodiments, the dexamethasone may be administered immediately after administration of the compositions comprising CAR-expressing immune effector cells. In certain embodiments, the CC-220 and dexamethasone may be administered 1 week, 2 weeks, 3 weeks, or 4 weeks after administration of the compositions comprising CAR-expressing immune effector cells. In certain embodiments, the CC-220 may be administered 1 week, 2 weeks, 3 weeks, or 4 weeks after administration of the compositions comprising CAR-expressing immune effector cells. In certain embodiments, the dexamethasone may be administered 1 week, 2 weeks, 3 weeks, or 4 weeks after administration of the compositions comprising CAR-expressing immune effector cells. In certain embodiments, the CC-220 and dexamethasone may be administered 1 month, 2 months, 3 months, 4 months, 5 months, 6 months, 7 months, 8 months, 9 months, 10 months, 11 months, or 12 months after administration of the compositions comprising CAR-expressing immune effector cells. In certain embodiments, the CC-220 may be administered 1 month, 2 months, 3 months, 4 months, 5 months, 6 months, 7 months, 8 months, 9 months, 10 months, 11 months, or 12 months after administration of the compositions comprising CAR-expressing immune effector cells. In certain embodiments, the dexamethasone may be administered 1 month, 2 months, 3 months, 4 months, 5 months, 6 months, 7 months, 8 months, 9 months, 10 months, 11 months, or 12 months after administration of the compositions comprising CAR-expressing immune effector cells. In certain embodiments, the CC-220 may be administered at a dosage of about 0.15 mg, 0.3 mg, 0.45 mg, 0.6 mg, 0.75 mg, 0.9 mg, 1.0 mg, 1.1 mg, or 1.2 mg. In certain embodiments, the dexamethasone may be administered at a dosage of about 20 mg, 25 mg, 30 mg, 35 mg, 40 mg, 45 mg, 50 mg, 55 mg, or 60 mg. In certain embodiments, the dexamethasone may be administered at a dosage of about 40 mg. In certain embodiments, the CC-220 may be administered orally. In certain embodiments, the CC-220 may be administered orally at a dosage of about 15 mg, 0.3 mg, 0.45 mg, 0.6 mg, 0.75 mg, 0.9 mg, 1.0 mg, 1.1 mg, or 1.2 mg daily for 21 days of a 28-day cycle, e.g., daily on days 1-21 of a 28-day cycle, with the 28-day cycles repeated as needed. In certain embodiments, the dexamethasone may be administered orally. In certain embodiments, the dexamethasone may be administered at a dose of about 20-60 mgs. In certain embodiments, the dexamethasone may be administered orally at a dosage of about 20 mg, 25 mg, 30 mg, 35 mg, 40 mg, 45 mg, 50 mg, 55 mg, or 60 mg on days 1, 8, 15, and 22 of a 28-day cycle, with the 28-day cycles repeated as needed. In certain embodiments, the CC-220 may be administered orally at a dosage of about 15 mg, 0.3 mg, 0.45 mg, 0.6 mg, 0.75 mg, 0.9 mg, 1.0 mg, 1.1 mg, or 1.2 mg daily for 21 days of a 28-day cycle, e.g., daily on days 1-21 of a 28-day cycle, with the 28-day cycles repeated as needed, and the dexamethasone may be administered orally at a dosage of about 20 mg, 25 mg, 30 mg, 35 mg, 40 mg, 45 mg, 50 mg, 55 mg, or 60 mg on days 1, 8, 15, and 22 of a 28-day cycle, with the 28-day cycles repeated as needed. In certain embodiments, the CC-220 and dexamethasone may be administered to a subject for treating Multiple Myeloma (MM). In a certain embodiment, CC-220 and dexamethasone maintenance therapy is recommended for all patients. In a certain embodiment, the CC-220 and dexamethasone maintenance therapy should be initiated upon adequate bone marrow recovery or from 90-day post-ide-cel infusion, whichever is later.

A variety of other therapeutic agents may be used in conjunction with the compositions described herein. In one embodiment, the composition comprising CAR-expressing immune effector cells is administered with an anti-inflammatory agent. Anti-inflammatory agents or drugs include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate.

Other exemplary NSAIDs are chosen from the group consisting of ibuprofen, naproxen, naproxen sodium, Cox-2 inhibitors such as VIOXX® (rofecoxib) and CELEBREX® (celecoxib), and sialylates. Exemplary analgesics are chosen from the group consisting of acetaminophen, oxycodone, tramadol, and propoxyphene hydrochloride. Exemplary glucocorticoids are chosen from the group consisting of cortisone, dexamethasone, hydrocortisone, methylprednisolone, prednisolone, and prednisone. Exemplary biological response modifiers include molecules directed against cell surface markers (e.g., CD4, CD5, etc.), cytokine inhibitors, such as the TNF antagonists (e.g., etanercept (ENBREL®), adalimumab (HUMIRA®) and infliximab (REMICADE®), chemokine inhibitors and adhesion molecule inhibitors. The biological response modifiers include monoclonal antibodies as well as recombinant forms of molecules. Exemplary DMARDs include azathioprine, cyclophosphamide, cyclosporine, methotrexate, penicillamine, leflunomide, sulfasalazine, hydroxychloroquine, Gold (oral (auranofin) and intramuscular) and minocycline.

Illustrative examples of therapeutic antibodies suitable for combination with the CAR modified T cells contemplated herein, include, but are not limited to, bavituximab, bevacizumab (avastin), bivatuzumab, blinatumomab, conatumumab, daratumumab, duligotumab, dacetuzumab, dalotuzumab, elotuzumab (HuLuc63), gemtuzumab, ibritumomab, indatuximab, inotuzumab, lorvotuzumab, lucatumumab, milatuzumab, moxetumomab, ocaratuzumab, ofatumumab, rituximab, siltuximab, teprotumumab, and ublituximab.

Antibodies against PD-1 or, PD-L1 and/or CTLA-4 may be used in combination with the CAR T cells disclosed herein, e.g., BCMA CAR T cells, e.g., CAR T cells expressing a chimeric antigen receptor comprising a BCMA-2 single chain Fv fragment, e.g., idecabtagene vicleucel cells. In particular embodiments, the PD-1 antibody is selected from the group consisting of: nivolumab, pembrolizumab, and pidilizumab. In particular embodiments, the PD-L1 antibody is selected from the group consisting of: atezolizumab, avelumab, durvalumab, and BMS-986559. In particular embodiments, the CTLA-4 antibody is selected from the group consisting of: ipilimumab and tremelimumab.

In certain embodiments, the compositions described herein are administered in conjunction with a cytokine. By “cytokine” as used herein is meant a generic term for proteins released by one cell population that act on another cell as intercellular mediators. Examples of such cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, IL-21, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines.

In certain embodiments, the compositions described herein are administered in conjunction with a therapy to treat Cytokine Release Syndrome (CRS). CRS is a systemic inflammatory immune response that can occur after administration of certain biologic therapeutics, e.g., chimeric antigen receptor-expressing T cells or NK cells (CAR T cells or CAR NK cells), e.g., BCMA CAR T cells. CRS can be distinguished from cytokine storm, a condition with a similar clinical phenotype and biomarker signature, as follows. In CRS, T cells become activated upon recognition of a tumor antigen, while in cytokine storm, the immune system is activated independently of tumor targeting; in CRS, IL-6 is a key mediator, and thus symptoms may be relieved using an anti-IL-6 or anti-IL-6 receptor (IL-6R) inhibitor, while in cytokine storm, Tumor Necrosis Factor alpha (TNFα) and interferon gamma (IFNγ) are the key mediators, and symptoms may be relieved using anti-inflammatory therapy, e.g., corticosteroids. An anti-IL-6 receptor (IL-6R) antibody such as tocilizumab may be used to manage CRS, optionally with supportive care. An anti-IL-6 antibody such as siltuximab may additionally or alternatively be used to manage CRS, optionally with supportive care. IL-6 blockade (e.g., using an anti-IL-6R antibody or anti-IL-6 antibody) can be used if a patient infused with CAR T cells or CAR NK cells displays any of grade 1, grade 2, grade 3 or grade 4 CRS, but is typically reserved for more severe grades (e.g., grade 3 or grade 4). Corticosteroids can be administered to manage neurotoxicities that accompany or are caused by CRS, or to patients treated with an IL-6 blockade, but are generally not used as a first-line treatment for CRS. Other modalities for the management of CRS are described in, e.g., Shimabukuro-Vornhagen et al., “Cytokine Release Syndrome,” J. Immunother. Cancer 6:56 (2018).

TABLE 4
CRS may be graded using the Penn grading scale:

| GRADE | SYMPTOMS | MANAGEMENT |
|---|---|---|
| 1 | Mild reaction (fever, nausea, | Supportive care, e.g., |
| | fatigue, headache, myalgia, | antiemetics, antipyretics |
| | malaise) | |
| 2 | Moderate reaction (some signs of | Hospitalization for fever |
| | organ dysfunction such as grade 2 | with neutropenia |
| | creatinine or grade 3 liver function | |
| | test (LFT)) | |
| 3 | Severe reaction (signs of worse | Hospitalization for one or more of IV fluids, |
| | organ dysfunction such as grade 4 | low-dose vasosuppressors, fresh frozen |
| | LFT, grade 3 creatinine; | plasma or fibrinogen concentrate; provision of |
| | coagulopathy; dyspnea or hypoxia) | oxygen or CPAP |
| 4 | Life-threatening reaction | Hospitalization for vasosuppressors, |
| | (hypotension, hypoxia) | mechanical ventilation |

TABLE 5
CRS may also be graded by the CTCAE (National Cancer Institute
Common Terminology Criteria for Adverse Events) v4.0:

| GRADE | SYMPTOMS | MANAGEMENT |
|---|---|---|
| 1 | Mild reaction (fever, nausea, | Supportive care, e.g., |
| | fatigue, headache, myalgia, | antiemetics, antipyretics - |
| | malaise) | infusion interruption not indicated |
| 2 | Moderate reaction; patient | Interruption of infusion |
| | responds promptly to supportive | |
| | care, e.g. antihistamines, NSAIDs, | |
| | narcotics, IV fluids | |
| 3 | Prolonged reaction; patient does | Interruption of infusion; |
| | not respond promptly to supportive | hospitalization for sequelae |
| | care, e.g. antihistamines, NSAIDs, | |
| | narcotics, IV fluids; recurrence of | |
| | symptoms following initial | |
| | improvement; renal impairment | |
| | and/or pulmonary infiltrates | |
| 4 | Life-threatening reaction | Hospitalization for vasopressors, |
| | (hypotension, hypoxia) | mechanical ventilation |

TABLE 6
CRS may also be graded by the system of Lee et al. (“Current concepts in the
diagnosis and management of cytokine release syndrome,” Blood, 2014, 124: 188-195):

| GRADE | SYMPTOMS | MANAGEMENT |
|---|---|---|
| 1 | Non-life-threatening symptoms | Supportive care, e.g., |
| | (fever, nausea, fatigue, headache, | antiemetics, antipyretics |
| | myalgia, malaise) | |
| 2 | Moderate reaction; symptoms | O2 requirement <40%, fluids |
| | require, and patient responds to, | for hypotension, |
| | intervention | vasopressors |
| 3 | More severe reaction (e.g., hypoxia | O2 requirement >40%; high-dose |
| | and/or hypotension; grade 3 organ | vasopressors for hypotension |
| | toxicity, grade 4 transaminitis); | |
| | symptoms require and respond to | |
| | aggressive intervention. | |
| 4 | Life-threatening reaction | Hospitalization for vasopressors, |
| | (hypotension, hypoxia) | mechanical ventilation |

In particular embodiments, a composition comprises CAR T cells contemplated herein that are cultured in the presence of a PI3K inhibitor as disclosed herein and express one or more of the following markers: CD3, CD4, CD8, CD28, CD45RA, CD45RO, CD62, CD127, and HLA-DR can be further isolated by positive or negative selection techniques. In one embodiment, a composition comprises a specific subpopulation of T cells, expressing one or more of the markers selected from the group consisting of CD62L, CCR7, CD28, CD27, CD122, CD127, CD197; and CD38 or CD62L, CD127, CD197, and CD38, is further isolated by positive or negative selection techniques. In various embodiments, compositions do not express or do not substantially express one or more of the following markers: CD57, CD244, CD160, PD-1, CTLA4, TIM3, and LAG3.

In one embodiment, expression of one or more of the markers selected from the group consisting of CD62L, CD127, CD197, and CD38 is increased at least 1.5 fold, at least 2 fold, at least 3 fold, at least 4 fold, at least 5 fold, at least 6 fold, at least 7 fold, at least 8 fold, at least 9 fold, at least 10 fold, at least 25 fold, or more compared to a population of T cells activated and expanded without a PI3K inhibitor.

In one embodiment, expression of one or more of the markers selected from the group consisting of CD57, CD244, CD160, PD-1, CTLA4, TIM3, and LAG3 is decreased at least 1.5 fold, at least 2 fold, at least 3 fold, at least 4 fold, at least 5 fold, at least 6 fold, at least 7 fold, at least 8 fold, at least 9 fold, at least 10 fold, at least 25 fold, or more compared to a population of T cells activated and expanded with a PI3K inhibitor.

6.10. Therapeutic Methods

The genetically modified immune effector cells contemplated herein provide improved methods of adoptive immunotherapy for use in the treatment of a tumor or a cancer, or in the treatment of B cell related conditions that include, but are not limited to immunoregulatory conditions and hematological malignancies.

6.10.1. General Embodiments

In particular embodiments, the specificity of a primary immune effector cell is redirected to a tumor or a cancer by genetically modifying the primary immune effector cell with a CAR contemplated herein. In various embodiments, a viral vector is used to genetically modify an immune effector cell with a particular polynucleotide encoding a CAR comprising a domain that binds an antigen, e.g., a tumor antigen; a hinge domain; a transmembrane (TM) domain, a short oligo- or polypeptide linker, that links the TM domain to the intracellular signaling domain of the CAR; and one or more intracellular co-stimulatory signaling domains; and a primary signaling domain.

In particular embodiments, the specificity of a primary immune effector cell is redirected to B cells by genetically modifying the primary immune effector cell with a CAR contemplated herein. In various embodiments, a viral vector is used to genetically modify an immune effector cell with a particular polynucleotide encoding a CAR comprising a murine anti-BCMA antigen binding domain that binds a BCMA polypeptide, e.g., a human BCMA polypeptide; a hinge domain; a transmembrane (TM) domain, a short oligo- or polypeptide linker, that links the TM domain to the intracellular signaling domain of the CAR; and one or more intracellular co-stimulatory signaling domains; and a primary signaling domain.

In one embodiment, a type of cellular therapy is included where T cells are genetically modified to express a CAR that targets tumor or cancer cells. In another embodiment, CAR T cells are cultured in the presence of IL-2 and a PI3K inhibitor to increase the therapeutic properties and persistence of the CAR T cells. The CAR T cell are then infused to a recipient in need thereof. The infused cell is able to kill disease causing tumor or cancer cells in the recipient. Unlike antibody therapies, CAR T cells are able to replicate in vivo resulting in long-term persistence that can lead to sustained cancer therapy.

In one embodiment, a type of cellular therapy is included where T cells are genetically modified to express a CAR that targets BCMA expressing B cells. In another embodiment, anti-BCMA CAR T cells are cultured in the presence of IL-2 and a PI3K inhibitor to increase the therapeutic properties and persistence of the CAR T cells. The CAR T cell are then infused to a recipient in need thereof. The infused cell is able to kill disease causing B cells in the recipient. Unlike antibody therapies, CAR T cells are able to replicate in vivo resulting in long-term persistence that can lead to sustained cancer therapy.

In one embodiment, the CAR T cells can undergo robust in vivo T cell expansion and can persist for an extended amount of time. In another embodiment, the CAR T cells evolve into specific memory T cells that can be reactivated to inhibit any additional tumor formation or growth.

In particular embodiments, compositions comprising immune effector cells comprising the CARs contemplated herein are used in the treatment of a tumor or cancer.

In particular embodiments, compositions comprising immune effector cells comprising the CARs contemplated herein are used in the treatment of conditions associated with abnormal B cell activity.

Illustrative examples of conditions that can be treated, prevented or ameliorated using the immune effector cells comprising the CARs contemplated herein include, but are not limited to: systemic lupus erythematosus, rheumatoid arthritis, myasthenia gravis, autoimmune hemolytic anemia, idiopathic thrombocytopenia purpura, anti-phospholipid syndrome, Chagas' disease, Grave's disease, Wegener's granulomatosis, poly-arteritis nodosa, Sjogren's syndrome, pemphigus vulgaris, scleroderma, multiple sclerosis, anti-phospholipid syndrome, ANCA associated vasculitis, Goodpasture's disease, Kawasaki disease, and rapidly progressive glomerulonephritis.

The modified immune effector cells may also have application in plasma cell disorders such as heavy-chain disease, primary or immunocyte-associated amyloidosis, and monoclonal gammopathy of undetermined significance (MGUS).

As use herein, “B cell malignancy” refers to a type of cancer that forms in B cells (a type of immune system cell) as discussed infra.

In particular embodiments, compositions comprising CAR-modified T cells contemplated herein are used in the treatment of hematologic malignancies, including but not limited to B cell malignancies such as, for example, multiple myeloma (MM) and non-Hodgkin's lymphoma (NHL).

Multiple myeloma is a B cell malignancy of mature plasma cell morphology characterized by the neoplastic transformation of a single clone of these types of cells. These plasma cells proliferate in bone marrow (BM) and may invade adjacent bone and sometimes the blood. Variant forms of multiple myeloma include overt multiple myeloma, smoldering multiple myeloma, plasma cell leukemia, non-secretory myeloma, IgD myeloma, osteosclerotic myeloma, solitary plasmacytoma of bone, and extramedullary plasmacytoma (see, for example, Braunwald, et al. (eds), Harrison's Principles of Internal Medicine, 15th Edition (McGraw-Hill 2001)).

Multiple myeloma can be staged as follows:

TABLE 7
Durie-Salmon MM Staging Criteria

| Stage | Durie-Salmon Criteria |
|---|---|
| I | All of the following: |
| | Hemoglobin value >10 g/dL |
| | Serum calcium value normal or <12 mg/dL |
| | Bone x-ray, normal bone structure (scale 0), |
| | or solitary bone plasmacytoma only |
| | Low M-component production rates |
| | IgG value <5 g/dL; |
| | IgA value <3 g/dL |
| | Urine light chain M-component on |
| | electrophoresis <4 g/24 h |
| II | Neither Stage I nor Stage III |
| III | One or more of the following: |
| | Hemoglobin value <8.5 g/dL |
| | Serum calcium value normal or >12 mg/dL |
| | Advanced lytic bone lesions (scale 3) |
| | High M-component production rates |
| | IgG value >7 g/dL; |
| | IgA value >5 g/dL |
| | Urine light chain M-component on |
| | electrophoresis >12 g/24 h |

Subclassification Criteria
A Normal renal function (serum creatinine value <2.0 mg/dL)
B Abnormal renal function (serum creatinine value ≥2.0 mg/dL)

TABLE 8
International Staging System MM Staging Criteria

| | International Staging | Revised International Staging |
|---|---|---|
| Stage | System (ISS) Criteria | System (ISS) Criteria |
| I | Serum beta-2 microglobulin <3.5 mg/L | ISS stage I and standard-risk CA |
| | Serum albumin ≥3.5 g/dL | by iFISH and normal LDH |
| II | Neither Stage I nor Stage III | Neither Stage I nor Stage III |
| III | Serum beta-2 microglobulin ≥5.5 mg/L | ISS stage III and either high-risk |
| | | CA by iFISHe or high LDH |

Non-Hodgkin lymphoma encompasses a large group of cancers of lymphocytes (white blood cells). Non-Hodgkin lymphomas can occur at any age and are often marked by lymph nodes that are larger than normal, fever, and weight loss. There are many different types of non-Hodgkin lymphoma. For example, non-Hodgkin's lymphoma can be divided into aggressive (fast-growing) and indolent (slow-growing) types. Although non-Hodgkin lymphomas can be derived from B cells and T-cells, as used herein, the term “non-Hodgkin lymphoma” and “B cell non-Hodgkin lymphoma” are used interchangeably. B cell non-Hodgkin lymphomas (NHL) include Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, and mantle cell lymphoma. Lymphomas that occur after bone marrow or stem cell transplantation are usually B cell non-Hodgkin lymphomas.

Chronic lymphocytic leukemia (CLL) is an indolent (slow-growing) cancer that causes a slow increase in immature white blood cells called B lymphocytes, or B cells. Cancer cells spread through the blood and bone marrow, and can also affect the lymph nodes or other organs such as the liver and spleen. CLL eventually causes the bone marrow to fail. Sometimes, in later stages of the disease, the disease is called small lymphocytic lymphoma.

In particular embodiments, methods comprising administering a therapeutically effective amount of CAR-expressing immune effector cells contemplated herein or a composition comprising the same, to a patient in need thereof, alone or in combination with one or more therapeutic agents, are provided. In certain embodiments, the cells of the present disclosure are used in the treatment of patients at risk for developing a tumor or a cancer. Thus, in certain embodiments, presented herein are methods for the treatment or prevention of a tumor or a cancer comprising administering to a subject in need thereof, a therapeutically effective amount of the CAR-modified cells contemplated herein. In certain other embodiments, the cells of the present disclosure are used in the treatment of patients at risk for developing a condition associated with abnormal B cell activity or a B cell malignancy. Thus, in certain other embodiments, presented herein are methods for the treatment or prevention of a condition associated with abnormal B cell activity or a B cell malignancy comprising administering to a subject in need thereof, a therapeutically effective amount of the CAR-modified cells contemplated herein.

As used herein, the terms “individual” and “subject” are often used interchangeably and refer to any animal that exhibits a symptom of a disease, disorder, or condition that can be treated with the gene therapy vectors, cell-based therapeutics, and methods disclosed elsewhere herein. In specific embodiments, a subject includes any animal that exhibits symptoms of a tumor or a cancer that can be treated with the gene therapy vectors, cell-based therapeutics, and methods disclosed elsewhere herein. In specific embodiments, a subject includes any animal that exhibits symptoms of a disease, disorder, or condition of the hematopoietic system, e.g., a B cell malignancy, that can be treated with the gene therapy vectors, cell-based therapeutics, and methods disclosed elsewhere herein. Suitable subjects (e.g., patients) include laboratory animals (such as mouse, rat, rabbit, or guinea pig), farm animals, and domestic animals or pets (such as a cat or dog). Non-human primates and, preferably, human patients, are included. Typical subjects include human patients that have a tumor or cancer, have been diagnosed with a tumor or a cancer, or are at risk or having a tumor or a cancer. Typical subjects also include human patients that have a B cell malignancy, have been diagnosed with a B cell malignancy, or are at risk or having a B cell malignancy.

As used herein, the term “patient” refers to a subject that has been diagnosed with a particular disease, disorder, or condition that can be treated with the gene therapy vectors, cell-based therapeutics, and methods disclosed elsewhere herein.

As used herein “treatment” or “treating,” includes any beneficial or desirable effect on the symptoms or pathology of a disease or pathological condition, and may include even minimal reductions in one or more measurable markers of the disease or condition being treated. Treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

As used herein, “prevent,” and similar words such as “prevented,” “preventing” etc., indicate an approach for preventing, inhibiting, or reducing the likelihood of the occurrence or recurrence of, a disease or condition. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

By “enhance” or “promote,” or “increase” or “expand” refers generally to the ability of a composition contemplated herein, e.g., a genetically modified T cell or vector encoding a CAR, to produce, elicit, or cause a greater physiological response (i.e., downstream effects) compared to the response caused by either vehicle or a control molecule/composition. A measurable physiological response may include an increase in T cell expansion, activation, persistence, and/or an increase in cancer cell killing ability, among others apparent from the understanding in the art and the description herein. An “increased” or “enhanced” amount is typically a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response produced by vehicle or a control composition.

By “decrease” or “lower,” or “lessen,” or “reduce,” or “abate” refers generally to the ability of composition contemplated herein to produce, elicit, or cause a lesser physiological response (i.e., downstream effects) compared to the response caused by either vehicle or a control molecule/composition. A “decrease” or “reduced” amount is typically a “statistically significant” amount, and may include an decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response (reference response) produced by vehicle, a control composition, or the response in a particular cell lineage.

By “maintain,” or “preserve,” or “maintenance,” or “no change,” or “no substantial change,” or “no substantial decrease” refers generally to the ability of a composition contemplated herein to produce, elicit, or cause a substantially similar physiological response (i.e., downstream effects) in a cell, as compared to the response caused by either vehicle, a control molecule/composition, or the response in a particular cell lineage. A comparable response is one that is not significantly different or measurably different from the reference response.

In one embodiment, a method of treating a tumor or a cancer in a subject in need thereof comprises administering an effective amount, e.g., a therapeutically effective amount of a composition comprising genetically modified immune effector cells contemplated herein. The quantity and frequency of administration will be determined by such factors as the condition of the patient, and the type and severity of the patient's disease, although appropriate dosages may be determined by clinical trials.

In one embodiment, a method of treating a B cell related condition in a subject in need thereof comprises administering an effective amount, e.g., a therapeutically effective amount of a composition comprising genetically modified immune effector cells contemplated herein. The quantity and frequency of administration will be determined by such factors as the condition of the patient, and the type and severity of the patient's disease, although appropriate dosages may be determined by clinical trials.

In one embodiment, the amount of T cells in the composition administered to a subject is at least 0.1×10⁵ cells, at least 0.5×10⁵ cells, at least 1×10⁵ cells, at least 5×10⁵ cells, at least 1×10⁶ cells, at least 0.5×10⁷ cells, at least 1×10⁷ cells, at least 0.5×10⁸ cells, at least 1×10⁸ cells, at least 0.5×10⁹ cells, at least 1×10⁹ cells, at least 2×10⁹ cells, at least 3×10⁹ cells, at least 4×10⁹ cells, at least 5×10⁹ cells, or at least 1×10¹⁰ cells. In particular embodiments, about 1×10⁷ CAR T cells to about 1×10⁹ CAR T cells, about 2×10⁷ CAR T cells to about 0.9×10⁹ CAR T cells, about 3×10⁷ CAR T cells to about 0.8×10⁹ CAR T cells, about 4×10⁷ CAR T cells to about 0.7×10⁹ CAR T cells, about 5×10⁷ CAR T cells to about 0.6×10⁹ CAR T cells, or about 5×10⁷ CAR T cells to about 0.5×10⁹ CAR T cells are administered to a subject.

In one embodiment, the amount of T cells in the composition administered to a subject is at least 0.1×10⁴ cells/kg of bodyweight, at least 0.5×10⁴ cells/kg of bodyweight, at least 1×10⁴ cells/kg of bodyweight, at least 5×10⁴ cells/kg of bodyweight, at least 1×10⁵ cells/kg of bodyweight, at least 0.5×10⁶ cells/kg of bodyweight, at least 1×10⁶ cells/kg of bodyweight, at least 0.5×10⁷ cells/kg of bodyweight, at least 1×10⁷ cells/kg of bodyweight, at least 0.5×10⁸ cells/kg of bodyweight, at least 1×10⁸ cells/kg of bodyweight, at least 2×10⁸ cells/kg of bodyweight, at least 3×10⁸ cells/kg of bodyweight, at least 4×10⁸ cells/kg of bodyweight, at least 5×10⁸ cells/kg of bodyweight, or at least 1×10⁹ cells/kg of bodyweight. In particular embodiments, about 1×10⁶ CAR T cells/kg of bodyweight to about 1×10⁸ CAR T cells/kg of bodyweight, about 2×10⁶ CAR T cells/kg of bodyweight to about 0.9×10⁸ CAR T cells/kg of bodyweight, about 3×10⁶ CAR T cells/kg of bodyweight to about 0.8×10⁸ CAR T cells/kg of bodyweight, about 4×10⁶ CAR T cells/kg of bodyweight to about 0.7×10⁸ CAR T cells/kg of bodyweight, about 5×10⁶ CAR T cells/kg of bodyweight to about 0.6×10⁸ CAR T cells/kg of bodyweight, or about 5×10⁶ CAR T cells/kg of bodyweight to about 0.5×10⁸ CAR T cells/kg of bodyweight are administered to a subject.

One of ordinary skill in the art would recognize that multiple administrations of the compositions of the present disclosure may be required to effect the desired therapy. For example a composition may be administered 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more.

In certain embodiments, it may be desirable to administer activated immune effector cells to a subject and then subsequently redraw blood (or have an apheresis performed), activate immune effector cells therefrom according to the present disclosure, and reinfuse the patient with these activated and expanded immune effector cells. This process can be carried out multiple times every few weeks. In certain embodiments, immune effector cells can be activated from blood draws of from 10 cc to 400 cc. In certain embodiments, immune effector cells are activated from blood draws of 20 cc, 30 cc, 40 cc, 50 cc, 60 cc, 70 cc, 80 cc, 90 cc, 100 cc, 150 cc, 200 cc, 250 cc, 300 cc, 350 cc, or 400 cc or more. Not to be bound by theory, using this multiple blood draw/multiple reinfusion protocol may serve to select out certain populations of immune effector cells.

The administration of the compositions contemplated herein may be carried out in any convenient manner, including by aerosol inhalation, injection, ingestion, transfusion, implantation or transplantation. In one embodiment, compositions are administered parenterally. The phrases “parenteral administration” and “administered parenterally” as used herein refers to modes of administration other than enteral and topical administration, usually by injection, and includes, without limitation, intravascular, intravenous, intramuscular, intraarterial, intrathecal, intracapsular, intraorbital, intratumoral, intracardiac, intradermal, intraperitoneal, transtracheal, subcutaneous, subcuticular, intraarticular, subcapsular, subarachnoid, intraspinal and intrasternal injection and infusion. In one embodiment, the compositions contemplated herein are administered to a subject by direct injection into a tumor, lymph node, or site of infection.

In one embodiment, a subject in need thereof is administered an effective amount of a composition to increase a cellular immune response to a tumor or a cancer in the subject. The immune response may include cellular immune responses mediated by cytotoxic T cells capable of killing infected cells, regulatory T cells, and helper T cell responses. Humoral immune responses, mediated primarily by helper T cells capable of activating B cells thus leading to antibody production, may also be induced. A variety of techniques may be used for analyzing the type of immune responses induced by the compositions of the present disclosure, which are well described in the art; e.g., Current Protocols in Immunology, Edited by: John E. Coligan, Ada M. Kruisbeek, David H. Margulies, Ethan M. Shevach, Warren Strober (2001) John Wiley & Sons, NY, N.Y.

In one embodiment, a subject in need thereof is administered an effective amount of a composition to increase a cellular immune response to a B cell related condition in the subject. The immune response may include cellular immune responses mediated by cytotoxic T cells capable of killing infected cells, regulatory T cells, and helper T cell responses. Humoral immune responses, mediated primarily by helper T cells capable of activating B cells thus leading to antibody production, may also be induced. A variety of techniques may be used for analyzing the type of immune responses induced by the compositions of the present disclosure, which are well described in the art; e.g., Current Protocols in Immunology, Edited by: John E. Coligan, Ada M. Kruisbeek, David H. Margulies, Ethan M. Shevach, Warren Strober (2001) John Wiley & Sons, NY, N.Y.

In the case of T cell-mediated killing, CAR-ligand binding initiates CAR signaling to the T cell, resulting in activation of a variety of T cell signaling pathways that induce the T cell to produce or release proteins capable of inducing target cell apoptosis by various mechanisms. These T cell-mediated mechanisms include (but are not limited to) the transfer of intracellular cytotoxic granules from the T cell into the target cell, T cell secretion of pro-inflammatory cytokines that can induce target cell killing directly (or indirectly via recruitment of other killer effector cells), and up regulation of death receptor ligands (e.g. FasL) on the T cell surface that induce target cell apoptosis following binding to their cognate death receptor (e.g. Fas) on the target cell.

In one embodiment, provided herein is a method of treating a subject diagnosed with a tumor or a cancer comprising removing immune effector cells from a subject diagnosed with a tumor or a cancer, genetically modifying said immune effector cells with a vector comprising a nucleic acid encoding a CAR as contemplated herein, thereby producing a population of modified immune effector cells, and administering the population of modified immune effector cells to the same subject. In a particular embodiment, the immune effector cells comprise T cells.

In one embodiment, provided herein is a method of treating a subject diagnosed with a B cell related condition comprising removing immune effector cells from a subject diagnosed with a BCMA-expressing B cell related condition, genetically modifying said immune effector cells with a vector comprising a nucleic acid encoding a CAR as contemplated herein, thereby producing a population of modified immune effector cells, and administering the population of modified immune effector cells to the same subject. In a particular embodiment, the immune effector cells comprise T cells.

In certain embodiments, also provided herein are methods for stimulating an immune effector cell mediated immune modulator response to a target cell population in a subject comprising the steps of administering to the subject an immune effector cell population expressing a nucleic acid construct encoding a CAR molecule.

The methods for administering the cell compositions described herein includes any method which is effective to result in reintroduction of ex vivo genetically modified immune effector cells that either directly express a CAR of the present disclosure in the subject or on reintroduction of the genetically modified progenitors of immune effector cells that on introduction into a subject differentiate into mature immune effector cells that express the CAR. One method comprises transducing peripheral blood T cells ex vivo with a nucleic acid construct in accordance with the present disclosure and returning the transduced cells into the subject.

All publications, patent applications, and issued patents cited in this specification are hereby incorporated by reference herein in their entireties as if each individual publication, patent application, or issued patent were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### 7. EXAMPLES

### 7.1. Example 1: Construction of Exemplary BCMA CARs

CARs containing anti-BCMA scFv antibodies were designed to contain an MND promoter operably linked to anti-BMCA scFv, a hinge and transmembrane domain from CD8α and a CD137 co-stimulatory domain followed by the intracellular signaling domain of the CD3ζ chain. See, e.g., FIG. 1. See, also, International Publication No. WO 2016/094304, which is incorporated by reference herein in its entirety, and in particular incorporates the disclosure of BCMA CARs and their characterization. The BCMA CAR shown in FIG. 1 comprises a CD8α signal peptide (SP) sequence for the surface expression on immune effector cells. The polynucleotide sequence of an exemplary BCMA CAR is set forth in SEQ ID NO: 10 (polynucleotide sequence of anti-BCMA02 CAR); an exemplary polypeptide sequence of a BCMA CAR is set forth in SEQ ID NO: 9 (polypeptide sequence of anti-BCMA02 CAR); and a vector map of an exemplary CAR construct is shown in FIG. 1. Table 9 shows the identity, GenBank Reference (where applicable), Source Name and Citation for the various nucleotide segments of a BCMA CAR lentiviral vector that comprise a BCMA CAR construct as shown in FIG. 1.

TABLE 9

| Nucleotides | Identity | GenBank Reference | Source Name | Citation |
|---|---|---|---|---|
| 1-185 | pUC19 plasmid | Accession #L09137.2 | pUC19 | New England |
| | backbone | nt 1-185 | | Biolabs |
| 185-222 | Linker | Not applicable | Synthetic | Not applicable |
| 223-800 | CMV | Not Applicable | pHCMV | Yee, et al., |
| | | | | (1994) PNAS |
| | | | | 91: 9564-68 |
| 801-1136 | R, U5, PBS, and | Accession #M19921.2 | pNL4-3 | Maldarelli, |
| | packaging sequences | nt 454-789 | | et. al. (1991) |
| | | | | J Virol: |
| | | | | 65(11): 5732-43 |
| 1137-1139 | Gag start codon (ATG) | Not Applicable | Synthetic | Not applicable |
| | changed to stop codon | | | |
| | (TAG) | | | |
| 1140-1240 | HIV-1 gag sequence | Accession #M19921.2 | pNL4-3 | Maldarelli, |
| | | nt 793-893 | | et. al. (1991) |
| | | | | J Virol: |
| | | | | 65(11): 5732-43 |
| 1241-1243 | HIV-1 gag sequence | Not Applicable | Synthetic | Not applicable |
| | changed to a second | | | |
| | stop codon | | | |
| 1244-1595 | HIV-1 gag sequence | Accession #M19921.2 | pNL4-3 | Maldarelli, |
| | | nt 897-1248 | | et. al. (1991) |
| | | | | J Virol: |
| | | | | 65(11): 5732-43 |
| 1596-1992 | HIV-1 pol | Accession #M19921.2 | pNL4-3 | Maldarelli, |
| | cPPT/CTS | nt 4745-5125 | | et. al. (1991) |
| | | | | J Virol: |
| | | | | 65(11): 5732-43 |
| 1993-2517 | HIV-1, isolate HXB3 | Accession #M14100.1 | PgTAT-CMV | Malim, M. H. |
| | env region (RRE) | nt 1875-2399 | | Nature (1988) |
| | | | | 335: 181-183 |
| 2518-2693 | HIV-1 env sequences | Accession #M19921.2 | pNL4-3 | Maldarelli, |
| | S/A | nt 8290-8470 | | et. al. (1991) |
| | | | | J Virol: |
| | | | | 65(11): 5732-43 |
| 2694-2708 | Linker | Not applicable | Synthetic | Not applicable |
| 2709-3096 | MND | Not applicable | pccl-c- | Challita et al. |
| | | | MNDU3c-x2 | (1995) |
| | | | | J. Virol. 69: |
| | | | | 748-755 |
| 3097-3124 | Linker | Not applicable | Synthetic | Not applicable |
| 3125-3187 | Signal peptide | Accession # | CD8a signal | Not applicable |
| | | NM_001768 | peptide | |
| 3188-3934 | BCMA02 scFv | Not applicable | Synthetic | Not applicable |
| 3935-4141 | CD8a hinge and TM | Accession # | CD8a hinge | Milone et al |
| | | NM_001768 | and TM | (2009) |
| | | | | Mol Ther |
| | | | | 17(8): 1453-64 |
| 4144-4269 | CD137 (4-1BB) | Accession # | CD137 | Milone et al |
| | signaling domain | NM_001561 | signaling | (2009) |
| | | | domain | Mol Ther |
| | | | | 17(8): 1453-64 |
| 4270-4606 | CD3-ζ signaling | Accession # | CD3-ζ | Milone et al |
| | domain | NM_000734 | signaling | (2009) |
| | | | domain | Mol Ther |
| | | | | 17(8): 1453-64 |
| 4607-4717 | HIV-1 ppt and part of | Accession #M19921.2 | pNL4-3 | Maldarelli, |
| | 3′ U3 | nt 9005-9110 | | et. al. (1991) |
| | | | | J Virol: |
| | | | | 65(11): 5732-43 |
| 4718-4834 | HIV-1 part of U3 | Accession #M19921.2 | pNL4-3 | Maldarelli, |
| | (399 bp deletion) and R | nt 9511-9627 | | et. al. (1991) |
| | | | | J Virol: |
| | | | | 65(11): 5732-43 |
| 4835-4858 | Synthetic polyA | Not applicable | Synthetic | Levitt, N. Genes |
| | | | | & Dev (1989) |
| | | | | 3: 1019-1025 |
| 4859-4877 | Linker | Not applicable | Synthetic | Not Applicable |
| 4878-7350 | pUC19 backbone | Accession #L09137.2 | pUC19 | New England |
| | | nt 2636-2686 | | Biolabs |

### 7.2 Example 2: Effects of Prior Alkylating Therapies on Starting Material for Car T Cell Product Manufacturing and Preinfusion Patient Characteristics in Late-Line Multiple Myeloma

In the phase 2 pivotal KarMINa trial (NCT03361748) investigating the anti-BCMA CAR T cell therapy idecabtagene vicleucel (bb2121) in late-line multiple myeloma, 80% of patients had a history of prior anticancer treatment with ≥1 alkylating agents outside of stem cell transplant regimens. In this retrospective analysis of the Phase 2 KarMMa trial (NCT03361748), patient and PBMC characteristics associated with time from last dose of alkylating agent(s) until apheresis of PBMCs for CAR T cell manufacture were identified.

Methods: PBMCs isolated from patient apheresis material, which serve as starting material for CAR T cell manufacturing, were immunophenotyped by polychromatic flow cytometry for markers associated with T cell differentiation, memory, senescence, and exhaustion. Data from relevant prespecified clinical and exploratory endpoints were collected, and a novel implementation of left-censored time-to-event analysis (Biometrics, 1976, 32:459-463) was used to identify statistically significant relationships between washout time after prior alkylator exposure (encompassing bendamustine, busulfan, carboplatin, carmustine, chlorambucil, cisplatin, cyclophosphamide, evofosfamide, ifosfamide, oxaliplatin, platinum, procarbazine, and thiotepa) and patient and PBMC variables. Alkylating agent exposure associated with stem cell transplant was excluded from this analysis. Dosage amounts of prior alkylators were not considered due to sparse annotations in the patient histories. Optimal cutpoints were identified for each variable that maximized the proportional hazard of receiving an alkylator between patients above and below the cutpoint, and P values were adjusted for testing multiple cutpoints. Relationships were verified by nonparametric correlation, in which alkylator washout was encoded as 1/log(−washout).

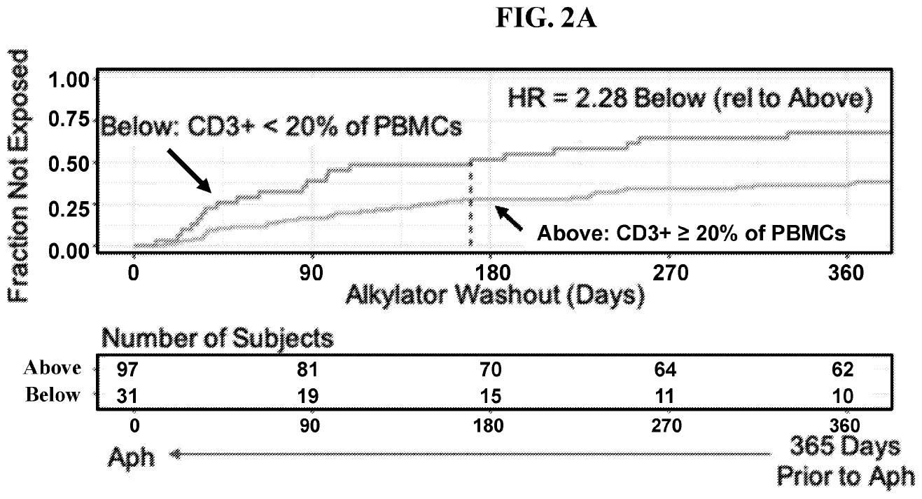

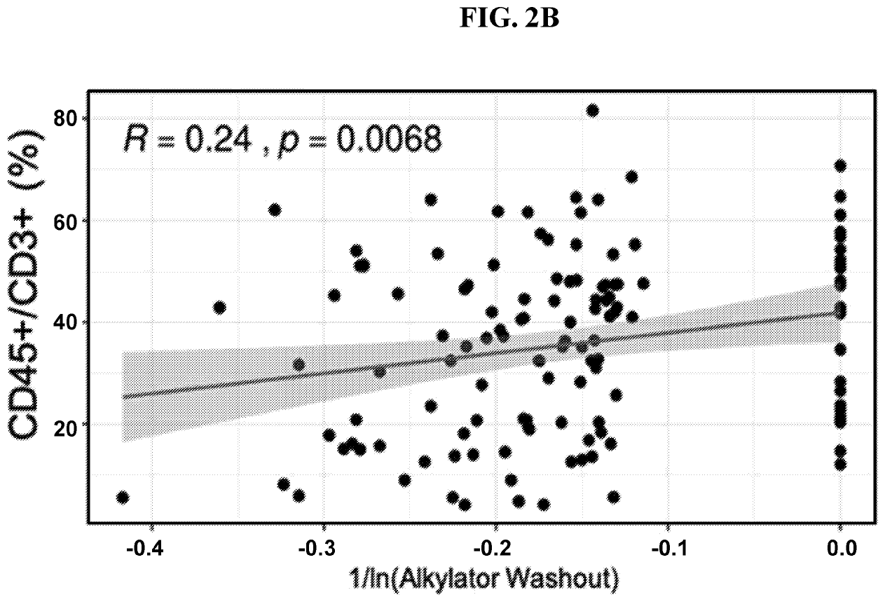

Results: FIG. 2A shows a novel inverted time-to-event model, which is broadly applicable to capture relationships between variables of interest and drug exposures and identifies which patient and PBMC variables are associated with time-since-last-exposure to alkylating agents prior to enrollment in the Phase 2 KarMMa trial. The table tabulates the number of patients with exposure to alkylating agents at each time interval. “Fraction Not Exposed” on the y-axis refers to the proportion of patients not yet exposed to alkylating agents at the time indicated on the x-axis. Since time is inverted, the x-axis should be read from right to left, e.g., moving from day 0 to 180 is going backwards in time. At 365 days before apheresis (right side of FIG. 2A), 70% of subjects with <20% PBMCs have not yet been exposed to alkylating agents, whereas approximately 35-40% of subjects with ≥20% PBMCs have not yet been exposed to alkylating agents. Moving towards the day of apheresis (i.e., to the left), a greater number of subjects will have been exposed to alkylating agents and these percentages continue to decrease. At the day of apheresis, all subjects are assumed to have been exposed, hence the curves converge to zero at day 0 (apheresis). The curve labeled “Below: CD3+<20% of PBMCs” being higher than the curve labeled “Above: CD3+≥20% of PBMCs” indicates that the subjects in the “Below: CD3+<20% of PBMCs” curve had more recent prior exposure to alkylating agents. In other words, subjects in the curve labeled “Below: CD3+<20% of PBMCs” are 2.28× more likely to have had exposure to alkylating agents than patients in the curve labeled “Above: CD3+≥20% of PBMCs.”

More recent exposure to an alkylating agent (after diagnosis but before apheresis) was associated with patients receiving more prior therapies per year to manage their disease (hazard ratio [HR]=2.63, p=−0.54, P<0.0001), having a lower body mass index (HR=0.93, p=0.27, P=0.0021), and having higher ferritin levels at baseline (log-scaled HR=1.33, p=−0.31, P=0.0004). Patients with more recent alkylator exposure also had fewer T cells in their PBMC material (HR=2.28, p=0.24, P=0.0068; FIGS. 2A and 2B) along with more CD8+ effector memory (TEM) (CCR7−/CD45RA−) and fewer CD8+ effector memory RA (TEMRA) (CCR7−/CD45RA+) T cells (HR=1.02 and 0.98, p=−0.2 and 0.21, P=0.023 and 0.016, respectively). FIG. 2B provides Spearman's coefficient using an encoded washout, showing that more recent alkylator exposure is associated with fewer CD3+ cells. Patients with <20% T cells in their PBMCs were 2.3× more likely to have had prior alkylator exposure. A 50% reduction in the median CD3+ T cell composition of patient PBMCs was detectable up to 9 months after the last dose of alkylator, relative to patients who never received this drug class. In a multivariate model evaluating the correlation between the T cell fraction in PBMCs and number of therapies per year and alkylator washout period, number of therapies per year did not significantly improve model performance compared with the null model including alkylator washout alone.

Conclusions: Associations between patient characteristics and alkylator washout indicate that patients who more recently received alkylating agents to manage their myeloma had a more aggressive disease course, having progressed more quickly through prior regimens, and were in overall poorer health (lower weight, elevated systemic inflammation). Although these factors suggest a suboptimal patient profile, the depletion of T cells by alkylator therapy may be especially disadvantageous for autologous CAR T cell therapies (Mol. Ther. Oncolytics, 2016, 3:16015; Biol Blood Marrow Transplant., 2018, 24:1135-1141). Our analysis found that the use of alkylators in patients prior to CAR T cell therapy exhibits a detrimental effect on the patients' apheresis PBMC material up to 6-9 months after the last dose. These results indicate that isolating PBMCs at a certain time (e.g., 6-9 months) after a patient has been administered an alkylating agent, such that the number or percentage of T cells in the patient's PBMCs is sufficient to manufacture a target number of CAR T cells for administering a CAR T cell therapy (e.g., at least 1×10⁷ to 1×10⁸ CAR T cells). For example, the results indicate a benefit of obtaining patient apheresis material at least 6-9 months (or more) after the patient has been administered an alkylating agent(s), wherein the apheresis is used as starting material from which PBMCs are isolated and subsequently used as the source of T cells in T cell manufacturing methods to manufacture CAR T cells (e.g., anti-BCMA CAR T cells idecabtagene vicleucel (bb2121)).

### 7.3 Example 3: Effects of Prior Alkylating Therapies on Starting Material for Car T Cell Product Manufacturing and Preinfusion Patient Characteristics in Late-Line Multiple Myeloma

Understanding how a patient's treatment history affects their clinical and immune profiles is an important part of optimizing autologous cell therapies. Identifying prior therapy exposures that affect the patient or their PBMC material may help optimize outcomes with CAR T cell therapy. In this retrospective analysis of the Phase 2 KarMMa trial (NCT03361748), patient and PBMC characteristics associated with time from last dose of alkylating agent(s) until apheresis of PBMCs for CAR T cell manufacture were identified.

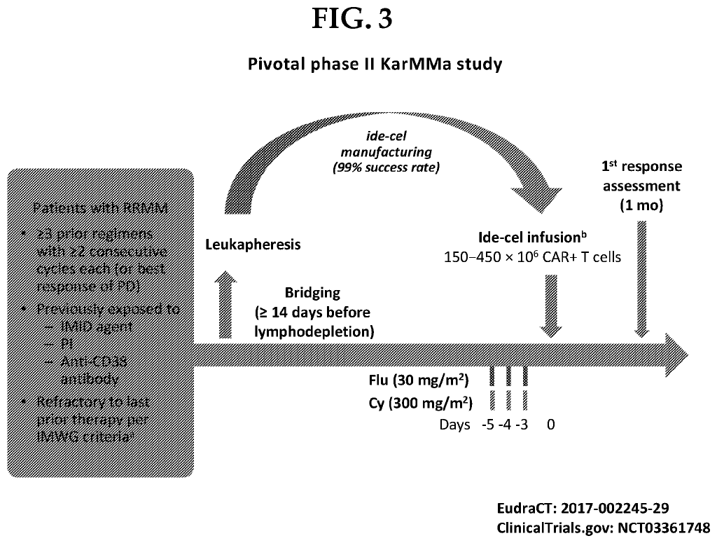

Methods: FIG. 3 depicts the general protocol by which ide-cel CAR T cells were manufactured and infused into relapsed and refractory multiple myeloma (RRMM) patients, who were subsequently assessed for response to ide-cel therapy. PBMCs isolated from patient apheresis material, which serves as starting material for CAR T cell manufacturing, were immunophenotyped by polychromatic flow cytometry for markers associated with T cell differentiation, memory, senescence, and exhaustion. Data from relevant prespecified clinical and exploratory endpoints were collected, and a novel implementation of left-censored time-to-event analysis (Biometrics, 1976, 32:459-463) was used to identify statistically significant relationships between washout time after prior alkylator exposure (encompassing bendamustine, busulfan, carboplatin, carmustine, chlorambucil, cisplatin, cyclophosphamide, evofosfamide, ifosfamide, oxaliplatin, platinum, procarbazine, and thiotepa, which were not associated with Stem Cell Transplantation) and patient and PBMC variables. Patients with no prior alkylator exposure were censored at their earliest prior therapy date; the novel time-to-event model assumes all patients were exposed to alkylating agent at some point in the past. The Benjamini-Hochberg correction was applied to P values to account for the multiple testing used to determine an optimal cutpoint for separating the regression curves. Dosage amounts of prior alkylators were not considered due to sparse annotations in the patient histories. Optimal cutpoints were identified for each variable that maximized the proportional hazard of receiving an alkylator between patients above and below the cutpoint, and P values were adjusted for testing multiple cutpoints. Relationships were verified by nonparametric correlation, in which alkylator washout was encoded as 1/log(−washout).

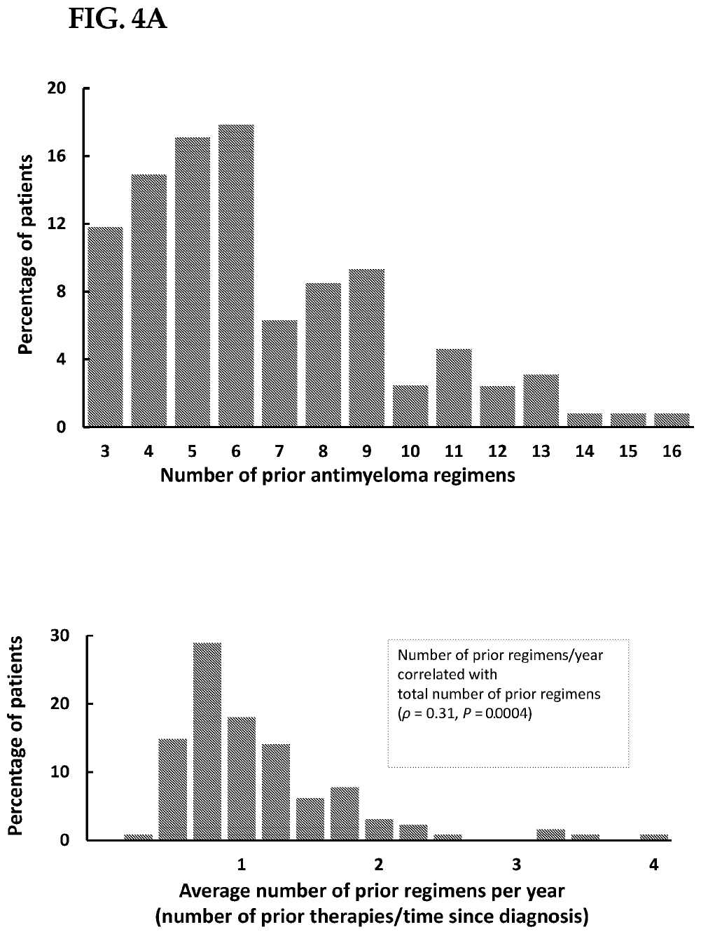

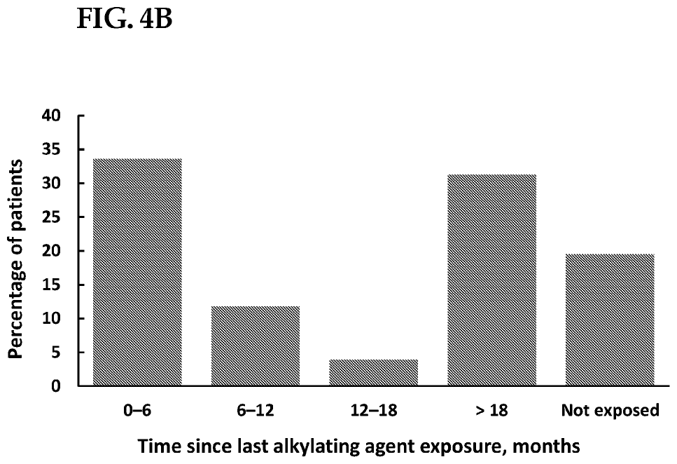

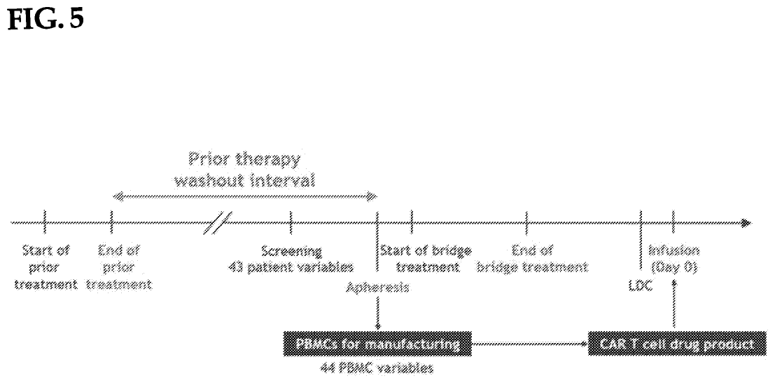

Results: Exposure of patients to prior therapies is a key upstream variable in the context of subsequently administered autologous cell therapies. This retrospective analysis showed that 80% of RRMM patients undergoing CAR T cell therapy had prior alkylating agent exposure (FIG. 4A). Patients had a median of 6 prior regimens at an average rate of 0.94 regimens per year since RRMM diagnosis (FIG. 4A). The number of prior regimens per year correlated with the total number of prior regimens (p=0.31, P=0.0004). Approximately one third of patients were exposed to alkylating agents within 6 months of apheresis (FIG. 4B). A total of 87 variables, including 43 patient variables (e.g., demographics, blood chemistries, complete blood count (CBC), soluble blood factors) and 44 PBMC variables (e.g., CD4/CD8 memory, activation, and exhaustion) were analyzed to identify associations with time-since-last-exposure to alkylating agents. FIG. 5 depicts the general pre-infusion clinical timeline with annotations for the prior therapy washout period, the parallel timeline of CAR T manufacturing, and when/where data collection occurred for screening and apheresis variables included in the analysis.

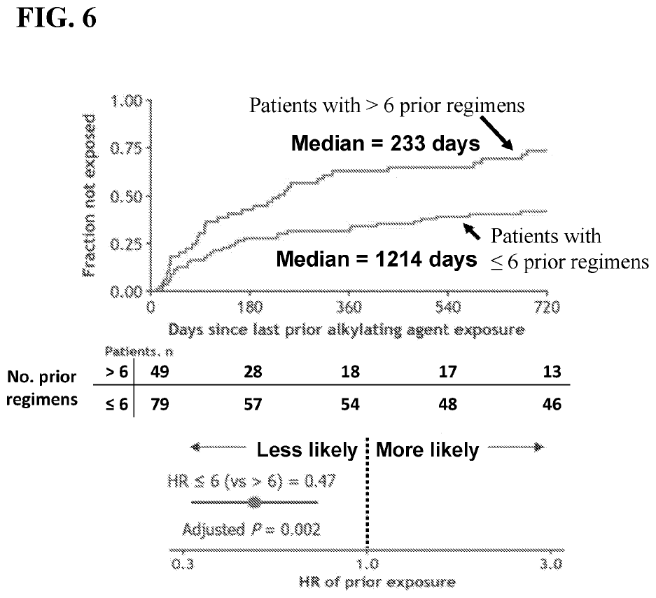

FIG. 6 shows a novel inverted time-to-event model, which is broadly applicable to capture relationships between variables of interest and drug exposures. The table below the graph tabulates the number of patients with exposure to either >6 or ≤6 alkylating agents at each time interval. Patients with >6 prior regimens had more recent prior alkylating agent exposure (median=233 days) relative to patients with ≤6 prior regimens (median 1214 days) (See FIG. 6; HR≤6 (vs. >6)=0.47, adjusted P=0.002).

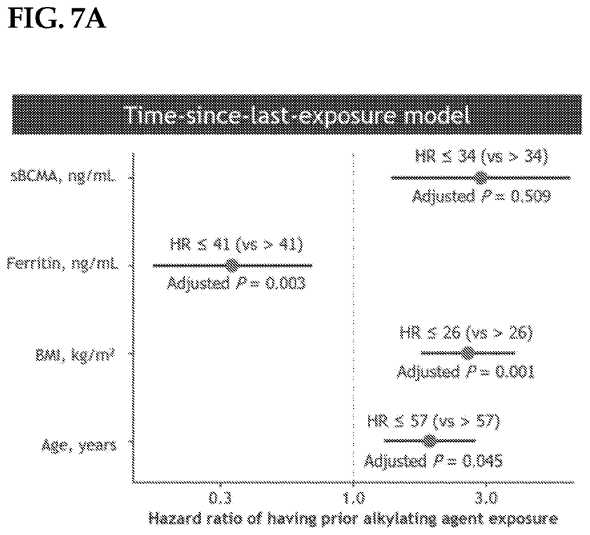

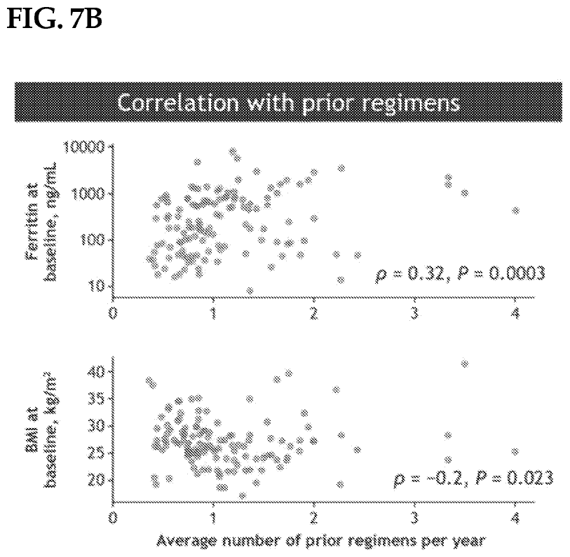

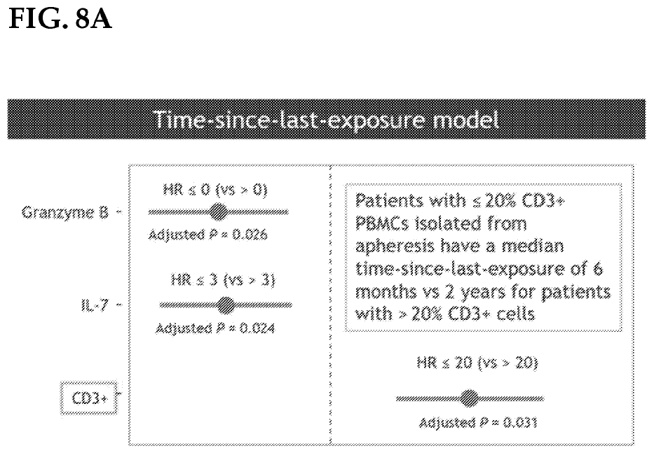

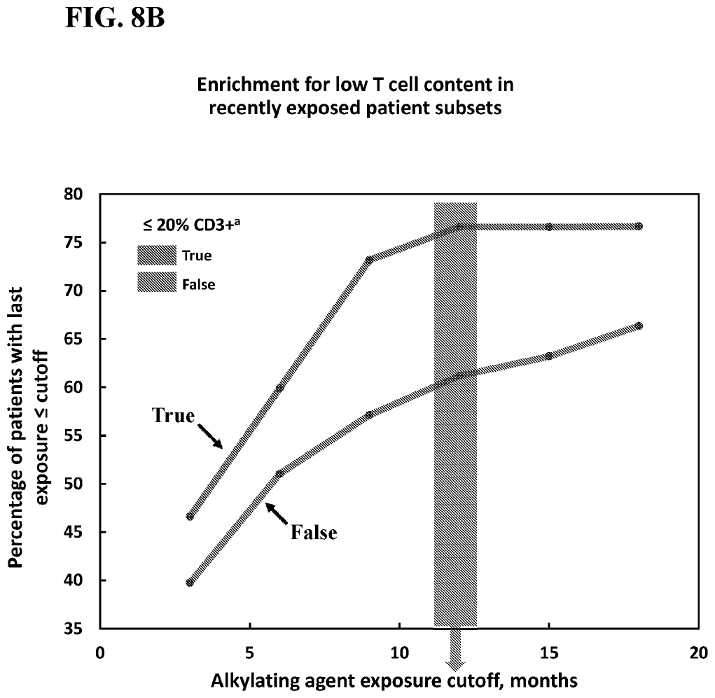

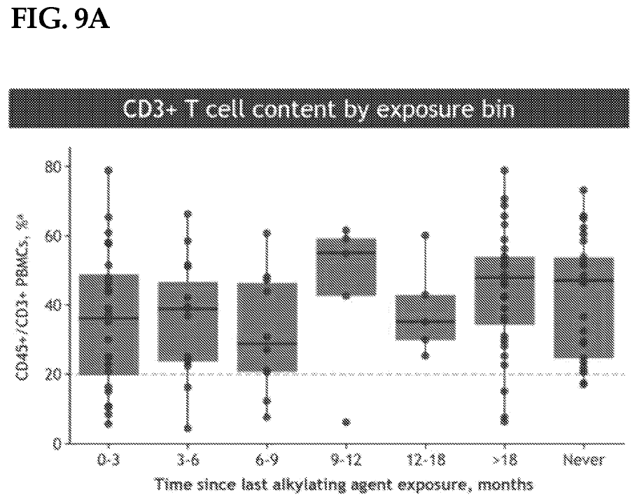

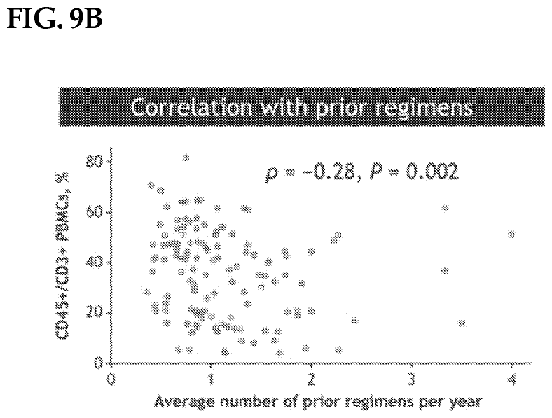

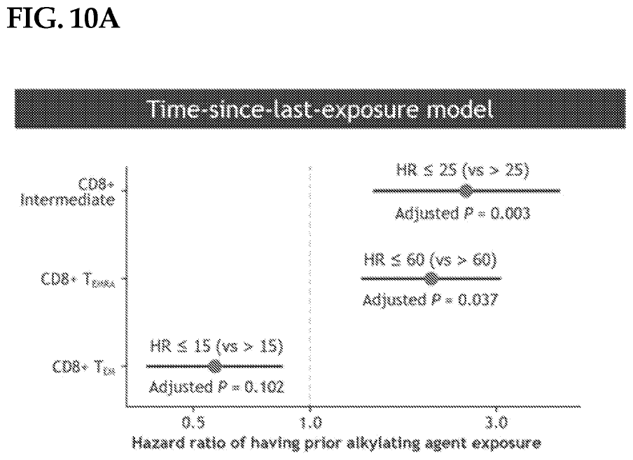

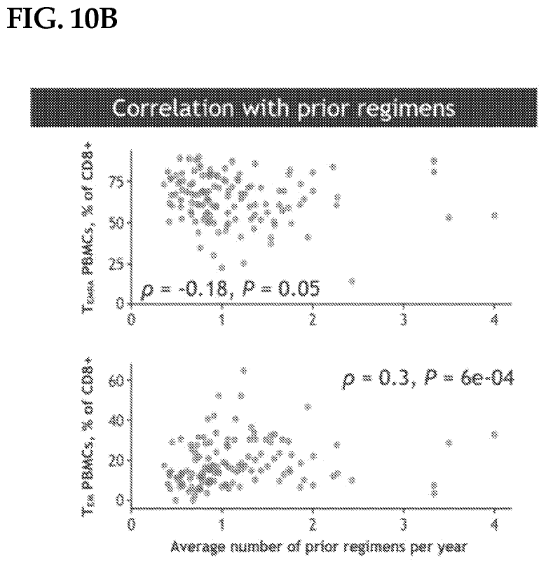

Patient features associated with alkylating agent exposure were analyzed. Higher pre-lymphodepleting chemotherapy (LDC) baseline ferritin, lower BMI, and younger age in patients were independently associated with more recent exposure to alkylating agents, whereas lower tumor burden (by sBCMA at screening) was not significant after multiple testing correction (See FIG. 7A). Higher ferritin and lower BMI were also moderately correlated with more treatment regimens per year (See FIG. 7B). Immune factors associated with alkylating agent exposure were also analyzed. Patients with ≤20% CD3+ PBMCs isolated from apheresis have a median time-since-last-exposure of 6 months compared to 2 years for patients with >20% CD3+ cells (See FIG. 8A, HR≤20% vs >20%; adjusted P=0.031). More recent exposure of patients to alkylating agents was associated with fewer T cells and higher levels of soluble granzyme B (HR≤0 (vs >0), adjusted P=0.026) and IL-7 (HR≤3 (vs >3), adjusted P=0.024) at the time of screening, indicating an immune profile consistent with (tumor) cell killing and recovery towards homeostasis (See FIG. 8A). Alkylating agent exposure within 12 months of apheresis was present in >75% of patients with ≤20% CD3+ PBMCs vs 60% of patients with >20% CD3+ PBMCs (See FIG. 8B). In addition, T cell depletion was detectable ≥6 months after last alkylating agent exposure (See FIG. 9). Patients with alkylating agent exposure within the last 9 months had a 20% to 40% lower median CD3+ T cell percentage relative to patients with exposure more than 9 months ago or with no history of exposure (See FIG. 9A). A greater number of treatment regimens per year was also inversely correlated with T cell content in PBMCs (i.e., PBMCs from apheresis material used for CAR T cell manufacturing) (See FIG. 9B). However, the association between time since alkylating agent exposure and CD3+ T-cell content in PBMCs remained independently significant (P=0.02) in a linear model including prior regimens per year (P=0.39). FIG. 10 shows T cell memory phenotypes associated with alkylating agent exposure. In particular, more recent exposure to alkylating agents was associated with fewer T_{EMRA} cells and a greater number of TEM/intermediate CD8+ T cells in PBMCs obtained from apheresis material (See FIG. 10A). No association was observed between alkylator exposure and CD4+ T cell memory populations. In addition, a greater number of treatment regimens per year was also correlated with fewer T_{EMRA} and a greater number of T_{EM} CD8+ T cells in patient PBMCs (See FIG. 10B).

Conclusions: Prior exposure of patients to alkylating agent therapy for treatment of myeloma that reduce the quantity or quality of harvestable T cells from these patients may be disadvantageous for subsequent autologous T cell therapies. One third of patients in the Phase 2 study cohort had a non-transplant-related exposure to alkylating agents within 6 months of apheresis (for CAR T cell manufacturing). Recent exposure was found to be associated with suboptimal patient and PBMC characteristics.

In some variations, time-to-event (TTE) modeling can be used to determine an optimal washout period for a particular therapy to a disease such as the therapies described above. Such time-to-event modeling can be based on populations of subjects which might have been given prior treatment for such a disease prior to joining a particular study. The results of such time-to-event modeling can be used for a variety of applications including, when to administer therapy for a particular individual or a subset of a population, whether a particular individual or a subset of a population is suited to enroll in a particular clinical trial or other project, and/or whether a particular individual or a subset of a population should be given the therapy in combination with one or more other therapies/treatments. The current time-to-event modeling techniques can also be used to, for example, provide insight into timing and other strategies relating to particular therapies such as CAR-T cell therapy and the like (e.g., day of apheresis for procuring CAR-T manufacturing material).

As provided herein, patient treatment history or prior anti-cancer exposures (i.e., therapies, treatments, etc.), in the context of time-to-event modeling, comprises left-censored data. With the current subject matter, time can be inverted to convert left-censored data into right-censored data. This right-censored data can then be analyzed using a time-to-event model which, in turn, can comprise one or more regression models. One example, of such a regression model is a Cox proportional-hazards model.

The TTE model as provided herein can be applied to any drug or drug class to identify variables associated with time-since-last-exposure. One example, is the application to situations in which the last exposure to alkylating agents is used in anti-myeloma regimens (excluding use in the stem cell transplant setting). With this scenario, an event was the day of last known exposure to an alkylating agent; if a patient had no prior history of alkylating agent exposure, then they were censored at their earliest known anti-myeloma treatment exposure, regardless of drug class. The time-since-last-exposure can be calculated as the interval from the last day of their prior regimen at which the exposure occurred and the day of apheresis for procuring CAR T manufacturing material.

Relationships between a continuous variable of interest, X, and time-since-last alkylating exposure can then be analyzed by computing the maximally selected log-rank statistic (strongest p-value) for cutpoints between the 20% and 80% quantiles of X variable (R library: maxstat). If the minimum p-value for this procedure (after adjusting for multiple testing) was ≈0.05, then variable X was flagged as having an association with time-since-last-exposure. Similarly, relationships between an ordinal or categorical variable of interest, Y, and time-since-last alkylating exposure can then be analyzed by computing the Cox proportional-hazards statistic for each (n−1) strata in Y relative to a reference stratum. If the p-value for any strata relative to the reference strata was ≈0.05, then variable Y was flagged as having an association with time-since-last-exposure.

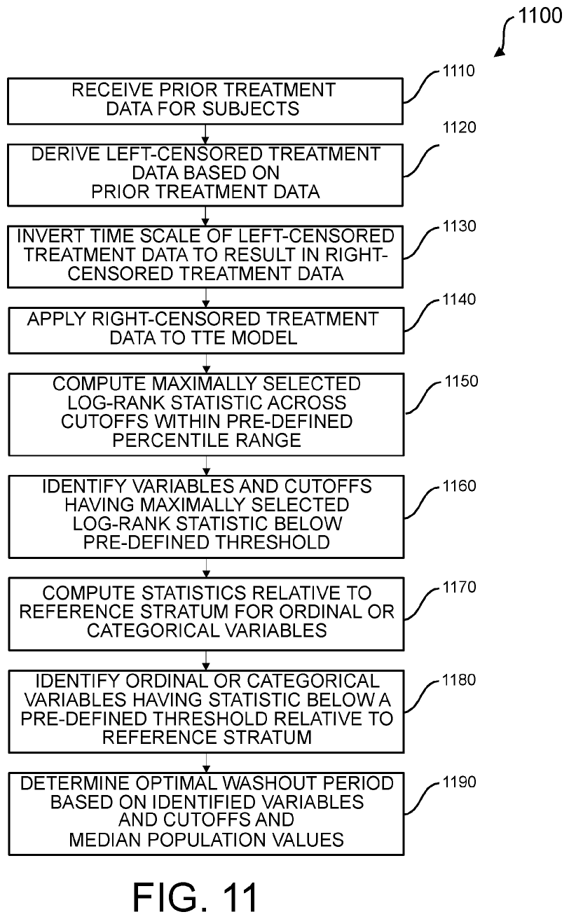

FIG. 11 is a process flow diagram 1000 in which an optimal washout period for commencing a therapy for the treatment of a condition in a subject after a prior exposure can be determined by, at 1110, receiving, for each of a plurality of subjects, prior treatment history data. Left-censored data can then be derived, at 1120, from the prior treatment history data for each of the subjects that includes a washout period and event or censor. A time scale of the left-censored treatment data is then inverted, at 1130 to result in right-censored treatment data. The right-censored treatment data is then applied, at 1140, to a time-to-event (TTE) model that associates one or more variables of interest with a time since exposure to the prior exposure. A maximally selected log-rank statistic across a plurality of cutoffs within a pre-defined percentile range is computed, at 1150, for continuous variables within the one or more variables of interest. One or more variables and associated cutoffs for the continuous variables having a maximally selected log-rank statistic below a first pre-defined threshold are, at 1160, then identified. A test statistic of each (n−1) strata relative to a reference stratum is then computed, at 1170, for ordinal or categorical variables within the one or more variables of interest. One or more ordinary or categorical variables and associated strata having a test statistic below a second pre-defined threshold, relative to the reference stratum are, at 1180, then identified. An optimal washout period is then determined, at 1190, for the therapy based on the cutoff having a lowest value below the pre-defined threshold and relative to a median of subject values below the pre-defined threshold and a median of subject values above the pre-defined threshold.

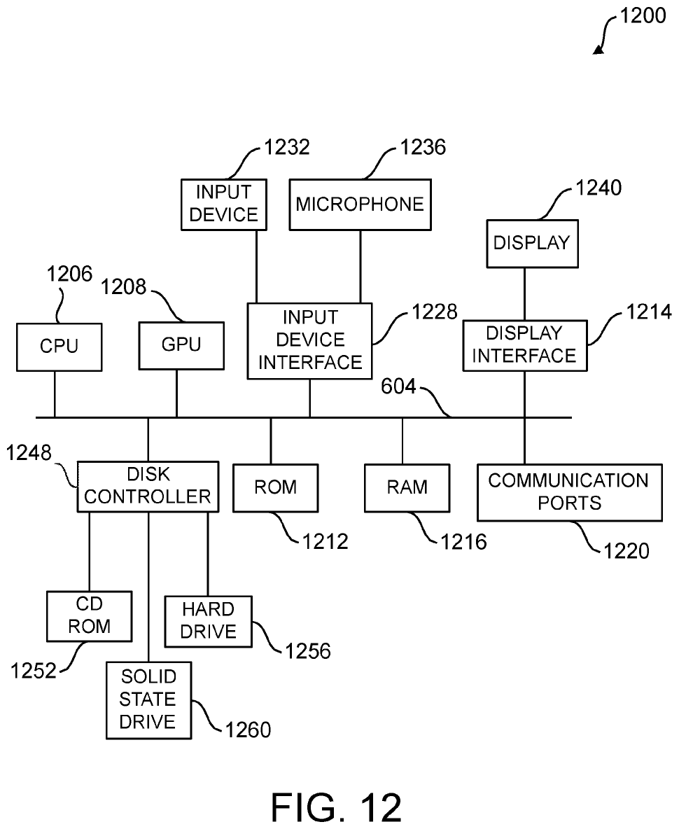

FIG. 12 is a diagram 1200 illustrating a sample computing device architecture for implementing various aspects described herein. A bus 1204 can serve as the information highway interconnecting the other illustrated components of the hardware. A processing system 1208 labeled CPU (central processing unit) (e.g., one or more computer processors/data processors at a given computer or at multiple computers), can perform calculations and logic operations required to execute a program. A non-transitory processor-readable storage medium, such as read only memory (ROM) 1212 and random access memory (RAM) 1216, can be in communication with the processing system 1208 and can include one or more programming instructions for the operations specified here. Optionally, program instructions can be stored on a non-transitory computer-readable storage medium such as a magnetic disk, optical disk, recordable memory device, flash memory, or other physical storage medium.

In one example, a disk controller 1248 can interface with one or more optional disk drives to the system bus 1204. These disk drives can be external or internal floppy disk drives such as 1260, external or internal CD-ROM, CD-R, CD-RW or DVD, or solid state drives such as 1252, or external or internal hard drives 1256. As indicated previously, these various disk drives 1252, 1256, 1260 and disk controllers are optional devices. The system bus 1204 can also include at least one communication port 1220 to allow for communication with external devices either physically connected to the computing system or available externally through a wired or wireless network. In some cases, the at least one communication port 1220 includes or otherwise comprises a network interface.

To provide for interaction with a user, the subject matter described herein can be implemented on a computing device having a display device 1240 (e.g., a CRT (cathode ray tube) or LCD (liquid crystal display) monitor) for displaying information obtained from the bus 1204 via a display interface 1214 to the user and an input device 1232 such as keyboard and/or a pointing device (e.g., a mouse or a trackball) and/or a touchscreen by which the user can provide input to the computer. Other kinds of input devices 1232 can be used to provide for interaction with a user as well; for example, feedback provided to the user can be any form of sensory feedback (e.g., visual feedback, auditory feedback by way of a microphone 1236, or tactile feedback); and input from the user can be received in any form, including acoustic, speech, or tactile input. The input device 1232 and the microphone 1236 can be coupled to and convey information via the bus 1204 by way of an input device interface 1228. Other computing devices, such as dedicated servers, can omit one or more of the display 1240 and display interface 1214, the input device 1232, the microphone 1236, and input device interface 1228.

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure. All references cited herein, whether patent or non-patent, are incorporated by reference herein in their entireties.

What is claimed is:

1. A method of treating a tumor or a cancer in a subject in need thereof, comprising:
(a) administering to the subject an alkylating agent;
(b) isolating peripheral blood mononuclear cells (PBMCs) from the subject at least about six (6) months after step (a);
(c) determining that at least about 20% of the PBMCs are T cells;
(d) on the basis of the determination in step (c), subsequently manufacturing chimeric antigen receptor (CAR) T cells from the PBMCs; and
(e) administering the CAR T cells to the subject.

2. The method of claim 1, wherein step (b) is performed at least about seven (7) months, at least about eight (8) months, at least about nine (9) months, at least about ten (10) months, at least about eleven (11) months, at least about twelve (12) months, at least about thirteen (13) months, or at least about fourteen (14) months after step (a).

3. A method of treating a tumor or a cancer in a subject in need thereof, comprising:
(a) isolating peripheral blood mononuclear cells (PBMCs) from the subject, and determining that at least about 20% of the PBMCs are T cells;
(b) on the basis of the determination in step (a), subsequently manufacturing chimeric antigen receptor (CAR) T cells from the PBMCs; and
(c) administering to the subject the CAR T cells, wherein, prior to step (a), the subject had previously received an alkylating agent for treatment of the cancer.

4. The method of claim 3, wherein the subject had previously received the alkylating agent at least about six (6) months prior to step (a).

5. The method of claim 3 or claim 4, wherein the subject had previously received the alkylating agent at least about seven (7) months, at least about eight (8) months, at least about nine (9) months, at least about ten (10) months, at least about eleven (11) months, at least about twelve (12) months, at least about thirteen (13) months, or at least about fourteen (14) months prior to step (a).

6. A method of treating a tumor or a cancer in a subject in need thereof, comprising:
(a) isolating peripheral blood mononuclear cells (PBMCs) from the subject;
(b) manufacturing chimeric antigen receptor (CAR) T cells from the PBMCs; and
(c) administering to the subject the CAR T cells, wherein the subject had previously received an alkylating agent for treatment of the cancer;
wherein step (a) occurs at least about six months after the subject received the alkylating agent.

7. The method of claim 6, wherein step (a) further comprises determining that at least about 20% of the PBMCs are T cells; and wherein step (b) further comprises subsequently manufacturing chimeric antigen receptor (CAR) T cells from the PBMCs on the basis of the determination in step (a).

8. The method of claim 6 or claim 7, wherein step (a) occurs at least about seven (7) months, at least about eight (8) months, at least about nine (9) months, at least about ten (10) months, at least about eleven (11) months, at least about twelve (12) months, at least about thirteen (13) months, or at least about fourteen (14) months after the subject received the alkylating agent.

9. A method of treating a tumor or a cancer in a subject in need thereof, wherein the subject has been administered an alkylating agent, comprising:
(a) determining that the subject has not been administered the alkylating agent less than about six (6) months prior to the determining step;
(b) isolating peripheral blood mononuclear cells (PBMCs) from the subject;
(c) manufacturing chimeric antigen receptor (CAR) T cells from the PBMCs; and
(d) administering to the subject the CAR T cells.

10. The method of claim 9, wherein in step (a) the subject has not been administered the alkylating agent less than about seven (7) months, less than about eight (8) months, less than about nine (9) months, less than about ten (10) months, less than about eleven (11) months, less than about twelve (12) months, less than about thirteen (13) months, or less than about fourteen (14) months prior to the determining step.

11. A method of treating a tumor or a cancer in a subject in need thereof, wherein the subject has been administered an alkylating agent, comprising:
(a) isolating peripheral blood mononuclear cells (PBMCs) from the subject;
(b) manufacturing chimeric antigen receptor (CAR) T cells from the PBMCs; and
(c) administering to the subject the CAR T cells, wherein, at the time of the isolating, the subject has been determined to have been administered the alkylating agent at least about six (6) months prior.

12. The method of claim 11, wherein the subject has been determined to have been administered the alkylating agent at least about seven (7) months, at least about eight (8) months, at least about nine (9) months, at least about ten (10) months, at least about eleven (11) months, at least about twelve (12) months, at least about thirteen (13) months, or at least about fourteen (14) months prior.

13. A method of treating a tumor or a cancer in a subject in need thereof, wherein the subject has been administered an alkylating agent, comprising administering to the subject T cells expressing a chimeric antigen receptor (CAR T cells) manufactured from peripheral blood mononuclear cells PBMCs isolated from the patient, wherein, at the time said PBMCs are isolated, the subject has last received the alkylating agent at least about six (6) months prior to the time the PBMCs are isolated.

14. The method of claim 13, wherein the subject has last received the alkylating agent at least about seven (7) months, at least about eight (8) months, at least about nine (9) months, at least about ten (10) months, at least about eleven (11) months, at least about twelve (12) months, at least about thirteen (13) months, or at least about fourteen (14) months prior to the time the PBMCs are isolated.

15. The method of any one of claims 1-14, wherein the tumor or cancer is lymphoma, lung cancer, breast cancer, prostate cancer, adrenocortical carcinoma, thyroid carcinoma, nasopharyngeal carcinoma, melanoma, skin carcinoma, colorectal carcinoma, a desmoid tumor, a desmoplastic small round cell tumor, an endocrine tumor, a Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipomachronic lymphocytic leukemia (small lymphocytic lymphoma), B-cell prolymphocytic leukemia, lymphoplasmacytic lymphoma, Waldenström macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, a non-Hodgkin lymphoma, or multiple myeloma.

16. The method of claim 15, wherein the cancer is multiple myeloma, chronic lymphocytic leukemia, or a non-Hodgkins lymphoma.

17. The method of claim 16, wherein the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma.

18. The method of claim 16, wherein the cancer is multiple myeloma.

19. The method of claim 18, wherein the multiple myeloma is high-risk multiple myeloma or relapsed and refractory multiple myeloma.

20. The method of claim 18, wherein the multiple myeloma is high risk multiple myeloma, and the high risk multiple myeloma is R-ISS stage III disease and/or a disease characterized by early relapse.

21. The method of any one of claims 1-20, wherein the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan, oxaliplatin, platinum, procarbazine, streptozocin, temozolomide, thiotepa, and trabectedin.

22. The method of any one of claims 1-21, wherein the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa.

23. The method of any one of claims 1-22, wherein the alkylating agent is cyclophosphamide.

24. A method of treating a cancer caused by B Cell Maturation Agent (BCMA) expressing cells in a subject in need thereof, comprising:
(a) administering to the subject an alkylating agent;
(b) isolating peripheral blood mononuclear cells (PBMCs) from the subject at least about six months after step (a);
(c) determining that at least about 20% of the PBMCs are T cells;
(d) on the basis of the determination in step (c), subsequently manufacturing chimeric antigen receptor (CAR) T cells directed to BCMA (BCMA CAR T cells) from the PBMCs; and
(e) administering to the subject the CAR T cells to the subject.

25. The method of claim 24, wherein step (b) is performed at least about seven (7) months, at least about eight (8) months, at least about nine (9) months, at least about ten (10) months, at least about eleven (11) months, at least about twelve (12) months, at least about thirteen (13) months, or at least about fourteen (14) months after step (a) of administering to the subject an alkylating agent.

26. A method of treating a cancer caused by B Cell Maturation Agent (BCMA) expressing cells in a subject in need thereof, comprising:
(a) isolating peripheral blood mononuclear cells (PBMCs) from the subject, and determining that at least about 20% of the PBMCs are T cells;
(b) on the basis of the determination in step (a), subsequently manufacturing chimeric antigen receptor (CAR) T cells directed to BCMA (BCMA CAR T cells) from the PBMCs; and
(c) administering to the subject the CAR T cells, wherein, prior to step (a), the subject had previously received an alkylating agent for treatment of the cancer.

27. The method of claim 26, wherein the subject had previously received the alkylating agent at least about six months prior to step (a).

28. The method of claim 26 or claim 27, wherein the subject had previously received the alkylating agent at least about seven (7) months, at least about eight (8) months, at least about nine (9) months, at least about ten (10) months, at least about eleven (11) months, at least about twelve (12) months, at least about thirteen (13) months, or at least about fourteen (14) months prior to step (a).

29. A method of treating a cancer caused by B Cell Maturation Agent (BCMA) expressing cells in a subject in need thereof, comprising:
(a) isolating peripheral blood mononuclear cells (PBMCs) from the subject;
(b) manufacturing chimeric antigen receptor (CAR) T cells directed to BCMA (BCMA CAR T cells) from the PBMCs;
(c) administering to the subject the BCMA CAR T cells, wherein the subject had previously received an alkylating agent for treatment of the cancer, and
wherein step (a) occurs at least about six months after the subject received the alkylating agent.

30. The method of claim 29, wherein step (a) further comprises determining that at least about 20% of the PBMCs are T cells; and wherein step (b) further comprises subsequently manufacturing chimeric antigen receptor (CAR) T cells from the PBMCs on the basis of the determination in step (a).

31. The method of claim 29 or claim 30, wherein step (a) occurs at least about seven (7) months, at least about eight (8) months, at least about nine (9) months, at least about ten (10) months, at least about eleven (11) months, at least about twelve (12) months, at least about thirteen (13) months, or at least about fourteen (14) months after the subject received the alkylating agent.

32. A method of treating a cancer caused by B Cell Maturation Agent (BCMA) expressing cells in a subject in need thereof, wherein the subject has been administered an alkylating agent for treatment of a cancer, comprising:
(a) determining that the subject has not been administered the alkylating agent less than about six (6) months prior to the determining step;
(b) isolating peripheral blood mononuclear cells (PBMCs) from the subject, wherein the isolating is performed at least six (6) months after the alkylating agent has been administered to the subject;
(c) manufacturing chimeric antigen receptor (CAR) T cells directed to BCMA (BCMA CAR T cells) from the PBMCs; and
(d) administering to the subject the BCMA CAR T cells.

33. The method of claim 32, wherein in step (a) the subject has not been administered the alkylating agent less than about seven (7) months, less than about eight (8) months, less than about nine (9) months, less than about ten (10) months, less than about eleven (11) months, less than about twelve (12) months, less than about thirteen (13) months, or less than about fourteen (14) months prior to the determining step.

34. A method of treating a cancer caused by B Cell Maturation Agent (BCMA) expressing cells in a subject in need thereof, wherein the subject has been administered an alkylating agent, comprising:
(a) isolating peripheral blood mononuclear cells (PBMCs) from the subject;
(b) manufacturing chimeric antigen receptor (CAR) T cells directed to BCMA (BCMA CAR T cells) from the PBMCs; and
(c) administering to the subject the BCMA CAR T cells, wherein, at the time of the isolating, the subject has been determined to have been administered the alkylating agent at least about six (6) months prior.

35. The method of claim 34, wherein the subject has been determined to have been administered the alkylating agent at least about seven (7) months, at least about eight (8) months, at least about nine (9) months, at least about ten (10) months, at least about eleven (11) months, at least about twelve (12) months, at least about thirteen (13) months, or at least about fourteen (14) months prior.

36. A method of treating a cancer caused by B Cell Maturation Agent (BCMA) expressing cells in a subject in need thereof, wherein the subject has been administered an alkylating agent, comprising administering to the subject chimeric antigen receptor (CAR) T cells directed to BCMA (BCMA CAR T cells) manufactured from peripheral blood mononuclear cells PBMCs isolated from the patient, wherein, at the time said PBMCs are isolated, the subject has last received the alkylating agent at least about six (6) months prior to the time the PBMCs are isolated.

37. The method of claim 36, wherein the subject has last received the alkylating agent at least about seven (7) months, at least about eight (8) months, at least about nine (9) months, at least about ten (10) months, at least about eleven (11) months, at least about twelve (12) months, at least about thirteen (13) months, or at least about fourteen (14) months prior to the time the PBMCs are isolated.

38. The method of any one of claims 24-37, wherein the cancer is multiple myeloma, chronic lymphocytic leukemia, or a non-Hodgkins lymphoma.

39. The method of claim 38, wherein the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma.

40. The method of claim 38, wherein the cancer is multiple myeloma.

41. The method of claim 40, wherein the multiple myeloma is high-risk multiple myeloma or relapsed and refractory multiple myeloma.

42. The method of claim 40, wherein the multiple myeloma is high risk multiple myeloma, and the high risk multiple myeloma is R-ISS stage III disease and/or a disease characterized by early relapse.

43. The method of any one of claims 24-42, wherein the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan, oxaliplatin, platinum, procarbazine, streptozocin, temozolomide, thiotepa, and trabectedin.

44. The method of any one of claims 24-43 wherein the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa.

45. The method of any one of claims 24-44, wherein the alkylating agent is cyclophosphamide.

46. A method of manufacturing chimeric antigen receptor (CAR) T cells from a subject, comprising:
(a) administering to the subject an alkylating agent for treatment of a tumor or a cancer;
(b) isolating peripheral blood mononuclear cells (PBMCs) from the subject at least about six months after step (a); and
(c) manufacturing chimeric antigen receptor (CAR) T cells from the PBMCs.

47. The method of claim 46, wherein step (b) is performed at least about seven (7) months, at least about eight (8) months, at least about nine (9) months, at least about ten (10) months, at least about eleven (11) months, at least about twelve (12) months, at least about thirteen (13) months, or at least about fourteen (14) months after step (a).

48. A method of manufacturing chimeric antigen receptor (CAR) T cells from a subject, comprising:
(a) isolating peripheral blood mononuclear cells (PBMCs) from the subject, and determining that at least about 20% of the PBMCs are T cells; and
(b) on the basis of the determination in step (b), subsequently manufacturing chimeric antigen receptor (CAR) T cells from the PBMCs;
wherein, prior to step (a), the subject had previously received an alkylating agent for treatment of a tumor or a cancer.

49. The method of claim 48, wherein the subject had previously received the alkylating agent at least about six months prior to step (a).

50. The method of claim 48 or claim 49, wherein the subject had previously received the alkylating agent at least about seven (7) months, at least about eight (8) months, at least about nine (9) months, at least about ten (10) months, at least about eleven (11) months, at least about twelve (12) months, at least about thirteen (13) months, or at least about fourteen (14) months prior to step (a).

51. A method of manufacturing chimeric antigen receptor (CAR) T cells from a subject, comprising:
(a) isolating peripheral blood mononuclear cells (PBMCs) from the subject; and
(b) manufacturing chimeric antigen receptor (CAR) T cells from the PBMCs;
wherein the subject had previously received an alkylating agent for treatment of a tumor or a cancer;
wherein step (a) occurs at least about six months after the subject received the alkylating agent.

52. The method of claim 51, wherein step (a) further comprises determining that at least about 20% of the PBMCs are T cells; and wherein step (b) further comprises subsequently manufacturing chimeric antigen receptor (CAR) T cells from the PBMCs on the basis of the determination in step (a).

53. The method of claim 51 or claim 52, wherein step (a) occurs at least about seven (7) months, at least about eight (8) months, at least about nine (9) months, at least about ten (10) months, at least about eleven (11) months, at least about twelve (12) months, at least about thirteen (13) months, or at least about fourteen (14) months after the subject received the alkylating agent.

54. A method of manufacturing chimeric antigen receptor (CAR) T cells from a subject, wherein the subject has been administered an alkylating agent for treatment of a tumor or a cancer, comprising:
a. determining that the subject has not been administered the alkylating agent less than about six (6) months prior to the determining step;
b. isolating peripheral blood mononuclear cells (PBMCs) from the subject; and
c. manufacturing chimeric antigen receptor (CAR) T cells from the PBMCs.

55. The method of claim 54, wherein in step (a) the subject has not been administered the alkylating agent less than about seven (7) months, less than about eight (8) months, less than about nine (9) months, less than about ten (10) months, less than about eleven (11) months, less than about twelve (12) months, less than about thirteen (13) months, or less than about fourteen (14) months prior to the determining step.

56. A method of manufacturing chimeric antigen receptor (CAR) T cells from a subject, wherein the subject has been administered an alkylating agent for treatment of a tumor or a cancer, comprising:
(a) isolating peripheral blood mononuclear cells (PBMCs) from the subject; and
(b) manufacturing chimeric antigen receptor (CAR) T cells from the PBMCs;
wherein, at the time of the isolating, the subject has been determined to have been administered the alkylating agent at least about six (6) months prior.

57. The method of claim 56, wherein the subject has been determined to have been administered the alkylating agent at least about seven (7) months, at least about eight (8) months, at least about nine (9) months, at least about ten (10) months, at least about eleven (11) months, at least about twelve (12) months, at least about thirteen (13) months, or at least about fourteen (14) months prior.

58. The method of any one of claims 46-57, wherein the tumor or cancer is lymphoma, lung cancer, breast cancer, prostate cancer, adrenocortical carcinoma, thyroid carcinoma, nasopharyngeal carcinoma, melanoma, skin carcinoma, colorectal carcinoma, a desmoid tumor, a desmoplastic small round cell tumor, an endocrine tumor, a Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipomachronic lymphocytic leukemia (small lymphocytic lymphoma), B-cell prolymphocytic leukemia, lymphoplasmacytic lymphoma, Waldenström macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, a non-Hodgkin lymphoma, or multiple myeloma.

59. The method of claim 58, wherein the cancer is multiple myeloma, chronic lymphocytic leukemia, or a non-Hodgkins lymphoma.

60. The method of claim 59, wherein the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma.

61. The method of claim 59, wherein the cancer is multiple myeloma.

62. The method of claim 61, wherein the multiple myeloma is high-risk multiple myeloma or relapsed and refractory multiple myeloma.

63. The method of claim 61, wherein the multiple myeloma is high risk multiple myeloma, and the high risk multiple myeloma is R-ISS stage III disease and/or a disease characterized by early relapse.

64. The method of any one of claims 46-63 wherein the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan, oxaliplatin, platinum, procarbazine, streptozocin, temozolomide, thiotepa, and trabectedin.

65. The method of any one of claims 46-64, wherein the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa.

66. The method of any one of claims 46-64, wherein the alkylating agent is cyclophosphamide.

67. A method of manufacturing chimeric antigen receptor (CAR) T cells directed to BCMA (BCMA CAR T cells) from a subject, comprising:
(a) administering to the subject an alkylating agent for treatment of a cancer;
(b) isolating peripheral blood mononuclear cells (PBMCs) from the subject at least about six months after step (a); and
(c) manufacturing BCMA CAR T cells from the PBMCs.

68. The method of claim 67, wherein step (b) is performed at least about seven (7) months, at least about eight (8) months, at least about nine (9) months, at least about ten (10) months, at least about eleven (11) months, at least about twelve (12) months, at least about thirteen (13) months, or at least about fourteen (14) months after step (a).

69. A method of manufacturing chimeric antigen receptor (CAR) T cells directed to BCMA (BCMA CAR T cells) from a subject, comprising:
(a) isolating peripheral blood mononuclear cells (PBMCs) from the subject, and determining that at least about 20% of the PBMCs are T cells; and
(b) on the basis of the determination in step (a), subsequently manufacturing BCMA CAR T cells from the PBMCs;
wherein, prior to step (a), the subject had previously received an alkylating agent for treatment of a cancer.

70. The method of claim 69, wherein the subject had previously received the alkylating agent at least about six months prior to step (a).

71. The method of claim 69 or claim 70, wherein the subject had previously received the alkylating agent at least about seven (7) months, at least about eight (8) months, at least about nine (9) months, at least about ten (10) months, at least about eleven (11) months, at least about twelve (12) months, at least about thirteen (13) months, or at least about fourteen (14) months prior to step (a).

72. A method of manufacturing chimeric antigen receptor (CAR) T cells directed to BCMA (BCMA CAR T cells) from a subject, comprising:
(a) isolating peripheral blood mononuclear cells (PBMCs) from the subject; and
(b) manufacturing BCMA CAR T cells from the PBMCs;
wherein the subject had previously received an alkylating agent for treatment of a cancer;
wherein step (a) occurs at least about six months after the subject received the alkylating agent.

73. The method of claim 72, wherein step (a) further comprises determining that at least about 20% of the PBMCs are T cells; and wherein step (b) further comprises subsequently manufacturing chimeric antigen receptor (CAR) T cells from the PBMCs on the basis of the determination in step (a).

74. The method of claim 72 or claim 73, wherein step (a) occurs at least about seven (7) months, at least about eight (8) months, at least about nine (9) months, at least about ten (10) months, at least about eleven (11) months, at least about twelve (12) months, at least about thirteen (13) months, or at least about fourteen (14) months after the subject received the alkylating agent.

75. A method of manufacturing chimeric antigen receptor (CAR) T cells directed to BCMA (BCMA CAR T cells) from a subject, wherein the subject has been administered an alkylating agent for treatment of a cancer, comprising:
a. determining that the subject has not been administered the alkylating agent less than about six (6) months prior to the determining step;
b. isolating peripheral blood mononuclear cells (PBMCs) from the subject; and
c. manufacturing BCMA CAR T cells from the PBMCs.

76. The method of claim 75, wherein step (a) occurs at least about seven (7) months, at least about eight (8) months, at least about nine (9) months, at least about ten (10) months, at least about eleven (11) months, at least about twelve (12) months, at least about thirteen (13) months, or at least about fourteen (14) months after the subject received the alkylating agent.

77. A method of manufacturing chimeric antigen receptor (CAR) T cells directed to BCMA (BCMA CAR T cells) from a subject, wherein the subject has been administered an alkylating agent for treatment of a cancer, comprising:
(a) isolating peripheral blood mononuclear cells (PBMCs) from the subject; and
(b) manufacturing BCMA CAR T cells from the PBMCs;
wherein, at the time of the isolating, the subject has been determined to have been administered the alkylating agent at least about six (6) months prior.

78. The method of claim 77, wherein the subject has been determined to have been administered the alkylating agent at least about seven (7) months, at least about eight (8) months, at least about nine (9) months, at least about ten (10) months, at least about eleven (11) months, at least about twelve (12) months, at least about thirteen (13) months, or at least about fourteen (14) months prior.

79. The method of any one of claims 67-78, wherein the cancer is multiple myeloma, chronic lymphocytic leukemia, or a non-Hodgkins lymphoma.

80. The method of claim 79, wherein the cancer is a non-Hodgkins lymphoma, and the non-Hodgkins lymphoma is Burkitt's lymphoma, chronic lymphocytic leukemia/small lymphocytic lymphoma (CLL/SLL), diffuse large B cell lymphoma, follicular lymphoma, immunoblastic large cell lymphoma, precursor B-lymphoblastic lymphoma, or mantle cell lymphoma.

81. The method of claim 79, wherein the cancer is multiple myeloma.

82. The method of claim 81, wherein the multiple myeloma is high-risk multiple myeloma or relapsed and refractory multiple myeloma.

83. The method of claim 81, wherein the multiple myeloma is high risk multiple myeloma, and the high risk multiple myeloma is R-ISS stage III disease and/or a disease characterized by early relapse.

84. The method of any one of claims 67-83 wherein the alkylating agent is one or more of: altretamine, bendamustine, busulfan, carboplatin, carmustine, chlorambucil, cisplatin, cyclophosphamide, dacarbazine, evofosfamide, ifosfamide, lomustine, mechlorethamine, melphalan, oxaliplatin, platinum, procarbazine, streptozocin, temozolomide, thiotepa, and trabectedin.

85. The method of any one of claims 67-84 wherein the alkylating agent is one or more of: bendamustine, busulfan, carboplatin, carmustine, chlorambucil, cisplatin, cyclophosphamide, evofosfamide, ifosfamide, oxaliplatin, platinum, procarbazine, and thiotepa.

86. The method of any one of claims 67-85, wherein the alkylating agent is cyclophosphamide.

87. The method of any one of claims 1-86, wherein the subject is a human.

88. The method of any one of claims 24-45 or 67-87, wherein the BCMA CAR T cells comprise a CAR directed to BCMA, wherein the CAR directed to BCMA comprises an antibody or antibody fragment that targets BCMA.

89. The method of any one of claims 24-45 or 67-87, wherein the BCMA CAR T cells comprise a CAR directed to BCMA, wherein the CAR directed to BCMA comprises a single chain Fv antibody or antibody fragment (scFv).

90. The method of any one of claims 24-45 or 67-87, wherein the BCMA CAR T cells comprise a CAR directed to BCMA, wherein the CAR directed to BCMA comprises a BCMA02 scFv.

91. The method of any one of claims 24-45 or 67-87, wherein the BCMA CAR T cells are idecabtagene vicleucel cells.

92. The method of any one of claims 1-23 or 46-66, wherein the subject undergoes a leukapharesis procedure to collect the PBMCs for the manufacture of the CAR T cells prior to their administration to the subject.

93. The method of any one of claims 24-45 or 67-87, wherein the subject undergoes a leukapharesis procedure to collect the PBMCs for the manufacture of the BCMA CAR T cells prior to their administration to the subject.

94. The method of any one of claims 1-23 or 92, wherein the CAR T cells are administered by an intravenous infusion.

95. The method of any one of claims 24-45 or 93, wherein the BCMA CAR T cells are administered by an intravenous infusion.

96. A method of determining whether a subject having cancer can be treated with a CAR T therapy, comprising determining the percentage of CD3+ T cells in the subject relative to the number of PMBCs, wherein, (i) if the subject has less than about 20% CD3+ T cells relative to the number of PBMCs, not administering the CAR T therapy to the subject, and (ii) if the subject has more than about 20% CD3+ T cells relative to the number of PBMCs, then administering the CAR T therapy to the subject.

97. A method of treating a cancer in a subject, comprising having determined that the percentage of CD3+ T cells in the subject relative to the number of PMBCs is higher than about 20%, and administering a CAR T therapy to the subject.

98. The method of claim 96 or 97, wherein the CD3+ T cells are CD45+/CD3+ T cells.

99. A method for determining an optimal washout period for commencing a therapy for treatment of a condition in a subject after a prior exposure, the method comprising:
receiving, for each of a plurality of subjects, prior treatment history data;
deriving, from the prior treatment history data, left-censored treatment data for each of the subjects comprising a washout period and event or censor;
inverting a time scale of the left-censored treatment data to result in right-censored treatment data;
applying the right-censored treatment data to a time-to-event (TTE) model that associates one or more variables of interest with a time since exposure to the prior exposure;
computing, for continuous variables within the one or more variables of interest, a maximally selected log-rank statistic across a plurality of cutoffs within a pre-defined percentile range;
identifying, for the continuous variables within the one or more variables of interest, the one or more variables and associated cutoff having a maximally selected log-rank statistic below a first pre-defined threshold;
computing, for ordinal or categorical variables within the one or more variables of interest, a test statistic of each (n−1) strata relative to a reference stratum;
identifying, for the ordinal or categorical variables within the one or more variables of interest, the one or more variables and associated strata having a test statistic below a second pre-defined threshold, relative to the reference stratum; and
determining an optimal washout period for the therapy based on the cutoff having a lowest value below the pre-defined threshold and relative to a median of subject values below the pre-defined threshold and a median of subject values above the pre-defined threshold.

100. The method of claim 99 further comprising:
providing the determined optimal washout period.

101. The method of claim 100, wherein providing the determined optimal washout period comprises one or more: causing the determined optimal washout period to be displayed in an electronic visual display, storing the determined optimal washout period in physical persistence, loading the determined optimal washout period into memory, or transmitting the determined optimal washout period over a network to a remote computing device.

102. The method of any of claims 99 to 101, wherein the TTE model is a proportional-hazards model.

103. The method of any of claims 99 to 102, wherein the TTE model is a Cox proportional-hazards model.

104. The method of any of claims 99 to 103, wherein one or more of the receiving, deriving, inverting, applying, first or second computing, first or second identifying, and determining is executed by at least one data processor forming part of at least one computing device.

105. The method of any of claims 99 to 103, wherein the prior exposure is a prior therapy.

106. The method of claim 105, wherein the prior exposure and the therapy are different types of therapies.

107. The method of claim 106, wherein the prior exposure is for treating a condition that is different from the condition treated with the therapy.

108. The method of claim 106, wherein the prior exposure is for treating a condition that is the same as the condition treated with the therapy.

109. The method of claim 108, wherein the prior exposure and the therapy are for treating the same condition and are the same type of therapy.

110. The method of claim 108, wherein the therapy is a second or later (e.g., 3rd, 4th, 5th, 6th, 7th or later) line of therapy and the prior exposure is an earlier line of therapy.

111. The method of any of claims 99 to 110, which informs (i) the time that a subject should receive the therapy after having had the prior exposure or (ii) exclusion criteria in a clinical trial.

112. The method of any of claims 99 to 111, wherein the therapy and/or the prior exposure is radiotherapy, chemotherapy, immunotherapy, surgery, a transplant, gene therapy or cell therapy.

113. The method of any of claims 99 to 112, wherein the condition is cancer, an immune disease (e.g., an autoimmune disease), a cardiovascular disease, fibrosis, an infectious disease or a neurological condition.

114. The method of any of claims 99 to 113, wherein the condition is a condition that can be treated by stimulating the immune system, e.g., cancer and infectious diseases, and the therapy is a therapy that stimulates or enhances the immune system, e.g., immunotherapy and cell therapy.

115. The method of any of claims 99 to 114, wherein the condition is cancer and the therapy is cell therapy, e.g., CAR T.

116. The method of claim 115, wherein the prior exposure is a prior therapy, and the prior therapy is a prior cancer treatment.

117. The method of claim 116, wherein the cancer is multiple myeloma, the treatment is a BCMA CAR T (e.g., a CAR comprising SEQ ID NO: 37 or a nucleic acid encoding a CAR of SEQ ID NO: 9 or 37), and the prior treatment is a prior cancer treatment for multiple myeloma.

118. The method of claim 117, wherein the prior exposure is an alkylator therapy.

119. The method of any of claims 99 to 118, wherein the prior exposure is not a prior therapy.

120. The method of claim 119, wherein the prior exposure is an event that can negatively impact the therapy.

121. The method of claim 120, wherein the prior exposure is a prior condition.

122. The method of claim 121, wherein the prior exposure is an inflammatory condition or an infectious disease (e.g., a viral infection, such as COVID-19 infection).

123. A method for treating a condition in a subject comprising:
administering to the subject a therapy for treating the condition, wherein the therapy is administered after a prior exposure, and wherein the time to administer the therapy after the prior exposure (i.e., the washout period) was calculated using a method as in any of claims 99 to 122.

124. A method for treating a condition in a subject comprising:
administering to the subject a therapy for treating the condition, wherein the therapy is administered after a prior exposure, at a time after the prior exposure that was determined using a method as in any of claims 99 to 122.

125. A system comprising:
at least one data processor; and
memory storing instructions which, when executed by the at least one data processor, implement a method as in any of claims 99 to 122.

126. A non-transitory computer program product storing instructions which, when executed by at least one computing device, implement a method as in any of claims 99 to 122.
